(19) United States
(12) Patent Application Publication
(10) Pub. No.: US 2025/0161471 A1
(43) Pub. Date: May 22, 2025

(54) ANTI-HER2 ANTIBODY-DRUG CONJUGATES AND USES THEREOF

(71) Applicants: Feng TIAN, San Diego, CA (US); Sulan YAO, San Diego, CA (US); Jinchun YAN, Princeton, NJ (US); Gang XIA, San Diego, CA (US); Yanping JI, Shaoxing City (CN); Gaozhun XIONG, Shaoxing City (CN); Xuejun LIANG, Shaoxing City (CN)

(72) Inventors: Feng TIAN, San Diego, CA (US); Sulan YAO, San Diego, CA (US); Jinchun YAN, Princeton, NJ (US); Gang XIA, San Diego, CA (US); Yanping JI, Shaoxing City (CN); Gaozhun XIONG, Shaoxing City (CN); Xuejun LIANG, Shaoxing City (CN)

(21) Appl. No.: 18/553,674

(22) PCT Filed: Apr. 1, 2022
(86) PCT No.: PCT/US2022/023141

§ 371 Date: Oct. 2, 2023

Related U.S. Application Data
(60) Provisional application No. 63/299,879, filed on Jan. 14, 2022.
(60) Provisional application No. 63/257,999, filed on Oct. 20, 2021.
(60) Provisional application No. 63/210,481, filed on Jun. 14, 2021.
(60) Provisional application No. 63/194,929, filed on May 28, 2021.
(60) Provisional application No. 63/170,446, filed on Apr. 3, 2021.

(51) Int. Cl. A61K 47/68; A61K 45/06; A61K 47/22; A61K 47/26; A61P 35/04; C07K 16/32

(52) U.S. Cl. CPC A61K 47/68031; A61K 45/06; A61K 47/22; A61K 47/26; A61K 47/6855; A61P 35/04; C07K 16/32

(57) ABSTRACT

Disclosed herein are antibody-drug conjugates (ADCs) whose antibodies include at least one non-natural amino acid, and methods for making such non-natural amino acids and polypeptides. The ADCs can include a wide range of possible functionalities, but typically have at least one oxime, carbonyl, dicarbonyl, and/or hydroxylamine group. Also disclosed herein are non-natural amino acid ADCs that are further modified post-translationally, methods for effecting such modifications, and methods for purifying such ADCs. Typically, the modified ADCs include at least one oxime, carbonyl, dicarbonyl, and/or hydroxylamine group. Further disclosed are methods for using such non-natural amino acid ADCs and modified non-natural amino acid ADCs, including therapeutic, diagnostic, and other biotechnology use.

### REFERENCE TO RELATED APPLICATIONS

This application claims the benefit of U.S. Provisional Patent Application No. 63/170,446, filed Apr. 3, 2021, U.S. Provisional Patent Application No. 63/194,929, filed May 28, 2021, U.S. Provisional Patent Application No. 63/210,481, filed Jun. 14, 2021, U.S. Provisional Patent Application No. 63/257,999, filed Oct. 20, 2021, U.S. Provisional Patent Application No. 63/299,879, filed Jan. 14, 2022, each of which applications is entirely incorporated herein by reference.

### BACKGROUND OF THE INVENTION

HER2 is a member of the epithelial growth factor receptor family of transmembrane tyrosine kinase. Amplification or overexpression of HER2 occurs in approximately 25-30% of primary breast cancers and less frequently in a large variety of other solid tumor types (Mitri et al., Chemother Res Pract, 2012:743193), and is associated with a negative prognosis, including shorter relapse-free survival and overall survival (OS) time in breast cancer (Spears et al., Breast Cancer Res Treat. 2012; 134(2):701-8) and gastric cancer (Bang et al., Lancet, 2010; 376(9742): 687-97; Boku et al., Gastric Cancer. 2014; 17(1): 1-12), as well as shorter OS times in lung, ovarian, colon, and pancreatic cancers (Sithanandam et al., Cancer Gene Ther. 2008; 15(7):413-48).

### SUMMARY OF THE INVENTION

One aspect of the disclosure relates to an antibody-drug conjugate (ADC) comprising: a cytotoxic tubulin analog 269 having the structure

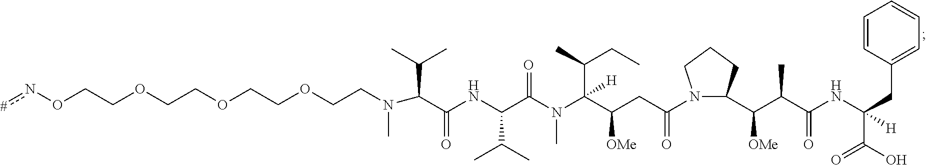

and an anti-HER2 antibody or antibody fragment comprising SEQ ID NO: 2 and SEQ ID NO: 3, wherein [symbol] represents a single bond or a double bond, and wherein # represents a connection to the anti-HER2 antibody or antibody fragment.

In some embodiments, the anti-HER2 antibody or antibody fragment comprises one or more non-naturally encoded amino acids incorporated in the heavy chain, light chain, or both the heavy and light chains.

Another aspect of the disclosure relates to an antibody-drug conjugate (ADC) comprising:
a cytotoxic tubulin analog having the structure

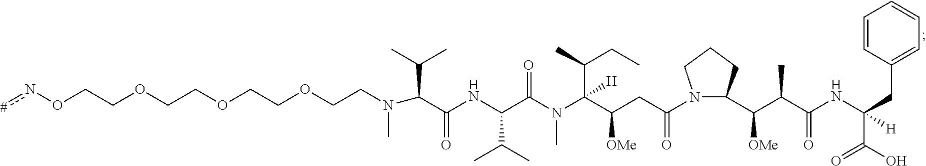

and
an anti-HER2 antibody or antibody fragment comprising SEQ ID NO: 2,
wherein the anti-HER2 antibody or antibody fragment comprises one or more non-naturally encoded amino acids incorporated in a heavy chain, a light chain, or both the heavy chain and the light chain, wherein [symbol] represents a single bond or a double bond, and wherein # represents a connection to the anti-HER2 antibody or antibody fragment.

In some embodiments, the anti-HER2 antibody or antibody fragment comprises one or more non-naturally encoded amino acids incorporated in the heavy chain. In some embodiments, [symbol] represents a double bond, and wherein the tubulin analog is covalently bonded to the anti-HER2 antibody via the double bond. In some embodiments, the double bond is between the tubulin analog and a member of the one or more non-naturally encoded amino acids. In some embodiments, the ADC comprises two heavy chains, wherein each heavy chain comprises SEQ ID NO: 2, and wherein each heavy chain is individually conjugated to a different tubulin analog having the structure

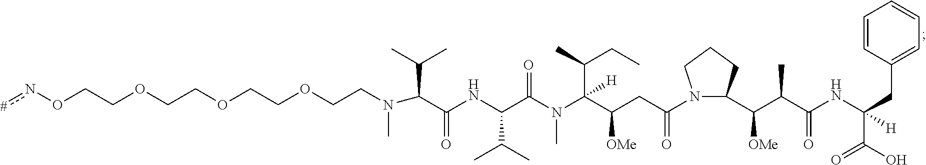

wherein # represents a connection to the anti-HER2 antibody or antibody fragment. In some embodiments, the anti-HER2 antibody or antibody fragment is humanized. In some embodiments, the one or more non-naturally encoded amino acids are para-acetyl phenylalanine. In some embodiments, a light chain of the anti-HER2 antibody or antibody fragment is any one of SEQ ID NOs: 4, 5, 6, 7, 8, 9, 10, 11, 12 and 13.

Still another aspect of the disclosure relates to a composition comprising: an antibody-drug conjugate (ADC) disclosed herein.

In some embodiments, the composition further comprises an additional therapeutic agent. In some embodiments, the additional therapeutic agent is an immunotherapeutic agent, chemotherapeutic agent, hormonal agent, antitumor agent, immunostimulatory agent or immunomodulator, or a combination thereof. In some embodiments, the additional therapeutic agent is a HER2 targeted therapeutic. In some embodiments, the additional therapeutic agent is a checkpoint inhibitor, a HER2 kinase inhibitor, cyclin-dependent kinase inhibitor, tyrosine kinase inhibitor, small-molecule kinase inhibitor or a platinum-based therapeutic, or a combination thereof.

Another aspect of the disclosure relates to a method killing a cell comprising contacting a cell with an ADC disclosed herein.

In some embodiments, the cell is a tumor or cancer cell.

Still another aspect of the disclosure relates to a pharmaceutical composition comprising an ADC disclosed herein.

In some embodiments, the pharmaceutical composition further comprises a pharmaceutically acceptable excipient.

Another aspect of the disclosure relates to a method of treating a tumor or cancer in a human subject in need thereof, wherein the method comprising administering to the human subject an effective amount of an ADC disclosed herein, or a pharmaceutical composition disclosed herein.

In some embodiments, the effective amount is about 0.22, 0.33, 0.44, 0.55, 0.66, 0.77, 0.88, 0.99, 1.0, 1.1, 1.2, 1.3, 1.4, 1.5, 1.6, 1.7, 1.8, 1.9 or 2.0 mg per kilogram body weight (mg/kg) of the human subject. In some embodiments, the effective amount is about 0.33, 0.66, 0.88, 1.1, 1.3, 1.5, 1.6, 1.7 or 1.8 mg/kg of the human subject. In some embodiments, wherein the administering is once every 1, 2, 3, 4, 5, 6, 7, or 8-week. In some embodiments, the administering lasts two or more 1, 2, 3, 4, 5, 6, 7 or 8-week cycles. In some embodiments, the administering lasts at least four weeks. In some embodiments, the administering lasts at least three weeks. In some embodiments, the administering is more than once within a 1, 2, 3, 4, 5, 6, 7, or 8-week cycle or more. In some embodiments, the administering is more than once within a 4-week cycle. In some embodiments, the administering is more than once within a 3-week cycle. In some embodiments, the dosage administered is the same on different days of the administering. In some embodiments, the dosage administered is different on different days of the administering. In some embodiments, the ADC is administered once every two weeks for more than 8 weeks. In some embodiments, the ADC is administered once every four weeks for more than 8 weeks. In some embodiments, the tumor or cancer is breast cancer, ovarian cancer, gastric cancer, gastro-esophageal junction adenocarcinoma, cervical cancer, uterine cancer, endometrial cancer, testicular cancer, prostate cancer, colorectal cancer, esophageal cancer, bladder cancer, non-small cell lung cancer (NSCLC), urothelial carcinoma, cholangiocarcinoma, colon biliary tract cancer, pancreatic cancer, or solid tumor. In some embodiments, the tumor or cancer is breast cancer, ovarian cancer or gastric cancer.

In some embodiments, the administering comprises about 1.8 mg/kg dosage of the ADC on day 1 of the first 4-week cycle. In some embodiments, the administering further comprises about 1.7 mg/kg, 1.5 mg/kg, 1.3 mg/kg or lower dosage of the ADC on day 1 of the second 4-week cycle. In some embodiments, the administering comprises about 1.7 mg/kg dosage of the ADC on day 1 of the first 4-week cycle. In some embodiments, the administering further comprises about 1.5 mg/kg, 1.3 mg/kg or lower dosage of the ADC on day 1 of the second 4-week cycle. In some embodiments, the administering comprises about 1.6 mg/kg dosage of the ADC on day 1 of the first 4-week cycle. In some embodiments, the administering further comprises about 1.5 mg/kg, 1.3 mg/kg, 1.1 mg/kg or lower dosage of the ADC on day 1 of the second 4-week cycle. In some embodiments, the administering comprises about 1.5 mg/kg dosage of the ADC on day 1 of the first 4-week cycle. In some embodiments, the administering further comprises about 1.3 mg/kg, 1.1 mg/kg or lower dosage of the ADC on day 1 of the second 4-week cycle.

In some embodiments, wherein the administering last two or more 3-week cycles. In some embodiments, the administering comprises about 1.8 mg/kg dosage of the ADC on day 1 of the first 3-week cycle. In some embodiments, the administering comprises about 1.7 mg/kg dosage of the ADC on day 1 of the first 3-week cycle. In some embodiments, administering comprises about 1.5 mg/kg dosage of the ADC on day 1 of the first 3-week cycle. In some embodiments, the administering comprises about 1.3 mg/kg dosage of the ADC on day 1 of the first 3-week cycle. In some embodiments, the administering comprises about 1.1 mg/kg dosage of the ADC on day 1 of the first 3-week cycle. In some embodiments, the administering comprises about 0.88 mg/kg dosage of the ADC on day 1 of the first 3-week cycle. In some embodiments, the administering comprises about 0.66 mg/kg dosage of the ADC on day 1 of the first 3-week cycle.

In some embodiments, the method further comprises administering to the human subject an effective amount of an additional therapeutic agent. In some embodiments, the additional therapeutic agent is a chemotherapeutic agent, a hormonal agent, an antitumor agent, an immunostimulatory agent, an immunomodulator or an immunotherapeutic agent, or a combination thereof. In some embodiments, the additional therapeutic agent is a checkpoint inhibitor, a HER2 kinase inhibitor, cyclin-dependent kinase inhibitor, tyrosine kinase inhibitor, small-molecule kinase inhibitor or a platinum-based therapeutic, or a combination thereof. In some embodiments, the therapeutic agent is a HER2 targeted therapeutic. In some embodiments, the method improves or optimizes cancer cell kill. In some embodiments, the method delays progression or recurrence of the tumor or cancer. In some embodiments, the administering is orally, intradermally, intratumorally, intravenously, or subcutaneously. In some embodiments, the tumor or cancer is a HER-2 expressing cancer. In some embodiments, the cancer is a HER-2 low expressing cancer, HER2 moderate expressing cancer, or HER2 high expressing cancer. In some embodiments, the subject to be treated has a HER-2 expressing cancer and cancer metastases from a same or different cancer.

Still another aspect of the disclosure relates to a formulation comprising: (i) about 20 mg/mL ADC disclosed herein; (ii) about 5 mM histidine buffer at about pH 6.0, (iii) about 6% w/v trehalose, and (iv) about 0.02% w/v polysorbate 80.

In some embodiments, the histidine is L-histidine.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DISCLOSURE OF THE DRAWINGS

FIG. 1 depicts an example antibody-drug conjugate (ADC) comprising a cytotoxic tubulin inhibitor AS269 suitable for use in the present disclosure.

FIG. 2 depicts a waterfall plot of the best change in the sum of the target lesions from baseline in the 1.5 mg/kg and 1.3 mg/kg cohorts.

FIG. 3 depicts spider plot change in the sum of target lesions from baseline over time.

FIG. 4 depicts durable response in cohort dosed at 1.5 mg/kg.

FIG. 5 depicts a waterfall plot of Pan tumor in the 1.5 mg/kg Q4W cohorts.

FIG. 6 depicts durable response in breast cohort dosed at 1.5 mg/kg

FIG. 7 depicts a waterfall plot change from baseline in the 1.5 mg/kg and 1.3 mg/kg.

FIG. 8 depicts durable response in breast cohort dosed at 1.3 mg/kg.

FIG. 9 depicts survival probability at 1.5 mg/kg and 1.3 mg/kg Q3W/Q4W.

FIG. 10 depicts survival probability at 1.5 mg/kg Q3W and 1.3 mg/kg Q3W.

FIG. 11 depicts survival probability at 1.5 mg/kg and 1.3 mg/kg Q3W/Q4W.

FIG. 12 depicts survival probability at 1.5 mg/kg Q3W and 1.3 mg/kg Q3W.

FIG. 13 depicts HR+/HER2-low metastatic breast cancer patient with partial response after 2 cycles of ADC.

### DETAILED DESCRIPTION OF THE INVENTION

The overexpression and/or amplification of human epidermal growth factor receptor 2 (HER2) occurs in approximately 20% of breast cancers (BC) and is a major driver of tumor development and progression. This HER2 subtype confers aggressive tumor behavior and the HER2 receptor remains a valuable target for antibodies, bi-specifics, and antibody drug conjugates (ADC). With advances in targeted therapy, patients with HER2-positive breast cancer (HER2+BC) may experience an improved prognosis, including survival. Novel HER2-targeted therapies are being investigated to overcome drug resistance and to help mitigate adverse events (e.g., cardiotoxicity). The present disclosure provides a next-generation ADC using a technology platform whereby a HER2 specific monoclonal antibody is conjugated with Amberstatin269 (AS269), a potent cytotoxic tubulin inhibitor. Site-specificity, high homogeneity, and stable covalent conjugation of the ADC leads to its slow release and prolonged peak of serum pAF-AS269, which may contribute to the lower systemic toxicity and increased targeted delivery of payload to tumor cells at a lower effective dose compared to other HER2 ADCs. The ADC of the disclosure was designed to inhibit the growth of HER2 overexpressing cells through multiple sequential steps, including binding to HER2 on the surface of cancer cells, rapidly internalizing, trafficking to the lysosome, and metabolizing inside the lysosome to release pAF-AS269, which binds to microtubules and induces cancer cell cycle arrest and cell death.

Disclosed herein are antibody-drug conjugates (ADCs) comprising a targeting moiety such as an antibody and one or more drugs. The drug may further comprise one or more linker(s). The ADCs of the present disclosure may comprise drugs linked to non-natural amino acids in the targeting moiety. Also included are methods for making such ADCs comprising non-natural amino acids incorporated into the targeting moiety polypeptides.

In certain embodiments, a pharmaceutical composition is provided comprising any of the compounds described and a pharmaceutically acceptable carrier, excipient, or binder.

In further or alternative embodiments are methods for detecting the presence of a polypeptide in a patient, the method comprising administering a polypeptide comprising at least one heterocycle-containing non-natural amino acid and the resulting heterocycle-containing non-natural amino acid polypeptide modulates the immunogenicity of the polypeptide relative to the homologous naturally-occurring amino acid polypeptide.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods and compositions described herein, which will be limited only by the appended claims.

### Definitions

Unless otherwise defined herein, scientific and technical terms used in connection with the present disclosure shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting.

Generally, nomenclatures used in connection with, and techniques of, cell and tissue culture, molecular biology, immunology, microbiology, genetics and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients. The present disclosure may be more readily understood, select terms are defined below.

As used herein and in the appended claims, the singular forms “a,” “an,” and “the” include plural reference unless the context clearly indicates otherwise.

The terms “aldol-based linkage” or “mixed aldol-based linkage” refers to the acid- or base-catalyzed condensation of one carbonyl compound with the enolate/enol of another carbonyl compound, which may or may not be the same, to generate a P-hydroxy carbonyl compound—an aldol.

The term “affinity label,” as used herein, refers to a label which reversibly or irreversibly binds another molecule, either to modify it, destroy it, or form a compound with it. By way of example, affinity labels include enzymes and their substrates, or antibodies and their antigens.

The terms “alkoxy,” “alkylamino” and “alkylthio” (or thioalkoxy) are used in their conventional sense and refer to those alkyl groups linked to molecules via an oxygen atom, an amino group, or a sulfur atom, respectively.

The term “alkyl,” by itself or as part of another molecule means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail herein, such as “heteroalkyl”, “haloalkyl” and “homoalkyl”.

The term “alkylene” by itself or as part of another molecule means a divalent radical derived from an alkane, as exemplified, by (—CH₂—)ₙ, wherein n may be 1 to about 24. By way of example only, such groups include, but are not limited to, groups having 10 or fewer carbon atoms such as the structures —CH₂CH₂— and —CH₂CH₂CH₂CH₂—. A “lower alkyl” or “lower alkylene” is a shorter chain alkyl or alkylene group, generally having eight or fewer carbon atoms. The term “alkylene,” unless otherwise noted, is also meant to include those groups described herein as “heteroalkylene.”

The term “amino acid” refers to naturally occurring and non-natural amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrrolysine and selenocysteine. Amino acid analogs refer to compounds that have the same basic chemical structure as a naturally occurring amino acid, by way of example only, an a-carbon that is bound to a hydrogen, a carboxyl group, an amino group, and an R group. Such analogs may have modified R groups (by way of example, norleucine) or may have modified peptide backbones while still retaining the same basic chemical structure as a naturally occurring amino acid. Non-limiting examples of amino acid analogs include homoserine, norleucine, methionine sulfoxide, methionine methyl sulfonium.

Amino acids may be referred to herein by either their name, their commonly known three letter symbols or by the one-letter symbols recommended by the IUPAC-IUB Biochemical Nomenclature Commission. Additionally, nucleotides, may be referred to by their commonly accepted single-letter codes.

An “amino terminus modification group” refers to any molecule that can be attached to a terminal amine group. By way of example, such terminal amine groups may be at the end of polymeric molecules, wherein such polymeric molecules include, but are not limited to, polypeptides, polynucleotides, and polysaccharides. Terminus modification groups include but are not limited to, various water soluble polymers, peptides or proteins. By way of example only, terminus modification groups include polyethylene glycol or serum albumin. Terminus modification groups may be used to modify therapeutic characteristics of the polymeric molecule, including but not limited to increasing the serum half-life of peptides.

By “antibody” herein is meant a protein consisting of one or more polypeptides substantially encoded by all or part of the antibody genes. The immunoglobulin genes include, but are not limited to, the kappa, lambda, alpha, gamma (IgG1, IgG2, IgG3, and IgG4), delta, epsilon and mu constant region genes, as well as the myriad immunoglobulin variable region genes. Antibody herein is meant to include full-length antibodies and antibody fragments and include antibodies that exist naturally in any organism or are engineered (e.g. are variants).

By “antibody fragment” is meant any form of an antibody other than the full-length form. Antibody fragments herein include antibodies that are smaller components that exist within full-length antibodies, and antibodies that have been engineered. Antibody fragments include but are not limited to Fv, Fc, Fab, and (Fab′)2, single chain Fv (scFv), diabodies, triabodies, tetrabodies, bifunctional hybrid antibodies, CDR1, CDR2, CDR3, combinations of CDR's, variable regions, framework regions, constant regions, heavy chains, light chains, and variable regions, and alternative scaffold non-antibody molecules, bispecific antibodies, and the like (Maynard & Georgiou, 2000, Annu. Rev. Biomed. Eng. 2:339-76; Hudson, 1998, Curr. Opin. Biotechnol. 9:395-402). Another functional substructure is a single chain Fv (scFv), comprised of the variable regions of the immunoglobulin heavy and light chain, covalently connected by a peptide linker (S-z Hu et al., 1996, Cancer Research, 56, 3055-3061). These small (Mr 25,000) proteins generally retain specificity and affinity for antigen in a single polypeptide and can provide a convenient building block for larger, antigen-specific molecules. Unless specifically noted otherwise, statements and claims that use the term “antibody” or “antibodies” specifically includes “antibody fragment” and “antibody fragments”.

By “antibody-drug conjugate”, or “ADC”, as used herein, refers to an antibody molecule, or fragment thereof, that is covalently bonded to one or more biologically active molecule(s). The biologically active molecule may be conjugated to the antibody through a linker, polymer, or other covalent bond.

The term “aromatic” or “aryl”, as used herein, refers to a closed ring structure which has at least one ring having a conjugated pi electron system and includes both carbocyclic aryl and heterocyclic aryl (or “heteroaryl” or “heteroaromatic”) groups. The carbocyclic or heterocyclic aromatic group may contain from 5 to 20 ring atoms. The term includes monocyclic rings linked covalently or fused-ring polycyclic (i.e., rings which share adjacent pairs of carbon atoms) groups. An aromatic group can be unsubstituted or substituted. Non-limiting examples of “aromatic” or “aryl”, groups include phenyl, 1-naphthyl, 2-naphthyl, 4-biphenyl, anthracenyl, and phenanthracenyl. Substituents for each of the above noted aryl and heteroaryl ring systems are selected from the group of acceptable substituents described herein.

For brevity, the term “aromatic” or “aryl” when used in combination with other terms (including but not limited to, aryloxy, arylthioxy, aralkyl) includes both aryl and heteroaryl rings as defined above. Thus, the term “aralkyl” or “alkaryl” is meant to include those radicals in which an aryl group is attached to an alkyl group (including but not limited to, benzyl, phenethyl, pyridylmethyl and the like) including those alkyl groups in which a carbon atom (including but not limited to, a methylene group) has been replaced by a heteroatom, by way of example only, by an oxygen atom. Examples of such aryl groups include, but are not limited to, phenoxymethyl, 2-pyridyloxymethyl, 3-(1-naphthyloxy)propyl, and the like.

The term “arylene”, as used herein, refers to a divalent aryl radical. Non-limiting examples of “arylene” include phenylene, pyridinylene, pyrimidinylene and thiophenylene. Substituents for arylene groups are selected from the group of acceptable substituents described herein.

A “bifunctional polymer”, also referred to as a “bifunctional linker”, refers to a polymer comprising two functional groups that are capable of reacting specifically with other moieties to form covalent or non-covalent linkages. Such moieties may include, but are not limited to, the side groups on natural or non-natural amino acids or peptides which contain such natural or non-natural amino acids. The other moieties that may be linked to the bifunctional linker or bifunctional polymer may be the same or different moieties. By way of example only, a bifunctional linker may have a functional group reactive with a group on a first peptide, and another functional group which is reactive with a group on a second peptide, whereby forming a conjugate that includes the first peptide, the bifunctional linker and the second peptide. Many procedures and linker molecules for attachment of various compounds to peptides are known. See, e.g., European Patent Application No. 188,256; U.S. Pat. Nos. 4,671,958, 4,659,839, 4,414,148, 4,699,784; 4,680,338; and 4,569,789 which are incorporated by reference herein in their entirety. A “multi-functional polymer” also referred to as a “multi-functional linker”, refers to a polymer comprising two or more functional groups that are capable of reacting with other moieties. Such moieties may include, but are not limited to, the side groups on natural or non-natural amino acids or peptides which contain such natural or non-natural amino acids. (including but not limited to, amino acid side groups) to form covalent or non-covalent linkages. A bi-functional polymer or multi-functional polymer may be any desired length or molecular weight, and may be selected to provide a particular desired spacing or conformation between one or more molecules linked to a compound and molecules it binds to or the compound.

The term “bioavailability,” as used herein, refers to the rate and extent to which a substance or its active moiety is delivered from a pharmaceutical dosage form and becomes available at the site of action or in the general circulation. Increases in bioavailability refers to increasing the rate and extent a substance or its active moiety is delivered from a pharmaceutical dosage form and becomes available at the site of action or in the general circulation. By way of example, an increase in bioavailability may be indicated as an increase in concentration of the substance or its active moiety in the blood when compared to other substances or active moieties. Methods to evaluate increases in bioavailability are known in the art and may be used for evaluating the bioavailability of any polypeptide.

The term “biologically active molecule”, “biologically active moiety” or “biologically active agent” when used herein means any substance which can affect any physical or biochemical properties of a biological system, pathway, molecule, or interaction relating to an organism, including but not limited to, viruses, bacteria, bacteriophage, transposon, prion, insects, fungi, plants, animals, and humans. In particular, as used herein, biologically active molecules include but are not limited to any substance intended for diagnosis, cure, mitigation, treatment, or prevention of disease in humans or other animals, or to otherwise enhance physical or mental well-being of humans or animals. Examples of biologically active molecules include, but are not limited to, peptides, proteins, enzymes, small molecule drugs, hard drugs, soft drugs, prodrugs, carbohydrates, inorganic atoms or molecules, dyes, lipids, nucleosides, radionuclides, oligonucleotides, toxins, cells, viruses, liposomes, microparticles and micelles. Classes of biologically active agents that are suitable for use with the methods and compositions described herein include, but are not limited to, drugs, prodrugs, radionuclides, imaging agents, polymers, antibiotics, fungicides, anti-viral agents, anti-inflammatory agents, anti-tumor agents, cardiovascular agents, anti-anxiety agents, hormones, growth factors, steroidal agents, microbially derived toxins, and the like.

By “modulating biological activity” is meant increasing or decreasing the reactivity of a polypeptide, altering the selectivity of the polypeptide, enhancing or decreasing the substrate selectivity of the polypeptide. Analysis of modified biological activity can be performed by comparing the biological activity of the non-natural polypeptide to that of the natural polypeptide.

The term “biomaterial,” as used herein, refers to a biologically-derived material, including but not limited to material obtained from bioreactors and/or from recombinant methods and techniques.

The term “biophysical probe,” as used herein, refers to probes which can detect or monitor structural changes in molecules. Such molecules include, but are not limited to, proteins and the “biophysical probe” may be used to detect or monitor interaction of proteins with other macromolecules. Examples of biophysical probes include, but are not limited to, spin-labels, a fluorophores, and photoactivatible groups.

The term “biosynthetically,” as used herein, refers to any method utilizing a translation system (cellular or non-cellular), including use of at least one of the following components: a polynucleotide, a codon, a tRNA, and a ribosome. By way of example, non-natural amino acids may be “biosynthetically incorporated” into non-natural amino acid polypeptides using the methods and techniques described in WO 2002/085923, incorporated herein by reference in its entirety. Additionally, the methods for the selection of useful non-natural amino acids which may be “biosynthetically incorporated” into non-natural amino acid polypeptides are described in WO 2002/085923, incorporated herein by reference in its entirety.

The term “biotin analogue,” or also referred to as “biotin mimic”, as used herein, is any molecule, other than biotin, which bind with high affinity to avidin and/or streptavidin.

The term “carbonyl” as used herein refers to a group containing at a moiety selecting from the group consisting of —C(O)—, —S(O)—, —S(O)2-, and —C(S)—, including, but not limited to, groups containing a least one ketone group, and/or at least one aldehyde groups, and/or at least one ester group, and/or at least one carboxylic acid group, and/or at least one thioester group. Such carbonyl groups include ketones, aldehydes, carboxylic acids, esters, and thioesters. In addition, such groups may be part of linear, branched, or cyclic molecules.

The term “carboxy terminus modification group” refers to any molecule that can be attached to a terminal carboxy group. By way of example, such terminal carboxy groups may be at the end of polymeric molecules, wherein such polymeric molecules include, but are not limited to, polypeptides, polynucleotides, and polysaccharides. Terminus modification groups include but are not limited to, various water soluble polymers, peptides or proteins. By way of example only, terminus modification groups include polyethylene glycol or serum albumin. Terminus modification groups may be used to modify therapeutic characteristics of the polymeric molecule, including but not limited to increasing the serum half-life of peptides.

The term “chemically cleavable group,” also referred to as “chemically labile”, as used herein, refers to a group which breaks or cleaves upon exposure to acid, base, oxidizing agents, reducing agents, chemical initiators, or radical initiators.

“Cofolding,” as used herein, refers to refolding processes, reactions, or methods which employ at least two molecules which interact with each other and result in the transformation of unfolded or improperly folded molecules to properly folded molecules. By way of example only, “cofolding,” employ at least two polypeptides which interact with each other and result in the transformation of unfolded or improperly folded polypeptides to native, properly folded polypeptides. Such polypeptides may contain natural amino acids and/or at least one non-natural amino acid.

“Conjugate”, as used herein, refers to a polypeptide that is linked, e.g., covalently linked, either directly or through a linker to a compound or compound-linker described herein. The “targeting moiety” refers to a structure that has a selective affinity for a target molecule relative to other non-target molecules. A targeting moiety of the disclosure binds to a target molecule. A targeting moiety may include, for example, an antibody, a peptide, a ligand, a receptor, or a binding portion thereof. A target biological molecule may be a biological receptor or other structure of a cell such as a tumor antigen. As used herein, the term “conjugate of the invention,” “conjugate of the disclosure”, “targeting moiety conjugate” “targeting conjugate,” or “targeting moiety-active molecule conjugate” refers to a targeting polypeptide or a portion, analog or derivative thereof that binds to a target present on a cell or subunit thereof conjugated to a biologically active molecule, a portion thereof or an analog thereof, including but not limited to a cytotoxic tubulin inhibitor, such as AS269. As used herein, the term “tumor-targeting moiety conjugate”, or “tumor-targeting moiety-biologically active molecule conjugate” refers to a tumor targeting polypeptide or a portion, analog or derivative thereof that binds to a target present on tumor cells or subunit thereof conjugated to a biologically active molecule, a portion thereof or an analog thereof, including but not limited to a cytotoxic tubulin inhibitor, such as AS269. Unless otherwise indicated, the terms “compound of the invention”, “compound of the disclosure”, “composition of the disclosure”, and “composition of the invention” are used as alternatives for the term “conjugate of the invention.”

The term “conservatively modified variants” applies to both natural and non-natural amino acid and natural and non-natural nucleic acid sequences, and combinations thereof. With respect to particular nucleic acid sequences, “conservatively modified variants” refers to those natural and non-natural nucleic acids which encode identical or essentially identical natural and non-natural amino acid sequences, or where the natural and non-natural nucleic acid does not encode a natural and non-natural amino acid sequence, to essentially identical sequences. By way of example, because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Thus, by way of example, every natural or non-natural nucleic acid sequence herein which encodes a natural or non-natural polypeptide also describes every possible silent variation of the natural or non-natural nucleic acid. One of ordinary skill in the art can recognize that each codon in a natural or non-natural nucleic acid (except AUG, which is ordinarily the only codon for methionine, and TGG, which is ordinarily the only codon for tryptophan) can be modified to yield a functionally identical molecule. Accordingly, each silent variation of a natural and non-natural nucleic acid which encodes a natural and non-natural polypeptide is implicit in each described sequence.

As to amino acid sequences, individual substitutions, deletions or additions to a nucleic acid, peptide, polypeptide, or protein sequence which alters, adds or deletes a single natural and non-natural amino acid or a small percentage of natural and non-natural amino acids in the encoded sequence is a “conservatively modified variant” where the alteration results in the deletion of an amino acid, addition of an amino acid, or substitution of a natural and non-natural amino acid with a chemically similar amino acid. Conservative substitution tables providing functionally similar natural amino acids are well known in the art. Such conservatively modified variants are in addition to and do not exclude polymorphic variants, interspecies homologs, and alleles of the methods and compositions described herein.

Conservative substitution tables providing functionally similar amino acids are known to those of ordinary skill in the art. The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M). (See, e.g., Creighton, Proteins:Structures and Molecular Properties (W H Freeman & Co.; 2nd edition (December 1993).

The terms “cycloalkyl” and “heterocycloalkyl”, by themselves or in combination with other terms, represent, unless otherwise stated, cyclic versions of “alkyl” and “heteroalkyl”, respectively. Thus, a cycloalkyl or heterocycloalkyl include saturated, partially unsaturated and fully unsaturated ring linkages. Additionally, for heterocycloalkyl, a heteroatom can occupy the position at which the heterocycle is attached to the remainder of the molecule. The heteroatom may include, but is not limited to, oxygen, nitrogen or sulfur. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. Additionally, the term encompasses multicyclic structures, including but not limited to, bicyclic and tricyclic ring structures. Similarly, the term “heterocycloalkylene” by itself or as part of another molecule means a divalent radical derived from heterocycloalkyl, and the term “cycloalkylene” by itself or as part of another molecule means a divalent radical derived from cycloalkyl.

The term “cyclodextrin,” as used herein, refers to cyclic carbohydrates consisting of at least six to eight glucose molecules in a ring formation. The outer part of the ring contains water soluble groups; at the center of the ring is a relatively nonpolar cavity able to accommodate small molecules.

The term “cytotoxic,” as used herein, refers to a compound which harms cells.

“Denaturing agent” or “denaturant,” as used herein, refers to any compound or material which will cause a reversible unfolding of a polymer. By way of example only, “denaturing agent” or “denaturants,” may cause a reversible unfolding of a protein. The strength of a denaturing agent or denaturant will be determined both by the properties and the concentration of the particular denaturing agent or denaturant. By way of example, denaturing agents or denaturants include, but are not limited to, chaotropes, detergents, organic, water miscible solvents, phospholipids, or a combination thereof. Non-limiting examples of chaotropes include, but are not limited to, urea, guanidine, and sodium thiocyanate. Non-limiting examples of detergents may include, but are not limited to, strong detergents such as sodium dodecyl sulfate, or polyoxyethylene ethers (e.g. Tween or Triton detergents), Sarkosyl, mild non-ionic detergents (e.g., digitonin), mild cationic detergents such as N->2,3-(Dioleyoxy)-propyl-N,N,N-trimethylammonium, mild ionic detergents (e.g. sodium cholate or sodium deoxycholate) or zwitterionic detergents including, but not limited to, sulfobetaines (Zwittergent), 3-(3-chlolamidopropyl)dimethylammonio-1-propane sulfate (CHAPS), and 3-(3-chlolamidopropyl)dimethylammonio-2-hydroxy-1-propane sulfonate (CHAPSO). Non-limiting examples of organic, water miscible solvents include, but are not limited to, acetonitrile, lower alkanols (especially C2-C4 alkanols such as ethanol or isopropanol), or lower alkanediols (C2-C4 alkanediols such as ethylene-glycol) may be used as denaturants. Non-limiting examples of phospholipids include, but are not limited to, naturally occurring phospholipids such as phosphatidylethanolamine, phosphatidylcholine, phosphatidylserine, and phosphatidylinositol or synthetic phospholipid derivatives or variants such as dihexanoylphosphatidylcholine or diheptanoylphosphatidylcholine.

The term “diamine,” as used herein, refers to groups/molecules comprising at least two amine functional groups, including, but not limited to, a hydrazine group, an amidine group, an imine group, a 1,1-diamine group, a 1,2-diamine group, a 1,3-diamine group, and a 1,4-diamine group. In addition, such groups may be part of linear, branched, or cyclic molecules.

The term “detectable label,” as used herein, refers to a label which may be observable using analytical techniques including, but not limited to, fluorescence, chemiluminescence, electron-spin resonance, ultraviolet/visible absorbance spectroscopy, mass spectrometry, nuclear magnetic resonance, magnetic resonance, and electrochemical methods.

The term “dicarbonyl”, as used herein refers to a group containing at least two moieties selected from the group consisting of —C(O)—, —S(O)—, —S(O)₂—, and —C(S)—, including, but not limited to, 1,2-dicarbonyl groups, a 1,3-dicarbonyl groups, and 1,4-dicarbonyl groups, and groups containing a least one ketone group, and/or at least one aldehyde groups, and/or at least one ester group, and/or at least one carboxylic acid group, and/or at least one thioester group. Such dicarbonyl groups include diketones, ketoaldehydes, ketoacids, ketoesters, and ketothioesters. In addition, such groups may be part of linear, branched, or cyclic molecules. The two moieties in the dicarbonyl group may be the same or different, and may include substituents that would produce, by way of example only, an ester, a ketone, an aldehyde, a thioester, or an amide, at either of the two moieties.

The term “drug,” as used herein, refers to any substance used in the prevention, diagnosis, alleviation, treatment, or cure of a disease or condition.

The term “effective amount,” as used herein, refers to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. By way of example, an agent or a compound being administered includes, but is not limited to, a natural amino acid polypeptide, non-natural amino acid polypeptide, modified natural amino acid polypeptide, or modified non-amino acid polypeptide. Compositions containing such natural amino acid polypeptides, non-natural amino acid polypeptides, modified natural amino acid polypeptides, or modified non-natural amino acid polypeptides can be administered for prophylactic, enhancing, and/or therapeutic treatments. An appropriate “effective” amount in any individual case may be determined using techniques, such as a dose escalation study.

The terms “enhance” or “enhancing” means to increase or prolong either in potency or duration a desired effect. By way of example, “enhancing” the effect of therapeutic agents refers to the ability to increase or prolong, either in potency or duration, the effect of therapeutic agents on during treatment of a disease, disorder or condition. An “enhancing-effective amount,” as used herein, refers to an amount adequate to enhance the effect of a therapeutic agent in the treatment of a disease, disorder or condition. When used in a patient, amounts effective for this use will depend on the severity and course of the disease, disorder or condition, previous therapy, the patient's health status and response to the drugs, and the judgment of the treating physician.

As used herein, the term “eukaryote” refers to organisms belonging to the phylogenetic domain Eucarya, including but not limited to animals (including but not limited to, mammals, insects, reptiles, birds, etc.), ciliates, plants (including but not limited to, monocots, dicots, and algae), fungi, yeasts, flagellates, microsporidia, and protists.

The term “fatty acid,” as used herein, refers to carboxylic acids with about C6 or longer hydrocarbon side chain.

The term “fluorophore,” as used herein, refers to a molecule which upon excitation emits photons and is thereby fluorescent.

The terms “functional group”, “active moiety”, “activating group”, “leaving group”, “reactive site”, “chemically reactive group” and “chemically reactive moiety,” as used herein, refer to portions or units of a molecule at which chemical reactions occur. The terms are somewhat synonymous in the chemical arts and are used herein to indicate the portions of molecules that perform some function or activity and are reactive with other molecules.

The term “halogen” includes fluorine, chlorine, iodine, and bromine.

The term “haloacyl,” as used herein, refers to acyl groups which contain halogen moieties, including, but not limited to, —C(O)CH₃, —C(O)CF₃, —C(O)CH₂OCH₃, and the like.

The term “haloalkyl,” as used herein, refers to alkyl groups which contain halogen moieties, including, but not limited to, —CF₃ and —CH₂CF₃ and the like.

The term “heteroalkyl,” as used herein, refers to straight or branched chain, or cyclic hydrocarbon radicals, or combinations thereof, consisting of an alkyl group and at least one heteroatom selected from the group consisting of O, N, Si and S, and wherein the nitrogen and sulfur atoms may optionally be oxidized, and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N-OCH₃, and —CH═CH—N(CH₃)—CH₃. In addition, up to two heteroatoms may be consecutive, such as, by way of example, —CH₂—NH—OCH₃ and —CH₂—O—Si(CH₃)₃.

The terms “heterocyclic-based linkage” or “heterocycle linkage” refers to a moiety formed from the reaction of a dicarbonyl group with a diamine group. The resulting reaction product is a heterocycle, including a heteroaryl group or a heterocycloalkyl group. The resulting heterocycle group serves as a chemical link between a non-natural amino acid or non-natural amino acid polypeptide and another functional group. In one embodiment, the heterocycle linkage includes a nitrogen-containing heterocycle linkage, including by way of example only a pyrazole linkage, a pyrrole linkage, an indole linkage, a benzodiazepine linkage, and a pyrazalone linkage.

Similarly, the term “heteroalkylene” refers to a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, the same or different heteroatoms can also occupy either or both of the chain termini (including but not limited to, alkyleneoxy, alkylenedioxy, alkyleneamino, alkylenediamino, aminooxyalkylene, and the like). Still further, for alkylene and heteroalkylene linking groups, no orientation of the linking group is implied by the direction in which the formula of the linking group is written. By way of example, the formula —C(O)₂R′— represents both —C(O)₂R′— and —R′C(O)₂—.

The term “heteroaryl” or “heteroaromatic,” as used herein, refers to aryl groups which contain at least one heteroatom selected from N, O, and S; wherein the nitrogen and sulfur atoms may be optionally oxidized, and the nitrogen atom(s) may be optionally quaternized. Heteroaryl groups may be substituted or unsubstituted. A heteroaryl group may be attached to the remainder of the molecule through a heteroatom. Non-limiting examples of heteroaryl groups include 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl.

The term “homoalkyl,” as used herein refers to alkyl groups which are hydrocarbon groups.

The term “identical,” as used herein, refers to two or more sequences or subsequences which are the same. In addition, the term “substantially identical,” as used herein, refers to two or more sequences which have a percentage of sequential units which are the same when compared and aligned for maximum correspondence over a comparison window, or designated region as measured using comparison algorithms or by manual alignment and visual inspection. By way of example only, two or more sequences may be “substantially identical” if the sequential units are about 60% identical, about 65% identical, about 70% identical, about 75% identical, about 80% identical, about 85% identical, about 90% identical, or about 95% identical over a specified region. Such percentages to describe the “percent identity” of two or more sequences. The identity of a sequence can exists over a region that is at least about 75-100 sequential units in length, over a region that is about 50 sequential units in length, or, where not specified, across the entire sequence. This definition also refers to the complement of a test sequence. By way of example only, two or more polypeptide sequences are identical when the amino acid residues are the same, while two or more polypeptide sequences are “substantially identical” if the amino acid residues are about 60% identical, about 65% identical, about 70% identical, about 75% identical, about 80% identical, about 85% identical, about 90% identical, or about 95% identical over a specified region. The identity can exist over a region that is at least about 75 to about 100 amino acids in length, over a region that is about 50 amino acids in length, or, where not specified, across the entire sequence of a polypeptide sequence. In addition, by way of example only, two or more polynucleotide sequences are identical when the nucleic acid residues are the same, while two or more polynucleotide sequences are “substantially identical” if the nucleic acid residues are about 60% identical, about 65% identical, about 70% identical, about 75% identical, about 80% identical, about 85% identical, about 90% identical, or about 95% identical over a specified region. The identity can exist over a region that is at least about 75 to about 100 nucleic acids in length, over a region that is about 50 nucleic acids in length, or, where not specified, across the entire sequence of a polynucleotide sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

The term “immunogenicity,” as used herein, refers to an antibody response to administration of a therapeutic drug. The immunogenicity toward therapeutic non-natural amino acid polypeptides can be obtained using quantitative and qualitative assays for detection of anti-non-natural amino acid polypeptides antibodies in biological fluids. Such assays include, but are not limited to, Radioimmunoassay (RIA), Enzyme-linked immunosorbent assay (ELISA), luminescent immunoassay (LIA), and fluorescent immunoassay (FIA). Analysis of immunogenicity toward therapeutic non-natural amino acid polypeptides involves comparing the antibody response upon administration of therapeutic non-natural amino acid polypeptides to the antibody response upon administration of therapeutic natural amino acid polypeptides.

The term “isolated,” as used herein, refers to separating and removing a component of interest from components not of interest. Isolated substances can be in either a dry or semi-dry state, or in solution, including but not limited to an aqueous solution. The isolated component can be in a homogeneous state or the isolated component can be a part of a pharmaceutical composition that comprises additional pharmaceutically acceptable carriers and/or excipients. Purity and homogeneity may be determined using analytical chemistry techniques including, but not limited to, polyacrylamide gel electrophoresis or high performance liquid chromatography. In addition, when a component of interest is isolated and is the predominant species present in a preparation, the component is described herein as substantially purified. The term “purified,” as used herein, may refer to a component of interest which is at least 85% pure, at least 90% pure, at least 95% pure, at least 99% or greater pure. By way of example only, nucleic acids or proteins are “isolated” when such nucleic acids or proteins are free of at least some of the cellular components with which it is associated in the natural state, or that the nucleic acid or protein has been concentrated to a level greater than the concentration of its in vivo or in vitro production. Also, by way of example, a gene is isolated when separated from open reading frames which flank the gene and encode a protein other than the gene of interest.

The term “label,” as used herein, refers to a substance which is incorporated into a compound and is readily detected, whereby its physical distribution may be detected and/or monitored.

The term “linkage” or “linker” as used herein to refer to bonds or chemical moiety formed from a chemical reaction between the functional group of a payload, drug and/or linker and another molecule. Such bonds may include, but are not limited to, covalent linkages and non-covalent bonds, while such chemical moieties may include, but are not limited to, oximes, esters, carbonates, imines phosphate esters, hydrazones, acetals, orthoesters, peptide linkages, and oligonucleotide linkages. Hydrolytically stable linkages mean that the linkages are substantially stable in water and do not react with water at useful pH values, including but not limited to, under physiological conditions for an extended period of time, perhaps even indefinitely. Hydrolytically unstable or degradable linkages mean that the linkages are degradable in water or in aqueous solutions, including for example, blood. Enzymatically unstable or degradable linkages mean that the linkage can be degraded by one or more enzymes. By way of example only, PEG and related polymers may include degradable linkages in the polymer backbone or in the linker group between the polymer backbone and one or more of the terminal functional groups of the polymer molecule. Such degradable linkages include, but are not limited to, ester linkages formed by the reaction of PEG carboxylic acids or activated PEG carboxylic acids with alcohol groups on a biologically active agent, wherein such ester groups generally hydrolyze under physiological conditions to release the biologically active agent. Other hydrolytically degradable linkages include but are not limited to carbonate linkages; imine linkages resulted from reaction of an amine and an aldehyde; phosphate ester linkages formed by reacting an alcohol with a phosphate group; hydrazone linkages which are reaction product of a hydrazide and an aldehyde; acetal linkages that are the reaction product of an aldehyde and an alcohol; orthoester linkages that are the reaction product of a formate and an alcohol; peptide linkages formed by an amine group, including but not limited to, at an end of a polymer such as PEG, and a carboxyl group of a peptide; and oligonucleotide linkages formed by a phosphoramidite group, including but not limited to, at the end of a polymer, and a 5′ hydroxyl group of an oligonucleotide. Linkers include but are not limited to short linear, branched, multi-armed, or dendrimeric molecules such as polymers. In some embodiments of the disclosure the linker may be branched. In other embodiments the linker may be a bifunctional linker. In some embodiments, the linker may be a trifunctional linker. A number of different cleavable linkers are known to those of skill in the art. See U.S. Pat. Nos. 4,618,492; 4,542,225, and 4,625,014. The mechanisms for release of an agent from these linker groups include, for example, irradiation of a photolabile bond and acid-catalyzed hydrolysis. U.S. Pat. No. 4,671,958, for example, includes a description of immunoconjugates comprising linkers which are cleaved at the target site in vivo by the proteolytic enzymes of the patient's complement system. The length of the linker may be predetermined or selected depending upon a desired spatial relationship between the polypeptide and the molecule linked to it. In view of the large number of methods that have been reported for attaching a variety of radiodiagnostic compounds, radiotherapeutic compounds, drugs, toxins, and other agents to antibodies one skilled in the art will be able to determine a suitable method for attaching a given agent or molecule to a polypeptide.

The term “modified,” as used herein refers to the presence of a change to a natural amino acid, a non-natural amino acid, a natural amino acid polypeptide or a non-natural amino acid polypeptide. Such changes, or modifications, may be obtained by post synthesis modifications of natural amino acids, non-natural amino acids, natural amino acid polypeptides or non-natural amino acid polypeptides, or by co-translational, or by post-translational modification of natural amino acids, non-natural amino acids, natural amino acid polypeptides or non-natural amino acid polypeptides. The form “modified or unmodified” means that the natural amino acid, non-natural amino acid, natural amino acid polypeptide or non-natural amino acid polypeptide being discussed are optionally modified, that is, he natural amino acid, non-natural amino acid, natural amino acid polypeptide or non-natural amino acid polypeptide under discussion can be modified or unmodified.

As used herein, the term “modulated serum half-life” refers to positive or negative changes in the circulating half-life of a modified biologically active molecule relative to its non-modified form. By way of example, the modified biologically active molecules include, but are not limited to, natural amino acid, non-natural amino acid, natural amino acid polypeptide or non-natural amino acid polypeptide. By way of example, serum half-life is measured by taking blood samples at various time points after administration of the biologically active molecule or modified biologically active molecule, and determining the concentration of that molecule in each sample. Correlation of the serum concentration with time allows calculation of the serum half-life. By way of example, modulated serum half-life may be an increased in serum half-life, which may enable improved dosing regimens or avoid toxic effects. Such increases in serum may be at least about two fold, at least about three-fold, at least about five-fold, or at least about ten-fold. Methods to evaluate increases in serum half-life of any polypeptide are well known to the skilled artisan.

The term “modulated therapeutic half-life,” as used herein, refers to positive or negative change in the half-life of the therapeutically effective amount of a modified biologically active molecule, relative to its non-modified form. By way of example, the modified biologically active molecules include, but are not limited to, natural amino acid, non-natural amino acid, natural amino acid polypeptide or non-natural amino acid polypeptide. By way of example, therapeutic half-life is measured by measuring pharmacokinetic and/or pharmacodynamic properties of the molecule at various time points after administration. Increased therapeutic half-life may enable a particular beneficial dosing regimen, a particular beneficial total dose, or avoids an undesired effect. By way of example, the increased therapeutic half-life may result from increased potency, increased or decreased binding of the modified molecule to its target, an increase or decrease in another parameter or mechanism of action of the non-modified molecule, or an increased or decreased breakdown of the molecules by enzymes such as, by way of example only, proteases. Methods to evaluate increases in therapeutic half-life of any polypeptide are well known to the skilled artisan.

A “non-natural amino acid” refers to an amino acid that is not one of the 20 common amino acids or pyrrolysine or selenocysteine. Other terms that may be used synonymously with the term “non-natural amino acid” is “non-naturally encoded amino acid,” “unnatural amino acid,” “non-naturally-occurring amino acid,” and variously hyphenated and non-hyphenated versions thereof. The term “non-natural amino acid” includes, but is not limited to, amino acids which occur naturally by modification of a naturally encoded amino acid (including but not limited to, the 20 common amino acids or pyrrolysine and selenocysteine) but are not themselves incorporated into a growing polypeptide chain by the translation complex. Examples of such amino acids include, but are not limited to, N-acetylglucosaminyl-L-serine, N-acetylglucosaminyl-L-threonine, and O-phosphotyrosine. Additionally, the term “non-natural amino acid” includes, but is not limited to, amino acids which do not occur naturally and may be obtained synthetically or may be obtained by modification of non-natural amino acids. In some embodiments, non-natural amino acids comprise a lysine analog, for example, N6-azidoethoxy-L-lysine (AzK), N6-propargylethoxy-L-lysine (PraK), BCN-L-lysine, norbornene lysine, TCO-lysine, methyltetrazine lysine, or allyloxycarbonyl lysine. In some embodiments, non-natural amino acids comprise a saccharide moiety. Examples of such amino acids include N-acetyl-L-glucosaminyl-L-serine, N-acetyl-L-galactosaminyl-L-serine, N-acetyl-L-glucosaminyl-L-threonine, N-acetyl-L-glucosaminyl-L-asparagine and O-mannosaminyl-L-serine. Examples of such amino acids also include examples where the naturally-occurring N- or O-linkage between the amino acid and the saccharide is replaced by a covalent linkage not commonly found in nature—including but not limited to, an alkene, an oxime, a thioether, an amide and the like. Examples of such amino acids also include saccharides that are not commonly found in naturally-occurring proteins such as 2-deoxy-glucose, 2-deoxygalactose and the like. Specific examples of non-natural amino acids include, but are not limited to, a p-acetyl-L-phenylalanine, a p-propargyloxyphenylalanine, O-methyl-L-tyrosine, an L-3-(2-naphthyl)alanine, a 3-methyl-phenylalanine, an O-4-allyl-L-tyrosine, a 4-propyl-L-tyrosine, a tri-O-acetyl-GlcNAcp-serine, an L-Dopa, a fluorinated phenylalanine, a isopropyl-L-phenylalanine, a p-azido-L-phenylalanine, a p-acyl-L-phenylalanine, a p-benzoyl-L-phenylalanine, a L-phosphoserine, a phosphonoserine, a phosphonotyrosine, a p-iodo-phenylalanine, a p-bromophenylalanine, a p-amino-L-phenylalanine, a p-propargyloxy-L-phenylalanine, a 4-azido-L-phenylalanine, a para-azidoethoxy phenylalanine, and a para-azidomethyl-phenylalanine, and the like. In some embodiments, the non-natural amino acid is selected from a group consisting of para-acetyl-phenylalanine, 4-azido-L-phenylalanine, para-azidoethoxy phenylalanine or para-azidomethyl-phenylalanine.

The term “nucleic acid,” as used herein, refers to deoxyribonucleotides, deoxyribonucleosides, ribonucleosides or ribonucleotides and polymers thereof in either single- or double-stranded form. By way of example only, such nucleic acids and nucleic acid polymers include, but are not limited to, (i) analogues of natural nucleotides which have similar binding properties as a reference nucleic acid and are metabolized in a manner similar to naturally occurring nucleotides; (ii) oligonucleotide analogs including, but are not limited to, PNA (peptidonucleic acid), analogs of DNA used in antisense technology (phosphorothioates, phosphoroamidates, and the like); (iii) conservatively modified variants thereof (including but not limited to, degenerate codon substitutions) and complementary sequences and sequence explicitly indicated. By way of example, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “oxidizing agent,” as used herein, refers to a compound or material which is capable of removing an electron from a compound being oxidized. By way of example oxidizing agents include, but are not limited to, oxidized glutathione, cystine, cystamine, oxidized dithiothreitol, oxidized erythreitol, and oxygen. A wide variety of oxidizing agents are suitable for use in the methods and compositions described herein.

The term “pharmaceutically acceptable”, as used herein, refers to a material, including but not limited, to a salt, carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The term “polyalkylene glycol,” or “poly(alkene glycol)” as used herein, refers to linear or branched polymeric polyether polyols. Such polyalkylene glycols, including, but are not limited to, polyethylene glycol, polypropylene glycol, polybutylene glycol, and derivatives thereof. Other exemplary embodiments are listed, for example, in commercial supplier catalogs, such as Shearwater Corporation's catalog “Polyethylene Glycol and Derivatives for Biomedical Applications” (2001). By way of example only, such polymeric polyether polyols have average molecular weights from about 0.1 kDa to about 100 kDa. By way of example, such polymeric polyether polyols include, but are not limited to, from about 100 Da and about 100,000 Da or more. The molecular weight of the polymer may be from about 100 Da and about 100,000 Da, including but not limited to, about 100,000 Da, about 95,000 Da, about 90,000 Da, about 85,000 Da, about 80,000 Da, about 75,000 Da, about 70,000 Da, about 65,000 Da, about 60,000 Da, about 55,000 Da, about 50,000 Da, about 45,000 Da, about 40,000 Da, about 35,000 Da, about 30,000 Da, about 25,000 Da, about 20,000 Da, about 15,000 Da, about 10,000 Da, about 9,000 Da, about 8,000 Da, about 7,000 Da, about 6,000 Da, about 5,000 Da, about 4,000 Da, about 3,000 Da, about 2,000 Da, about 1,000 Da, about 900 Da, about 800 Da, about 700 Da, about 600 Da, about 500 Da, 400 Da, about 300 Da, about 200 Da, and about 100 Da. In some embodiments molecular weight of the polymer is from about 100 Da to about 50,000 Da. In some embodiments, the molecular weight of the polymer is from about 100 Da to about 40,000 Da. In some embodiments, the molecular weight of the polymer is from about 1,000 Da to about 40,000 Da. In some embodiments, the molecular weight of the polymer is from about 2,000 to about 50,000 Da. In some embodiments, the molecular weight of the polymer is from about 5,000 Da to about 40,000 Da. In some embodiments, the molecular weight of the polymer is from about 10,000 Da to about 40,000 Da. In some embodiments, the poly(ethylene glycol) molecule is a branched polymer. The molecular weight of the branched chain PEG may be from about 1,000 Da to about 100,000 Da, including but not limited to, about 100,000 Da, about 95,000 Da, about 90,000 Da, about 85,000 Da, about 80,000 Da, about 75,000 Da, about 70,000 Da, about 65,000 Da, about 60,000 Da, about 55,000 Da, about 50,000 Da, about 45,000 Da, about 40,000 Da, about 35,000 Da, about 30,000 Da, about 25,000 Da, about 20,000 Da, about 15,000 Da, about 10,000 Da, about 9,000 Da, about 8,000 Da, about 7,000 Da, about 6,000 Da, about 5,000 Da, about 4,000 Da, about 3,000 Da, about 2,000 Da, and about 1,000 Da. In some embodiments, the molecular weight of the branched chain PEG is from about 1,000 Da to about 50,000 Da. In some embodiments, the molecular weight of the branched chain PEG is from about 1,000 Da to about 40,000 Da. In some embodiments, the molecular weight of the branched chain PEG is from about 5,000 Da to about 40,000 Da. In some embodiments, the molecular weight of the branched chain PEG is from about 5,000 Da to about 20,000 Da. In other embodiments, the molecular weight of the branched chain PEG is from about 2,000 to about 50,000 Da.

The term “polymer,” as used herein, refers to a molecule composed of repeated subunits. Such molecules include, but are not limited to, polypeptides, polynucleotides, or polysaccharides or polyalkylene glycols.

The terms “polypeptide,” “peptide” and “protein” are used interchangeably herein to refer to a polymer of amino acid residues. That is, a description directed to a polypeptide applies equally to a description of a peptide and a description of a protein, and vice versa. The terms apply to naturally occurring amino acid polymers as well as amino acid polymers in which one or more amino acid residues is a non-natural amino acid. Additionally, such “polypeptides,” “peptides” and “proteins” include amino acid chains of any length, including full length proteins, wherein the amino acid residues are linked by covalent peptide bonds.

The term “post-translationally modified” refers to any modification of a natural or non-natural amino acid which occurs after such an amino acid has been translationally incorporated into a polypeptide chain. Such modifications include, but are not limited to, co-translational in vivo modifications, co-translational in vitro modifications (such as in a cell-free translation system), post-translational in vivo modifications, and post-translational in vitro modifications.

The terms “prodrug” or “pharmaceutically acceptable prodrug,” as used herein, refers to an agent that is converted into the parent drug in vivo or in vitro, wherein which does not abrogate the biological activity or properties of the drug, and is relatively nontoxic, i.e., the material may be administered to an individual without causing undesirable biological effects or interacting in a deleterious manner with any of the components of the composition in which it is contained. Prodrugs are generally drug precursors that, following administration to a subject and subsequent absorption, are converted to an active, or a more active species via some process, such as conversion by a metabolic pathway. Some prodrugs have a chemical group present on the prodrug that renders it less active and/or confers solubility or some other property to the drug. Once the chemical group has been cleaved and/or modified from the prodrug the active drug is generated. Prodrugs are converted into active drug within the body through enzymatic or non-enzymatic reactions. Prodrugs may provide improved physiochemical properties such as better solubility, enhanced delivery characteristics, such as specifically targeting a particular cell, tissue, organ or ligand, and improved therapeutic value of the drug. The benefits of such prodrugs include, but are not limited to, (i) ease of administration compared with the parent drug; (ii) the prodrug may be bioavailable by oral administration whereas the parent is not; and (iii) the prodrug may also have improved solubility in pharmaceutical compositions compared with the parent drug. A pro-drug includes a pharmacologically inactive, or reduced-activity, derivative of an active drug. Prodrugs may be designed to modulate the amount of a drug or biologically active molecule that reaches a desired site of action through the manipulation of the properties of a drug, such as physiochemical, biopharmaceutical, or pharmacokinetic properties. An example, without limitation, of a prodrug would be a non-natural amino acid polypeptide which is administered as an ester (the “prodrug”) to facilitate transmittal across a cell membrane where water solubility is detrimental to mobility, but which then is metabolically hydrolyzed to the carboxylic acid, the active entity, once inside the cell where water solubility is beneficial. Prodrugs may be designed as reversible drug derivatives, for use as modifiers to enhance drug transport to site-specific tissues.

The term “prophylactically effective amount,” as used herein, refers that amount of a composition containing at least one non-natural amino acid polypeptide or at least one modified non-natural amino acid polypeptide prophylactically applied to a patient which will relieve to some extent one or more of the symptoms of a disease, condition or disorder being treated. In such prophylactic applications, such amounts may depend on the patient's state of health, weight, and the like. It is considered well within the skill of the art for one to determine such prophylactically effective amounts by routine experimentation, including, but not limited to, a dose escalation clinical trial.

The term “protected,” as used herein, refers to the presence of a “protecting group” or moiety that prevents reaction of the chemically reactive functional group under certain reaction conditions. The protecting group will vary depending on the type of chemically reactive group being protected. By way of example only, (i) if the chemically reactive group is an amine or a hydrazide, the protecting group may be selected from tert-butyloxycarbonyl (t-Boc) and 9-fluorenylmethoxycarbonyl (Fmoc); (ii) if the chemically reactive group is a thiol, the protecting group may be orthopyridyldisulfide; and (iii) if the chemically reactive group is a carboxylic acid, such as butanoic or propionic acid, or a hydroxyl group, the protecting group may be benzyl or an alkyl group such as methyl, ethyl, or tert-butyl.

By way of example only, blocking/protecting groups may be selected from:

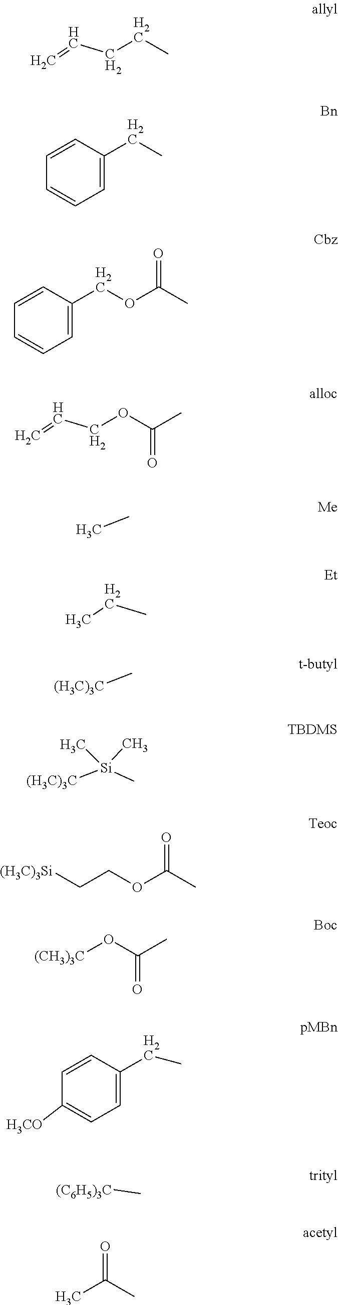

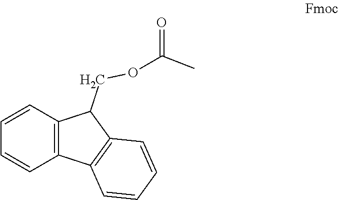 [SMILES: CC(=O)OCC1c2ccccc2-c2ccccc21]

Additionally, protecting groups include, but are not limited to, including photolabile groups such as Nvoc and MeNvoc and other protecting groups known in the art. Other protecting groups are described in Greene and Wuts, Protective Groups in Organic Synthesis, 3rd Ed., John Wiley & Sons, New York, NY, 1999, which is incorporated herein by reference in its entirety.

The term “recombinant host cell,” also referred to as “host cell,” refers to a cell which includes an exogenous polynucleotide, wherein the methods used to insert the exogenous polynucleotide into a cell include, but are not limited to, direct uptake, transduction, f-mating, or other methods known in the art to create recombinant host cells. By way of example only, such exogenous polynucleotide may be a nonintegrated vector, including but not limited to a plasmid, or may be integrated into the host genome.

The term “redox-active agent,” as used herein, refers to a molecule which oxidizes or reduces another molecule, whereby the redox active agent becomes reduced or oxidized. Examples of redox active agent include, but are not limited to, ferrocene, quinones, Ru^{2+/3+} complexes, Co^{2+/3+} complexes, and Os^{2+/3+} complexes.

The term “reducing agent,” as used herein, refers to a compound or material which is capable of adding an electron to a compound being reduced. By way of example reducing agents include, but are not limited to, dithiothreitol (DTT), 2-mercaptoethanol, dithioerythritol, cysteine, cysteamine (2-aminoethanethiol), and reduced glutathione. Such reducing agents may be used, by way of example only, to maintain sulfhydryl groups in the reduced state and to reduce intra- or intermolecular disulfide bonds.

“Refolding,” as used herein describes any process, reaction or method which transforms an improperly folded or unfolded state to a native or properly folded conformation. By way of example only, refolding transforms disulfide bond containing polypeptides from an improperly folded or unfolded state to a native or properly folded conformation with respect to disulfide bonds. Such disulfide bond containing polypeptides may be natural amino acid polypeptides or non-natural amino acid polypeptides.

The term “safety” or “safety profile,” as used herein, refers to side effects that might be related to administration of a drug relative to the number of times the drug has been administered. By way of example, a drug which has been administered many times and produced only mild or no side effects is said to have an excellent safety profile. Methods used for evaluating the safety profile of any polypeptide are known in the art.

The phrase “selectively hybridizes to” or “specifically hybridizes to,” as used herein, refers to the binding, duplexing, or hybridizing of a molecule to a particular nucleotide sequence under stringent hybridization conditions when that sequence is present in a complex mixture including but not limited to, total cellular or library DNA or RNA.

The phrase “stringent hybridization conditions” refers to hybridization of sequences of DNA, RNA, PNA or other nucleic acid mimics, or combinations thereof, under conditions of low ionic strength and high temperature. By way of example, under stringent conditions a probe will hybridize to its target subsequence in a complex mixture of nucleic acid (including but not limited to, total cellular or library DNA or RNA) but does not hybridize to other sequences in the complex mixture. Stringent conditions are sequence-dependent and will be different in different circumstances. By way of example, longer sequences hybridize specifically at higher temperatures. Stringent hybridization conditions include, but are not limited to, (i) about 5-10° C. lower than the thermal melting point (Tm) for the specific sequence at a defined ionic strength and pH; (ii) the salt concentration is about 0.01 M to about 1.0 M at about pH 7.0 to about pH 8.3 and the temperature is at least about 30° C. for short probes (including but not limited to, about 10 to about 50 nucleotides) and at least about 60° C. for long probes (including but not limited to, greater than 50 nucleotides); (iii) the addition of destabilizing agents including, but not limited to, formamide, (iv) 50% formamide, 5×SSC, and 1% SDS, incubating at 42° C., or 5×SSC, about 1% SDS, incubating at 65° C., with wash in 0.2×SSC, and about 0.1% SDS at 65° C. for from about 5 minutes to about 120 minutes. By way of example only, detection of selective or specific hybridization, includes, but is not limited to, a positive signal at least two times background. An extensive guide to the hybridization of nucleic acids is found in Tijssen, Laboratory Techniques in Biochemistry and Molecular Biology--Hybridization with Nucleic Probes, “Overview of principles of hybridization and the strategy of nucleic acid assays” (1993).

The term “subject” as used herein, refers to an animal which is the object of treatment, observation or experiment. By way of example only, a subject may be, but is not limited to, a mammal including, but not limited to, a human.

The term “substantially purified,” as used herein, refers to a component of interest that may be substantially or essentially free of other components which normally accompany or interact with the component of interest prior to purification. By way of example only, a component of interest may be “substantially purified” when the preparation of the component of interest contains less than about 30%, less than about 25%, less than about 20%, less than about 15%, less than about 10%, less than about 5%, less than about 4%, less than about 3%, less than about 2%, or less than about 1% (by dry weight) of contaminating components. Thus, a “substantially purified” component of interest may have a purity level of about 70%, about 75%, about 80%, about 85%, about 90%, about 95%, about 96%, about 97%, about 98%, about 99% or greater. By way of example only, a natural amino acid polypeptide or a non-natural amino acid polypeptide may be purified from a native cell, or host cell in the case of recombinantly produced natural amino acid polypeptides or non-natural amino acid polypeptides. By way of example a preparation of a natural amino acid polypeptide or a non-natural amino acid polypeptide may be “substantially purified” when the preparation contains less than about 30%, less than about 25%, less than about 20%, less than about 15%, less than about 10%, less than about 5%, less than about 4%, less than about 3%, less than about 2%, or less than about 1% (by dry weight) of contaminating material. By way of example when a natural amino acid polypeptide or a non-natural amino acid polypeptide is recombinantly produced by host cells, the natural amino acid polypeptide or non-natural amino acid polypeptide may be present at about 30%, about 25%, about 20%, about 15%, about 10%, about 5%, about 4%, about 3%, about 2%, or about 1% or less of the dry weight of the cells. By way of example when a natural amino acid polypeptide or a non-natural amino acid polypeptide is recombinantly produced by host cells, the natural amino acid polypeptide or non-natural amino acid polypeptide may be present in the culture medium at about 5 g/L, about 4 g/L, about 3 g/L, about 2 g/L, about 1 g/L, about 750 mg/L, about 500 mg/L, about 250 mg/L, about 100 mg/L, about 50 mg/L, about 10 mg/L, or about 1 mg/L or less of the dry weight of the cells. By way of example, “substantially purified” natural amino acid polypeptides or non-natural amino acid polypeptides may have a purity level of about 30%, about 35%, about 40%, about 45%, about 50%, about 55%, about 60%, about 65%, about 70%, about 75%, about 80%, about 85%, about 90%, about 95%, about 99% or greater as determined by appropriate methods, including, but not limited to, SDS/PAGE analysis, RP-HPLC, SEC, and capillary electrophoresis.

The term “substituents” also referred to as “non-interfering substituents” “refers to groups which may be used to replace another group on a molecule. Such groups include, but are not limited to, halo, C₁-C₁₀ alkyl, C₂-C₁₀ alkenyl, C₂-C₁₀ alkynyl, C₁-C₁₀ alkoxy, C₅-C₁₂ aralkyl, C₃-C₁₂ cycloalkyl, C₄-C₁₂ cycloalkenyl, phenyl, substituted phenyl, toluolyl, xylenyl, biphenyl, C₂-C₁₂ alkoxyalkyl, C₅-C₁₂ alkoxyaryl, C₅-C₁₂ aryloxyalkyl, C₇-C₁₂ oxyaryl, C₁-C₆ alkylsulfinyl, C₁-C₁₀ alkylsulfonyl, -(CH₂)m-O—(C₁-C₁₀ alkyl) wherein m is from 1 to 8, aryl, substituted aryl, substituted alkoxy, fluoroalkyl, heterocyclic radical, substituted heterocyclic radical, nitroalkyl, —NO₂, —CN, —NRC(O)-(C₁-C₁₀ alkyl), —C(O)-(C₁-C₁₀ alkyl), C₂-C₁₀ alkthioalkyl, —C(O)O—(C₁-C₁₀ alkyl), —OH, —SO₂, ═S, —COOH, —NR₂, carbonyl, —C(O)—(C₁-C₁₀ alkyl)-CF₃, —C(O)—CF₃, —C(O)NR₂, —(C₁-C₁₀ aryl)-S—(C₆-C₁₀ aryl), —C(O)—(C₆-C₁₀ aryl), —(CH₂)ₘ—O—(CH₂)ₘ—O—(C₁-C₁₀ alkyl) wherein each m is from 1 to 8, —C(O)NR₂, —C(S)NR₂, —SO₂NR₂, —NRC(O)NR₂, —NRC(S)NR₂, salts thereof, and the like. Each R group in the preceding list includes, but is not limited to, H, alkyl or substituted alkyl, aryl or substituted aryl, or alkaryl. Where substituent groups are specified by their conventional chemical formulas, written from left to right, they equally encompass the chemically identical substituents that would result from writing the structure from right to left; for example, —CH₂O— is equivalent to —OCH₂—.

By way of example only, substituents for alkyl and heteroalkyl radicals (including those groups referred to as alkylene, alkenyl, heteroalkylene, heteroalkenyl, alkynyl, cycloalkyl, heterocycloalkyl, cycloalkenyl, and heterocycloalkenyl) includes, but is not limited to: —OR, ═O, ═NR, ═N—OR, —NR₂, —SR, -halogen, —SiR₃, —OC(O)R, —C(O)R, —CO₂R, —CONR₂, —OC(O)NR₂, —NRC(O)R, —NRC(O)NR₂, —NR(O)₂R, —NR—C(NR₂)=NR, —S(O)R, —S(O)₂R, —S(O)₂NR₂, —NRSO₂R, —CN and —NO₂. Each R group in the preceding list includes, but is not limited to, hydrogen, substituted or unsubstituted heteroalkyl, substituted or unsubstituted aryl, including but not limited to, aryl substituted with 1-3 halogens, substituted or unsubstituted alkyl, alkoxy or thioalkoxy groups, or aralkyl groups. When two R groups are attached to the same nitrogen atom, they can be combined with the nitrogen atom to form a 5-, 6-, or 7-membered ring. For example, —NR₂ is meant to include, but not be limited to, 1-pyrrolidinyl and 4-morpholinyl.

By way of example, substituents for aryl and heteroaryl groups include, but are not limited to, —OR, ═O, ═NR, ═N—OR, —NR₂, —SR, -halogen, —SiR₃, —OC(O)R, —C(O)R, —CO₂R, —CONR₂, —OC(O)NR₂, —NRC(O)R, —NRC(O)NR₂, —NR(O)₂R, —NR—C(NR₂)=NR, —S(O)R, —S(O)₂R, —S(O)₂NR₂, —NRSO₂R, —CN, —NO₂, —R, —N₃, —CH(Ph)₂, fluoro(C₁-C₄)alkoxy, and fluoro(C₁-C₄)alkyl, in a number ranging from zero to the total number of open valences on the aromatic ring system; and where each R group in the preceding list includes, but is not limited to, hydrogen, alkyl, heteroalkyl, aryl and heteroaryl.

The term “therapeutically effective amount,” as used herein, refers to the amount of a composition containing at least one non-natural amino acid polypeptide and/or at least one modified non-natural amino acid polypeptide administered to a patient already suffering from a disease, condition or disorder, sufficient to cure or at least partially arrest, or relieve to some extent one or more of the symptoms of the disease, disorder or condition being treated. The effectiveness of such compositions depends on conditions including, but not limited to, the severity and course of the disease, disorder or condition, previous therapy, the patient's health status and response to the drugs, and the judgment of the treating physician. By way of example only, therapeutically effective amounts may be determined by routine experimentation, including but not limited to a dose escalation clinical trial.

The term “thioalkoxy,” as used herein, refers to sulfur containing alkyl groups linked to molecules via an oxygen atom.

The term “toxic moiety” or “toxic group” as used herein, refers to a compound which can cause harm, disturbances, or death. Toxic moieties include, but are not limited to, auristatin, DNA minor groove binding agent, DNA minor groove alkylating agent, enediyne, lexitropsin, duocarmycin, taxane, puromycin, cytotoxic tubulin inhibitor, maytansinoid, vinca alkaloid, AFP, MMAF, MMAE, AEB, AEVB, auristatin E, paclitaxel, docetaxel, CC-1065, SN-38, topotecan, morpholino-doxorubicin, rhizoxin, cyanomorpholino-doxorubicin, echinomycin, combretatstatin, chalicheamicin, maytansine, DM-1, netropsin, podophyllotoxin (e.g. etoposide, teniposide, etc.), baccatin and its derivatives, anti-tubulin agents, cryptophysin, combretastatin, vincristine, vinblastine, vindesine, vinorelbine, VP-16, camptothecin, epothilone A, epothilone B, nocodazole, colchicines, colcimid, estramustine, cemadotin, discodermolide, maytansine, eleutherobin, mechlorethamine, cyclophosphamide, melphalan, carmustine, lomustine, semustine, streptozocin, chlorozotocin, uracil mustard, chlormethine, ifosfamide, chlorambucil, pipobroman, triethylenemelamine, triethylenethiophosphoramine, busulfan, dacarbazine, and temozolomide, ytarabine, cytosine arabinoside, fluorouracil, floxuridine, 6-thioguanine, 6-mercaptopurine, pentostatin, 5-fluorouracil, methotrexate, 10-propargyl-5,8-dideazafolate, 5,8-dideazatetrahydrofolic acid, leucovorin, fludarabine phosphate, pentostatine, gemcitabine, Ara-C, paclitaxel, docetaxel, deoxycoformycin, mitomycin-C, L-asparaginase, azathioprine, brequinar, antibiotics (e.g., anthracycline, gentamicin, cefalotin, vancomycin, telavancin, daptomycin, azithromycin, erythromycin, rocithromycin, furazolidone, amoxicillin, ampicillin, carbenicillin, flucloxacillin, methicillin, penicillin, ciprofloxacin, moxifloxacin, ofloxacin, doxycycline, minocycline, oxytetracycline, tetracycline, streptomycin, rifabutin, ethambutol, rifaximin, etc.), antiviral drugs (e.g., abacavir, acyclovir, ampligen, cidofovir, delavirdine, didanosine, efavirenz, entecavir, fosfonet, ganciclovir, ibacitabine, imunovir, idoxuridine, inosine, lopinavir, methisazone, nexavir, nevirapine, oseltamivir, penciclovir, stavudine, trifluridine, truvada, valaciclovir, zanamivir, etc.), daunorubicin hydrochloride, daunomycin, rubidomycin, cerubidine, idarubicin, doxorubicin, epirubicin and morpholino derivatives, phenoxizone biscyclopeptides (e.g., dactinomycin), basic glycopeptides (e.g., bleomycin), anthraquinone glycosides (e.g., plicamycin, mithramycin), anthracenediones (e.g., mitoxantrone), azirinopyrrolo indolediones (e.g., mitomycin), macrocyclic immunosuppressants (e.g., cyclosporine, FK-506, tacrolimus, prograf, rapamycin etc.), navelbene, CPT-11, anastrazole, letrazole, capecitabine, reloxafine, ifosamide, droloxafine, allocolchicine, Halichondrin B, colchicine, colchicine derivatives, maytansine, rhizoxin, paclitaxel, paclitaxel derivatives, docetaxel, thiocolchicine, trityl cysterin, vinblastine sulfate, vincristine sulfate, cisplatin, carboplatin, hydroxyurea, N-methylhydrazine, epidophyllotoxin, procarbazine, mitoxantrone, leucovorin, and tegafur. “Taxanes” include paclitaxel, as well as any active taxane derivative or pro-drug.

The terms “treat,” “treating” or “treatment”, as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms “treat,” “treating” or “treatment”, include, but are not limited to, prophylactic and/or therapeutic treatments.

As used herein, the term “water soluble polymer” refers to any polymer that is soluble in aqueous solvents. Such water soluble polymers include, but are not limited to, polyethylene glycol, polyethylene glycol propionaldehyde, mono C₁-C₁₀ alkoxy or aryloxy derivatives thereof (described in U.S. Pat. No. 5,252,714 which is incorporated by reference herein), monomethoxy-polyethylene glycol, polyvinyl pyrrolidone, polyvinyl alcohol, polyamino acids, divinylether maleic anhydride, N-(2-Hydroxypropyl)-methacrylamide, dextran, dextran derivatives including dextran sulfate, polypropylene glycol, polypropylene oxide/ethylene oxide copolymer, polyoxyethylated polyol, heparin, heparin fragments, polysaccharides, oligosaccharides, glycans, cellulose and cellulose derivatives, including but not limited to methylcellulose and carboxymethyl cellulose, serum albumin, starch and starch derivatives, polypeptides, polyalkylene glycol and derivatives thereof, copolymers of polyalkylene glycols and derivatives thereof, polyvinyl ethyl ethers, and alpha-beta-poly[(2-hydroxyethyl)-DL-aspartamide, and the like, or mixtures thereof. By way of example only, coupling of such water soluble polymers to natural amino acid polypeptides or non-natural polypeptides may result in changes including, but not limited to, increased water solubility, increased or modulated serum half-life, increased or modulated therapeutic half-life relative to the unmodified form, increased bioavailability, modulated biological activity, extended circulation time, modulated immunogenicity, modulated physical association characteristics including, but not limited to, aggregation and multimer formation, altered receptor binding, activity modulator, or other targeting polypeptide binding, altered binding to one or more binding partners, and altered targeting polypeptide receptor dimerization or multimerization. In addition, such water soluble polymers may or may not have their own biological activity, and may be utilized as a linker for attaching targeting polypeptide to other substances, including but not limited to one or more targeting polypeptides, or one or more biologically active molecules.

### General Description of Synthesis/Testing

Unless otherwise indicated, conventional methods of mass spectroscopy, NMR, HPLC, protein chemistry, biochemistry, recombinant DNA techniques and pharmacology, within the skill of the art are employed.

Compounds, (including, but not limited to non-natural amino acids, non-natural amino acid polypeptides, modified non-natural amino acid polypeptides, and reagents for producing the aforementioned compounds) presented herein include isotopically-labeled compounds, which are identical to those recited in the various formulas and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, fluorine and chlorine, such as ²H, ³H, ¹³C, ¹⁴C, ¹⁵N, ¹⁸O, ¹⁷O, S, ¹⁸F, ³⁶Cl, respectively. Certain isotopically-labeled compounds described herein, for example those into which radioactive isotopes such as ³H and ¹⁴C are incorporated, are useful in drug and/or substrate tissue distribution assays. Further, substitution with isotopes such as deuterium, i.e., ²H, can afford certain therapeutic advantages resulting from greater metabolic stability, for example increased in vivo half-life or reduced dosage requirements.

Some of the compounds herein (including, but not limited to non-natural amino acids, non-natural amino acid polypeptides and modified non-natural amino acid polypeptides, and reagents for producing the aforementioned compounds) have asymmetric carbon atoms and can therefore exist as enantiomers or diastereomers. Diasteromeric mixtures can be separated into their individual diastereomers on the basis of their physical chemical differences by methods known, for example, by chromatography and/or fractional crystallization. Enantiomers can be separated by converting the enantiomeric mixture into a diastereomeric mixture by reaction with an appropriate optically active compound (e.g., alcohol), separating the diastereomers and converting (e.g., hydrolyzing) the individual diastereomers to the corresponding pure enantiomers. All such isomers, including diastereomers, enantiomers, and mixtures thereof are considered as part of the compositions described herein.

In additional or further embodiments, the compounds described herein (including, but not limited to non-natural amino acids, non-natural amino acid polypeptides and modified non-natural amino acid polypeptides, and reagents for producing the aforementioned compounds) are used in the form of pro-drugs. In additional or further embodiments, the compounds described herein (including, but not limited to non-natural amino acids, non-natural amino acid polypeptides and modified non-natural amino acid polypeptides, and reagents for producing the aforementioned compounds) are metabolized upon administration to an organism in need to produce a metabolite that is then used to produce a desired effect, including a desired therapeutic effect. In further or additional embodiments are active metabolites of non-natural amino acids and “modified or unmodified” non-natural amino acid polypeptides.

The methods and formulations described herein include the use of N-oxides, crystalline forms (also known as polymorphs), or pharmaceutically acceptable salts of non-natural amino acids, non-natural amino acid polypeptides and modified non-natural amino acid polypeptides. In certain embodiments, non-natural amino acids, non-natural amino acid polypeptides and modified non-natural amino acid polypeptides may exist as tautomers. All tautomers are included within the scope of the non-natural amino acids, non-natural amino acid polypeptides and modified non-natural amino acid polypeptides presented herein. In addition, the non-natural amino acids, non-natural amino acid polypeptides and modified non-natural amino acid polypeptides described herein can exist in unsolvated as well as solvated forms with pharmaceutically acceptable solvents such as water, ethanol, and the like. The solvated forms of the non-natural amino acids, non-natural amino acid polypeptides and modified non-natural amino acid polypeptides presented herein are also considered to be disclosed herein.

Some of the compounds herein (including, but not limited to non-natural amino acids, non-natural amino acid polypeptides and modified non-natural amino acid polypeptides and reagents for producing the aforementioned compounds) may exist in several tautomeric forms. All such tautomeric forms are considered as part of the compositions described herein. Also, for example all enol-keto forms of any compounds (including, but not limited to non-natural amino acids, non-natural amino acid polypeptides and modified non-natural amino acid polypeptides and reagents for producing the aforementioned compounds) herein are considered as part of the compositions described herein.

Some of the compounds herein (including, but not limited to non-natural amino acids, non-natural amino acid polypeptides and modified non-natural amino acid polypeptides and reagents for producing either of the aforementioned compounds) are acidic and may form a salt with a pharmaceutically acceptable cation. Some of the compounds herein (including, but not limited to non-natural amino acids, non-natural amino acid polypeptides and modified non-natural amino acid polypeptides and reagents for producing the aforementioned compounds) can be basic and accordingly, may form a salt with a pharmaceutically acceptable anion. All such salts, including di-salts are within the scope of the compositions described herein and they can be prepared by conventional methods. For example, salts can be prepared by contacting the acidic and basic entities, in either an aqueous, non-aqueous or partially aqueous medium. The salts are recovered by using at least one of the following techniques: filtration, precipitation with a non-solvent followed by filtration, evaporation of the solvent, or, in the case of aqueous solutions, lyophilization.

Pharmaceutically acceptable salts of the non-natural amino acid polypeptides disclosed herein may be formed when an acidic proton present in the parent non-natural amino acid polypeptides either is replaced by a metal ion, by way of example an alkali metal ion, an alkaline earth ion, or an aluminum ion; or coordinates with an organic base. In addition, the salt forms of the disclosed non-natural amino acid polypeptides can be prepared using salts of the starting materials or intermediates. The non-natural amino acid polypeptides described herein may be prepared as a pharmaceutically acceptable acid addition salt (which is a type of a pharmaceutically acceptable salt) by reacting the free base form of non-natural amino acid polypeptides described herein with a pharmaceutically acceptable inorganic or organic acid. Alternatively, the non-natural amino acid polypeptides described herein may be prepared as pharmaceutically acceptable base addition salts (which are a type of a pharmaceutically acceptable salt) by reacting the free acid form of non-natural amino acid polypeptides described herein with a pharmaceutically acceptable inorganic or organic base.

The type of pharmaceutical acceptable salts, include, but are not limited to: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine, tromethamine, N-methylglucamine, and the like. Acceptable inorganic bases include aluminum hydroxide, calcium hydroxide, potassium hydroxide, sodium carbonate, sodium hydroxide, and the like.

The corresponding counterions of the non-natural amino acid polypeptide pharmaceutical acceptable salts may be analyzed and identified using various methods including, but not limited to, ion exchange chromatography, ion chromatography, capillary electrophoresis, inductively coupled plasma, atomic absorption spectroscopy, mass spectrometry, or any combination thereof. In addition, the therapeutic activity of such non-natural amino acid polypeptide pharmaceutical acceptable salts may be tested using the techniques and methods described in the examples.

It should be understood that a reference to a salt includes the solvent addition forms or crystal forms thereof, particularly solvates or polymorphs. Solvates contain either stoichiometric or non-stoichiometric amounts of a solvent, and are often formed during the process of crystallization with pharmaceutically acceptable solvents such as water, ethanol, and the like. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

The screening and characterization of non-natural amino acid polypeptide pharmaceutical acceptable salts polymorphs and/or solvates may be accomplished using a variety of techniques including, but not limited to, thermal analysis, x-ray diffraction, spectroscopy, vapor sorption, and microscopy. Thermal analysis methods address thermo chemical degradation or thermo physical processes including, but not limited to, polymorphic transitions, and such methods are used to analyze the relationships between polymorphic forms, determine weight loss, to find the glass transition temperature, or for excipient compatibility studies. Such methods include, but are not limited to, Differential scanning calorimetry (DSC), Modulated Differential Scanning Calorimetry (MDCS), Thermogravimetric analysis (TGA), and Thermogravi-metric and Infrared analysis (TG/IR). X-ray diffraction methods include, but are not limited to, single crystal and powder diffractometers and synchrotron sources. The various spectroscopic techniques used include, but are not limited to, Raman, FTTR, UVIS, and NMR (liquid and solid state). The various microscopy techniques include, but are not limited to, polarized light microscopy, Scanning Electron Microscopy (SEM) with Energy Dispersive X-Ray Analysis (EDX), Environmental Scanning Electron Microscopy with EDX (in gas or water vapor atmosphere), IR microscopy, and Raman microscopy.

While preferred embodiments of the present disclosure have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Site-Specific Anti-HER2 Antibody-Drug Conjugates

Using a unique, clinically validated conjugation technology, drugs can be site specifically attached to an antibody via a stable oxime bond formed by conjugating a aminooxy containing drug-linker with a nonnatural amino acid, para-acetyl-phenylalanine (pAF), which can be incorporated into an antibody. This can result in precisely two drug-linkers conjugated site specifically to one antibody. pAF can be incorporated into the most surface-exposed sites of an antibody for screening optimal conjugation sites, an advantage compared with other site-specific conjugation technologies.

In some cases, the present disclosure provides a next-generation, site-specific anti-HER2 ADC comprising a humanized HER2 targeting monoclonal antibody (mAb) conjugated to a cytotoxic tubulin inhibitor using a nonnatural amino acid incorporation technology platform. The ADC thus synthesized can be homogeneous and highly stable, thereby leading to a wider therapeutic window by delivering drug to target tumor cells with higher efficiency, maximizing on-target efficacy and minimizing off-target toxicity.

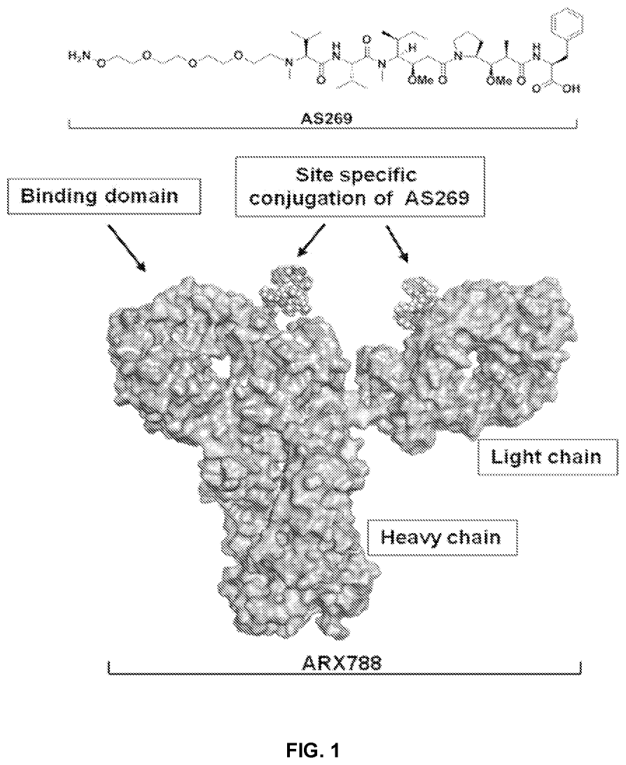

In some cases, the ADC comprises a short, noncleavable aminooxy-PEG4 linker attached to the N-terminus of monomethyl auristatin F (MMAF) to produce a cytotoxic tubulin inhibitor linker derivative AS269, which is a cytotoxic payload. MMAF is a highly potent synthetic auristatin derivative that inhibits cellular proliferation by disrupting tubulin polymerization. In some cases, the ADC contains two AS269 cytotoxic payloads site-specifically conjugated to a trastuzumab-based antibody comprising non-natural amino acids. In some cases, the ADC may display anti-tumor activity by optimizing the number and position of the payloads and the chemical bonds that conjugate the payloads to the antibody. In some cases, AS269 is a generally non-cell permeable tubulin inhibitor specifically designed to form a highly stable covalent bond with an antibody and kill tumor cells only upon entry into the cell when aided by the conjugated targeting antibody, thereby limiting off-target effects on healthy tissue. In some cases, AS269 displays limited to no permeability to cross the cell membrane without being conjugated with the targeting antibody, thereby reducing off-target toxicity. In some cases, AS269 is a poor substrate for multidrug resistance pumps (MDRs), thereby retaining and enriching the drug inside the cancer cell, which might lead to more potent killing of cancer cells. Combining AS269's unique characteristics with an optimized number and position of the payloads and the chemical bonds that conjugate the payloads to the antibody with a DAR of 2 can result in enhanced in vivo stability, potency and low payload exposure in serum, which in turn may contribute to the ADC's observed anti-tumor activity and tolerability profile. In some cases, an ADC comprises a structure synthesized according to FIG. 1. In some cases, the ADC comprises AS269 as the payload, and an anti-HER2 antibody comprising one or more unnatural amino acids disclosed herein. In some cases, the AS269 payload is specifically and stably conjugated to the non-natural amino acid pAF on unique sites in the heavy chains of the mAb (one payload per heavy chain), the anti-HER2 antibody comprising the heavy chain constant region of anti-HER2 Fab with corresponding amino acid sequence, SEQ ID NO: 2 (Kabat numbering), and a light chain sequence with corresponding amino acid sequence, SEQ ID NO: 3. In some cases, the ADC comprises AS269 as the payload, and an anti-HER2 antibody comprising the heavy chain or light chain constant region of anti-HER2 Fab with their Kabat numbering and the corresponding amino acid heavy chain sequences, SEQ ID NO: 2, and light chain sequences SEQ ID Nos: 4 to 11.

### Cytotoxic Tubulin Inhibitor Linker Derivatives

At one level, described herein are the tools (methods, compositions, techniques) for creating and using a targeting polypeptide of the ADCs or analogs comprising at least one non-natural amino acid or modified non-natural amino acid with a carbonyl, dicarbonyl, oxime, aminooxy or hydroxylamine group. Such targeting polypeptide of the ADCs comprising non-natural amino acids may contain further functionality, including but not limited to, a polymer; a water-soluble polymer; a derivative of polyethylene glycol; a second protein or polypeptide or polypeptide analog; an antibody or antibody fragment; and any combination thereof. Note that the various aforementioned functionalities are not meant to imply that the members of one functionality cannot be classified as members of another functionality. Indeed, there will be overlap depending upon the particular circumstances. By way of example only, a water-soluble polymer overlaps in scope with a derivative of polyethylene glycol, however the overlap is not complete and thus both functionalities are cited above.

In one aspect are methods for selecting and designing a cytotoxic tubulin inhibitor linker derivative, and the targeting polypeptide, to be modified using the methods, compositions and techniques described herein. The new cytotoxic tubulin inhibitor linker derivative and the targeting polypeptide may be designed de novo, including by way of example only, as part of high-throughput screening process (in which case numerous polypeptides may be designed, synthesized, characterized and/or tested) or based on the interests of the researcher. The new cytotoxic tubulin inhibitor linker derivative and the targeting polypeptide may also be designed based on the structure of a known or partially characterized polypeptide. The principles for selecting which amino acid(s) to substitute and/or modify are described separately herein. The choice of which modification to employ is also described herein and can be used to meet the need of the experimenter or end user. Such needs may include, but are not limited to, manipulating the therapeutic effectiveness of the polypeptide, improving the safety profile of the polypeptide, adjusting the pharmacokinetics, pharmacologics and/or pharmacodynamics of the polypeptide, such as, by way of example only, increasing water solubility, bioavailability, increasing serum half-life, increasing therapeutic half-life, modulating immunogenicity, modulating biological activity, or extending the circulation time. In addition, such modifications include, by way of example only, providing additional functionality to the polypeptide, incorporating an antibody, and any combination of the aforementioned modifications.

Also described herein are cytotoxic tubulin inhibitor linker derivatives and the targeting polypeptide that have or can be modified to contain an oxime, carbonyl, dicarbonyl, aminooxy or hydroxylamine group. Included with this aspect are methods for producing, purifying, characterizing and using such cytotoxic tubulin inhibitor linker derivatives and the targeting polypeptides.

The cytotoxic tubulin inhibitor linker derivative or the targeting polypeptide may contain at least one, at least two, at least three, at least four, at least five, at least six, at least seven, at least eight, at least nine, or ten or more of a carbonyl or dicarbonyl group, oxime group, aminooxy group, hydroxylamine group, or protected forms thereof. The cytotoxic tubulin inhibitor linker derivative or the targeting polypeptide can be the same or different, for example, there can be 1, 2, 3, 4, 5, 6, 7, 8, 9, 10, 11, 12, 13, 14, 15, 16, 17, 18, 19, 20, or more different sites in the derivative that comprise 1, 2, 3, 4, 5, 6, 7, 8, 9, 10, 11, 12, 13, 14, 15, 16, 17, 18, 19, 20, or more different reactive groups.

As described herein, the present disclosures provide targeting polypeptides coupled to another molecule having the formula “targeting polypeptide-L-M”, wherein L is a linking group or a chemical bond, and M is any other molecule including but not limited to another targeting polypeptide. In some embodiments, L is stable in vivo. In some embodiments, L is hydrolyzable in vivo. In some embodiments, L is metastable in vivo.

Targeting polypeptide and M can be linked together through L using standard linking agents and procedures known to those skilled in the art. In some aspects, targeting polypeptide and M are fused directly and L is a bond. In other aspects, targeting polypeptide and M are fused through a linking group L. For example, in some embodiments, targeting polypeptide and M are linked together via a peptide bond, optionally through a peptide or amino acid spacer. In some embodiments, targeting polypeptide and M are linked together through chemical conjugation, optionally through a linking group (L). In some embodiments, L is directly conjugated to each of targeting polypeptide and M.

Chemical conjugation can occur by reacting a nucleophilic reactive group of one compound to an electrophilic reactive group of another compound. In some embodiments when L is a bond, targeting polypeptide is conjugated to M either by reacting a nucleophilic reactive moiety on targeting polypeptide with an electrophilic reactive moiety on Y, or by reacting an electrophilic reactive moiety on targeting polypeptide with a nucleophilic reactive moiety on M. In embodiments when L is a group that links targeting polypeptide and M together, targeting polypeptide and/or M can be conjugated to L either by reacting a nucleophilic reactive moiety on targeting polypeptide and/or M with an electrophilic reactive moiety on L, or by reacting an electrophilic reactive moiety on targeting polypeptide and/or M with a nucleophilic reactive moiety on L. Nonlimiting examples of nucleophilic reactive groups include amino, thiol, and hydroxyl. Nonlimiting examples of electrophilic reactive groups include carboxyl, acyl chloride, anhydride, ester, succinimide ester, alkyl halide, sulfonate ester, maleimido, haloacetyl, and isocyanate. In embodiments where targeting polypeptide and M are conjugated together by reacting a carboxylic acid with an amine, an activating agent can be used to form an activated ester of the carboxylic acid.

The activated ester of the carboxylic acid can be, for example, N-hydroxysuccinimide (NHS), tosylate (Tos), mesylate, triflate, a carbodiimide, or a hexafluorophosphate. In some embodiments, the carbodiimide is 1,3-dicyclohexylcarbodiimide (DCC), 1,1′-carbonyldiimidazole (CDI), 1-ethyl-3-(3-dimethylaminopropyl)carbodiimide hydrochloride (EDC), or 1,3-diisopropylcarbodiimide (DICD). In some embodiments, the hexafluorophosphate is selected from a group consisting of hexafluorophosphate benzotriazol-1-yl-oxy-tris(dimethylamino)phosphonium hexafluorophosphate (BOP), benzotriazol-1-yl-oxytripyrrolidinophosphonium hexafluorophosphate (PyBOP), 2-(1H-7-azabenzotriazol-1-yl)-1,1,3,3-tetramethyl uronium hexafluorophosphate (HATU), and o-benzotriazole-N,N,N′,N′-tetramethyl-uronium-hexafluoro-phosphate (HBTU).

In some embodiments, targeting polypeptide comprises a nucleophilic reactive group (e.g. the amino group, thiol group, or hydroxyl group of the side chain of lysine, cysteine or serine) that is capable of conjugating to an electrophilic reactive group on M or L. In some embodiments, targeting polypeptide comprises an electrophilic reactive group (e.g. the carboxylate group of the side chain of Asp or Glu) that is capable of conjugating to a nucleophilic reactive group on M or L. In some embodiments, targeting polypeptide is chemically modified to comprise a reactive group that is capable of conjugating directly to M or to L. In some embodiments, targeting polypeptide is modified at the N-terminus or C-terminus to comprise a natural or non-natural amino acid with a nucleophilic side chain. In exemplary embodiments, the N-terminus or C-terminus amino acid of targeting polypeptide is selected from the group consisting of lysine, ornithine, serine, cysteine, and homocysteine. For example, the N-terminus or C-terminus amino acid of targeting polypeptide can be modified to comprise a lysine residue. In some embodiments, targeting polypeptide is modified at the N-terminus or C-terminus amino acid to comprise a natural or non-natural amino acid with an electrophilic side chain such as, for example, Asp and Glu. In some embodiments, an internal amino acid of targeting polypeptide is substituted with a natural or non-natural amino acid having a nucleophilic side chain, as previously described herein. In exemplary embodiments, the internal amino acid of targeting polypeptide that is substituted is selected from the group consisting of lysine, ornithine, serine, cysteine, and homocysteine. For example, an internal amino acid of targeting polypeptide can be substituted with a lysine residue. In some embodiments, an internal amino acid of targeting polypeptide is substituted with a natural or non-natural amino acid with an electrophilic side chain, such as, for example, Asp and Glu.

In some embodiments, M comprises a reactive group that is capable of conjugating directly to targeting polypeptide or to L. In some embodiments, M comprises a nucleophilic reactive group (e.g. amine, thiol, hydroxyl) that is capable of conjugating to an electrophilic reactive group on targeting polypeptide or L. In some embodiments, M comprises electrophilic reactive group (e.g. carboxyl group, activated form of a carboxyl group, compound with a leaving group) that is capable of conjugating to a nucleophilic reactive group on targeting polypeptide or L. In some embodiments, M is chemically modified to comprise either a nucleophilic reactive group that is capable of conjugating to an electrophilic reactive group on targeting polypeptide or L. In some embodiments, M is chemically modified to comprise an electrophilic reactive group that is capable of conjugating to a nucleophilic reactive group on targeting polypeptide or L.

In some embodiments, conjugation can be carried out through organosilanes, for example, aminosilane treated with glutaraldehyde; carbonyldiimidazole (CDI) activation of silanol groups; or utilization of dendrimers. A variety of dendrimers are known in the art and include poly (amidoamine) (PAMAM) dendrimers, which are synthesized by the divergent method starting from ammonia or ethylenediamine initiator core reagents; a sub-class of PAMAM dendrimers based on a tris-aminoethylene-imine core; radially layered poly(amidoamine-organosilicon) dendrimers (PAMAMOS), which are inverted unimolecular micelles that consist of hydrophilic, nucleophilic polyamidoamine (PAMAM) interiors and hydrophobic organosilicon (OS) exteriors; Poly (Propylene Imine) (PPI) dendrimers, which are generally poly-alkyl amines having primary amines as end groups, while the dendrimer interior consists of numerous of tertiary tris-propylene amines; Poly (Propylene Amine) (POPAM) dendrimers; Diaminobutane (DAB) dendrimers; amphiphilic dendrimers; micellar dendrimers which are unimolecular micelles of water soluble hyper branched polyphenylenes; polylysine dendrimers; and dendrimers based on poly-benzyl ether hyper branched skeleton.

In some embodiments, conjugation can be carried out through olefin metathesis. In some embodiments, M and targeting polypeptide, M and L, or targeting polypeptide and L both comprise an alkene or alkyne moiety that is capable of undergoing metathesis. In some embodiments a suitable catalyst (e.g. copper, ruthenium) is used to accelerate the metathesis reaction. Suitable methods of performing olefin metathesis reactions are described in the art. See, for example, Schafmeister et al., J. Am. Chem. Soc. 122: 5891-5892 (2000), Walensky et al., Science 305: 1466-1470 (2004), and Blackwell et al., Angew, Chem., Int. Ed. 37: 3281-3284 (1998).

In some embodiments, conjugation can be carried out using click chemistry. A “click reaction” is wide in scope and easy to perform, uses only readily available reagents, and is insensitive to oxygen and water. In some embodiments, the click reaction is a cycloaddition reaction between an alkynyl group and an azido group to form a triazolyl group. In some embodiments, the click reaction uses a copper or ruthenium catalyst. Suitable methods of performing click reactions are described in the art. See, for example, Kolb et al., Drug Discovery Today 8: 1128 (2003); Kolb et al., Angew. Chem. Int. Ed. 40:2004 (2001); Rostovtsev et al., Angew. Chem. Int. Ed. 41:2596 (2002); Tornoe et al., J. Org. Chem. 67:3057 (2002); Manetsch et al., J. Am. Chem. Soc. 126: 12809 (2004); Lewis et al., Angew. Chem. Int. Ed. 41: 1053 (2002); Speers, J. Am. Chem. Soc. 125:4686 (2003); Chan et al. Org. Lett. 6:2853 (2004); Zhang et al., J. Am. Chem. Soc. 127: 15998 (2005); and Waser et al., J Am. Chem. Soc. 127:8294 (2005).

Indirect conjugation via high affinity specific binding partners, e.g. streptavidin/biotin or avidin/biotin or lectin/carbohydrate is also contemplated.

In some embodiments, targeting polypeptide and/or M are functionalized to comprise a nucleophilic reactive group or an electrophilic reactive group with an organic derivatizing agent. This derivatizing agent is capable of reacting with selected side chains or the N- or C-terminal residues of targeted amino acids on targeting polypeptide and functional groups on M. Reactive groups on targeting polypeptide and/or M include, e.g., aldehyde, amino, ester, thiol, a-haloacetyl, maleimido or hydrazino group. Derivatizing agents include, for example, maleimidobenzoyl sulfosuccinimide ester (conjugation through cysteine residues), N-hydroxysuccinimide (through lysine residues), glutaraldehyde, succinic anhydride or other agents known in the art. Alternatively, targeting polypeptide and/or M can be linked to each other indirectly through intermediate carriers, such as polysaccharide or polypeptide carriers. Examples of polysaccharide carriers include aminodextran. Examples of suitable polypeptide carriers include polylysine, polyglutamic acid, polyaspartic acid, co-polymers thereof, and mixed polymers of these amino acids and others, e.g., serines, to confer desirable solubility properties on the resultant loaded carrier.

Cysteinyl residues most commonly are reacted with a-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, alpha-bromo-β-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylpyrocarbonate at pH 5.5-7.0 because this agent is relatively specific for the histidyl side chain. Para-bromophenacyl bromide also is useful; the reaction is preferably performed in 0.1 M sodium cacodylate at pH 6.0.

Lysinyl and amino-terminal residues are reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate, pyridoxal phosphate, pyridoxal, chloroborohydride, trinitrobenzenesulfonic acid, O-methylisourea, 2,4-pentanedione, and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin.

Derivatization of arginine residues requires that the reaction be performed in alkaline conditions because of the high pKa of the guanidine functional group. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively.

Carboxyl side groups (aspartyl or glutamyl) are selectively modified by reaction with carbodiimides (R—N═C═N—R′), where R and R′ are different alkyl groups, such as 1-cyclohexyl-3-(2-morpholinyl-4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Other modifications include hydroxylation of proline and lysine, phosphorylation of hydroxyl groups of seryl or threonyl residues, methylation of the alpha-amino groups of lysine, arginine, and histidine side chains (T. E. Creighton, Proteins: Structure and Molecular Properties, W.H. Freeman & Co., San Francisco, pp. 79-86 (1983)), deamidation of asparagine or glutamine, acetylation of the N-terminal amine, and/or amidation or esterification of the C-terminal carboxylic acid group.

Another type of covalent modification involves chemically or enzymatically coupling glycosides to the peptide. Sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of tyrosine, or tryptophan, or (f) the amide group of glutamine. These methods are described in WO1987/05330, and in Aplin and Wriston, CRC Crit. Rev. Biochem., pp. 259-306 (1981).

In some embodiments, L is a bond. In these embodiments, targeting polypeptide and M are conjugated together by reacting a nucleophilic reactive moiety on targeting polypeptide with an electrophilic reactive moiety on M. In alternative embodiments, targeting polypeptide and M are conjugated together by reacting an electrophilic reactive moiety on targeting polypeptide with a nucleophilic moiety on M. In exemplary embodiments, L is an amide bond that forms upon reaction of an amine on targeting polypeptide (e.g. an ε-amine of a lysine residue) with a carboxyl group on M. In alternative embodiments, targeting polypeptide and or M is derivatized with a derivatizing agent before conjugation.

In some embodiments, L is a linking group. In some embodiments, L is a bifunctional linker and comprises only two reactive groups before conjugation to targeting polypeptide and M. In embodiments where both targeting polypeptide and M have electrophilic reactive groups, L comprises two of the same or two different nucleophilic groups (e.g. amine, hydroxyl, thiol) before conjugation to targeting polypeptide and M. In embodiments where both targeting polypeptide and M have nucleophilic reactive groups, L comprises two of the same or two different electrophilic groups (e.g. carboxyl group, activated form of a carboxyl group, compound with a leaving group) before conjugation to targeting polypeptide and M. In embodiments where one of targeting polypeptide or M has a nucleophilic reactive group and the other of targeting polypeptide or M has an electrophilic reactive group, L comprises one nucleophilic reactive group and one electrophilic group before conjugation to targeting polypeptide and M.

L can be any molecule with at least two reactive groups (before conjugation to targeting polypeptide and M) capable of reacting with each of targeting polypeptide and M. In some embodiments L has only two reactive groups and is bifunctional. L (before conjugation to the peptides) can be represented by Formula VI:

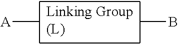

wherein A and B are independently nucleophilic or electrophilic reactive groups. In some embodiments, A and B are either both nucleophilic groups or both electrophilic groups. In some embodiments one of A or B is a nucleophilic group and the other of A or B is an electrophilic group. Nonlimiting combinations of A and B are shown below in Table 1.

TABLE 1
Nonlimiting combinations of Nucleophilic and Electrophilic Groups

| Both Nucleophilic | | Both Electrophilic | | Nucleophilic/Electrophilic | |
|---|---|---|---|---|---|
| A | B | A | B | A | B |
| Amino | Amino | Carboxyl | Carboxyl | Amino | Carboxyl |
| Amino | Thiol | Carboxyl | Acyl chloride | Amino | Acyl chloride |
| Amino | Hydroxyl | Carboxyl | Anhydride | Amino | Anhydride |
| Thiol | Amino | Carboxyl | Ester | Amino | Ester |
| Thiol | Thiol | Carboxyl | NHS | Amino | NHS |
| Thiol | Hydroxyl | Carboxyl | Halogen | Amino | Halogen |
| Hydroxyl | Amino | Carboxyl | Sulfonate ester | Amino | Sulfonate ester |
| Hydroxyl | Thiol | Carboxyl | Maleimido | Amino | Maleimido |
| Hydroxyl | Hydroxyl | Carboxyl | Haloacetyl | Amino | Haloacetyl |
| | | Carboxyl | Isocyanate | Amino | Isocyanate |
| | | Acyl chloride | Carboxyl | Thiol | Carboxyl |
| | | Acyl chloride | Acyl chloride | Thiol | Acyl chloride |
| | | Acyl chloride | Anhydride | Thiol | Anhydride |
| | | Acyl chloride | Ester | Thiol | Ester |
| | | Acyl chloride | NHS | Thiol | NHS |
| | | Acyl chloride | Halogen | Thiol | Halogen |
| | | Acyl chloride | Sulfonate ester | Thiol | Sulfonate ester |
| | | Acyl chloride | Maleimido | Thiol | Maleimido |
| | | Acyl chloride | Haloacetyl | Thiol | Haloacetyl |
| | | Acyl chloride | Isocyanate | Thiol | Isocyanate |
| | | Anhydride | Carboxyl | Hydroxyl | Carboxyl |
| | | Anhydride | Acyl chloride | Hydroxyl | Acyl chloride |
| | | Anhydride | Anhydride | Hydroxyl | Anhydride |
| | | Anhydride | Ester | Hydroxyl | Ester |
| | | Anhydride | NHS | Hydroxyl | NHS |
| | | Anhydride | Halogen | Hydroxyl | Halogen |
| | | Anhydride | Sulfonate ester | Hydroxyl | Sulfonate ester |
| | | Anhydride | Maleimido | Hydroxyl | Maleimido |
| | | Anhydride | Haloacetyl | Hydroxy1 | Haloacetyl |
| | | Anhydride | Isocyanate | Hydroxyl | Isocyanate |
| | | Ester | Carboxyl | | |
| | | Ester | Acyl chloride | | |
| | | Ester | Anhydride | | |
| | | Ester | Ester | | |
| | | Ester | NHS | | |
| | | Ester | Halogen | | |
| | | Ester | Sulfonate ester | | |
| | | Ester | Maleimido | | |
| | | Ester | Haloacetyl | | |
| | | Ester | Isocyanate | | |
| | | NHS | Carboxyl | | |
| | | NHS | Acyl chloride | | |
| | | NHS | Anhydride | | |
| | | NHS | Ester | | |
| | | NHS | NHS | | |
| | | NHS | Halogen | | |
| | | NHS | Sulfonate ester | | |
| | | NHS | Maleimido | | |
| | | NHS | Haloacetyl | | |
| | | NHS | Isocyanate | | |
| | | Halogen | Carboxyl | | |
| | | Halogen | Acyl chloride | | |
| | | Halogen | Anhydride | | |
| | | Halogen | Ester | | |
| | | Halogen | NHS | | |
| | | Halogen | Halogen | | |
| | | Halogen | Sulfonate ester | | |
| | | Halogen | Maleimido | | |
| | | Halogen | Haloacetyl | | |
| | | Halogen | Isocyanate | | |
| | | Sulfonate ester | Carboxyl | | |
| | | Sulfonate ester | Acyl chloride | | |
| | | Sulfonate ester | Anhydride | | |
| | | Sulfonate ester | Ester | | |
| | | Sulfonate ester | NHS | | |
| | | Sulfonate ester | Halogen | | |
| | | Sulfonate ester | Sulfonate ester | | |
| | | Sulfonate ester | Maleimido | | |
| | | Sulfonate ester | Haloacetyl | | |
| | | Sulfonate ester | Isocyanate | | |
| | | Maleimido | Carboxyl | | |
| | | Maleimido | Acyl chloride | | |
| | | Maleimido | Anhydride | | |
| | | Maleimido | Ester | | |
| | | Maleimido | NHS | | |
| | | Maleimido | Halogen | | |
| | | Maleimido | Sulfonate ester | | |
| | | Maleimido | Maleimido | | |
| | | Maleimido | Haloacetyl | | |
| | | Maleimido | Isocyanate | | |
| | | Haloacetyl | Carboxyl | | |
| | | Haloacetyl | Acyl chloride | | |
| | | Haloacetyl | Anhydride | | |
| | | Haloacetyl | Ester | | |
| | | Haloacetyl | NHS | | |
| | | Haloacetyl | Halogen | | |
| | | Haloacetyl | Sulfonate ester | | |
| | | Haloacetyl | Maleimido | | |
| | | Haloacetyl | Haloacetyl | | |
| | | Haloacetyl | Isocyanate | | |
| | | Isocyanate | Carboxyl | | |
| | | Isocyanate | Acyl chloride | | |
| | | Isocyanate | Anhydride | | |
| | | Isocyanate | Ester | | |
| | | Isocyanate | NHS | | |
| | | Isocyanate | Halogen | | |
| | | Isocyanate | Sulfonate ester | | |
| | | Isocyanate | Maleimido | | |
| | | Isocyanate | Haloacetyl | | |
| | | Isocyanate | Isocyanate | | |

In some embodiments, A and B may include alkene and/or alkyne functional groups that are suitable for olefin metathesis reactions. In some embodiments, A and B include moieties that are suitable for click chemistry (e.g. alkene, alkynes, nitriles, azides). Other nonlimiting examples of reactive groups (A and B) include pyridyldithiol, aryl azide, diazirine, carbodiimide, and hydrazide.

In some embodiments, L is hydrophobic. Hydrophobic linkers are known in the art. See, e.g., Bioconjugate Techniques, G. T. Hermanson (Academic Press, San Diego, CA, 1996), which is incorporated by reference in its entirety. Suitable hydrophobic linking groups known in the art include, for example, 8-hydroxy octanoic acid and 8-mercaptooctanoic acid. Before conjugation to the peptides of the composition, the hydrophobic linking group comprises at least two reactive groups (A and B), as described herein and as shown below:

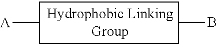

In some embodiments, the hydrophobic linking group comprises either a maleimido or an iodoacetyl group and either a carboxylic acid or an activated carboxylic acid (e.g. NHS ester) as the reactive groups. In these embodiments, the maleimido or iodoacetyl group can be coupled to a thiol moiety on targeting polypeptide or M and the carboxylic acid or activated carboxylic acid can be coupled to an amine on targeting polypeptide or M with or without the use of a coupling reagent. Any coupling agent known to one skilled in the art can be used to couple the carboxylic acid with the free amine such as, for example, DCC, DIC, HATU, HBTU, TBTU, and other activating agents described herein. In specific embodiments, the hydrophilic linking group comprises an aliphatic chain of 2 to 100 methylene groups wherein A and B are carboxyl groups or derivatives thereof (e.g. succinic acid). In other specific embodiments the L is iodoacetic acid.

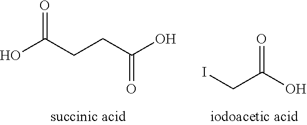 [SMILES: O=C(O)CCC(=O)O ; O=C(O)CI]

In some embodiments, the linking group is hydrophilic such as, for example, polyalkylene glycol. Before conjugation to the peptides of the composition, the hydrophilic linking group comprises at least two reactive groups (A and B), as described herein and as shown below:

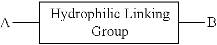

In specific embodiments, the linking group is polyethylene glycol (PEG). The PEG in certain embodiments has a molecular weight of about 100 Daltons to about 10,000 Daltons, e.g. about 500 Daltons to about 5000 Daltons. The PEG in some embodiments has a molecular weight of about 10,000 Daltons to about 40,000 Daltons.

In some embodiments, the hydrophilic linking group comprises either a maleimido or an iodoacetyl group and either a carboxylic acid or an activated carboxylic acid (e.g. NHS ester) as the reactive groups. In these embodiments, the maleimido or iodoacetyl group can be coupled to a thiol moiety on targeting polypeptide or M and the carboxylic acid or activated carboxylic acid can be coupled to an amine on targeting polypeptide or M with or without the use of a coupling reagent. Any appropriate coupling agent known to one skilled in the art can be used to couple the carboxylic acid with the amine such as, for example, DCC, DIC, HATU, HBTU, TBTU, and other activating agents described herein. In some embodiments, the linking group is maleimido-polymer(20-40 kDa)-COOH, iodoacetyl-polymer(20-40 kDa)-COOH, maleimido-polymer(20-40 kDa)-NHS, or iodoacetyl-polymer(20-40 kDa)-NHS.

In some embodiments, the linking group is comprised of an amino acid, a dipeptide, a tripeptide, or a polypeptide, wherein the amino acid, dipeptide, tripeptide, or polypeptide comprises at least two activating groups, as described herein. In some embodiments, the linking group (L) comprises a moiety selected from the group consisting of. amino, ether, thioether, maleimido, disulfide, amide, ester, thioester, alkene, cycloalkene, alkyne, trizoyl, carbamate, carbonate, cathepsin B-cleavable, and hydrazone.

In some embodiments, L comprises a chain of atoms from 1 to about 60, or 1 to 30 atoms or longer, 2 to 5 atoms, 2 to 10 atoms, 5 to 10 atoms, or 10 to 20 atoms long. In some embodiments, the chain atoms are all carbon atoms. In some embodiments, the chain atoms in the backbone of the linker are selected from the group consisting of C, O, N, and S. Chain atoms and linkers may be selected according to their expected solubility (hydrophilicity) so as to provide a more soluble conjugate. In some embodiments, L provides a functional group that is subject to cleavage by an enzyme or other catalyst or hydrolytic conditions found in the target tissue or organ or cell. In some embodiments, the length of L is long enough to reduce the potential for steric hindrance.

In some embodiments, L is stable in biological fluids such as blood or blood fractions. In some embodiments, L is stable in blood serum for at least 5 minutes, e.g. less than 25%, 20%, 15%, 10% or 5% of the conjugate is cleaved when incubated in serum for a period of 5 minutes. In other embodiments, L is stable in blood serum for at least 10, or 20, or 25, or 30, or 60, or 90, or 120 minutes, or 3, 4, 5, 6, 7, 8, 9, 10, 12, 15, 18 or 24 hours. In these embodiments, L does not comprise a functional group that is capable of undergoing hydrolysis in vivo. In some exemplary embodiments, L is stable in blood serum for at least about 72 hours. Nonlimiting examples of functional groups that are not capable of undergoing significant hydrolysis in vivo include amides, ethers, and thioethers. For example, the following compound (Q and Y represent two groups linked by the linker group L) does not undergoing significant hydrolysis in vivo:

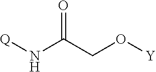

In some embodiments, L is hydrolyzable in vivo. In these embodiments, L comprises a functional group that is capable of undergoing hydrolysis in vivo. Nonlimiting examples of functional groups that are capable of undergoing hydrolysis in vivo include esters, anhydrides, and thioesters. For example the following compound (Q and Y represent two groups linked by the linker group L) is capable of undergoing hydrolysis in vivo because it comprises an ester group:

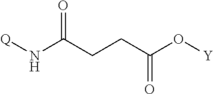

In some exemplary embodiments L is labile and undergoes substantial hydrolysis within 3 hours in blood plasma at 37° C., with complete hydrolysis within 6 hours. In some exemplary embodiments, L is not labile.

In some embodiments, L is metastable in vivo. In these embodiments, L comprises a functional group that is capable of being chemically or enzymatically cleaved in vivo (e.g., an acid-labile, reduction-labile, or enzyme-labile functional group), optionally over a period of time. In these embodiments, L can comprise, for example, a hydrazone moiety, a disulfide moiety, or a cathepsin-cleavable moiety. When L is metastable, and without intending to be bound by any particular theory, the targeting polypeptide-L-M conjugate is stable in an extracellular environment, e.g., stable in blood serum for the time periods described above, but labile in the intracellular environment or conditions that mimic the intracellular environment, so that it cleaves upon entry into a cell. In some embodiments when L is metastable, L is stable in blood serum for at least about 24, 25, 26, 27, 28, 29, 30, 31, 32, 33, 34, 35, 36, 42, or 48 hours, for example, at least about 48, 54, 60, 66, or 72 hours, or about 24-48, 48-72, 24-60, 36-48, 36-72, or 48-72 hours.

In another embodiment, the polymer derivatives of the disclosure comprise a polymer backbone having the structure:

X—CH₂CH₂O—(CH₂CH₂O)ₙ—CH₂CH₂—O—(CH₂)ₘ—W—N═N=N wherein:
W is an aliphatic or aromatic linker moiety comprising from 1 to 10 carbon atoms;
n is from 1 to about 4000; and X is a functional group as described above; m is from 1 to 10.

The azide-containing polymer derivatives of the disclosure can be prepared by a variety of methods known in the art and/or disclosed herein. In one method, shown below, a water soluble polymer backbone having an average molecular weight from about 800 Da to about 100,000 Da, the polymer backbone having a first terminus bonded to a first functional group and a second terminus bonded to a suitable leaving group, is reacted with an azide anion (which may be paired with any of a number of suitable counter-ions, including sodium, potassium, tert-butylammonium and so forth). The leaving group undergoes a nucleophilic displacement and is replaced by the azide moiety, affording the desired azide-containing polymer;

X-polymer-LY+N₃⁻→X-polymer-L N₃

As illustrated, a suitable polymer backbone for use in the present disclosure has the formula X-polymer-LY, wherein polymer is poly(ethylene glycol) and X is a functional group which does not react with azide groups and Y is a suitable leaving group. Examples of suitable functional groups include, but are not limited to, hydroxyl, protected hydroxyl, acetal, alkenyl, amine, aminooxy, protected amine, protected hydrazide, protected thiol, carboxylic acid, protected carboxylic acid, maleimide, dithiopyridine, and vinylpyridine, and ketone. Examples of suitable leaving groups include, but are not limited to, chloride, bromide, iodide, mesylate, tresylate, and tosylate.

In another method for preparation of the azide-containing polymer derivatives of the present disclosure, a linking agent bearing an azide functionality is contacted with a water soluble polymer backbone having an average molecular weight from about 800 Da to about 100,000 Da, wherein the linking agent bears a chemical functionality that will react selectively with a chemical functionality on the polymer to form an azide-containing polymer derivative product wherein the azide is separated from the polymer backbone by a linking group.

An exemplary reaction scheme is shown below:

X-polymer-Y+N-linker-N═N═N→PG-X-polymer-linker-N═N═N wherein:
polymer is poly(ethylene glycol) and X is a capping group such as alkoxy or a functional group as described above; and Y is a functional group that is not reactive with the azide functionality but that will react efficiently and selectively with the N functional group.

Examples of suitable functional groups include, but are not limited to, Y being a carboxylic acid, carbonate or active ester if N is an amine; Y being a ketone if N is a hydrazide or aminooxy moiety; Y being a leaving group if N is a nucleophile. Purification of the crude product may be accomplished by known methods including, but are not limited to, precipitation of the product followed by chromatography, if necessary.

A more specific example is shown below in the case of polymer diamine, in which one of the amines is protected by a protecting group moiety such as tert-butyl-Boc and the resulting mono-protected polymer diamine is reacted with a linking moiety that bears the azide functionality: BocHN-polymer-NH₂+HO₂C—(CH₂)₃—N═N═N

In this instance, the amine group can be coupled to the carboxylic acid group using a variety of activating agents such as thionyl chloride or carbodiimide reagents and N-hydroxysuccinimide or N-hydroxybenzotriazole to create an amide bond between the monoamine polymer derivative and the azide-bearing linker moiety. After successful formation of the amide bond, the resulting N-tert-butyl-Boc-protected azide-containing derivative can be used directly to modify bioactive molecules or it can be further elaborated to install other useful functional groups. For instance, the N-t-Boc group can be hydrolyzed by treatment with strong acid to generate an omega-amino-polymer-azide. The resulting amine can be used as a synthetic handle to install other useful functionality such as maleimide groups, activated disulfides, activated esters and so forth for the creation of valuable heterobifunctional reagents.

Heterobifunctional derivatives are particularly useful when it is desired to attach different molecules to each terminus of the polymer. For example, the omega-N-amino-N-azido polymer would allow the attachment of a molecule having an activated electrophilic group, such as an aldehyde, ketone, activated ester, activated carbonate and so forth, to one terminus of the polymer and a molecule having an acetylene group to the other terminus of the polymer.

In another embodiment of the present disclosure, A is an aliphatic linker of between 1-10 carbon atoms or a substituted aryl ring of between 6-14 carbon atoms. X is a functional group which does not react with azide groups and Y is a suitable leaving group.

Multiple targeting polypeptides may be joined by a linker polypeptide, wherein the linker polypeptide optionally is 6-14, 7-13, 8-12, 7-11, 9-11, or 9 amino acids in length. Other linkers include but are not limited to small polymers such as PEG, which may be multi-armed allowing for multiple targeting polypeptide molecules to be linked together. Multiple targeting polypeptides and modified targeting polypeptides may be linked to each other via their N-termini in a head-to-head configuration through the use of such a linker or by direct chemical bonding between the respective N-terminus of each polypeptide. For example, two targeting polypeptides may be linked to form a dimer by chemical bonding between their N-terminal amino groups or modified N-terminal amino groups. Also, a linking molecule that is designed to comprise multiple chemical functional groups for bonding with the N-terminus of each targeting polypeptide may be used to join multiple targeting polypeptides each at their respective N-terminus. In addition, multiple targeting polypeptides may be linked through bonding between amino acids other than the N-terminal amino acid or C-terminal amino acid. An example of covalent bonds that may be utilized to form the dimmers and multimers of targeting polypeptide that are described herein include, but are not limited to disulfide or sulfhydryl or thiol bonds. In addition, certain enzymes, such as sortase, may be used to form covalent bonds between the targeting polypeptides and the linker, including at the N-termini of the targeting polypeptides.

The linker may have a wide range of molecular weight or molecular length. Larger or smaller molecular weight linkers may be used to provide a desired spatial relationship or conformation between targeting polypeptide and the linked entity or between the linked entity and its binding partner, if any. Linkers having longer or shorter molecular length may also be used to provide a desired space or flexibility between targeting polypeptide and the linked entity, or between the linked entity and its binding partner.

In some embodiments, the disclosure provides water-soluble bifunctional linkers that have a dumbbell structure that includes: a) an azide, an alkyne, a hydrazine, a hydrazide, an aminooxy, a hydroxylamine, or a carbonyl-containing moiety on at least a first end of a polymer backbone; and b) at least a second functional group on a second end of the polymer backbone. The second functional group can be the same or different as the first functional group. The second functional group, in some embodiments, is not reactive with the first functional group. The disclosure provides, in some embodiments, water-soluble compounds that comprise at least one arm of a branched molecular structure. For example, the branched molecular structure can be dendritic.

In exemplary embodiments, the polymer is linked to the targeting polypeptide or modified targeting polypeptide through a linker. For example, the linker can comprise one or two amino acids which at one end bind to the polymer—such as an albumin binding moiety—and at the other end bind to any available position on the polypeptide backbone. Additional exemplary linkers include a hydrophilic linker such as a chemical moiety which comprises at least 5 non-hydrogen atoms where 30-50% of these are either N or O. Additional exemplary linkers which may link a polymer to a targeting polypeptide or modified targeting polypeptide are disclosed in U.S. 2012/0295847 and WO/2012/168430, each of which is hereby incorporated by reference in its entirety.

Optionally, multiple targeting polypeptide or modified targeting polypeptide molecules may be joined by a linker polypeptide, wherein said linker polypeptide optionally is 1, 1-2, 1-3, 1-4, 1-5, 1-6, 1-7, 1-8, 1-9, 1-10, 1-11, 1-12 amino acids in length, and longer in length, wherein optionally the N-terminus of one targeting polypeptide is fused to the C-terminus of the linker polypeptide and the N-terminus of the linker polypeptide is fused to the N-terminus of another targeting polypeptide. Further exemplary linker polypeptides which may be utilized are disclosed in WO/2013/004607, which is hereby incorporated by reference in its entirety.

The terms “electrophilic group”, “electrophile” and the like as used herein refers to an atom or group of atoms that can accept an electron pair to form a covalent bond. The “electrophilic group” used herein includes but is not limited to halide, carbonyl and epoxide containing compounds. Common electrophiles may be halides such as thiophosgene, glycerin dichlorohydrin, phthaloyl chloride, succinyl chloride, chloroacetyl chloride, chlorosuccinyl chloride, etc.; ketones such as chloroacetone, bromoacetone, etc.; aldehydes such as glyoxal, etc.; isocyanates such as hexamethylene diisocyanate, toluene diisocyanate, meta-xylylene diisocyanate, cyclohexylmethane-4,4-diisocyanate, etc. and derivatives of these compounds.

The terms “nucleophilic group”, “nucleophile” and the like as used herein refers to an atom or group of atoms that have an electron pair capable of forming a covalent bond. Groups of this type may be ionizable groups that react as anionic groups. The “nucleophilic group” used herein includes but is not limited to hydroxyl, primary amines, secondary amines, tertiary amines and thiols.

Table 2 provides various starting electrophiles and nucleophiles which may be combined to create a desired functional group. The information provided is meant to be illustrative and not limiting to the synthetic techniques described herein.

TABLE 2
Examples of Covalent Linkages and Precursors Thereof

| Covalent Linkage | Electrophile | Nucleophile |
|---|---|---|
| Carboxamides | Activated esters | amines/anilines |
| Carboxamides | acyl azides | amines/anilines |
| Carboxamides | acyl halides | amines/anilines |
| Esters | acyl halides | alcohols/phenols |
| Esters | acyl nitriles | alcohols/phenols |
| Carboxamides | acyl nitriles | amines/anilines |
| Imines | Aldehydes | amines/anilines |
| Hydrazones | aldehydes or ketones | Hydrazines |
| Oximes | aldehydes or ketones | Hydroxylamines, |
| | | aminooxy |
| Alkyl amines | alkyl halides | amines/anilines |
| Esters | alkyl halides | carboxylic acids |
| Thioethers | alkyl halides | Thiols |
| Ethers | alkyl halides | alcohols/phenols |
| Thioethers | alkyl sulfonates | Thiols |
| Esters | alkyl sulfonates | carboxylic acids |
| Ethers | alkyl sulfonates | alcohols/phenols |
| Esters | Anhydrides | alcohols/phenols |
| Carboxamides | Anhydrides | amines/anilines |
| Thiophenols | aryl halides | Thiols |
| Aryl amines | aryl halides | Amines |
| Thioethers | Azindines | Thiols |
| Boronate esters | Boronates | Glycols |
| Carboxamides | carboxylic acids | amines/anilines |
| Esters | carboxylic acids | Alcohols |
| hydrazines | Hydrazides | carboxylic acids |
| N-acylureas or Anhydrides | Carbodiimides | carboxylic acids |
| Esters | Diazoalkanes | carboxylic acids |
| Thioethers | Epoxides | Thiols |
| Thioethers | Haloacetamides | Thiols |
| Ammotriazines | Halotriazines | amines/anilines |
| Triazinyl ethers | Halotriazines | alcohols/phenols |
| Amidines | imido esters | amines/anilines |
| Ureas | Isocyanates | amines/anilines |
| Urethanes | Isocyanates | alcohols/phenols |
| Thioureas | Isothiocyanates | amines/anilines |
| Thioethers | Maleimides | Thiols |
| Phosphite esters | Phosphoramidites | Alcohols |
| Silyl ethers | silyl halides | Alcohols |
| Alkyl amines | sulfonate esters | amines/anilines |
| Thioethers | sulfonate esters | Thiols |
| Esters | sulfonate esters | carboxylic acids |
| Ethers | sulfonate esters | Alcohols |
| Sulfonamides | sulfonyl halides | amines/anilines |
| Sulfonate esters | sulfonyl halides | phenols/alcohols |

In general, carbon electrophiles are susceptible to attack by complementary nucleophiles, including carbon nucleophiles, wherein an attacking nucleophile brings an electron pair to the carbon electrophile in order to form a new bond between the nucleophile and the carbon electrophile.

Non-limiting examples of carbon nucleophiles include, but are not limited to alkyl, alkenyl, aryl and alkynyl Grignard, organolithium, organozinc, alkyl-, alkenyl, aryl- and alkynyl-tin reagents (organostannanes), alkyl-, alkenyl-, aryl- and alkynyl-borane reagents (organoboranes and organoboronates); these carbon nucleophiles have the advantage of being kinetically stable in water or polar organic solvents. Other non-limiting examples of carbon nucleophiles include phosphorus ylide, enol and enolate reagents; these carbon nucleophiles have the advantage of being relatively easy to generate from precursors well known to those skilled in the art of synthetic organic chemistry. Carbon nucleophiles, when used in conjunction with carbon electrophiles, engender new carbon-carbon bonds between the carbon nucleophile and carbon electrophile.

Non-limiting examples of non-carbon nucleophiles suitable for coupling to carbon electrophiles include but are not limited to primary and secondary amines, thiols, thiolates, thioethers, alcohols, alkoxides, azides, semicarbazides, and the like. These non-carbon nucleophiles, when used in conjunction with carbon electrophiles, typically generate heteroatom linkages (C-X-C), wherein X is a heteroatom, including, but not limited to, oxygen, sulfur, or nitrogen.

In some cases, a polymer used in the disclosure terminates on one end with hydroxy or methoxy, i.e., X is H or CH₃ (“methoxy PEG”). Alternatively, the polymer can terminate with a reactive group, thereby forming a bifunctional polymer. Typical reactive groups can include those reactive groups that are commonly used to react with the functional groups found in the 20 common amino acids (including but not limited to, maleimide groups, activated carbonates (including but not limited to, p-nitrophenyl ester), activated esters (including but not limited to, N-hydroxysuccinimide, p-nitrophenyl ester) and aldehydes) as well as functional groups that are inert to the 20 common amino acids but that react specifically with complementary functional groups (including but not limited to, azide groups, alkyne groups). It is noted that the other end of the polymer, which is shown in the above formula by Y, will attach either directly or indirectly to a targeting polypeptide via a naturally-occurring or non-naturally encoded amino acid. For instance, Y may be an amide, carbamate or urea linkage to an amine group (including but not limited to, the epsilon amine of lysine or the N-terminus) of the polypeptide. Alternatively, Y may be a maleimide linkage to a thiol group (including but not limited to, the thiol group of cysteine). Alternatively, Y may be a linkage to a residue not commonly accessible via the 20 common amino acids. For example, an azide group on the polymer can be reacted with an alkyne group on the targeting polypeptide to form a Huisgen [3+2] cycloaddition product. Alternatively, an alkyne group on the polymer can be reacted with an azide group present in a targeting polypeptide to form a similar product. In some embodiments, a strong nucleophile (including but not limited to, hydrazine, hydrazide, hydroxylamine, semicarbazide) can be reacted with an aldehyde or ketone group present in a targeting polypeptide to form a hydrazone, oxime or semicarbazone, as applicable, which in some cases can be further reduced by treatment with an appropriate reducing agent. Alternatively, the strong nucleophile can be incorporated into the targeting polypeptide via a non-naturally encoded amino acid and used to react preferentially with a ketone or aldehyde group present in the water soluble polymer.

Any molecular mass for a polymer can be used as practically desired, including but not limited to, from about 100 Daltons (Da) to 100,000 Da or more as desired (including but not limited to, sometimes 0.1-50 kDa or 10-40 kDa). The molecular weight of polymer may be of a wide range, including but not limited to, between about 100 Da and about 100,000 Da or more. The polymer may be between about 100 Da and about 100,000 Da, including but not limited to, 100,000 Da, 95,000 Da, 90,000 Da, 85,000 Da, 80,000 Da, 75,000 Da, 70,000 Da, 65,000 Da, 60,000 Da, 55,000 Da, 50,000 Da, 45,000 Da, 40,000 Da, 35,000 Da, 30,000 Da, 25,000 Da, 20,000 Da, 15,000 Da, 10,000 Da, 9,000 Da, 8,000 Da, 7,000 Da, 6,000 Da, 5,000 Da, 4,000 Da, 3,000 Da, 2,000 Da, 1,000 Da, 900 Da, 800 Da, 700 Da, 600 Da, 500 Da, 400 Da, 300 Da, 200 Da, and 100 Da. In some embodiments, polymer is between about 100 Da and about 50,000 Da. Branched chain polymers, including but not limited to, polymer molecules with each chain having a molecular weight ranging from 1-100 kDa (including but not limited to, 1-50 kDa or 5-20 kDa) can also be used. The molecular weight of each chain of the branched chain polymer may be, including but not limited to, between about 1,000 Da and about 100,000 Da or more. The molecular weight of each chain of the branched chain polymer may be between about 1,000 Da and about 100,000 Da, including but not limited to, 100,000 Da, 95,000 Da, 90,000 Da, 85,000 Da, 80,000 Da, 75,000 Da, 70,000 Da, 65,000 Da, 60,000 Da, 55,000 Da, 50,000 Da, 45,000 Da, 40,000 Da, 35,000 Da, 30,000 Da, 25,000 Da, 20,000 Da, 15,000 Da, 10,000 Da, 9,000 Da, 8,000 Da, 7,000 Da, 6,000 Da, 5,000 Da, 4,000 Da, 3,000 Da, 2,000 Da, and 1,000 Da. In some embodiments, the molecular weight of each chain of the branched chain polymer is between about 1,000 Da and about 50,000 Da. In some embodiments, the molecular weight of each chain of the branched chain polymer is between about 1,000 Da and about 40,000 Da. In some embodiments, the molecular weight of each chain of the branched chain polymer is between about 5,000 Da and about 40,000 Da. In some embodiments, the molecular weight of each chain of the branched chain polymer is between about 5,000 Da and about 20,000 Da. A wide range of polymer molecules are described in, including but not limited to, the Shearwater Polymers, Inc. catalog, Nektar Therapeutics catalog, incorporated herein by reference.

The disclosure provides in some embodiments azide- and acetylene-containing polymer derivatives comprising a water soluble polymer backbone having an average molecular weight from about 800 Da to about 100,000 Da. The polymer backbone of the water-soluble polymer can be poly(ethylene glycol). However, it should be understood that a wide variety of water soluble polymers including but not limited to poly(ethylene)glycol and other related polymers, including poly(dextran) and poly(propylene glycol), are also suitable for use in the practice of this disclosure and that the use of the term PEG or poly(ethylene glycol) is intended to encompass and include all such molecules. The term PEG includes, but is not limited to, poly(ethylene glycol) in any of its forms, including bifunctional PEG, multiarmed PEG, derivatized PEG, forked PEG, branched PEG, pendent PEG (i.e. PEG or related polymers having one or more functional groups pendent to the polymer backbone), or PEG with degradable linkages therein.

In addition to these forms of polymer, the polymer can also be prepared with weak or degradable linkages in the backbone. For example, polymer can be prepared with ester linkages in the polymer backbone that are subject to hydrolysis. As shown below, this hydrolysis results in cleavage of the polymer into fragments of lower molecular weight: -polymer-CO₂-polymer-+H₂O→polymer-CO₂H+HO-polymer-

Many polymers are also suitable for use in the present disclosure. In some embodiments, polymer backbones that are water-soluble, with from 2 to about 300 termini, are particularly useful in the disclosure. Examples of suitable polymers include, but are not limited to, other poly(alkylene glycols), such as poly(propylene glycol) (“PPG”), copolymers thereof (including but not limited to copolymers of ethylene glycol and propylene glycol), terpolymers thereof, mixtures thereof, and the like. Although the molecular weight of each chain of the polymer backbone can vary, it is typically in the range of from about 800 Da to about 100,000 Da, often from about 6,000 Da to about 80,000 Da. The molecular weight of each chain of the polymer backbone may be between about 100 Da and about 100,000 Da, including but not limited to, 100,000 Da, 95,000 Da, 90,000 Da, 85,000 Da, 80,000 Da, 75,000 Da, 70,000 Da, 65,000 Da, 60,000 Da, 55,000 Da, 50,000 Da, 45,000 Da, 40,000 Da, 35,000 Da, 30,000 Da, 25,000 Da, 20,000 Da, 15,000 Da, 10,000 Da, 9,000 Da, 8,000 Da, 7,000 Da, 6,000 Da, 5,000 Da, 4,000 Da, 3,000 Da, 2,000 Da, 1,000 Da, 900 Da, 800 Da, 700 Da, 600 Da, 500 Da, 400 Da, 300 Da, 200 Da, and 100 Da. In some embodiments, the molecular weight of each chain of the polymer backbone is between about 100 Da and about 50,000 Da. In some embodiments, the molecular weight of each chain of the polymer backbone is between about 100 Da and about 40,000 Da. In some embodiments, the molecular weight of each chain of the polymer backbone is between about 1,000 Da and about 40,000 Da. In some embodiments, the molecular weight of each chain of the polymer backbone is between about 5,000 Da and about 40,000 Da. In some embodiments, the molecular weight of each chain of the polymer backbone is between about 10,000 Da and about 40,000 Da.

In one feature of this embodiment of the disclosure, the intact polymer-conjugate, prior to hydrolysis, is minimally degraded upon administration, such that hydrolysis of the cleavable bond is effective to govern the slow rate of release of active targeting polypeptide into the bloodstream, as opposed to enzymatic degradation of targeting polypeptide prior to its release into the systemic circulation.

Appropriate physiologically cleavable linkages include but are not limited to ester, carbonate ester, carbamate, sulfate, phosphate, acyloxyalkyl ether, acetal, and ketal. Such conjugates should possess a physiologically cleavable bond that is stable upon storage and upon administration. For instance, a targeting polypeptide or modified targeting polypeptide linked to a polymer should maintain its integrity upon manufacturing of the final pharmaceutical composition, upon dissolution in an appropriate delivery vehicle, if employed, and upon administration irrespective of route.

### Structure and Synthesis of Cytotoxic Tubulin Inhibitor Linker Derivatives: Electrophilic and Nucleophilic Groups

Cytotoxic tubulin inhibitor derivatives with linkers containing a hydroxylamine (also called an aminooxy) group allow for reaction with a variety of electrophilic groups to form conjugates (including but not limited to, with PEG or other water soluble polymers). Like hydrazines, hydrazides and semicarbazides, the enhanced nucleophilicity of the aminooxy group permits it to react efficiently and selectively with a variety of molecules that contain carbonyl- or dicarbonyl-groups, including but not limited to, ketones, aldehydes or other functional groups with similar chemical reactivity. See, e.g., Shao, J. and Tam, J., J. Am. Chem. Soc. 117:3893-3899 (1995); H. Hang and C. Bertozzi, Acc. Chem. Res. 34(9): 727-736 (2001). Whereas the result of reaction with a hydrazine group is the corresponding hydrazone, however, an oxime results generally from the reaction of an aminooxy group with a carbonyl- or dicarbonyl-containing group such as, by way of example, a ketones, aldehydes or other functional groups with similar chemical reactivity. In some embodiments, cytotoxic tubulin inhibitor derivatives with linkers comprising an azide, alkyne or cycloalkyne allow for linking of molecules via cycloaddition reactions (e.g., 1,3-dipolar cycloadditions, azide-alkyne Huisgen cycloaddition, etc.). (Described in U.S. Pat. No. 7,807,619 which is incorporated by reference herein to the extent relative to the reaction).

Thus, in certain embodiments described herein are cytotoxic tubulin inhibitor derivatives with linkers comprising a hydroxylamine, aldehyde, protected aldehyde, ketone, protected ketone, thioester, ester, dicarbonyl, hydrazine, amidine, imine, diamine, keto-amine, keto-alkyne, and ene-dione hydroxylamine group, a hydroxylamine-like group (which has reactivity similar to a hydroxylamine group and is structurally similar to a hydroxylamine group), a masked hydroxylamine group (which can be readily converted into a hydroxylamine group), or a protected hydroxylamine group (which has reactivity similar to a hydroxylamine group upon deprotection). In some embodiments, the cytotoxic tubulin inhibitor derivatives with linkers comprise azides, alkynes or cycloalkynes.

Such cytotoxic tubulin inhibitor linker derivatives or the targeting polypeptide may be in the form of a salt or may be incorporated into a non-natural amino acid polypeptide, polymer, polysaccharide, or a polynucleotide and optionally post translationally modified.

In certain embodiments, compounds of the cytotoxic tubulin inhibitor linker derivatives are stable in aqueous solution for at least 1 month under mildly acidic conditions. In certain embodiments, ADCs disclosed herein are stable for at least 2 weeks under mildly acidic conditions. In certain embodiments, ADCs disclosed here are stable for at least 5 days under mildly acidic conditions. In certain embodiments, such acidic conditions are pH 2 to 8. In certain embodiments, ADCs disclosed herein have a shelf-life of 1, 2, 3 or more years as liquid and lypholized powder formulations for injection at storage conditions between −25° C. to −15° C. and 2° C.-8° C. respectively.

The methods and compositions provided and described herein include polypeptides comprising non-natural amino acids having at least one carbonyl or dicarbonyl group, oxime group, hydroxylamine group, or protected or masked forms thereof. Introduction of at least one reactive group into a cytotoxic tubulin inhibitor linker derivative or the targeting polypeptide can allow for the application of conjugation chemistries that involve specific chemical reactions, including, but not limited to, with one or more targeting polypeptide(s) while not reacting with the commonly occurring amino acids. Once incorporated, the targeting polypeptide of the ADC side chains can also be modified by utilizing chemistry methodologies described herein or suitable for the particular functional groups or substituents present in the cytotoxic tubulin inhibitor linker derivative or the targeting polypeptide.

The cytotoxic tubulin inhibitor linker derivative and the targeting polypeptide methods and compositions described herein provide conjugates of substances having a wide variety of functional groups, substituents or moieties, with other substances including but not limited to a polymer; a water-soluble polymer; a derivative of polyethylene glycol; a second protein or polypeptide or polypeptide analog; an antibody or antibody fragment; and any combination thereof.

In certain embodiments, the cytotoxic tubulin inhibitor linker derivatives, the targeting polypeptide, ADCs, linkers and reagents described herein are stable in aqueous solution under mildly acidic conditions (including but not limited to pH 2 to 8). In other embodiments, such compounds are stable for at least one month under mildly acidic conditions. In other embodiments, such compounds are stable for at least 2 weeks under mildly acidic conditions. In other embodiments, such compounds are stable for at least 5 days under mildly acidic conditions.

In another aspect of the compositions, methods, techniques and strategies described herein are methods for studying or using any of the aforementioned “modified or unmodified” non-natural amino acid targeting polypeptide. Included within this aspect, by way of example only, are therapeutic, diagnostic, assay-based, industrial, cosmetic, plant biology, environmental, energy-production, consumer-products, and/or military uses which would benefit from a targeting polypeptide comprising a “modified or unmodified” non-natural amino acid polypeptide or protein.

ADC molecules comprising at least one non-natural amino acid are provided in the disclosure. In certain embodiments of the disclosure, the ADC with at least one non-natural amino acid includes at least one post-translational modification. In one embodiment, the at least one post-translational modification comprises attachment of a molecule including but not limited to, a label, a dye, a linker, another ADC polypeptide, a polymer, a water-soluble polymer, a derivative of polyethylene glycol, a photocrosslinker, a radionuclide, a cytotoxic compound, a drug, an affinity label, a photoaffinity label, a reactive compound, a resin, a second protein or polypeptide or polypeptide analog, an antibody or antibody fragment, a metal chelator, a cofactor, a fatty acid, a carbohydrate, a polynucleotide, a DNA, a RNA, an antisense polynucleotide, a saccharide, a cyclodextrin, an inhibitory ribonucleic acid, a biomaterial, a nanoparticle, a spin label, a fluorophore, a metal-containing moiety, a radioactive moiety, a novel functional group, a group that covalently or noncovalently interacts with other molecules, a photocaged moiety, an actinic radiation excitable moiety, a photoisomerizable moiety, biotin, a derivative of biotin, a biotin analogue, a moiety incorporating a heavy atom, a chemically cleavable group, a photocleavable group, an elongated side chain, a carbon-linked sugar, a redox-active agent, an amino thioacid, a toxic moiety, an isotopically labeled moiety, a biophysical probe, a phosphorescent group, a chemiluminescent group, an electron dense group, a magnetic group, an intercalating group, a chromophore, an energy transfer agent, a biologically active agent, a detectable label, a small molecule, a quantum dot, a nanotransmitter, a radionucleotide, a radiotransmitter, a neutron-capture agent, or any combination of the above or any other desirable compound or substance, comprising a second reactive group to at least one non-natural amino acid comprising a first reactive group utilizing chemistry methodology that is known to one of ordinary skill in the art to be suitable for the particular reactive groups. For example, the first reactive group is an alkynyl moiety (including but not limited to, in the non-natural amino acid p-propargyloxyphenylalanine, where the propargyl group is also sometimes referred to as an acetylene moiety) and the second reactive group is an azido moiety, and [3+2] cycloaddition chemistry methodologies are utilized. In another example, the first reactive group is the azido moiety (including but not limited to, in the non-natural amino acid p-azido-L-phenylalanine or pAZ as it is sometimes referred to within this specification) and the second reactive group is the alkynyl moiety. In certain embodiments of the modified ADC of the present disclosure, at least one non-natural amino acid (including but not limited to, non-natural amino acid containing a keto functional group) comprising at least one post-translational modification, is used where the at least one post-translational modification comprises a saccharide moiety. In certain embodiments, the post-translational modification is made in vivo in a eukaryotic cell or in a non-eukaryotic cell. A linker, polymer, water soluble polymer, or other molecule may attach the molecule to the polypeptide. In an additional embodiment the linker attached to the ADC is long enough to permit formation of a dimer. The molecule may also be linked directly to the polypeptide.

In certain embodiments, the ADC protein includes at least one post-translational modification that is made in vivo by one host cell, where the post-translational modification is not normally made by another host cell type. In certain embodiments, the protein includes at least one post-translational modification that is made in vivo by a eukaryotic cell, where the post-translational modification is not normally made by a non-eukaryotic cell. Examples of post-translational modifications include, but are not limited to, glycosylation, acetylation, acylation, lipid-modification, palmitoylation, palmitate addition, phosphorylation, glycolipid-linkage modification, and the like.

In some embodiments, the ADC comprise one or more non-naturally encoded amino acids for glycosylation, acetylation, acylation, lipid-modification, palmitoylation, palmitate addition, phosphorylation, or glycolipid-linkage modification of the polypeptide. In some embodiments, the ADC comprise one or more non-naturally encoded amino acids for glycosylation of the polypeptide. In some embodiments, the ADC comprise one or more naturally encoded amino acids for glycosylation, acetylation, acylation, lipid-modification, palmitoylation, palmitate addition, phosphorylation, or glycolipid-linkage modification of the polypeptide. In some embodiments, the ADC comprise one or more naturally encoded amino acids for glycosylation of the polypeptide.

In some embodiments, the ADC comprises one or more non-naturally encoded amino acid additions and/or substitutions that enhance glycosylation of the polypeptide. In some embodiments, the ADC comprises one or more deletions that enhance glycosylation of the polypeptide. In some embodiments, the ADC comprises one or more non-naturally encoded amino acid additions and/or substitutions that enhance glycosylation at a different amino acid in the polypeptide. In some embodiments, the ADC comprises one or more deletions that enhance glycosylation at a different amino acid in the polypeptide. In some embodiments, the ADC comprises one or more non-naturally encoded amino acid additions and/or substitutions that enhance glycosylation at a non-naturally encoded amino acid in the polypeptide. In some embodiments, the ADC comprises one or more non-naturally encoded amino acid additions and/or substitutions that enhance glycosylation at a naturally encoded amino acid in the polypeptide. In some embodiments, the ADC comprises one or more naturally encoded amino acid additions and/or substitutions that enhance glycosylation at a different amino acid in the polypeptide. In some embodiments, the ADC comprises one or more non-naturally encoded amino acid additions and/or substitutions that enhance glycosylation at a naturally encoded amino acid in the polypeptide. In some embodiments, the ADC comprises one or more non-naturally encoded amino acid additions and/or substitutions that enhance glycosylation at a non-naturally encoded amino acid in the polypeptide.

In one embodiment, the post-translational modification comprises attachment of an oligosaccharide to an asparagine by a GlcNAc-asparagine linkage (including but not limited to, where the oligosaccharide comprises (GlcNAc-Man)₂-Man-GlcNAc-GlcNAc, and the like). In another embodiment, the post-translational modification comprises attachment of an oligosaccharide (including but not limited to, Gal-GalNAc, Gal-GlcNAc, etc.) to a serine or threonine by a GalNAc-serine, a GalNAc-threonine, a GlcNAc-serine, or a GlcNAc-threonine linkage. In certain embodiments, a protein or polypeptide of the disclosure can comprise a secretion or localization sequence, an epitope tag, a FLAG tag, a polyhistidine tag, a GST fusion, and/or the like. Examples of secretion signal sequences include, but are not limited to, a prokaryotic secretion signal sequence, a eukaryotic secretion signal sequence, a eukaryotic secretion signal sequence 5′-optimized for bacterial expression, a novel secretion signal sequence, pectate lyase secretion signal sequence, Omp A secretion signal sequence, and a phage secretion signal sequence. Examples of secretion signal sequences include, but are not limited to, STII (prokaryotic), Fd GIII and M13 (phage), Bgl2 (yeast), and the signal sequence bla derived from a transposon. Any such sequence may be modified to provide a desired result with the polypeptide, including but not limited to, substituting one signal sequence with a different signal sequence, substituting a leader sequence with a different leader sequence, etc.

The protein or polypeptide of interest can contain at least one, at least two, at least three, at least four, at least five, at least six, at least seven, at least eight, at least nine, or ten or more non-natural amino acids. The non-natural amino acids can be the same or different, for example, there can be 1, 2, 3, 4, 5, 6, 7, 8, 9, 10 or more different sites in the protein that comprise 1, 2, 3, 4, 5, 6, 7, 8, 9, 10 or more different non-natural amino acids. In certain embodiments, at least one, but fewer than all, of a particular amino acid present in a naturally occurring version of the protein is substituted with a non-natural amino acid.

The present disclosure provides methods and compositions based on ADC comprising at least one non-naturally encoded amino acid. Introduction of at least one non-naturally encoded amino acid into ADC can allow for the application of conjugation chemistries that involve specific chemical reactions, including, but not limited to, with one or more non-naturally encoded amino acids while not reacting with the commonly occurring 20 amino acids. In some embodiments, ADC comprising the non-naturally encoded amino acid is linked to a water soluble polymer, such as polyethylene glycol (PEG), or a linker, via the side chain of the non-naturally encoded amino acid. This disclosure provides a highly efficient method for the selective modification of proteins with PEG derivatives or cytotoxic tubulin inhibitor-linker derivatives, which involves the selective incorporation of non-genetically encoded amino acids, including but not limited to, those amino acids containing functional groups or substituents not found in the 20 naturally incorporated amino acids, including but not limited to a ketone, an azide or acetylene moiety, into proteins in response to a selector codon and the subsequent modification of those amino acids with a suitably reactive PEG derivative. Once incorporated, the amino acid side chains can then be modified by utilizing chemistry methodologies known to those of ordinary skill in the art to be suitable for the particular functional groups or substituents present in the non-naturally encoded amino acid. Known chemistry methodologies of a wide variety are suitable for use in the present disclosure to incorporate a water soluble polymer into the protein. Such methodologies include but are not limited to a Huisgen [3+2] cycloaddition reaction (see, e.g., Padwa, A. in Comprehensive Organic Synthesis, Vol. 4, (1991) Ed. Trost, B. M., Pergamon, Oxford, p. 1069-1109; and, Huisgen, R. in 1,3-Dipolar Cycloaddition Chemistry, (1984) Ed. Padwa, A., Wiley, New York, p. 1-176) with, including but not limited to, acetylene or azide derivatives, respectively.

Because the Huisgen [3+2] cycloaddition method involves a cycloaddition rather than a nucleophilic substitution reaction, proteins can be modified with extremely high selectivity. The reaction can be carried out at room temperature in aqueous conditions with excellent regioselectivity (1,4 >1,5) by the addition of catalytic amounts of Cu(I) salts to the reaction mixture. See, e.g., Tornoe, et al., (2002) J. Org. Chem. 67:3057-3064; and, Rostovtsev, et al., (2002) Angew. Chem. Int. Ed. 41:2596-2599; and WO 03/101972. A molecule that can be added to a protein of the disclosure through a [3+2] cycloaddition includes virtually any molecule with a suitable functional group or substituent including but not limited to an azido or acetylene derivative. These molecules can be added to an non-natural amino acid with an acetylene group, including but not limited to, p-propargyloxyphenylalanine, or azido group, including but not limited to p-azido-phenylalanine, respectively.

The five-membered ring that results from the Huisgen [3+2] cycloaddition is not generally reversible in reducing environments and is stable against hydrolysis for extended periods in aqueous environments. Consequently, the physical and chemical characteristics of a wide variety of substances can be modified under demanding aqueous conditions with the active PEG derivatives or cytotoxic tubulin inhibitor-linker derivatives of the present disclosure. Even more importantly, because the azide and acetylene moieties are specific for one another (and do not, for example, react with any of the 20 common, genetically-encoded amino acids), proteins can be modified in one or more specific sites with extremely high selectivity.

The disclosure also provides water soluble and hydrolytically stable derivatives of PEG derivatives or cytotoxic tubulin inhibitor-linker derivatives and related hydrophilic polymers having one or more acetylene or azide moieties. The PEG polymer derivatives that contain acetylene moieties are highly selective for coupling with azide moieties that have been introduced selectively into proteins in response to a selector codon. Similarly, PEG polymer derivatives that contain azide moieties are highly selective for coupling with acetylene moieties that have been introduced selectively into proteins in response to a selector codon. More specifically, the azide moieties comprise, but are not limited to, alkyl azides, aryl azides and derivatives of these azides. The derivatives of the alkyl and aryl azides can include other substituents so long as the acetylene-specific reactivity is maintained. The acetylene moieties comprise alkyl and aryl acetylenes and derivatives of each. The derivatives of the alkyl and aryl acetylenes can include other substituents so long as the azide-specific reactivity is maintained.

The present disclosure provides conjugates of substances having a wide variety of functional groups, substituents or moieties, with other substances including but not limited to a label; a dye; a polymer; a water-soluble polymer; a derivative of polyethylene glycol; a photocrosslinker; a radionuclide; a cytotoxic compound; a drug; an affinity label; a photoaffinity label; a reactive compound; a resin; a second protein or polypeptide or polypeptide analog; an antibody or antibody fragment; a metal chelator; a cofactor; a fatty acid; a carbohydrate; a polynucleotide; a DNA; a RNA; an antisense polynucleotide; a saccharide; a water-soluble dendrimer; a cyclodextrin; an inhibitory ribonucleic acid; a biomaterial; a nanoparticle; a spin label; a fluorophore, a metal-containing moiety; a radioactive moiety; a novel functional group; a group that covalently or noncovalently interacts with other molecules; a photocaged moiety; an actinic radiation excitable moiety; a photoisomerizable moiety; biotin; a derivative of biotin; a biotin analogue; a moiety incorporating a heavy atom; a chemically cleavable group; a photocleavable group; an elongated side chain; a carbon-linked sugar; a redox-active agent; an amino thioacid; a toxic moiety; an isotopically labeled moiety; a biophysical probe; a phosphorescent group; a chemiluminescent group; an electron dense group; a magnetic group; an intercalating group; a chromophore; an energy transfer agent; a biologically active agent; a detectable label; a small molecule; a quantum dot; a nanotransmitter; a radionucleotide; a radiotransmitter; a neutron-capture agent; or any combination of the above, or any other desirable compound or substance. The present disclosure also includes conjugates of substances having azide or acetylene moieties with PEG polymer derivatives having the corresponding acetylene or azide moieties. For example, a PEG polymer containing an azide moiety can be coupled to a biologically active molecule at a position in the protein that contains a non-genetically encoded amino acid bearing an acetylene functionality. The linkage by which the PEG and the biologically active molecule are coupled includes but is not limited to the Huisgen [3+2] cycloaddition product.

It is well established in the art that PEG can be used to modify the surfaces of biomaterials (see, e.g., U.S. Pat. No. 6,610,281; Mehvar, R., J. Pharm Sci., 3(1):125-136 (2000) which are incorporated by reference herein). The disclosure also includes biomaterials comprising a surface having one or more reactive azide or acetylene sites and one or more of the azide- or acetylene-containing polymers of the disclosure coupled to the surface via the Huisgen [3+2] cycloaddition linkage. Biomaterials and other substances can also be coupled to the azide- or acetylene-activated polymer derivatives through a linkage other than the azide or acetylene linkage, such as through a linkage comprising a carboxylic acid, amine, alcohol or thiol moiety, to leave the azide or acetylene moiety available for subsequent reactions.

The disclosure includes a method of synthesizing the azide- and acetylene-containing polymers of the disclosure. In the case of the azide-containing PEG derivative, the azide can be bonded directly to a carbon atom of the polymer. Alternatively, the azide-containing PEG derivative can be prepared by attaching a linking agent that has the azide moiety at one terminus to a conventional activated polymer so that the resulting polymer has the azide moiety at its terminus. In the case of the acetylene-containing PEG derivative, the acetylene can be bonded directly to a carbon atom of the polymer. Alternatively, the acetylene-containing PEG derivative can be prepared by attaching a linking agent that has the acetylene moiety at one terminus to a conventional activated polymer so that the resulting polymer has the acetylene moiety at its terminus.

More specifically, in the case of the azide-containing PEG derivative, a water soluble polymer having at least one active hydroxyl moiety undergoes a reaction to produce a substituted polymer having a more reactive moiety, such as a mesylate, tresylate, tosylate or halogen leaving group, thereon. The preparation and use of PEG derivatives or cytotoxic tubulin inhibitor-linker derivatives containing sulfonyl acid halides, halogen atoms and other leaving groups are known to those of ordinary skill in the art. The resulting substituted polymer then undergoes a reaction to substitute for the more reactive moiety an azide moiety at the terminus of the polymer. Alternatively, a water soluble polymer having at least one active nucleophilic or electrophilic moiety undergoes a reaction with a linking agent that has an azide at one terminus so that a covalent bond is formed between the PEG polymer and the linking agent and the azide moiety is positioned at the terminus of the polymer. Nucleophilic and electrophilic moieties, including amines, thiols, hydrazides, hydrazines, alcohols, carboxylates, aldehydes, ketones, thioesters and the like, are known to those of ordinary skill.

More specifically, in the case of the acetylene-containing PEG derivative, a water soluble polymer having at least one active hydroxyl moiety undergoes a reaction to displace a halogen or other activated leaving group from a precursor that contains an acetylene moiety. Alternatively, a water soluble polymer having at least one active nucleophilic or electrophilic moiety undergoes a reaction with a linking agent that has an acetylene at one terminus so that a covalent bond is formed between the PEG polymer and the linking agent and the acetylene moiety is positioned at the terminus of the polymer. The use of halogen moieties, activated leaving group, nucleophilic and electrophilic moieties in the context of organic synthesis and the preparation and use of PEG derivatives or cytotoxic tubulin inhibitor-linker derivatives is well established to practitioners in the art.

The disclosure also provides a method for the selective modification of proteins to add other substances to the modified protein, including but not limited to water soluble polymers such as PEG and PEG derivatives or cytotoxic tubulin inhibitor-linker derivatives, linkers, or another ADC polypeptide, containing an azide or acetylene moiety. The azide- and acetylene-containing PEG derivatives or cytotoxic tubulin inhibitor-linker derivatives can be used to modify the properties of surfaces and molecules where biocompatibility, stability, solubility and lack of immunogenicity are important, while at the same time providing a more selective means of attaching the PEG derivatives or cytotoxic tubulin inhibitor-linker derivatives to proteins than was previously known in the art.

General Recombinant Nucleic Acid Methods for Use with the Disclosure

In numerous embodiments of the present disclosure, nucleic acids encoding a targeting polypeptide of the ADC of interest will be isolated, cloned and often altered using recombinant methods. Such embodiments are used, including but not limited to, for protein expression or during the generation of variants, derivatives, expression cassettes, or other sequences derived from a targeting polypeptide of the ADC. In some embodiments, the sequences encoding the polypeptides of the disclosure are operably linked to a heterologous promoter.

A nucleotide sequence encoding a targeting polypeptide of the ADC comprising a non-naturally encoded amino acid may be synthesized on the basis of the amino acid sequence of the parent polypeptide, and then changing the nucleotide sequence so as to effect introduction (i.e., incorporation or substitution) or removal (i.e., deletion or substitution) of the relevant amino acid residue(s). The nucleotide sequence may be conveniently modified by site-directed mutagenesis in accordance with conventional methods. Alternatively, the nucleotide sequence may be prepared by chemical synthesis, including but not limited to, by using an oligonucleotide synthesizer, wherein oligonucleotides are designed based on the amino acid sequence of the desired polypeptide, and preferably selecting those codons that are favored in the host cell in which the recombinant polypeptide will be produced. For example, several small oligonucleotides coding for portions of the desired polypeptide may be synthesized and assembled by PCR, ligation or ligation chain reaction. See, e.g., Barany, et al., Proc. Natl. Acad. Sci. 88: 189-193 (1991); U.S. Pat. No. 6,521,427 which are incorporated by reference herein.

This disclosure utilizes routine techniques in the field of recombinant genetics. Basic texts disclosing the general methods of use in this disclosure include Sambrook et al., Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

The disclosure also relates to eukaryotic host cells, non-eukaryotic host cells, and organisms for the in vivo incorporation of a non-natural amino acid via orthogonal tRNA/RS pairs. Host cells are genetically engineered (including but not limited to, transformed, transduced or transfected) with the polynucleotides of the disclosure or constructs which include a polynucleotide of the disclosure, including but not limited to, a vector of the disclosure, which can be, for example, a cloning vector or an expression vector.

Several well-known methods of introducing target nucleic acids into cells are available, any of which can be used in the disclosure. These include: fusion of the recipient cells with bacterial protoplasts containing the DNA, electroporation, projectile bombardment, and infection with viral vectors (discussed further, below), etc. Bacterial cells can be used to amplify the number of plasmids containing DNA constructs of this disclosure. The bacteria are grown to log phase and the plasmids within the bacteria can be isolated by a variety of methods known in the art (see, for instance, Sambrook). In addition, kits are commercially available for the purification of plasmids from bacteria, (see, e.g., EasyPrep™, FlexiPrep™, both from Pharmacia Biotech; StrataClean™ from Stratagene; and, QIAprep™ from Qiagen). The isolated and purified plasmids are then further manipulated to produce other plasmids, used to transfect cells or incorporated into related vectors to infect organisms. Typical vectors contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both, (including but not limited to, shuttle vectors) and selection markers for both prokaryotic and eukaryotic systems. Vectors are suitable for replication and integration in prokaryotes, eukaryotes, or both. See, Gillam & Smith, Gene 8:81 (1979); Roberts, et al., Nature, 328:731 (1987); Schneider, E., et al., Protein Expr. Purif. 6(1):10-14 (1995); Ausubel, Sambrook, Berger (all supra). A catalogue of bacteria and bacteriophages useful for cloning is provided, e.g., by the ATCC, e.g., The ATCC Catalogue of Bacteria and Bacteriophage (1992) Gherna et al. (eds) published by the ATCC. Additional basic procedures for sequencing, cloning and other aspects of molecular biology and underlying theoretical considerations are also found in Watson et al. (1992) Recombinant DNA Second Edition Scientific American Books, NY. In addition, essentially any nucleic acid (and virtually any labeled nucleic acid, whether standard or non-standard) can be custom or standard ordered from any of a variety of commercial sources, such as the Midland Certified Reagent Company (Midland, TX available on the World Wide Web at mcrc.com), The Great American Gene Company (Ramona, CA available on the World Wide Web at genco.com), ExpressGen Inc. (Chicago, IL available on the World Wide Web at expressgen.com), Operon Technologies Inc. (Alameda, CA) and many others.

### Selector Codons

Selector codons of the disclosure expand the genetic codon framework of protein biosynthetic machinery. For example, a selector codon includes, but is not limited to, a unique three base codon, a nonsense codon, such as a stop codon, including but not limited to, an amber codon (UAG), an ochre codon, or an opal codon (UGA), an unnatural codon, a four or more base codon, a rare codon, or the like. It is readily apparent to those of ordinary skill in the art that there is a wide range in the number of selector codons that can be introduced into a desired gene or polynucleotide, including but not limited to, one or more, two or more, three or more, 4, 5, 6, 7, 8, 9, 10 or more in a single polynucleotide encoding at least a portion of the ADC.

In one embodiment, the methods involve the use of a selector codon that is a stop codon for the incorporation of one or more non-natural amino acids in vivo. For example, an O-tRNA is produced that recognizes the stop codon, including but not limited to, UAG, and is aminoacylated by an O—RS with a desired non-natural amino acid. This O-tRNA is not recognized by the naturally occurring host's aminoacyl-tRNA synthetases. Conventional site-directed mutagenesis can be used to introduce the stop codon, including but not limited to, TAG, at the site of interest in a polypeptide of interest. See, e.g., Sayers, J. R., et al. (1988), 5′-3′ Exonucleases in phosphorothioate-based oligonucleotide-directed mutagenesis. Nucleic Acids Res, 16:791-802. When the O—RS, O-tRNA and the nucleic acid that encodes the polypeptide of interest are combined in vivo, the non-natural amino acid is incorporated in response to the UAG codon to give a polypeptide containing the non-natural amino acid at the specified position.

The incorporation of non-natural amino acids in vivo can be done without significant perturbation of the eukaryotic host cell. For example, because the suppression efficiency for the UAG codon depends upon the competition between the O-tRNA, including but not limited to, the amber suppressor tRNA, and a eukaryotic release factor (including but not limited to, eRF) (which binds to a stop codon and initiates release of the growing peptide from the ribosome), the suppression efficiency can be modulated by, including but not limited to, increasing the expression level of O-tRNA, and/or the suppressor tRNA.

Non-natural amino acids can also be encoded with rare codons. For example, when the arginine concentration in an in vitro protein synthesis reaction is reduced, the rare arginine codon, AGG, has proven to be efficient for insertion of Ala by a synthetic tRNA acylated with alanine. See, e.g., Ma et al., Biochemistry, 32:7939 (1993). In this case, the synthetic tRNA competes with the naturally occurring tRNAArg, which exists as a minor species in Escherichia coli. Some organisms do not use all triplet codons. An unassigned codon AGA in Micrococcus luteus has been utilized for insertion of amino acids in an in vitro transcription/translation extract. See, e.g., Kowal and Oliver, Nucl. Acid. Res., 25:4685 (1997). Components of the present disclosure can be generated to use these rare codons in vivo.

Selector codons also comprise extended codons, including but not limited to, four or more base codons, such as, four, five, six or more base codons. Examples of four base codons include, but are not limited to, AGGA, CUAG, UAGA, CCCU and the like. Examples of five base codons include, but are not limited to, AGGAC, CCCCU, CCCUC, CUAGA, CUACU, UAGGC and the like. A feature of the disclosure includes using extended codons based on frameshift suppression. Four or more base codons can insert, including but not limited to, one or multiple non-natural amino acids into the same protein. For example, in the presence of mutated O-tRNAs, including but not limited to, a special frameshift suppressor tRNAs, with anticodon loops, for example, with at least 8-10 nt anticodon loops, the four or more base codon is read as single amino acid. In other embodiments, the anticodon loops can decode, including but not limited to, at least a four-base codon, at least a five-base codon, or at least a six-base codon or more. Since there are 256 possible four-base codons, multiple non-natural amino acids can be encoded in the same cell using a four or more base codon. See, Anderson et al., (2002) Exploring the Limits of Codon and Anticodon Size, Chemistry and Biology, 9:237-244; Magliery, (2001) Expanding the Genetic Code: Selection of Efficient Suppressors of Four-base Codons and Identification of “Shifty” Four-base Codons with a Library Approach in Escherichia coli, J. Mol. Biol. 307: 755-769.

For example, four-base codons have been used to incorporate non-natural amino acids into proteins using in vitro biosynthetic methods. See, e.g., Ma et al., (1993) Biochemistry, 32:7939; and Hohsaka et al., (1999) J. Am. Chem. Soc., 121:34. CGGG and AGGU were used to simultaneously incorporate 2-naphthylalanine and an NBD derivative of lysine into streptavidin in vitro with two chemically acylated frameshift suppressor tRNAs. See, e.g., Hohsaka et al., (1999) J. Am. Chem. Soc., 121:12194. In an in vivo study, Moore et al. examined the ability of tRNALeu derivatives with NCUA anticodons to suppress UAGN codons (N can be U, A, G, or C), and found that the quadruplet UAGA can be decoded by a tRNALeu with a UCUA anticodon with an efficiency of 13 to 26% with little decoding in the 0 or -1 frame. See, Moore et al., (2000) J. Mol. Biol., 298:195. In one embodiment, extended codons based on rare codons or nonsense codons can be used in the present disclosure, which can reduce missense readthrough and frameshift suppression at other unwanted sites.

For a given system, a selector codon can also include one of the natural three base codons, where the endogenous system does not use (or rarely uses) the natural base codon. For example, this includes a system that is lacking a tRNA that recognizes the natural three base codon, and/or a system where the three base codon is a rare codon.

Selector codons optionally include unnatural base pairs. These unnatural base pairs further expand the existing genetic alphabet. One extra base pair increases the number of triplet codons from 64 to 125. Properties of third base pairs include stable and selective base pairing, efficient enzymatic incorporation into DNA with high fidelity by a polymerase, and the efficient continued primer extension after synthesis of the nascent unnatural base pair. Descriptions of unnatural base pairs which can be adapted for methods and compositions include, e.g., Hirao, et al., (2002) An unnatural base pair for incorporating amino acid analogues into protein, Nature Biotechnology, 20:177-182. See, also, Wu, Y., et al., (2002) J. Am. Chem. Soc. 124:14626-14630. Other relevant publications are listed below.

For in vivo usage, the unnatural nucleoside is membrane permeable and is phosphorylated to form the corresponding triphosphate. In addition, the increased genetic information is stable and not destroyed by cellular enzymes. Previous efforts by Benner and others took advantage of hydrogen bonding patterns that are different from those in canonical Watson-Crick pairs, the most noteworthy example of which is the iso-C:iso-G pair. See, e.g., Switzer et al., (1989) J. Am. Chem. Soc., 111:8322; and Piccirilli et al., (1990) Nature, 343:33; Kool, (2000) Curr. Opin. Chem. Biol., 4:602. These bases in general mispair to some degree with natural bases and cannot be enzymatically replicated. Kool and co-workers demonstrated that hydrophobic packing interactions between bases can replace hydrogen bonding to drive the formation of base pair. See, Kool, (2000) Curr. Opin. Chem. Biol., 4:602; and Guckian and Kool, (1998) Angew. Chem. Int. Ed. Engl., 36, 2825. In an effort to develop an unnatural base pair satisfying all the above requirements, Schultz, Romesberg and co-workers have systematically synthesized and studied a series of unnatural hydrophobic bases. A PICS:PICS self-pair is found to be more stable than natural base pairs and can be efficiently incorporated into DNA by Klenow fragment of Escherichia coli DNA polymerase I (KF). See, e.g., McMinn et al., (1999) J. Am. Chem. Soc., 121:11585-6; and Ogawa et al., (2000) J. Am. Chem. Soc., 122:3274. A 3MN:3MN self-pair can be synthesized by KF with efficiency and selectivity sufficient for biological function. See, e.g., Ogawa et al., (2000) J. Am. Chem. Soc., 122:8803. However, both bases act as a chain terminator for further replication. A mutant DNA polymerase has been recently evolved that can be used to replicate the PICS self pair. In addition, a 7AI self pair can be replicated. See, e.g., Tae et al., (2001) J. Am. Chem. Soc., 123:7439. A novel metallobase pair, Dipic:Py, has also been developed, which forms a stable pair upon binding Cu(II). See, Meggers et al., (2000) J. Am. Chem. Soc., 122:10714. Because extended codons and unnatural codons are intrinsically orthogonal to natural codons, the methods of the disclosure can take advantage of this property to generate orthogonal tRNAs for them.

A translational bypassing system can also be used to incorporate a non-natural amino acid in a desired polypeptide. In a translational bypassing system, a large sequence is incorporated into a gene but is not translated into protein. The sequence contains a structure that serves as a cue to induce the ribosome to hop over the sequence and resume translation downstream of the insertion.

Nucleic acid molecules encoding a protein of interest such as a targeting polypeptide of the ADC may be readily mutated to introduce a cysteine at any desired position of the polypeptide. Cysteine is widely used to introduce reactive molecules, water soluble polymers, proteins, or a wide variety of other molecules, onto a protein of interest. Methods suitable for the incorporation of cysteine into a desired position of a polypeptide are known to those of ordinary skill in the art, such as those described in U.S. Pat. No. 6,608,183, which is incorporated by reference herein, and standard mutagenesis techniques.

### Non-Naturally Encoded Amino Acids

A very wide variety of non-naturally encoded amino acids are suitable for use in the present disclosure. Any number of non-naturally encoded amino acids can be introduced into a ADC. In general, the introduced non-naturally encoded amino acids are substantially chemically inert toward the 20 common, genetically-encoded amino acids (i.e., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine). In some embodiments, the non-naturally encoded amino acids include side chain functional groups that react efficiently and selectively with functional groups not found in the 20 common amino acids (including but not limited to, azido, ketone, aldehyde and aminooxy groups) to form stable conjugates. For example, a targeting polypeptide of the ADC that includes a non-naturally encoded amino acid containing an azido functional group can be reacted with a polymer (including but not limited to, poly(ethylene glycol) or, alternatively, a second polypeptide or linker containing an alkyne moiety) to form a stable conjugate resulting from the selective reaction of the azide and the alkyne functional groups to form a Huisgen [3+2] cycloaddition product.

The generic structure of an alpha-amino acid is illustrated as follows (Formula I):

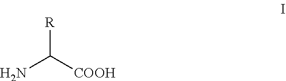

A non-naturally encoded amino acid is typically any structure having the above-listed formula wherein the R group is any substituent other than one used in the twenty natural amino acids, and may be suitable for use in the present disclosure. Because the non-naturally encoded amino acids of the disclosure typically differ from the natural amino acids only in the structure of the side chain, the non-naturally encoded amino acids form amide bonds with other amino acids, including but not limited to, natural or non-naturally encoded, in the same manner in which they are formed in naturally occurring polypeptides. However, the non-naturally encoded amino acids have side chain groups that distinguish them from the natural amino acids. For example, R optionally comprises an alkyl-, aryl-, acyl-, keto-, azido-, hydroxyl-, hydrazine, cyano-, halo-, hydrazide, alkenyl, alkynyl, ether, thiol, seleno-, sulfonyl-, borate, boronate, phospho, phosphono, phosphine, heterocyclic, enone, imine, aldehyde, ester, thioacid, hydroxylamine, amino group, or the like or any combination thereof. Other non-naturally occurring amino acids of interest that may be suitable for use in the present disclosure include, but are not limited to, amino acids comprising a photoactivatable cross-linker, spin-labeled amino acids, fluorescent amino acids, metal binding amino acids, metal-containing amino acids, radioactive amino acids, amino acids with novel functional groups, amino acids that covalently or noncovalently interact with other molecules, photocaged and/or photoisomerizable amino acids, amino acids comprising biotin or a biotin analogue, glycosylated amino acids such as a sugar substituted serine, other carbohydrate modified amino acids, keto-containing amino acids, amino acids comprising polyethylene glycol or polyether, heavy atom substituted amino acids, chemically cleavable and/or photocleavable amino acids, amino acids with an elongated side chains as compared to natural amino acids, including but not limited to, polyethers or long chain hydrocarbons, including but not limited to, greater than about 5 or greater than about 10 carbons, carbon-linked sugar-containing amino acids, redox-active amino acids, amino thioacid containing amino acids, and amino acids comprising one or more toxic moiety.

Exemplary non-naturally encoded amino acids that may be suitable for use in the present disclosure and that are useful for reactions with water soluble polymers include, but are not limited to, those with carbonyl, aminooxy, hydrazine, hydrazide, semicarbazide, azide and alkyne reactive groups. In some embodiments, non-naturally encoded amino acids comprise a saccharide moiety. Examples of such amino acids include N-acetyl-L-glucosaminyl-L-serine, N-acetyl-L-galactosaminyl-L-serine, N-acetyl-L-glucosaminyl-L-threonine, N-acetyl-L-glucosaminyl-L-asparagine and O-mannosaminyl-L-serine. Examples of such amino acids also include examples where the naturally-occurring N- or O-linkage between the amino acid and the saccharide is replaced by a covalent linkage not commonly found in nature—including but not limited to, an alkene, an oxime, a thioether, an amide and the like. Examples of such amino acids also include saccharides that are not commonly found in naturally-occurring proteins such as 2-deoxy-glucose, 2-deoxygalactose and the like.

Many of the non-naturally encoded amino acids provided herein are commercially available, e.g., from Sigma-Aldrich (St. Louis, MO, USA), Novabiochem (a division of EMD Biosciences, Darmstadt, Germany), or Peptech (Burlington, MA, USA). Those that are not commercially available are optionally synthesized as provided herein or using standard methods known to those of ordinary skill in the art. For organic synthesis techniques, see, e.g., Organic Chemistry by Fessendon and Fessendon, (1982, Second Edition, Willard Grant Press, Boston Mass.); Advanced Organic Chemistry by March (Third Edition, 1985, Wiley and Sons, New York); and Advanced Organic Chemistry by Carey and Sundberg (Third Edition, Parts A and B, 1990, Plenum Press, New York). See, also, U.S. Pat. Nos. 7,045,337 and 7,083,970, which are incorporated by reference herein. In addition to non-natural amino acids that contain novel side chains, non-natural amino acids that may be suitable for use in the present disclosure also optionally comprise modified backbone structures, including but not limited to, as illustrated by the structures of Formula II and III:

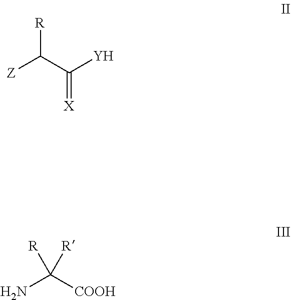

wherein Z typically comprises OH, NH₂, SH, NH—R′, or S—R′; X and Y, which can be the same or different, typically comprise S or O, and R and R′, which are optionally the same or different, are typically selected from the same list of constituents for the R group described above for the nonnatural amino acids having Formula I as well as hydrogen. For example, nonnatural amino acids of the disclosure optionally comprise substitutions in the amino or carboxyl group as illustrated by Formulas II and III. Nonnatural amino acids of this type include, but are not limited to, α-hydroxy acids, α-thioacids, α-aminothiocarboxylates, including but not limited to, with side chains corresponding to the common twenty natural amino acids or unnatural side chains. In addition, substitutions at the α-carbon optionally include, but are not limited to, L, D, or α-α-disubstituted amino acids such as D-glutamate, D-alanine, D-methyl-O-tyrosine, aminobutyric acid, and the like. Other structural alternatives include cyclic amino acids, such as proline analogues as well as 3, 4, 6, 7, 8, and 9 membered ring proline analogues, β and γ amino acids such as substituted β-alanine and γ-amino butyric acid.

Many nonnatural amino acids are based on natural amino acids, such as tyrosine, glutamine, phenylalanine, and the like, and are suitable for use in the present disclosure. Tyrosine analogs include, but are not limited to, para-substituted tyrosines, ortho-substituted tyrosines, and meta substituted tyrosines, where the substituted tyrosine comprises, including but not limited to, a keto group (including but not limited to, an acetyl group), a benzoyl group, an amino group, a hydrazine, an hydroxylamine, a thiol group, a carboxy group, an isopropyl group, a methyl group, a C₆-C₂₀ straight chain or branched hydrocarbon, a saturated or unsaturated hydrocarbon, an O-methyl group, a polyether group, a nitro group, an alkynyl group or the like. In addition, multiple substituted aryl rings are also contemplated. Glutamine analogs that may be suitable for use in the present disclosure include, but are not limited to, α-hydroxy derivatives, γ-substituted derivatives, cyclic derivatives, and amide substituted glutamine derivatives. Example phenylalanine analogs that may be suitable for use in the present disclosure include, but are not limited to, para-substituted phenylalanines, ortho-substituted phenylalanines, and meta-substituted phenylalanines, where the substituent comprises, including but not limited to, a hydroxy group, a methoxy group, a methyl group, an allyl group, an aldehyde, an azido, an iodo, a bromo, a keto group (including but not limited to, an acetyl group), a benzoyl, an alkynyl group, or the like. Specific examples of non-natural amino acids that may be suitable for use in the present disclosure include, but are not limited to, a p-acetyl-L-phenylalanine, an O-methyl-L-tyrosine, an L-3-(2-naphthyl)alanine, a 3-methyl-phenylalanine, an O-4-allyl-L-tyrosine, a 4-propyl-L-tyrosine, a tri-O-acetyl-GlcNAcβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, an isopropyl-L-phenylalanine, and a p-propargyloxy-phenylalanine, and the like. Examples of structures of a variety of nonnatural amino acids that may be suitable for use in the present disclosure are provided in, for example, WO 2002/085923 entitled “In vivo incorporation of unnatural amino acids.” See also Kiick et al., (2002) Incorporation of azides into recombinant proteins for chemoselective modification by the Staudinger ligation, PNAS 99:19-24, which is incorporated by reference herein, for additional methionine analogs. International Application No. PCT/US06/47822 entitled “Compositions Containing, Methods Involving, and Uses of Non-natural Amino Acids and Polypeptides,” which is incorporated by reference herein, describes reductive alkylation of an aromatic amine moieties, including but not limited to, p-amino-phenylalanine and reductive amination.

In another embodiment of the present disclosure, the ADC polypeptides with one or more non-naturally encoded amino acids are covalently modified. Selective chemical reactions that are orthogonal to the diverse functionality of biological systems are recognized as important tools in chemical biology. As relative newcomers to the repertoire of synthetic chemistry, these bioorthogonal reactions have inspired new strategies for compound library synthesis, protein engineering, functional proteomics, and chemical remodeling of cell surfaces. The azide has secured a prominent role as a unique chemical handle for bioconjugation. The Staudinger ligation has been used with phosphines to tag azidosugars metabolically introduced into cellular glycoconjugates. The Staudinger ligation can be performed in living animals without physiological harm; nevertheless, the Staudinger reaction is not without liabilities. The requisite phosphines are susceptible to air oxidation and their optimization for improved water solubility and increased reaction rate has proven to be synthetically challenging.

The azide group has an alternative mode of bioorthogonal reactivity: the [3+2] cycloaddition with alkynes described by Huisgen. In its classic form, this reaction has limited applicability in biological systems due to the requirement of elevated temperatures (or pressures) for reasonable reaction rates. Sharpless and coworkers surmounted this obstacle with the development of a copper(I)-catalyzed version, termed “click chemistry,” that proceeds readily at physiological temperatures and in richly functionalized biological environs. This discovery has enabled the selective modification of virus particles, nucleic acids, and proteins from complex tissue lysates. Unfortunately, the mandatory copper catalyst is toxic to both bacterial and mammalian cells, thus precluding applications wherein the cells must remain viable. Catalyst-free Huisgen cycloadditions of alkynes activated by electron-withdrawing substituents have been reported to occur at ambient temperatures. However, these compounds undergo Michael reaction with biological nucleophiles.

In one embodiment, compositions of a targeting polypeptide of the ADC that include a non-natural amino acid (such as p-(propargyloxy)-phenylalanine) are provided. Various compositions comprising p-(propargyloxy)-phenylalanine and, including but not limited to, proteins and/or cells, are also provided. In one aspect, a composition that includes the p-(propargyloxy)-phenylalanine non-natural amino acid, further includes an orthogonal tRNA. The non-natural amino acid can be bonded (including but not limited to, covalently) to the orthogonal tRNA, including but not limited to, covalently bonded to the orthogonal tRNA though an amino-acyl bond, covalently bonded to a 3′OH or a 2′OH of a terminal ribose sugar of the orthogonal tRNA, etc.

The chemical moieties via nonnatural amino acids that can be incorporated into proteins offer a variety of advantages and manipulations of the protein. For example, the unique reactivity of a keto functional group allows selective modification of proteins with any of a number of hydrazine- or hydroxylamine-containing reagents in vitro and in vivo. A heavy atom nonnatural amino acid, for example, can be useful for phasing X-ray structure data. The site-specific introduction of heavy atoms using nonnatural amino acids also provides selectivity and flexibility in choosing positions for heavy atoms. Photoreactive nonnatural amino acids (including but not limited to, amino acids with benzophenone and arylazides (including but not limited to, phenylazide) side chains), for example, allow for efficient in vivo and in vitro photocrosslinking of protein. Examples of photoreactive nonnatural amino acids include, but are not limited to, p-azido-phenylalanine and p-benzoyl-phenylalanine. The protein with the photoreactive nonnatural amino acids can then be crosslinked at will by excitation of the photoreactive group-providing temporal control. In one example, the methyl group of an nonnatural amino can be substituted with an isotopically labeled, including but not limited to, methyl group, as a probe of local structure and dynamics, including but not limited to, with the use of nuclear magnetic resonance and vibrational spectroscopy. Alkynyl or azido functional groups, for example, allow the selective modification of proteins with molecules through a [3+2] cycloaddition reaction.

A nonnatural amino acid incorporated into a polypeptide at the amino terminus can be composed of an R group that is any substituent other than one used in the twenty natural amino acids and a 2^{nd} reactive group different from the NH₂ group normally present in alpha-amino acids. A similar nonnatural amino acid can be incorporated at the C-terminus with a 2^{nd} reactive group different from the COOH group normally present in alpha-amino acids.

The nonnatural amino acids of the disclosure may be selected or designed to provide additional characteristics unavailable in the twenty natural amino acids. For example, nonnatural amino acid may be optionally designed or selected to modify the biological properties of a protein, e.g., into which they are incorporated. For example, the following properties may be optionally modified by inclusion of an nonnatural amino acid into a protein: toxicity, biodistribution, solubility, stability, e.g., thermal, hydrolytic, oxidative, resistance to enzymatic degradation, and the like, facility of purification and processing, structural properties, spectroscopic properties, chemical and/or photochemical properties, catalytic activity, redox potential, half-life, ability to react with other molecules, e.g., covalently or noncovalently, and the like.

In some embodiments the present disclosure provides ADC linked to a water soluble polymer, e.g., a PEG, by an oxime bond. Many types of non-naturally encoded amino acids are suitable for formation of oxime bonds. These include, but are not limited to, non-naturally encoded amino acids containing a carbonyl, dicarbonyl, or hydroxylamine group. Such amino acids are described in U.S. Patent Publication Nos. 2006/0194256, 2006/0217532, and 2006/0217289 and WO 2006/069246 entitled “Compositions containing, methods involving, and uses of non-natural amino acids and polypeptides,” which are incorporated herein by reference in their entirety. Non-naturally encoded amino acids are also described in U.S. Pat. Nos. 7,083,970 and 7,045,337, which are incorporated by reference herein in their entirety.

Some embodiments of the disclosure utilize ADC polypeptides that are substituted at one or more positions with a para-acetylphenylalanine amino acid. The synthesis of p-acetyl-(+/−)-phenylalanine and m-acetyl-(+/−)-phenylalanine are described in Zhang, Z., et al., Biochemistry 42: 6735-6746 (2003), incorporated by reference. Other carbonyl- or dicarbonyl-containing amino acids can be similarly prepared by one of ordinary skill in the art. Further, non-limiting exemplary syntheses of non-natural amino acid that are included herein are presented in U.S. Pat. No. 7,083,970, which is incorporated by reference herein in its entirety.

Amino acids with an electrophilic reactive group allow for a variety of reactions to link molecules via nucleophilic addition reactions among others. Such electrophilic reactive groups include a carbonyl group (including a keto group and a dicarbonyl group), a carbonyl-like group (which has reactivity similar to a carbonyl group (including a keto group and a dicarbonyl group) and is structurally similar to a carbonyl group), a masked carbonyl group (which can be readily converted into a carbonyl group (including a keto group and a dicarbonyl group)), or a protected carbonyl group (which has reactivity similar to a carbonyl group (including a keto group and a dicarbonyl group) upon deprotection). Such amino acids include amino acids having the structure of Formula (IV):

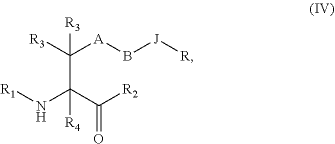

wherein:
A is optional, and when present is lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene;
B is optional, and when present is a linker selected from the group consisting of lower alkylene, substituted lower alkylene, lower alkenylene, substituted lower alkenylene, lower heteroalkylene, substituted lower heteroalkylene, —O—, —O-(alkylene or substituted alkylene)-, —S—, —S-(alkylene or substituted alkylene)-, —S(O)ₖ— where k is 1, 2, or 3, —S(O)ₖ(alkylene or substituted alkylene)-, —C(O)—, —C(O)-(alkylene or substituted alkylene)-, —C(S)—, —C(S)-(alkylene or substituted alkylene)-, —N(R′)—, —NR′-(alkylene or substituted alkylene)-, —C(O)N(R′)—, —CON(R′)-(alkylene or substituted alkylene)-, —CSN(R′)—, —CSN(R′)-(alkylene or substituted alkylene)-, —N(R′)CO-(alkylene or substituted alkylene)-, —N(R′)C(O)O—, —S(O)ₖN(R′)—, —N(R′)C(O)N(R′)—, —N(R′)C(S)N(R′)—, —N(R′)S(O)ₖN(R′)—, —N(R′)—N═, —C(R′)═N—, —C(R′)═N—N(R′)—, —C(R′)═N—N═, —C(R′)₂—N═N—, and —C(R′)₂—N(R′)—N(R′)—, where each R′ is independently H, alkyl, or substituted alkyl;
J is

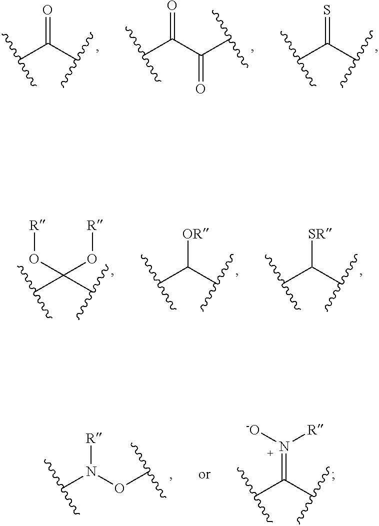

R is H, alkyl, substituted alkyl, cycloalkyl, or substituted cycloalkyl;
each R″ is independently H, alkyl, substituted alkyl, or a protecting group, or when more than one R″ group is present, two R″ optionally form a heterocycloalkyl;
R₁ is optional, and when present, is H, an amino protecting group, resin, amino acid, polypeptide, or polynucleotide; and
R₂ is optional, and when present, is OH, an ester protecting group, resin, amino acid, polypeptide, or polynucleotide;
each of R₃ and R₄ is independently H, halogen, lower alkyl, or substituted lower alkyl, or R₃ and R₄ or two R₃ groups optionally form a cycloalkyl or a heterocycloalkyl;
or the -A-B-J-R groups together form a bicyclic or tricyclic cycloalkyl or heterocycloalkyl comprising at least one carbonyl group, including a dicarbonyl group, protected carbonyl group, including a protected dicarbonyl group, or masked carbonyl group, including a masked dicarbonyl group;
or the -J-R group together forms a monocyclic or bicyclic cycloalkyl or heterocycloalkyl comprising at least one carbonyl group, including a dicarbonyl group, protected carbonyl group, including a protected dicarbonyl group, or masked carbonyl group, including a masked dicarbonyl group;
with a proviso that when A is phenylene and each R₃ is H, B is present; and that when A is —(CH₂)₄— and each R₃ is H, B is not —NHC(O)(CH₂CH₂)—; and that when A and B are absent and each R₃ is H, R is not methyl.

In addition, having the structure of Formula (V) are included:

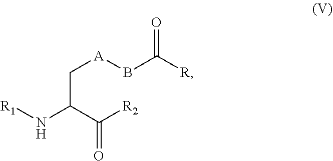

wherein:
A is optional, and when present is lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene;
B is optional, and when present is a linker selected from the group consisting of lower alkylene, substituted lower alkylene, lower alkenylene, substituted lower alkenylene, lower heteroalkylene, substituted lower heteroalkylene, —O—, —O-(alkylene or substituted alkylene)-, —S—, —S-(alkylene or substituted alkylene)-, —S(O)ₖ— where k is 1, 2, or 3, —S(O)ₖ(alkylene or substituted alkylene)-, —C(O)—, —C(O)-(alkylene or substituted alkylene)-, —C(S)—, —C(S)-(alkylene or substituted alkylene)-, —N(R′)—, —NR′-(alkylene or substituted alkylene)-, —C(O)N(R′)—, —CON(R′)-(alkylene or substituted alkylene)-, —CSN(R′)—, —CSN(R′)-(alkylene or substituted alkylene)-, —N(R′)CO-(alkylene or substituted alkylene)-, —N(R′)C(O)O—, —S(O)ₖN(R′)—, —N(R′)C(O)N(R′)—, —N(R′)C(S)N(R′)—, —N(R′)S(O)ₖN(R′)—, —N(R′)—N═, —C(R′)═N—, —C(R′)═N—N(R′)—, —C(R′)═N-N═, —C(R′)₂—N═N—, and —C(R′)₂—N(R′)—N(R′)—, where each R′ is independently H, alkyl, or substituted alkyl;
R is H, alkyl, substituted alkyl, cycloalkyl, or substituted cycloalkyl;
R₁ is optional, and when present, is H, an amino protecting group, resin, amino acid, polypeptide, or polynucleotide; and
R₂ is optional, and when present, is OH, an ester protecting group, resin, amino acid, polypeptide, or polynucleotide;
with a proviso that when A is phenylene, B is present; and that when A is —(CH₂)₄—, B is not —NHC(O)(CH₂CH₂)—; and that when A and B are absent, R is not methyl.

In addition, amino acids having the structure of Formula (VI) are included:

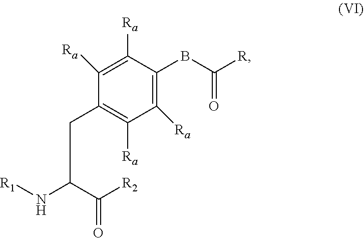

wherein:
B is a linker selected from the group consisting of lower alkylene, substituted lower alkylene, lower alkenylene, substituted lower alkenylene, lower heteroalkylene, substituted lower heteroalkylene, —O—, —O-(alkylene or substituted alkylene)-, —S—, —S-(alkylene or substituted alkylene)-, —S(O)ₖ— where k is 1, 2, or 3, —S(O)ₖ(alkylene or substituted alkylene)-, —C(O)—, —C(O)-(alkylene or substituted alkylene)-, —C(S)—, —C(S)-(alkylene or substituted alkylene)-, —N(R′)—, —NR′-(alkylene or substituted alkylene)-, —C(O)N(R′)—, —CON(R′)-(alkylene or substituted alkylene)-, —CSN(R′)—, —CSN(R′)-(alkylene or substituted alkylene)-, —N(R′)CO-(alkylene or substituted alkylene)-, —N(R′)C(O)O—, —S(O)ₖN(R′)—, —N(R′)C(O)N(R′)—, —N(R′)C(S)N(R′)—, —N(R′)S(O)ₖN(R′)—, —N(R′)—N═, —C(R′)═N—, —C(R′)═N—N(R′)—, —C(R′)═N-N═, —C(R′)₂—N═N—, and —C(R′)₂—N(R′)—N(R′)—, where each R′ is independently H, alkyl, or substituted alkyl;
R is H, alkyl, substituted alkyl, cycloalkyl, or substituted cycloalkyl;
R₁ is optional, and when present, is H, an amino protecting group, resin, amino acid, polypeptide, or polynucleotide; and
R₂ is optional, and when present, is OH, an ester protecting group, resin, amino acid, polypeptide, or polynucleotide;
each Rₐ is independently selected from the group consisting of H, halogen, alkyl, substituted alkyl, —N(R′)₂, —C(O)ₖR′ where k is 1, 2, or 3, —C(O)N(R′)₂, —OR′, and —S(O)ₖR′, where each R′ is independently H, alkyl, or substituted alkyl.

In addition, the following amino acids are included:

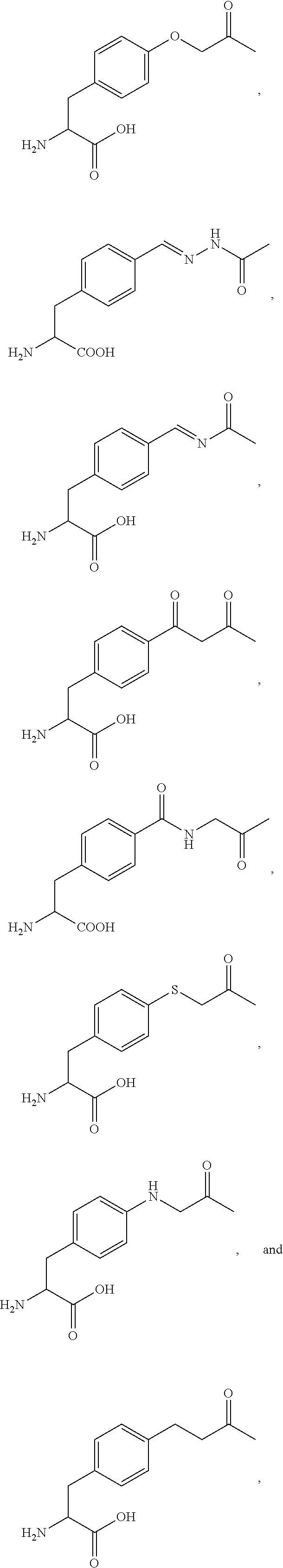

wherein such compounds are optionally amino protected group, carboxyl protected or a salt thereof. In addition, any of the following non-natural amino acids may be incorporated into a non-natural amino acid polypeptide.

In addition, the following amino acids having the structure of Formula (VII) are included:

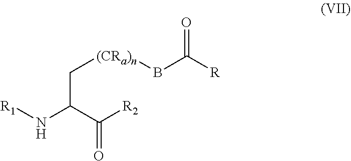

wherein
B is optional, and when present is a linker selected from the group consisting of lower alkylene, substituted lower alkylene, lower alkenylene, substituted lower alkenylene, lower heteroalkylene, substituted lower heteroalkylene, —O—, —O-(alkylene or substituted alkylene)-, —S—, —S-(alkylene or substituted alkylene)-, —S(O)ₖ— where k is 1, 2, or 3, —S(O)ₖ(alkylene or substituted alkylene)-, —C(O)—, —C(O)-(alkylene or substituted alkylene)-, —C(S)—, —C(S)-(alkylene or substituted alkylene)-, —N(R′)—, —NR′-(alkylene or substituted alkylene)-, —C(O)N(R′)—, —CON(R′)-(alkylene or substituted alkylene)-, —CSN(R′)—, —CSN(R′)-(alkylene or substituted alkylene)-, —N(R′)CO-(alkylene or substituted alkylene)-, —N(R′)C(O)O—, —S(O)ₖN(R′)—, —N(R′)C(O)N(R′)—, —N(R′)C(S)N(R′)—, —N(R′)S(O)ₖN(R′)—, —N(R′)—N═, —C(R′)═N—, —C(R′)═N—N(R′)—, —C(R′)═N—N═, —C(R′)₂—N═N—, and —C(R′)₂—N(R′)—N(R′)—, where each R′ is independently H, alkyl, or substituted alkyl;
R is H, alkyl, substituted alkyl, cycloalkyl, or substituted cycloalkyl;
R₁ is optional, and when present, is H, an amino protecting group, resin, amino acid, polypeptide, or polynucleotide; and
R₂ is optional, and when present, is OH, an ester protecting group, resin, amino acid, polypeptide, or polynucleotide;
each Rₐ is independently selected from the group consisting of H, halogen, alkyl, substituted alkyl, —N(R′)₂, —C(O)ₖR′ where k is 1, 2, or 3, —C(O)N(R′)₂, —OR′, and —S(O)ₖR′, where each R′ is independently H, alkyl, or substituted alkyl; and n is 0 to 8;
with a proviso that when A is —(CH₂)₄—, B is not —NHC(O)(CH₂CH₂)—.

In addition, the following amino acids are included:

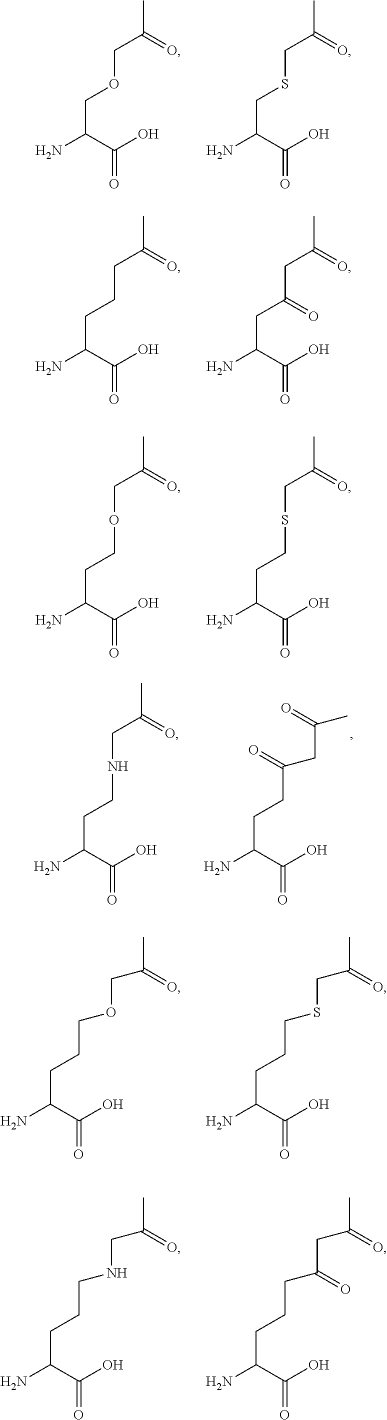

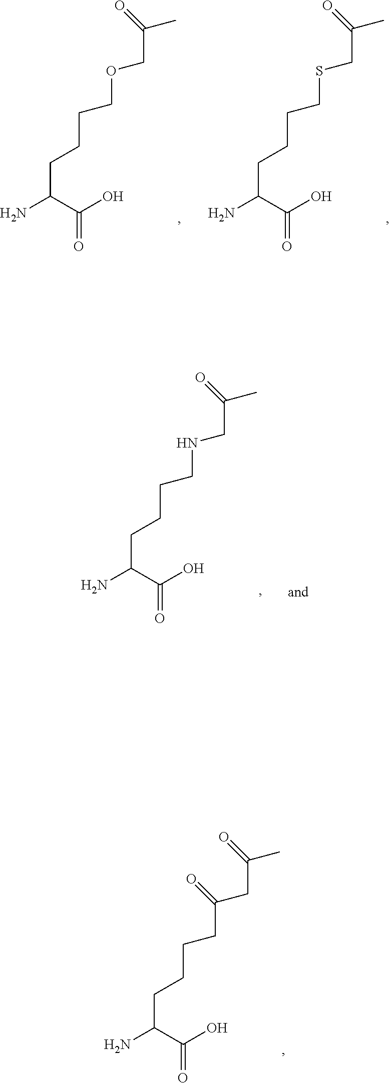

wherein such compounds are optionally amino protected, optionally carboxyl protected, optionally amino protected and carboxyl protected, or a salt thereof. In addition, these non-natural amino acids and any of the following non-natural amino acids may be incorporated into a non-natural amino acid polypeptide.

In addition, the following amino acids having the structure of Formula (VIII) are included:

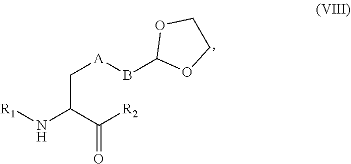

wherein A is optional, and when present is lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene;
B is optional, and when present is a linker selected from the group consisting of lower alkylene, substituted lower alkylene, lower alkenylene, substituted lower alkenylene, lower heteroalkylene, substituted lower heteroalkylene, —O—, —O-(alkylene or substituted alkylene)-, —S—, —S-(alkylene or substituted alkylene)-, —S(O)ₖ— where k is 1, 2, or 3, —S(O)ₖ(alkylene or substituted alkylene)-, —C(O)—, —C(O)-(alkylene or substituted alkylene)-, —C(S)—, —C(S)-(alkylene or substituted alkylene)-, —N(R′)—, —NR′-(alkylene or substituted alkylene)-, —C(O)N(R′)—, —CON(R′)-(alkylene or substituted alkylene)-, —CSN(R′)—, —CSN(R′)-(alkylene or substituted alkylene)-, —N(R′)CO-(alkylene or substituted alkylene)-, —N(R′)C(O)O—, —S(O)ₖN(R′)—, —N(R′)C(O)N(R′)—-N(R′)C(S)N(R′)—, —N(R′)S(O)ₖN(R′)—, —N(R′)—N═, —C(R′)═N—, —C(R′)═N—N(R′)—, —C(R′)═N—N═, —C(R′)₂—N═N—, and —C(R′)₂—N(R′)—N(R′)—, where each R′ is independently H, alkyl, or substituted alkyl;
R₁ is optional, and when present, is H, an amino protecting group, resin, amino acid, polypeptide, or polynucleotide; and
R₂ is optional, and when present, is OH, an ester protecting group, resin, amino acid, polypeptide, or polynucleotide.

In addition, the following amino acids having the structure of Formula (IX) are included:

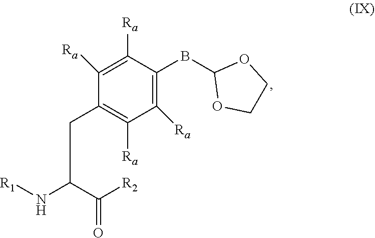

B is optional, and when present is a linker selected from the group consisting of lower alkylene, substituted lower alkylene, lower alkenylene, substituted lower alkenylene, lower heteroalkylene, substituted lower heteroalkylene, —O—, —O-(alkylene or substituted alkylene)-, —S—, —S-(alkylene or substituted alkylene)-, —S(O)ₖ— where k is 1, 2, or 3, —S(O)ₖ(alkylene or substituted alkylene)-, —C(O)—, —C(O)-(alkylene or substituted alkylene)-, —C(S)—, —C(S)-(alkylene or substituted alkylene)-, —N(R′)—, —NR′-(alkylene or substituted alkylene)-, —C(O)N(R′)—, —CON(R′)-(alkylene or substituted alkylene)-, —CSN(R′)—, —CSN(R′)-(alkylene or substituted alkylene)-, —N(R′)CO-(alkylene or substituted alkylene)-, —N(R′)C(O)O—, —S(O)ₖN(R′)—, —N(R′)C(O)N(R′)—, —N(R′)C(S)N(R′)—, —N(R′)S(O)ₖN(R′)—, —N(R′)—N═, —C(R′)═N—, —C(R′)═N—N(R′)—, —C(R′)═N—N═, —C(R′)₂—N═N—, and —C(R′)₂—N(R′)—N(R′)—, where each R′ is independently H, alkyl, or substituted alkyl;
R is H, alkyl, substituted alkyl, cycloalkyl, or substituted cycloalkyl;
R₁ is optional, and when present, is H, an amino protecting group, resin, amino acid, polypeptide, or polynucleotide; and
R₂ is optional, and when present, is OH, an ester protecting group, resin, amino acid, polypeptide, or polynucleotide;
wherein each Rₐ is independently selected from the group consisting of H, halogen, alkyl, substituted alkyl, —N(R′)₂, —C(O)ₖR′ where k is 1, 2, or 3, —C(O)N(R′)₂, —OR′, and —S(O)ₖR′, where each R′ is independently H, alkyl, or substituted alkyl.

In addition, the following amino acids are included:

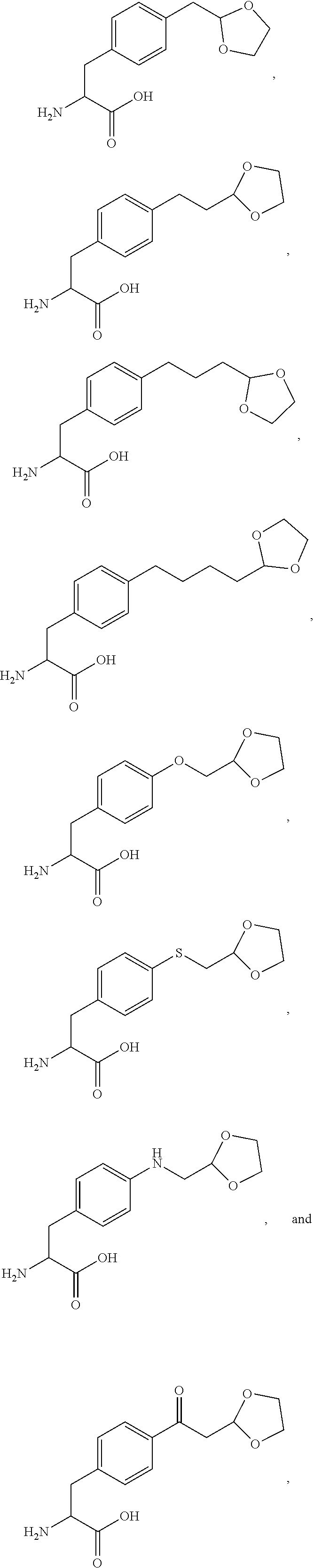

wherein such compounds are optionally amino protected, optionally carboxyl protected, optionally amino protected and carboxyl protected, or a salt thereof. In addition, these non-natural amino acids and any of the following non-natural amino acids may be incorporated into a non-natural amino acid polypeptide.

In addition, the following amino acids having the structure of Formula (X) are included:

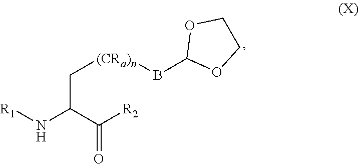

wherein B is optional, and when present is a linker selected from the group consisting of lower alkylene, substituted lower alkylene, lower alkenylene, substituted lower alkenylene, lower heteroalkylene, substituted lower heteroalkylene, —O—, —O-(alkylene or substituted alkylene)-, —S—, —S-(alkylene or substituted alkylene)-, —S(O)ₖ— where k is 1, 2, or 3, —S(O)ₖ(alkylene or substituted alkylene)-, —C(O)—, —C(O)-(alkylene or substituted alkylene)-, —C(S)—, —C(S)-(alkylene or substituted alkylene)-, —N(R′)—, —NR′-(alkylene or substituted alkylene)-, —C(O)N(R′)—, —CON(R′)-(alkylene or substituted alkylene)-, —CSN(R′)—, —CSN(R′)-(alkylene or substituted alkylene)-, —N(R′)CO-(alkylene or substituted alkylene)-, —N(R′)C(O)O—, —S(O)ₖN(R′)—, —N(R′)C(O)N(R′)—, —N(R′)C(S)N(R′)—, —N(R′)S(O)ₖN(R′)—, —N(R′)—N═, —C(R′)═N—, —C(R′)═N—N(R′)—, —C(R′)═N-N═, —C(R′)₂—N═N—, and —C(R′)₂—N(R′)—N(R′)—, where each R′ is independently H, alkyl, or substituted alkyl;
R is H, alkyl, substituted alkyl, cycloalkyl, or substituted cycloalkyl;
R₁ is optional, and when present, is H, an amino protecting group, resin, amino acid, polypeptide, or polynucleotide; and
R₂ is optional, and when present, is OH, an ester protecting group, resin, amino acid, polypeptide, or polynucleotide;
each Rₐ is independently selected from the group consisting of H, halogen, alkyl, substituted alkyl, —N(R′)₂, —C(O)ₖR′ where k is 1, 2, or 3, —C(O)N(R′)₂, —OR′, and —S(O)ₖR′, where each R′ is independently H, alkyl, or substituted alkyl; and n is 0 to 8.

In addition, the following amino acids are included:

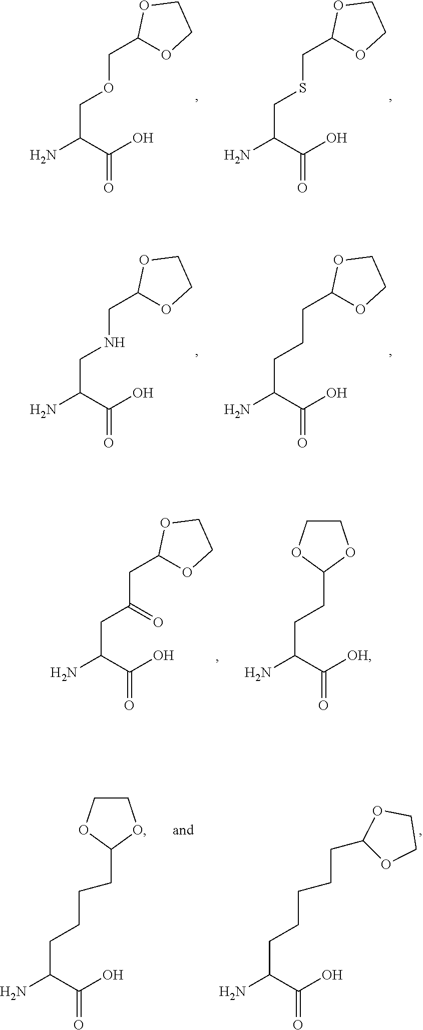

wherein such compounds are optionally amino protected, optionally carboxyl protected, optionally amino protected and carboxyl protected, or a salt thereof. In addition, these non-natural amino acids and any of the following non-natural amino acids may be incorporated into a non-natural amino acid polypeptide.

In addition to monocarbonyl structures, the non-natural amino acids described herein may include groups such as dicarbonyl, dicarbonyl like, masked dicarbonyl and protected dicarbonyl groups.

For example, the following amino acids having the structure of Formula (XI) are included:

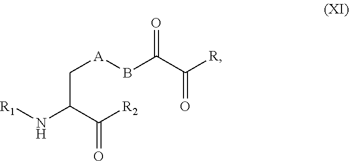

wherein A is optional, and when present is lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene;
B is optional, and when present is a linker selected from the group consisting of lower alkylene, substituted lower alkylene, lower alkenylene, substituted lower alkenylene, lower heteroalkylene, substituted lower heteroalkylene, —O—, —O-(alkylene or substituted alkylene)-, —S—, —S-(alkylene or substituted alkylene)-, —S(O)ₖ— where k is 1, 2, or 3, —S(O)ₖ(alkylene or substituted alkylene)-, —C(O)—, —C(O)-(alkylene or substituted alkylene)-, —C(S)—, —C(S)-(alkylene or substituted alkylene)-, —N(R′)—, —NR′-(alkylene or substituted alkylene)-, —C(O)N(R′)—, —CON(R′)-(alkylene or substituted alkylene)-, —CSN(R′)—, —CSN(R′)-(alkylene or substituted alkylene)-, —N(R′)CO-(alkylene or substituted alkylene)-, —N(R′)C(O)O—, —S(O)ₖN(R′)—, —N(R′)C(O)N(R′)—, —N(R′)C(S)N(R′)—, —N(R′)S(O)ₖN(R′)—, —N(R′)—N═, —C(R′)═N—, —C(R′)═N—N(R′)—, —C(R′)═N—N═, —C(R′)₂—N═N—, and —C(R′)₂—N(R′)—N(R′)—, where each R′ is independently H, alkyl, or substituted alkyl;
R is H, alkyl, substituted alkyl, cycloalkyl, or substituted cycloalkyl;
R₁ is optional, and when present, is H, an amino protecting group, resin, amino acid, polypeptide, or polynucleotide; and
R₂ is optional, and when present, is OH, an ester protecting group, resin, amino acid, polypeptide, or polynucleotide.

In addition, the following amino acids having the structure of Formula (XII) are included:

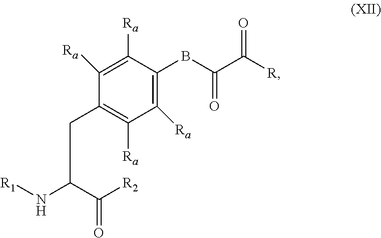

B is optional, and when present is a linker selected from the group consisting of lower alkylene, substituted lower alkylene, lower alkenylene, substituted lower alkenylene, lower heteroalkylene, substituted lower heteroalkylene, —O—, —O-(alkylene or substituted alkylene)-, —S—, —S-(alkylene or substituted alkylene)-, —S(O)ₖ— where k is 1, 2, or 3, —S(O)ₖ(alkylene or substituted alkylene)-, —C(O)—, —C(O)-(alkylene or substituted alkylene)-, —C(S)—, —C(S)-(alkylene or substituted alkylene)-, —N(R′)—, —NR′-(alkylene or substituted alkylene)-, —C(O)N(R′)—, —CON(R′)-(alkylene or substituted alkylene)-, —CSN(R′)—, —CSN(R′)-(alkylene or substituted alkylene)-, —N(R′)CO-(alkylene or substituted alkylene)-, —N(R′)C(O)O—, —S(O)ₖN(R′)—, —N(R′)C(O)N(R′)—, —N(R′)C(S)N(R′)—, —N(R′)S(O)ₖN(R′)—, —N(R′)—N═, —C(R′)═N—, —C(R′)═N—N(R′)—, —C(R′)═N—N═, —C(R′)₂—N═N—, and —C(R′)₂—N(R′)—N(R′)—, where each R′ is independently H, alkyl, or substituted alkyl;
R is H, alkyl, substituted alkyl, cycloalkyl, or substituted cycloalkyl;
R₁ is optional, and when present, is H, an amino protecting group, resin, amino acid, polypeptide, or polynucleotide; and
R₂ is optional, and when present, is OH, an ester protecting group, resin, amino acid, polypeptide, or polynucleotide;
wherein each Rₐ is independently selected from the group consisting of H, halogen, alkyl, substituted alkyl, —N(R′)₂, —C(O)ₖR′ where k is 1, 2, or 3, —C(O)N(R′)₂, —OR′, and —S(O)ₖR′, where each R′ is independently H, alkyl, or substituted alkyl.

In addition, the following amino acids are included:

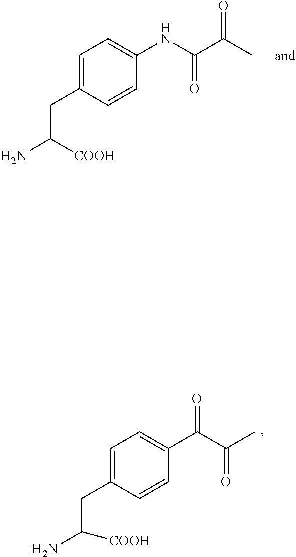 [SMILES: CC(=O)C(=O)Nc1ccc(CC(N)C(=O)O)cc1 ; CC(=O)C(=O)c1ccc(CC(N)C(=O)O)cc1]

wherein such compounds are optionally amino protected, optionally carboxyl protected, optionally amino protected and carboxyl protected, or a salt thereof. In addition, these non-natural amino acids and any of the following non-natural amino acids may be incorporated into a non-natural amino acid polypeptide.

In addition, the following amino acids having the structure of Formula (XIII) are included:

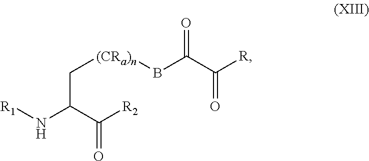

wherein is optional, an when present is a linker selected from the group consisting of lower alkylene, substituted lower alkylene, lower alkenylene, substituted lower alkenylene, lower heteroalkylene, substituted lower heteroalkylene, —O—, —O-(alkylene or substituted alkylene)-, —S—, —S-(alkylene or substituted alkylene)-, —S(O)ₖ— where k is 1, 2, or 3, —S(O)ₖ(alkylene or substituted alkylene)-, —C(O)—, —C(O)-(alkylene or substituted alkylene)-, —C(S)—, —C(S)-(alkylene or substituted alkylene)-, —N(R′)—, —NR′-(alkylene or substituted alkylene)-, —C(O)N(R′)—, —CON(R′)-(alkylene or substituted alkylene)-, —CSN(R′)—, —CSN(R′)-(alkylene or substituted alkylene)-, —N(R′)CO-(alkylene or substituted alkylene)-, —N(R′)C(O)O—, —S(O)ₖN(R′)—, —N(R′)C(O)N(R′)—, —N(R′)C(S)N(R′)—, —N(R′)S(O)ₖN(R′)—, —N(R′)—N═, —C(R′)═N—, —C(R′)═N—N(R′)—, —C(R′)═N-N═, —C(R′)₂—N═N—, and —C(R′)₂—N(R′)—N(R′)—, where each R′ is independently H, alkyl, or substituted alkyl;
R is H, alkyl, substituted alkyl, cycloalkyl, or substituted cycloalkyl;
R₁ is optional, and when present, is H, an amino protecting group, resin, amino acid, polypeptide, or polynucleotide; and
R₂ is optional, and when present, is OH, an ester protecting group, resin, amino acid, polypeptide, or polynucleotide;
each Rₐ is independently selected from the group consisting of H, halogen, alkyl, substituted alkyl, —N(R′)₂, —C(O)ₖR′ where k is 1, 2, or 3, —C(O)N(R′)₂, —OR′, and —S(O)ₖR′, where each R′ is independently H, alkyl, or substituted alkyl; and n is 0 to 8.

In addition, the following amino acids are included:

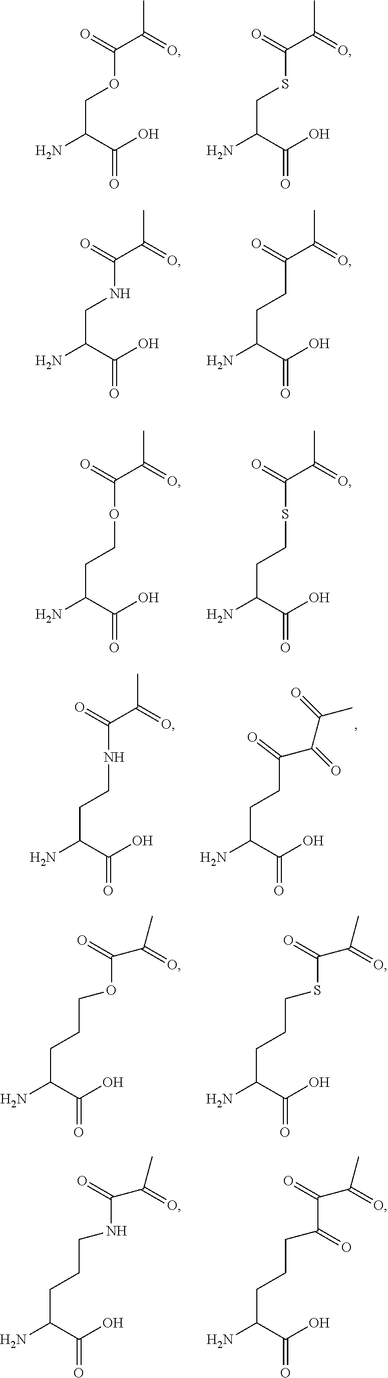

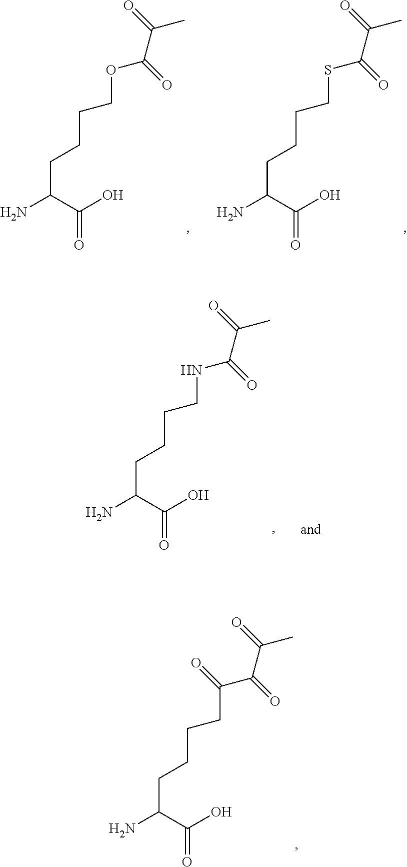 [SMILES: CC(=O)C(=O)OCCCCC(N)C(=O)O ; CC(=O)C(=O)SCCCCC(N)C(=O)O ; CC(=O)C(=O)NCCCCC(N)C(=O)O ; CC(=O)C(=O)C(=O)CCCCC(N)C(=O)O]

wherein such compounds are optionally amino protected, optionally carboxyl protected, optionally amino protected and carboxyl protected, or a salt thereof. In addition, these non-natural amino acids and any of the following non-natural amino acids may be incorporated into a non-natural amino acid polypeptide.

In addition, the following amino acids having the structure of Formula (XIV) are included:

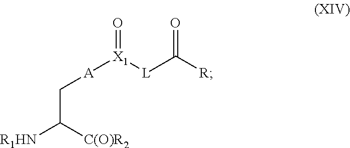

wherein:
A is optional, and when present is lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene;
R is H, alkyl, substituted alkyl, cycloalkyl, or substituted cycloalkyl;
R₁ is optional, and when present, is H, an amino protecting group, resin, amino acid, polypeptide, or polynucleotide; and
R₂ is optional, and when present, is OH, an ester protecting group, resin, amino acid, polypeptide, or polynucleotide;
X₁ is C, S, or S(O); and L is alkylene, substituted alkylene, N(R′)(alkylene) or N(R′)(substituted alkylene), where R′ is H, alkyl, substituted alkyl, cycloalkyl, or substituted cycloalkyl.

In addition, the following amino acids having the structure of Formula (XIV-A) are included:

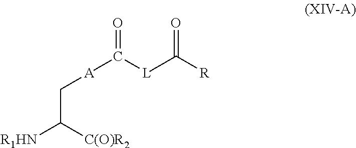

wherein:
A is optional, and when present is lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene;
R is H, alkyl, substituted alkyl, cycloalkyl, or substituted cycloalkyl;
R₁ is optional, and when present, is H, an amino protecting group, resin, amino acid, polypeptide, or polynucleotide; and
R₂ is optional, and when present, is OH, an ester protecting group, resin, amino acid, polypeptide, or polynucleotide;
L is alkylene, substituted alkylene, N(R′)(alkylene) or N(R′)(substituted alkylene), where R′ is H, alkyl, substituted alkyl, cycloalkyl, or substituted cycloalkyl.

In addition, the following amino acids having the structure of Formula (XIV-B) are included:

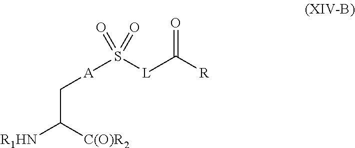

wherein:
A is optional, and when present is lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene;
R is H, alkyl, substituted alkyl, cycloalkyl, or substituted cycloalkyl;
R₁ is optional, and when present, is H, an amino protecting group, resin, amino acid, polypeptide, or polynucleotide; and
R₂ is optional, and when present, is OH, an ester protecting group, resin, amino acid, polypeptide, or polynucleotide;
L is alkylene, substituted alkylene, N(R′)(alkylene) or N(R′)(substituted alkylene), where R′ is H, alkyl, substituted alkyl, cycloalkyl, or substituted cycloalkyl.

In addition, the following amino acids having the structure of Formula (XV) are included:

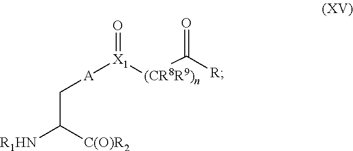

wherein:
A is optional, and when present is lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene;
R is H, alkyl, substituted alkyl, cycloalkyl, or substituted cycloalkyl;
R₁ is optional, and when present, is H, an amino protecting group, resin, amino acid, polypeptide, or polynucleotide; and
R₂ is optional, and when present, is OH, an ester protecting group, resin, amino acid, polypeptide, or polynucleotide;
X₁ is C, S, or S(O); and n is 0, 1, 2, 3, 4, or 5; and each R¹ and R⁹ on each CR⁸R⁹ group is independently selected from the group consisting of H, alkoxy, alkylamine, halogen, alkyl, aryl, or any R⁸ and R⁹ can together form ═O or a cycloalkyl, or any to adjacent R⁸ groups can together form a cycloalkyl.

In addition, the following amino acids having the structure of Formula (XV-A) are included:

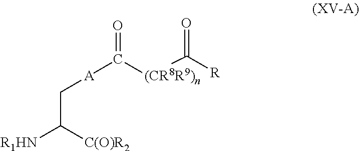

wherein:
A is optional, and when present is lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene;
R is H, alkyl, substituted alkyl, cycloalkyl, or substituted cycloalkyl;
R₁ is optional, and when present, is H, an amino protecting group, resin, amino acid, polypeptide, or polynucleotide; and
R₂ is optional, and when present, is OH, an ester protecting group, resin, amino acid, polypeptide, or polynucleotide;
n is 0, 1, 2, 3, 4, or 5; and each R¹ and R⁹ on each CR⁸R⁹ group is independently selected from the group consisting of H, alkoxy, alkylamine, halogen, alkyl, aryl, or any R¹ and R⁹ can together form ═O or a cycloalkyl, or any to adjacent R⁸ groups can together form a cycloalkyl.

In addition, the following amino acids having the structure of Formula (XV-B) are included:

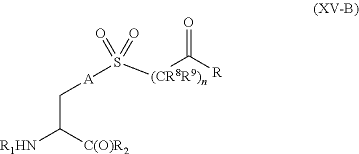

wherein:
A is optional, and when present is lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene;
R is H, alkyl, substituted alkyl, cycloalkyl, or substituted cycloalkyl;
R₁ is optional, and when present, is H, an amino protecting group, resin, amino acid, polypeptide, or polynucleotide; and
R₂ is optional, and when present, is OH, an ester protecting group, resin, amino acid, polypeptide, or polynucleotide;
n is 0, 1, 2, 3, 4, or 5; and each R¹ and R⁹ on each CR⁸R⁹ group is independently selected from the group consisting of H, alkoxy, alkylamine, halogen, alkyl, aryl, or any R¹ and R⁹ can together form ═O or a cycloalkyl, or any to adjacent R⁸ groups can together form a cycloalkyl.

In addition, the following amino acids having the structure of Formula (XVI) are included:

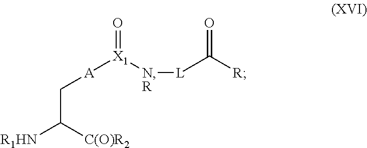

wherein:
A is optional, and when present is lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene;
R is H, alkyl, substituted alkyl, cycloalkyl, or substituted cycloalkyl;
R₁ is optional, and when present, is H, an amino protecting group, resin, amino acid, polypeptide, or polynucleotide; and
R₂ is optional, and when present, is OH, an ester protecting group, resin, amino acid, polypeptide, or polynucleotide;
X₁ is C, S, or S(O); and L is alkylene, substituted alkylene, N(R′)(alkylene) or N(R′)(substituted alkylene), where R′ is H, alkyl, substituted alkyl, cycloalkyl, or substituted cycloalkyl.

In addition, the following amino acids having the structure of Formula (XVI-A) are included:

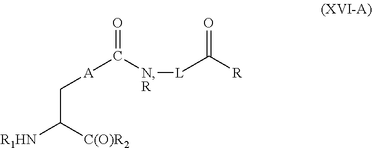

wherein:
A is optional, and when present is lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene;
R is H, alkyl, substituted alkyl, cycloalkyl, or substituted cycloalkyl;
R₁ is optional, and when present, is H, an amino protecting group, resin, amino acid, polypeptide, or polynucleotide; and
R₂ is optional, and when present, is OH, an ester protecting group, resin, amino acid, polypeptide, or polynucleotide;
L is alkylene, substituted alkylene, N(R′)(alkylene) or N(R′)(substituted alkylene), where R′ is H, alkyl, substituted alkyl, cycloalkyl, or substituted cycloalkyl.

In addition, the following amino acids having the structure of Formula (XVI-B) are included:

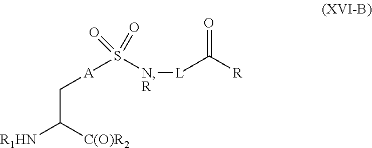

wherein:
A is optional, and when present is lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene;
R is H, alkyl, substituted alkyl, cycloalkyl, or substituted cycloalkyl;
R₁ is optional, and when present, is H, an amino protecting group, resin, amino acid, polypeptide, or polynucleotide; and
R₂ is optional, and when present, is OH, an ester protecting group, resin, amino acid, polypeptide, or polynucleotide;
L is alkylene, substituted alkylene, N(R′)(alkylene) or N(R′)(substituted alkylene), where R′ is H, alkyl, substituted alkyl, cycloalkyl, or substituted cycloalkyl.

In addition, amino acids having the structure of Formula (XVII) are included:

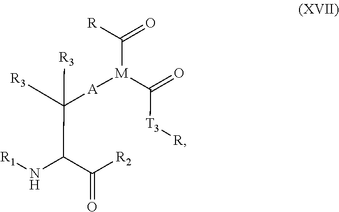

wherein:
A is optional, and when present is lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene; M is —C(R₃)—,

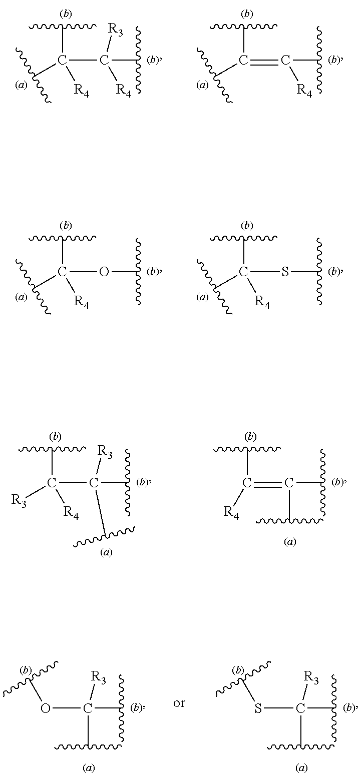

where (a) indicates bonding to the A group and (b) indicates bonding to respective carbonyl groups, R₃ and R₄ are independently chosen from H, halogen, alkyl, substituted alkyl, cycloalkyl, or substituted cycloalkyl, or R₃ and R₄ or two R₃ groups or two R₄ groups optionally form a cycloalkyl or a heterocycloalkyl;
R is H, halogen, alkyl, substituted alkyl, cycloalkyl, or substituted cycloalkyl;
T₃ is a bond, C(R)(R), O, or S, and R is H, halogen, alkyl, substituted alkyl, cycloalkyl, or substituted cycloalkyl;
R₁ is optional, and when present, is H, an amino protecting group, resin, amino acid, polypeptide, or polynucleotide; and
R₂ is optional, and when present, is OH, an ester protecting group, resin, amino acid, polypeptide, or polynucleotide.

In addition, amino acids having the structure of Formula (XVIII) are included:

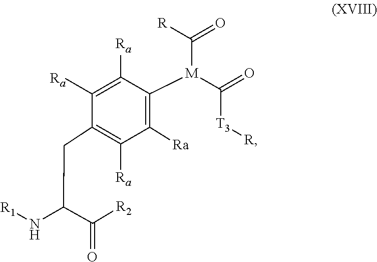

wherein:
M is —C(R₃)—,

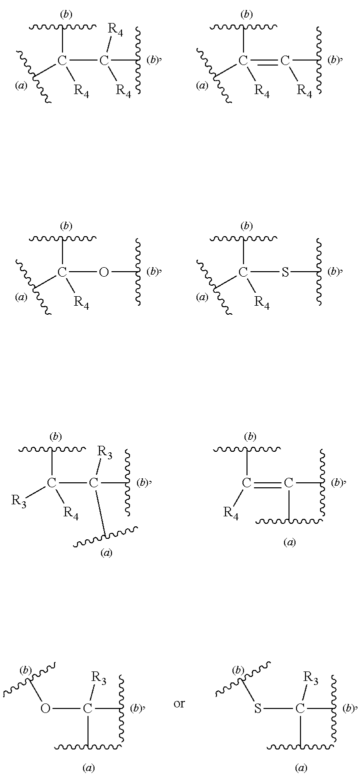

where (a) indicates bonding to the A group and (b) indicates bonding to respective carbonyl groups, R₃ and R₄ are independently chosen from H, halogen, alkyl, substituted alkyl, cycloalkyl, or substituted cycloalkyl, or R₃ and R₄ or two R₃ groups or two R₄ groups optionally form a cycloalkyl or a heterocycloalkyl;
R is H, halogen, alkyl, substituted alkyl, cycloalkyl, or substituted cycloalkyl;
T₃ is a bond, C(R)(R), O, or S, and R is H, halogen, alkyl, substituted alkyl, cycloalkyl, or substituted cycloalkyl;
R₁ is optional, and when present, is H, an amino protecting group, resin, amino acid, polypeptide, or polynucleotide; and
R₂ is optional, and when present, is OH, an ester protecting group, resin, amino acid, polypeptide, or polynucleotide;
each Rₐ is independently selected from the group consisting of H, halogen, alkyl, substituted alkyl, —N(R′)₂, —C(O)ₖR′ where k is 1, 2, or 3, —C(O)N(R′)₂, —OR′, and —S(O)ₖR′, where each R′ is independently H, alkyl, or substituted alkyl.

In addition, amino acids having the structure of Formula (XIX) are included:

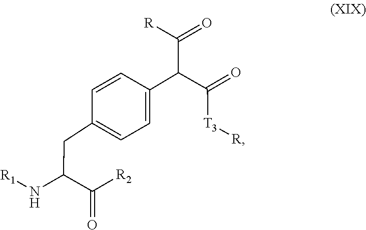

wherein:
R is H, halogen, alkyl, substituted alkyl, cycloalkyl, or substituted cycloalkyl; and
T₃ is O, or S.

In addition, amino acids having the structure of Formula (XX) are included:

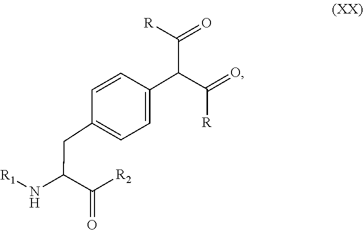

wherein:
R is H, halogen, alkyl, substituted alkyl, cycloalkyl, or substituted cycloalkyl.

In addition, the following amino acids having structures of Formula (XXI) are included:

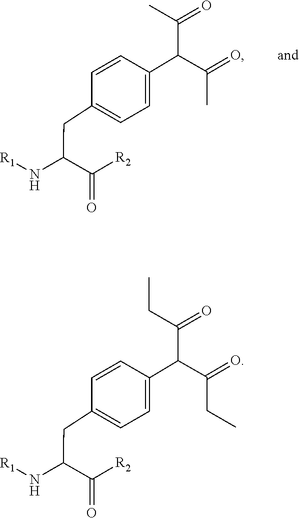

In some embodiments, a polypeptide comprising a non-natural amino acid is chemically modified to generate a reactive carbonyl or dicarbonyl functional group. For instance, an aldehyde functionality useful for conjugation reactions can be generated from a functionality having adjacent amino and hydroxyl groups. Where the biologically active molecule is a polypeptide, for example, an N-terminal serine or threonine (which may be normally present or may be exposed via chemical or enzymatic digestion) can be used to generate an aldehyde functionality under mild oxidative cleavage conditions using periodate. See, e.g., Gaertner, et. al., Bioconjug. Chem. 3: 262-268 (1992); Geoghegan, K. & Stroh, J., Bioconjug. Chem. 3:138-146 (1992); Gaertner et al., J. Biol. Chem. 269:7224-7230 (1994). However, methods known in the art are restricted to the amino acid at the N-terminus of the peptide or protein.

In the present disclosure, a non-natural amino acid bearing adjacent hydroxyl and amino groups can be incorporated into the polypeptide as a “masked” aldehyde functionality. For example, 5-hydroxylysine bears a hydroxyl group adjacent to the epsilon amine. Reaction conditions for generating the aldehyde typically involve addition of molar excess of sodium metaperiodate under mild conditions to avoid oxidation at other sites within the polypeptide. The pH of the oxidation reaction is typically about 7.0. A typical reaction involves the addition of about 1.5 molar excess of sodium meta periodate to a buffered solution of the polypeptide, followed by incubation for about 10 minutes in the dark. See, e.g. U.S. Pat. No. 6,423,685.

The carbonyl or dicarbonyl functionality can be reacted selectively with a hydroxylamine-containing reagent under mild conditions in aqueous solution to form the corresponding oxime linkage that is stable under physiological conditions. See, e.g., Jencks, W. P., J. Am. Chem. Soc. 81, 475-481 (1959); Shao, J. and Tam, J. P., J. Am. Chem. Soc. 117:3893-3899 (1995). Moreover, the unique reactivity of the carbonyl or dicarbonyl group allows for selective modification in the presence of the other amino acid side chains. See, e.g., Cornish, V. W., et al., J. Am. Chem. Soc. 118:8150-8151 (1996); Geoghegan, K. F. & Stroh, J. G., Bioconjug. Chem. 3:138-146 (1992); Mahal, L. K., et al., Science 276:1125-1128 (1997).

### A. Carbonyl Reactive Groups

Amino acids with a carbonyl reactive group allow for a variety of reactions to link molecules (including but not limited to, PEG or other water soluble molecules) via nucleophilic addition or aldol condensation reactions among others.

Exemplary carbonyl-containing amino acids can be represented as follows:

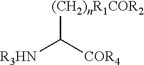

wherein n is 0-10; R₁ is an alkyl, aryl, substituted alkyl, or substituted aryl; R₂ is H, alkyl, aryl, substituted alkyl, and substituted aryl; and R₃ is H, an amino acid, a polypeptide, or an amino terminus modification group, and R₄ is H, an amino acid, a polypeptide, or a carboxy terminus modification group. In some embodiments, n is 1, R₁ is phenyl and R₂ is a simple alkyl (i.e., methyl, ethyl, or propyl) and the ketone moiety is positioned in the para position relative to the alkyl side chain. In some embodiments, n is 1, R₁ is phenyl and R₂ is a simple alkyl (i.e., methyl, ethyl, or propyl) and the ketone moiety is positioned in the meta position relative to the alkyl side chain.

The synthesis of p-acetyl-(+/−)-phenylalanine and m-acetyl-(+/−)-phenylalanine is described in Zhang, Z., et al., Biochemistry 42: 6735-6746 (2003), which is incorporated by reference herein. Other carbonyl-containing amino acids can be similarly prepared by one of ordinary skill in the art.

In some embodiments, a polypeptide comprising a non-naturally encoded amino acid is chemically modified to generate a reactive carbonyl functional group. For instance, an aldehyde functionality useful for conjugation reactions can be generated from a functionality having adjacent amino and hydroxyl groups. Where the biologically active molecule is a polypeptide, for example, an N-terminal serine or threonine (which may be normally present or may be exposed via chemical or enzymatic digestion) can be used to generate an aldehyde functionality under mild oxidative cleavage conditions using periodate. See, e.g., Gaertner, et al., Bioconjug. Chem. 3: 262-268 (1992); Geoghegan, K. & Stroh, J., Bioconjug. Chem. 3:138-146 (1992); Gaertner et al., J. Biol. Chem. 269:7224-7230 (1994). However, methods known in the art are restricted to the amino acid at the N-terminus of the peptide or protein.

In the present disclosure, a non-naturally encoded amino acid bearing adjacent hydroxyl and amino groups can be incorporated into the polypeptide as a “masked” aldehyde functionality. For example, 5-hydroxylysine bears a hydroxyl group adjacent to the epsilon amine. Reaction conditions for generating the aldehyde typically involve addition of molar excess of sodium metaperiodate under mild conditions to avoid oxidation at other sites within the polypeptide. The pH of the oxidation reaction is typically about 7.0. A typical reaction involves the addition of about 1.5 molar excess of sodium meta periodate to a buffered solution of the polypeptide, followed by incubation for about 10 minutes in the dark. See, e.g. U.S. Pat. No. 6,423,685, which is incorporated by reference herein.

The carbonyl functionality can be reacted selectively with a hydrazine-, hydrazide-, hydroxylamine-, or semicarbazide-containing reagent under mild conditions in aqueous solution to form the corresponding hydrazone, oxime, or semicarbazone linkages, respectively, that are stable under physiological conditions. See, e.g., Jencks, W. P., J. Am. Chem. Soc. 81, 475-481 (1959); Shao, J. and Tam, J. P., J. Am. Chem. Soc. 117:3893-3899 (1995). Moreover, the unique reactivity of the carbonyl group allows for selective modification in the presence of the other amino acid side chains. See, e.g., Cornish, V. W., et al., J. Am. Chem. Soc. 118:8150-8151 (1996); Geoghegan, K. F. & Stroh, J. G., Bioconjug. Chem. 3:138-146 (1992); Mahal, L. K., et al., Science 276:1125-1128 (1997).

### B. Hydrazine, Hydrazide or Semicarbazide Reactive Groups

Non-naturally encoded amino acids containing a nucleophilic group, such as a hydrazine, hydrazide or semicarbazide, allow for reaction with a variety of electrophilic groups to form conjugates (including but not limited to, with PEG or other water soluble polymers).

Exemplary hydrazine, hydrazide or semicarbazide-containing amino acids can be represented as follows:

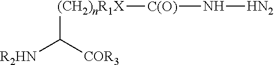

wherein n is 0-10; R₁ is an alkyl, aryl, substituted alkyl, or substituted aryl or not present; X, is O, N, or S or not present; R₂ is H, an amino acid, a polypeptide, or an amino terminus modification group, and R₃ is H, an amino acid, a polypeptide, or a carboxy terminus modification group.

In some embodiments, n is 4, R₁ is not present, and X is N. In some embodiments, n is 2, R₁ is not present, and X is not present. In some embodiments, n is 1, R₁ is phenyl, X is 0, and the oxygen atom is positioned para to the aliphatic group on the aryl ring.

Hydrazide-, hydrazine-, and semicarbazide-containing amino acids are available from commercial sources. For instance, L-glutamate-y-hydrazide is available from Sigma Chemical (St. Louis, MO). Other amino acids not available commercially can be prepared by one of ordinary skill in the art. See, e.g., U.S. Pat. No. 6,281,211, which is incorporated by reference herein.

Polypeptides containing non-naturally encoded amino acids that bear hydrazide, hydrazine or semicarbazide functionalities can be reacted efficiently and selectively with a variety of molecules that contain aldehydes or other functional groups with similar chemical reactivity. See, e.g., Shao, J. and Tam, J., J. Am. Chem. Soc. 117:3893-3899 (1995). The unique reactivity of hydrazide, hydrazine and semicarbazide functional groups makes them significantly more reactive toward aldehydes, ketones and other electrophilic groups as compared to the nucleophilic groups present on the 20 common amino acids (including but not limited to, the hydroxyl group of serine or threonine or the amino groups of lysine and the N-terminus).

### C. Aminooxy-Containing Amino Acids

Non-naturally encoded amino acids containing an aminooxy (also called a hydroxylamine) group allow for reaction with a variety of electrophilic groups to form conjugates (including but not limited to, with PEG or other water soluble polymers). Like hydrazines, hydrazides and semicarbazides, the enhanced nucleophilicity of the aminooxy group permits it to react efficiently and selectively with a variety of molecules that contain aldehydes or other functional groups with similar chemical reactivity. See, e.g., Shao, J. and Tam, J., J. Am. Chem. Soc. 117:3893-3899 (1995); H. Hang and C. Bertozzi, Acc. Chem. Res. 34: 727-736 (2001). Whereas the result of reaction with a hydrazine group is the corresponding hydrazone, however, an oxime results generally from the reaction of an aminooxy group with a carbonyl-containing group such as a ketone.

Exemplary amino acids containing aminooxy groups can be represented as follows:

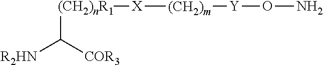

wherein n is 0-10; R₁ is an alkyl, aryl, substituted alkyl, or substituted aryl or not present; X is 0, N, S or not present; m is 0-10; Y═C(O) or not present; R₂ is H, an amino acid, a polypeptide, or an amino terminus modification group, and R₃ is H, an amino acid, a polypeptide, or a carboxy terminus modification group. In some embodiments, n is 1, R₁ is phenyl, X is 0, m is 1, and Y is present. In some embodiments, n is 2, R₁ and X are not present, m is 0, and Y is not present.

Aminooxy-containing amino acids can be prepared from readily available amino acid precursors (homoserine, serine and threonine). See, e.g., M. Carrasco and R. Brown, J. Org. Chem. 68: 8853-8858 (2003). Certain aminooxy-containing amino acids, such as L-2-amino-4-(aminooxy)butyric acid), have been isolated from natural sources (Rosenthal, G., Life Sci. 60: 1635-1641 (1997). Other aminooxy-containing amino acids can be prepared by one of ordinary skill in the art.

### D. Azide and Alkyne Reactive Groups

The unique reactivity of azide and alkyne functional groups makes them extremely useful for the selective modification of polypeptides and other biological molecules. Organic azides, particularly aliphatic azides, and alkynes are generally stable toward common reactive chemical conditions. In particular, both the azide and the alkyne functional groups are inert toward the side chains (i.e., R groups) of the 20 common amino acids found in naturally-occurring polypeptides. When brought into close proximity however, the “spring-loaded” nature of the azide and alkyne groups is revealed, and they react selectively and efficiently via Huisgen [3+2] cycloaddition reaction to generate the corresponding triazole. See, e.g., Chin J., et al., Science 301:964-7 (2003); Wang, Q., et al., J. Am. Chem. Soc. 125, 3192-3193 (2003); Chin, J. W., et al., J. Am. Chem. Soc. 124:9026-9027 (2002).

Because the Huisgen cycloaddition reaction involves a selective cycloaddition reaction (see, e.g., Padwa, A., in COMPREHENSIVE ORGANIC SYNTHESIS, Vol. 4, (ed. Trost, B. M., 1991), p. 1069-1109; Huisgen, R. in 1,3-DIPOLAR CYCLOADDITION CHEMISTRY, (ed. Padwa, A., 1984), p. 1-176) rather than a nucleophilic substitution, the incorporation of non-naturally encoded amino acids bearing azide and alkyne-containing side chains permits the resultant polypeptides to be modified selectively at the position of the non-naturally encoded amino acid. Cycloaddition reaction involving azide or alkyne-containing ADC can be carried out at room temperature under aqueous conditions by the addition of Cu(II) (including but not limited to, in the form of a catalytic amount of CuSO₄) in the presence of a reducing agent for reducing Cu(II) to Cu(I), in situ, in catalytic amount. See, e.g., Wang, Q., et al., J. Am. Chem. Soc. 125, 3192-3193 (2003); Tornoe, C. W., et al., J. Org. Chem. 67:3057-3064 (2002); Rostovtsev, et al., Angew. Chem. Int. Ed. 41:2596-2599 (2002). Exemplary reducing agents include, including but not limited to, ascorbate, metallic copper, quinine, hydroquinone, vitamin K, glutathione, cysteine, Fe²⁺, Co²⁺, and an applied electric potential.

In some cases, where a Huisgen [3+2] cycloaddition reaction between an azide and an alkyne is desired, the ADC comprises a non-naturally encoded amino acid comprising an alkyne moiety and the water soluble polymer to be attached to the amino acid comprises an azide moiety. Alternatively, the converse reaction (i.e., with the azide moiety on the amino acid and the alkyne moiety present on the water soluble polymer) can also be performed.

The azide functional group can also be reacted selectively with a water soluble polymer containing an aryl ester and appropriately functionalized with an aryl phosphine moiety to generate an amide linkage. The aryl phosphine group reduces the azide in situ and the resulting amine then reacts efficiently with a proximal ester linkage to generate the corresponding amide. See, e.g., E. Saxon and C. Bertozzi, Science 287, 2007-2010 (2000). The azide-containing amino acid can be either an alkyl azide (including but not limited to, 2-amino-6-azido-1-hexanoic acid) or an aryl azide (p-azido-phenylalanine).

Exemplary water soluble polymers containing an aryl ester and a phosphine moiety can be represented as follows:

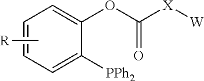

wherein X can be O, N, S or not present, Ph is phenyl, W is a water soluble polymer and R can be H, alkyl, aryl, substituted alkyl and substituted aryl groups. Exemplary R groups include but are not limited to —CH₂, —C(CH₃)₃, —OR′, —NR′R″, —SR′, -halogen, —C(O)R′, —CONR′R″, —S(O)₂R′, —S(O)₂NR′R″, —CN and —NO₂. R′, R″, R′″ and R″″ each independently refer to hydrogen, substituted or unsubstituted heteroalkyl, substituted or unsubstituted aryl, including but not limited to, aryl substituted with 1-3 halogens, substituted or unsubstituted alkyl, alkoxy or thioalkoxy groups, or arylalkyl groups. When a compound of the disclosure includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (including but not limited to, —CF₃ and —CH₂CF₃) and acyl (including but not limited to, —C(O)CH₃, —C(O)CF₃, —C(O)CH₂OCH₃, and the like).

The azide functional group can also be reacted selectively with a water soluble polymer containing a thioester and appropriately functionalized with an aryl phosphine moiety to generate an amide linkage. The aryl phosphine group reduces the azide in situ and the resulting amine then reacts efficiently with the thioester linkage to generate the corresponding amide. Exemplary water soluble polymers containing a thioester and a phosphine moiety can be represented as follows:

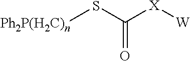

wherein n is 1-10; X can be O, N, S or not present, Ph is phenyl, and W is a water soluble polymer.

Exemplary alkyne-containing amino acids can be represented as follows:

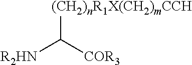

wherein n is 0-10; R₁ is an alkyl, aryl, substituted alkyl, or substituted aryl or not present; X is O, N, S or not present; m is 0-10, R₂ is H, an amino acid, a polypeptide, or an amino terminus modification group, and R₃ is H, an amino acid, a polypeptide, or a carboxy terminus modification group. In some embodiments, n is 1, R₁ is phenyl, X is not present, m is 0 and the acetylene moiety is positioned in the para position relative to the alkyl side chain. In some embodiments, n is 1, R₁ is phenyl, X is 0, m is 1 and the propargyloxy group is positioned in the para position relative to the alkyl side chain (i.e., 0-propargyl-tyrosine). In some embodiments, n is 1, R₁ and X are not present, and m is 0 (i.e., propargylglycine).

Alkyne-containing amino acids are commercially available. For example, propargylglycine is commercially available from Peptech (Burlington, MA). Alternatively, alkyne-containing amino acids can be prepared according to standard methods. For instance, p-propargyloxyphenylalanine can be synthesized, for example, as described in Deiters, A., et al., J. Am. Chem. Soc. 125: 11782-11783 (2003), and 4-alkynyl-L-phenylalanine can be synthesized as described in Kayser, B., et al., Tetrahedron 53(7): 2475-2484 (1997). Other alkyne-containing amino acids can be prepared by one of ordinary skill in the art.

Exemplary azide-containing amino acids can be represented as follows:

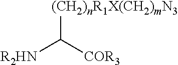

wherein n is 0-10; R₁ is an alkyl, aryl, substituted alkyl, substituted aryl or not present; X is 0, N, S or not present; m is 0-10; R₂ is H, an amino acid, a polypeptide, or an amino terminus modification group, and R₃ is H, an amino acid, a polypeptide, or a carboxy terminus modification group. In some embodiments, n is 1, R₁ is phenyl, X is not present, m is 0 and the azide moiety is positioned para to the alkyl side chain. In some embodiments, n is 0-4 and R₁ and X are not present, and m=0. In some embodiments, n is 1, R₁ is phenyl, X is 0, m is 2 and the -azidoethoxy moiety is positioned in the para position relative to the alkyl side chain.

Azide-containing amino acids are available from commercial sources. For instance, 4-azidophenylalanine can be obtained from Chem-Impex International, Inc. (Wood Dale, IL). For those azide-containing amino acids that are not commercially available, the azide group can be prepared relatively readily using standard methods known to those of ordinary skill in the art, including but not limited to, via displacement of a suitable leaving group (including but not limited to, halide, mesylate, tosylate) or via opening of a suitably protected lactone. See, e.g., Advanced Organic Chemistry by March (Third Edition, 1985, Wiley and Sons, New York).

### E. Aminothiol Reactive Groups

The unique reactivity of beta-substituted aminothiol functional groups makes them extremely useful for the selective modification of polypeptides and other biological molecules that contain aldehyde groups via formation of the thiazolidine. See, e.g., J. Shao and J. Tam, J. Am. Chem. Soc. 1995, 117 (14) 3893-3899. In some embodiments, beta-substituted aminothiol amino acids can be incorporated into ADC polypeptides and then reacted with water soluble polymers comprising an aldehyde functionality. In some embodiments, a water soluble polymer, drug conjugate or other payload can be coupled to a targeting polypeptide of the ADC comprising a beta-substituted aminothiol amino acid via formation of the thiazolidine.

### F. Additional Reactive Groups

Additional reactive groups and non-naturally encoded amino acids, including but not limited to para-amino-phenylalanine, that can be incorporated into ADC polypeptides of the disclosure are described in the following patent applications which are all incorporated by reference in their entirety herein: U.S. Patent Publication No. 2006/0194256, U.S. Patent Publication No. 2006/0217532, U.S. Patent Publication No. 2006/0217289, U.S. Provisional Patent No. 60/755,338; U.S. Provisional Patent No. 60/755,711; U.S. Provisional Patent No. 60/755,018; International Patent Application No. PCT/US06/49397; WO 2006/069246; U.S. Provisional Patent No. 60/743,041; U.S. Provisional Patent No. 60/743,040; International Patent Application No. PCT/US06/47822; U.S. Provisional Patent No. 60/882,819; U.S. Provisional Patent No. 60/882,500; and U.S. Provisional Patent No. 60/870,594. These applications also discuss reactive groups that may be present on PEG or other polymers, including but not limited to, hydroxylamine (aminooxy) groups for conjugation.

### Location of Non-Natural Amino Acids in ADC Polypeptides

The methods and compositions described herein include incorporation of one or more non-natural amino acids into a targeting polypeptide to make a ADC of the present disclosure. One or more non-natural amino acids may be incorporated at one or more particular positions which do not disrupt activity of the targeting polypeptide. This can be achieved by making “conservative” substitutions, including but not limited to, substituting hydrophobic amino acids with non-natural or natural hydrophobic amino acids, bulky amino acids with non-natural or natural bulky amino acids, hydrophilic amino acids with non-natural or natural hydrophilic amino acids) and/or inserting the non-natural amino acid in a location that is not required for activity.

A variety of biochemical and structural approaches can be employed to select the desired sites for substitution with a non-natural amino acid within the targeting polypeptide of the ADC. In some embodiments, the non-natural amino acid is linked at the C-terminus of the cytotoxic tubulin inhibitor derivative. In other embodiments, the non-natural amino acid is linked at the N-terminus of the cytotoxic tubulin inhibitor derivative. Any position of the targeting polypeptide of the ADC is suitable for selection to incorporate a non-natural amino acid, and selection may be based on rational design or by random selection for any or no particular desired purpose. Selection of desired sites may be based on producing a non-natural amino acid polypeptide (which may be further modified or remain unmodified) having any desired property or activity, including but not limited to a receptor binding modulators, receptor activity modulators, modulators of binding to binder partners, binding partner activity modulators, binding partner conformation modulators, dimer or multimer formation, no change to activity or property compared to the native molecule, or manipulating any physical or chemical property of the polypeptide such as solubility, aggregation, or stability. Alternatively, the sites identified as critical to biological activity may also be good candidates for substitution with a non-natural amino acid, again depending on the desired activity sought for the polypeptide. Another alternative would be to simply make serial substitutions in each position on the polypeptide chain with a non-natural amino acid and observe the effect on the activities of the polypeptide. Any means, technique, or method for selecting a position for substitution with a non-natural amino acid into any polypeptide is suitable for use in the methods, techniques and compositions described herein.

The structure and activity of naturally-occurring mutants of a polypeptide that contain deletions can also be examined to determine regions of the protein that are likely to be tolerant of substitution with a non-natural amino acid. Once residues that are likely to be intolerant to substitution with non-natural amino acids have been eliminated, the impact of proposed substitutions at each of the remaining positions can be examined using methods including, but not limited to, the three-dimensional structure of the relevant polypeptide, and any associated ligands or binding proteins. X-ray crystallographic and NMR structures of many polypeptides are available in the Protein Data Bank (PDB, on the worldwide web at resb.org), a centralized database containing three-dimensional structural data of large molecules of proteins and nucleic acids, one can be used to identify amino acid positions that can be substituted with non-natural amino acids. In addition, models may be made investigating the secondary and tertiary structure of polypeptides, if three-dimensional structural data is not available. Thus, the identity of amino acid positions that can be substituted with non-natural amino acids can be readily obtained.

Exemplary sites of incorporation of a non-natural amino acid include, but are not limited to, those that are excluded from potential receptor binding regions, or regions for binding to binding proteins or ligands may be fully or partially solvent exposed, have minimal or no hydrogen-bonding interactions with nearby residues, may be minimally exposed to nearby reactive residues, and/or may be in regions that are highly flexible as predicted by the three-dimensional crystal structure of a particular polypeptide with its associated receptor, ligand or binding proteins.

A wide variety of non-natural amino acids can be substituted for, or incorporated into, a given position in a polypeptide. By way of example, a particular non-natural amino acid may be selected for incorporation based on an examination of the three-dimensional crystal structure of a polypeptide with its associated ligand, receptor and/or binding proteins, a preference for conservative substitutions

In one embodiment, the methods described herein include incorporating a non-natural amino acid into the targeting polypeptide of the ADC, where the targeting polypeptide of the ADC comprises a first reactive group; and contacting the targeting polypeptide of the ADC with a molecule (including but not limited to a second protein or polypeptide or polypeptide analog; an antibody or antibody fragment; and any combination thereof) that comprises a second reactive group. In certain embodiments, the first reactive group is a hydroxylamine moiety and the second reactive group is a carbonyl or dicarbonyl moiety, whereby an oxime linkage is formed. In certain embodiments, the first reactive group is a carbonyl or dicarbonyl moiety and the second reactive group is a hydroxylamine moiety, whereby an oxime linkage is formed. In certain embodiments, the first reactive group is a carbonyl or dicarbonyl moiety and the second reactive group is an oxime moiety, whereby an oxime exchange reaction occurs. In certain embodiments, the first reactive group is an oxime moiety and the second reactive group is carbonyl or dicarbonyl moiety, whereby an oxime exchange reaction occurs.

In some cases, the targeting polypeptide of the ADC incorporation(s) of a non-natural amino acid will be combined with other additions, substitutions, or deletions within the polypeptide to affect other chemical, physical, pharmacologic and/or biological traits. In some cases, the other additions, substitutions or deletions may increase the stability (including but not limited to, resistance to proteolytic degradation) of the polypeptide or increase affinity of the polypeptide for its appropriate receptor, ligand and/or binding proteins. In some cases, the other additions, substitutions or deletions may increase the solubility (including but not limited to, when expressed in E. coli or other host cells) of the polypeptide. In some embodiments, sites are selected for substitution with a naturally encoded or non-natural amino acid in addition to another site for incorporation of a non-natural amino acid for the purpose of increasing the polypeptide solubility following expression in E. coli, or other recombinant host cells. In some embodiments, the polypeptides comprise another addition, substitution, or deletion that modulates affinity for the associated ligand, binding proteins, and/or receptor, modulates (including but not limited to, increases or decreases) receptor dimerization, stabilizes receptor dimers, modulates circulating half-life, modulates release or bio-availability, facilitates purification, or improves or alters a particular route of administration. Similarly, the non-natural amino acid polypeptide can comprise chemical or enzyme cleavage sequences, protease cleavage sequences, reactive groups, antibody-binding domains (including but not limited to, FLAG or poly-His) or other affinity based sequences (including but not limited to, FLAG, poly-His, GST, etc.) or linked molecules (including but not limited to, biotin) that improve detection (including but not limited to, GFP), purification, transport thru tissues or cell membranes, prodrug release or activation, size reduction, or other traits of the polypeptide.

### Anti-HER2 Antibody as Exemplar for Targeting Moiety

The methods, compositions, strategies and techniques described herein are not limited to a particular type, class or family of targeting moiety polypeptides or proteins. Indeed, virtually any targeting moiety polypeptides may be designed or modified to include at least one “modified or unmodified” non-natural amino acids containing targeting polypeptide of the ADC described herein. By way of example only, the targeting moiety polypeptide can be homologous to a therapeutic protein selected from the group consisting of: alpha-1 antitrypsin, angiostatin, antihemolytic factor, antibody, antibody fragment, monoclonal antibody (e.g., bevacizumab, cetuximab, panitumumab, infliximab, adalimumab, basiliximab, daclizumab, omalizumab, ustekinumab, etanercept, gemtuzumab, alemtuzumab, rituximab, trastuzumab, nimotuzumab, palivizumab, and abciximab), apolipoprotein, apoprotein, atrial natriuretic factor, atrial natriuretic polypeptide, atrial peptide, C-X-C chemokine, T39765, NAP-2, ENA-78, gro-a, gro-b, gro-c, IP-10, GCP-2, NAP-4, SDF-1, PF4, MIG, calcitonin, c-kit ligand, CC chemokine, monocyte chemoattractant protein-1, monocyte chemoattractant protein-2, monocyte chemoattractant protein-3, monocyte inflammatory protein-1 alpha, monocyte inflammatory protein-i beta, RANTES, 1309, R83915, R91733, HCC1, T58847, D31065, T64262, CD40, CD40 ligand, collagen, colony stimulating factor (CSF), complement factor 5a, complement inhibitor, complement receptor 1, cytokine, epithelial neutrophil activating peptide-78, MIP-16, MCP-1, epidermal growth factor (EGF), epithelial neutrophil activating peptide, erythropoietin (EPO), exfoliating toxin, Factor IX, Factor VII, Factor VIII, Factor X, fibroblast growth factor (FGF), fibrinogen, fibronectin, four-helical bundle protein, G-CSF, glp-1, GM-CSF, glucocerebrosidase, gonadotropin, growth factor, growth factor receptor, growth hormone releasing factor, hedgehog protein, hemoglobin, hepatocyte growth factor (hGF), hirudin, human growth hormone (hGH), human serum albumin, ICAM-1, ICAM-1 receptor, LFA-1, LFA-1 receptor, insulin, insulin-like growth factor (IGF), IGF-I, IGF-II, interferon (IFN), IFN-alpha, IFN-beta, IFN-gamma, interleukin (IL), IL-1, IL-2, IL-3, IL-4, IL-5, IL-6, IL-7, IL-8, IL-9, IL-10, IL-11, IL-12, keratinocyte growth factor (KGF), lactoferrin, leukemia inhibitory factor, luciferase, neurturin, neutrophil inhibitory factor (NIF), oncostatin M, osteogenic protein, oncogene product, paracitonin, parathyroid hormone (PTH), PD-ECGF, PDGF, peptide hormone, pleiotropin, protein A, protein G, pyrogenic exotoxin A, pyrogenic exotoxin B, pyrogenic exotoxin C, Peptide YY (PYY), relaxin, renin, SCF, small biosynthetic protein, soluble complement receptor I, soluble I-CAM 1, soluble interleukin receptor, soluble TNF receptor, somatomedin, somatostatin, somatotropin, streptokinase, superantigens, staphylococcal enterotoxin, SEA, SEB, SEC1, SEC2, SEC3, SED, SEE, steroid hormone receptor, superoxide dismutase, toxic shock syndrome toxin, thymosin alpha 1, tissue plasminogen activator, tumor growth factor (TGF), tumor necrosis factor, tumor necrosis factor alpha, tumor necrosis factor beta, tumor necrosis factor receptor (TNFR), VLA-4 protein, VCAM-1 protein, vascular endothelial growth factor (VEGF), urokinase, mos, ras, raf, met, p53, tat, fos, myc, jun, myb, rel, estrogen receptor, progesterone receptor, testosterone receptor, aldosterone receptor, LDL receptor, and corticosterone.

In one embodiment is a method for treating a solid tumor which overexpresses HER-2, has a HER-2 mutation, or has HER-2 gene amplification, selected from the group consisting of breast cancer, small cell lung carcinoma, ovarian cancer, endometrial cancer, bladder cancer, head and neck cancer, prostate cancer, gastric carcinoma, cervical cancer, uterine cancer, esophageal carcinoma, and colon cancer. In another embodiment, the solid tumor is breast cancer. In a further embodiment the solid tumor is ovarian cancer. In a further embodiment the solid tumor is gastric cancer.

Thus, the following description of trastuzumab is provided for illustrative purposes and by way of example only, and not as a limit on the scope of the methods, compositions, strategies and techniques described herein. Further, reference to trastuzumab in this application is intended to use the generic term as an example of any antibody. Thus, it is understood that the modifications and chemistries described herein with reference to trastuzumab can be equally applied to any antibody or monoclonal antibody, including those specifically listed herein.

Trastuzumab is a humanized monoclonal antibody that binds to the domain IV of the extracellular segment of the HER2/neu receptor. The HER2 gene (also known as HER2/neu and ErbB2 gene) is amplified in 20-30% of early-stage breast cancers, which makes it overexpressed. Also, in cancer, HER2 may send signals without mitogens arriving and binding to any receptor, making it overactive.

HER2 extends through the cell membrane and carries signals from outside the cell to the inside. In healthy people, signaling compounds called mitogens arrive at the cell membrane, and bind to the outside part of other members of the HER family of receptors. Those bound receptors then link (dimerize) with HER2, activating it. HER2 then sends a signal to the inside of the cell. The signal passes through different biochemical pathways. This includes the PI3K/Akt pathway and the MAPK pathway. These signals promote invasion, survival and growth of blood vessels (angiogenesis) of cells.

Cells treated with trastuzumab undergo arrest during the G1 phase of the cell cycle so there is reduced proliferation. It has been suggested that trastuzumab induces some of its effect by downregulation of HER2/neu leading to disruption of receptor dimerization and signaling through the downstream PI3K cascade. P27Kip1 is then not phosphorylated and is able to enter the nucleus and inhibit cdk2 activity, causing cell cycle arrest. Also, trastuzumab suppresses angiogenesis by both induction of antiangiogenic factors and repression of proangiogenic factors. It is thought that a contribution to the unregulated growth observed in cancer could be due to proteolytic cleavage of HER2/neu that results in the release of the extracellular domain. Trastuzumab has been shown to inhibit HER2/neu ectodomain cleavage in breast cancer cells.

### Expression in Non-Eukaryotes and Eukaryotes

To obtain high level expression of a cloned ADC polynucleotide, one typically subclones polynucleotides encoding a targeting polypeptide of the ADC polypeptide of the disclosure into an expression vector that contains a strong promoter to direct transcription, a transcription/translation terminator, and if for a nucleic acid encoding a protein, a ribosome binding site for translational initiation. Suitable bacterial promoters are known to those of ordinary skill in the art and described, e.g., in Sambrook et al. and Ausubel et al.

Bacterial expression systems for expressing ADC polypeptides of the disclosure are available in, including but not limited to, E. coli, Bacillus sp., Pseudomonas fluorescens, Pseudomonas aeruginosa, Pseudomonas putida, and Salmonella (Palva et al., Gene 22:229-235 (1983); Mosbach et al., Nature 302:543-545 (1983)). Kits for such expression systems are commercially available. Eukaryotic expression systems for mammalian cells, yeast, and insect cells are known to those of ordinary skill in the art and are also commercially available. In cases where orthogonal tRNAs and aminoacyl tRNA synthetases (described above) are used to express the ADC polypeptides of the disclosure, host cells for expression are selected based on their ability to use the orthogonal components. Exemplary host cells include Gram-positive bacteria (including but not limited to B. brevis, B. subtilis, or Streptomyces) and Gram-negative bacteria (E. coli, Pseudomonas fluorescens, Pseudomonas aeruginosa, Pseudomonas putida), as well as yeast and other eukaryotic cells. Cells comprising O-tRNA/O-RS pairs can be used as described herein.

A eukaryotic host cell or non-eukaryotic host cell of the present disclosure provides the ability to synthesize proteins that comprise non-natural amino acids in large useful quantities. In one aspect, the composition optionally includes, including but not limited to, at least 10 micrograms, at least 50 micrograms, at least 75 micrograms, at least 100 micrograms, at least 200 micrograms, at least 250 micrograms, at least 500 micrograms, at least 1 milligram, at least 10 milligrams, at least 100 milligrams, at least one gram, or more of the protein that comprises an non-natural amino acid, or an amount that can be achieved with in vivo protein production methods (details on recombinant protein production and purification are provided herein). In another aspect, the protein is optionally present in the composition at a concentration of, including but not limited to, at least 10 micrograms of protein per liter, at least 50 micrograms of protein per liter, at least 75 micrograms of protein per liter, at least 100 micrograms of protein per liter, at least 200 micrograms of protein per liter, at least 250 micrograms of protein per liter, at least 500 micrograms of protein per liter, at least 1 milligram of protein per liter, or at least 10 milligrams of protein per liter or more, in, including but not limited to, a cell lysate, a buffer, a pharmaceutical buffer, or other liquid suspension (including but not limited to, in a volume of, including but not limited to, anywhere from about 1 nL to about 100 L or more). The production of large quantities (including but not limited to, greater that that typically possible with other methods, including but not limited to, in vitro translation) of a protein in a eukaryotic cell including at least one non-natural amino acid is a feature of the disclosure.

The nucleotide sequence encoding a targeting polypeptide of the ADC polypeptide may or may not also include sequence that encodes a signal peptide. The signal peptide is present when the polypeptide is to be secreted from the cells in which it is expressed. Such signal peptide may be any sequence. The signal peptide may be prokaryotic or eukaryotic. Coloma, M (1992) J. Imm. Methods 152:89 104) describe a signal peptide for use in mammalian cells (murine Ig kappa light chain signal peptide). Other signal peptides include but are not limited to, the alpha-factor signal peptide from S. cerevisiae (U.S. Pat. No. 4,870,008 which is incorporated by reference herein), the signal peptide of mouse salivary amylase (O. Hagenbuchle et al., Nature 289, 1981, pp. 643-646), a modified carboxypeptidase signal peptide (L. A. Valls et al., Cell 48, 1987, pp. 887-897), the yeast BAR1 signal peptide (WO 87/02670, which is incorporated by reference herein), and the yeast aspartic protease 3 (YAP3) signal peptide (cf. M. Egel-Mitani et al., Yeast 6, 1990, pp. 127-137).

Examples of suitable mammalian host cells are known to those of ordinary skill in the art. Such host cells may be Chinese hamster ovary (CHO) cells, (e.g. CHO-K1; ATCC CCL-61), Green Monkey cells (COS) (e.g. COS 1 (ATCC CRL-1650), COS 7 (ATCC CRL-1651)); mouse cells (e.g. NS/O), Baby Hamster Kidney (BHK) cell lines (e.g. ATCC CRL-1632 or ATCC CCL-10), and human cells (e.g. HEK 293 (ATCC CRL-1573)), as well as plant cells in tissue culture. These cell lines and others are available from public depositories such as the American Type Culture Collection, Rockville, Md. In order to provide improved glycosylation of the ADC polypeptide, a mammalian host cell may be modified to express sialyltransferase, e.g. 1,6-sialyltransferase, e.g. as described in U.S. Pat. No. 5,047,335, which is incorporated by reference herein.

Methods for the introduction of exogenous DNA into mammalian host cells include but are not limited to, calcium phosphate-mediated transfection, electroporation, DEAE-dextran mediated transfection, liposome-mediated transfection, viral vectors and the transfection methods described by Life Technologies Ltd, Paisley, UK using Lipofectamin 2000 and Roche Diagnostics Corporation, Indianapolis, USA using FuGENE 6. These methods are well known in the art and are described by Ausbel et al. (eds.), 1996, Current Protocols in Molecular Biology, John Wiley & Sons, New York, USA. The cultivation of mammalian cells may be performed according to established methods, e.g. as disclosed in (Animal Cell Biotechnology, Methods and Protocols, Edited by Nigel Jenkins, 1999, Human Press Inc. Totowa, N.J., USA and Harrison Mass. and Rae IF, General Techniques of Cell Culture, Cambridge University Press 1997).

E. coli, Pseudomonas Species, and Other Prokaryotes

Bacterial expression techniques are known to those of ordinary skill in the art. A wide variety of vectors are available for use in bacterial hosts. The vectors may be single copy or low or high multicopy vectors. Vectors may serve for cloning and/or expression. In view of the ample literature concerning vectors, commercial availability of many vectors, and even manuals describing vectors and their restriction maps and characteristics, no extensive discussion is required here. As is well-known, the vectors normally involve markers allowing for selection, which markers may provide for cytotoxic agent resistance, prototrophy or immunity. Frequently, a plurality of markers is present, which provide for different characteristics.

A bacterial promoter is any DNA sequence capable of binding bacterial RNA polymerase and initiating the downstream (3′) transcription of a coding sequence (e.g. structural gene) into mRNA. A promoter will have a transcription initiation region which is usually placed proximal to the 5′ end of the coding sequence. This transcription initiation region typically includes an RNA polymerase binding site and a transcription initiation site. A bacterial promoter may also have a second domain called an operator that may overlap an adjacent RNA polymerase binding site at which RNA synthesis begins. The operator permits negative regulated (inducible) transcription, as a gene repressor protein may bind the operator and thereby inhibit transcription of a specific gene. Constitutive expression may occur in the absence of negative regulatory elements, such as the operator. In addition, positive regulation may be achieved by a gene activator protein binding sequence, which, if present is usually proximal (5′) to the RNA polymerase binding sequence. An example of a gene activator protein is the catabolite activator protein (CAP), which helps initiate transcription of the lac operon in Escherichia coli (E. coli) (see, Raibaud et al., ANNU. REV. GENET. (1984) 18:173). Regulated expression may therefore be either positive or negative, thereby either enhancing or reducing transcription.

The term “bacterial host” or “bacterial host cell” refers to bacteria that can be, or has been, used as a recipient for recombinant vectors or other transfer DNA. The term includes the progeny of the original bacterial host cell that has been transfected. It is understood that the progeny of a single parental cell may not necessarily be completely identical in morphology or in genomic or total DNA complement to the original parent, due to accidental or deliberate mutation. Progeny of the parental cell that are sufficiently similar to the parent to be characterized by the relevant property, such as the presence of a nucleotide sequence encoding a ADC polypeptide, are included in the progeny intended by this definition.

The selection of suitable host bacteria for expression of ADC polypeptides is known to those of ordinary skill in the art. In selecting bacterial hosts for expression, suitable hosts may include those shown to have, inter alia, good inclusion body formation capacity, low proteolytic activity, and overall robustness. Bacterial hosts are generally available from a variety of sources including, but not limited to, the Bacterial Genetic Stock Center, Department of Biophysics and Medical Physics, University of California (Berkeley, CA); and the American Type Culture Collection (“ATCC”) (Manassas, VA). Industrial/pharmaceutical fermentation generally use bacterial derived from K strains (e.g. W3110) or from bacteria derived from B strains (e.g. BL21). These strains are particularly useful because their growth parameters are extremely well known and robust. In addition, these strains are non-pathogenic, which is commercially important for safety and environmental reasons. Other examples of suitable E. coli hosts include, but are not limited to, strains of BL21, DH10B, or derivatives thereof. In another embodiment of the methods of the present disclosure, the E. coli host is a protease minus strain including, but not limited to, OMP- and LON-. The host cell strain may be a species of Pseudomonas, including but not limited to, Pseudomonas fluorescens, Pseudomonas aeruginosa, and Pseudomonas putida. Pseudomonas fluorescens biovar 1, designated strain MB101, is known to be useful for recombinant production and is available for therapeutic protein production processes. Examples of a Pseudomonas expression system include the system available from The Dow Chemical Company as a host strain (Midland, MI available on the worldwide web at dow.com).

Once a recombinant host cell strain has been established (i.e., the expression construct has been introduced into the host cell and host cells with the proper expression construct are isolated), the recombinant host cell strain is cultured under conditions appropriate for production of ADC polypeptides. As will be apparent to one of skill in the art, the method of culture of the recombinant host cell strain will be dependent on the nature of the expression construct utilized and the identity of the host cell. Recombinant host strains are normally cultured using methods that are known to those of ordinary skill in the art. Recombinant host cells are typically cultured in liquid medium containing assimilable sources of carbon, nitrogen, and inorganic salts and, optionally, containing vitamins, amino acids, growth factors, and other proteinaceous culture supplements known to those of ordinary skill in the art. Liquid media for culture of host cells may optionally contain antibiotics or anti-fungals to prevent the growth of undesirable microorganisms and/or compounds including, but not limited to, antibiotics to select for host cells containing the expression vector.

Recombinant host cells may be cultured in batch or continuous formats, with either cell harvesting (in the case where the ADC polypeptide accumulates intracellularly) or harvesting of culture supernatant in either batch or continuous formats. For production in prokaryotic host cells, batch culture and cell harvest are preferred.

The ADC polypeptides of the present disclosure are normally purified after expression in recombinant systems. The ADC polypeptide may be purified from host cells or culture medium by a variety of methods known to the art. ADC polypeptides produced in bacterial host cells may be poorly soluble or insoluble (in the form of inclusion bodies). In one embodiment of the present disclosure, amino acid substitutions may readily be made in the ADC polypeptide that are selected for the purpose of increasing the solubility of the recombinantly produced protein utilizing the methods disclosed herein as well as those known in the art. In the case of insoluble protein, the protein may be collected from host cell lysates by centrifugation and may further be followed by homogenization of the cells. In the case of poorly soluble protein, compounds including, but not limited to, polyethylene imine (PEI) may be added to induce the precipitation of partially soluble protein. The precipitated protein may then be conveniently collected by centrifugation. Recombinant host cells may be disrupted or homogenized to release the inclusion bodies from within the cells using a variety of methods known to those of ordinary skill in the art. Host cell disruption or homogenization may be performed using well known techniques including, but not limited to, enzymatic cell disruption, sonication, dounce homogenization, or high pressure release disruption. In one embodiment of the method of the present disclosure, the high pressure release technique is used to disrupt the E. coli host cells to release the inclusion bodies of the ADC polypeptides. When handling inclusion bodies of ADC polypeptide, it may be advantageous to minimize the homogenization time on repetitions in order to maximize the yield of inclusion bodies without loss due to factors such as solubilization, mechanical shearing or proteolysis.

Insoluble or precipitated ADC polypeptide may then be solubilized using any of a number of suitable solubilization agents known to the art. The ADC polypeptide may be solubilized with urea or guanidine hydrochloride. The volume of the solubilized ADC polypeptide should be minimized so that large batches may be produced using conveniently manageable batch sizes. This factor may be significant in a large-scale commercial setting where the recombinant host may be grown in batches that are thousands of liters in volume. In addition, when manufacturing ADC polypeptide in a large-scale commercial setting, in particular for human pharmaceutical uses, the avoidance of harsh chemicals that can damage the machinery and container, or the protein product itself, should be avoided, if possible. It has been shown in the method of the present disclosure that the milder denaturing agent urea can be used to solubilize the ADC polypeptide inclusion bodies in place of the harsher denaturing agent guanidine hydrochloride. The use of urea significantly reduces the risk of damage to stainless steel equipment utilized in the manufacturing and purification process of ADC polypeptide while efficiently solubilizing the ADC polypeptide inclusion bodies.

In the case of soluble targeting polypeptide of the ADC protein, the targeting polypeptide of the ADC may be secreted into the periplasmic space or into the culture medium. In addition, soluble ADC may be present in the cytoplasm of the host cells. It may be desired to concentrate soluble ADC prior to performing purification steps. Standard techniques known to those of ordinary skill in the art may be used to concentrate soluble targeting polypeptide from, for example, cell lysates or culture medium. In addition, standard techniques known to those of ordinary skill in the art may be used to disrupt host cells and release soluble ADC from the cytoplasm or periplasmic space of the host cells.

In general, it is occasionally desirable to denature and reduce expressed polypeptides and then to cause the polypeptides to re-fold into the preferred conformation. For example, guanidine, urea, DTT, DTE, and/or a chaperonin can be added to a translation product of interest. Methods of reducing, denaturing and renaturing proteins are known to those of ordinary skill in the art (see, the references above, and Debinski, et al. (1993) J. Biol. Chem., 268: 14065-14070; Kreitman and Pastan (1993) Bioconjug. Chem., 4: 581-585; and Buchner, et al., (1992) Anal. Biochem., 205: 263-270). Debinski, et al., for example, describe the denaturation and reduction of inclusion body proteins in guanidine-DTE. The proteins can be refolded in a redox buffer containing, including but not limited to, oxidized glutathione and L-arginine. Refolding reagents can be flowed or otherwise moved into contact with the one or more polypeptide or other expression product, or vice-versa.

In the case of prokaryotic production of ADC polypeptide, the ADC polypeptide thus produced may be misfolded and thus lacks or has reduced biological activity. The bioactivity of the protein may be restored by “refolding”. In general, misfolded ADC polypeptide is refolded by solubilizing (where the ADC polypeptide is also insoluble), unfolding and reducing the polypeptide chain using, for example, one or more chaotropic agents (e.g. urea and/or guanidine) and a reducing agent capable of reducing disulfide bonds (e.g. dithiothreitol, DTT or 2-mercaptoethanol, 2-ME). At a moderate concentration of chaotrope, an oxidizing agent is then added (e.g., oxygen, cystine or cystamine), which allows the reformation of disulfide bonds. ADC polypeptide may be refolded using standard methods known in the art, such as those described in U.S. Pat. Nos. 4,511,502, 4,511,503, and 4,512,922, which are incorporated by reference herein. The ADC polypeptide may also be cofolded with other proteins to form heterodimers or heteromultimers.

After refolding, the targeting polypeptide of the ADC may be further purified. Purification of ADC may be accomplished using a variety of techniques known to those of ordinary skill in the art, including hydrophobic interaction chromatography, size exclusion chromatography, ion exchange chromatography, reverse-phase high performance liquid chromatography, affinity chromatography, and the like or any combination thereof. Additional purification may also include a step of drying or precipitation of the purified protein.

After purification, the targeting polypeptide of the ADC may be exchanged into different buffers and/or concentrated by any of a variety of methods known to the art, including, but not limited to, diafiltration and dialysis. ADC that is provided as a single purified protein may be subject to aggregation and precipitation.

The purified targeting polypeptide of the ADC may be at least 90% pure (as measured by reverse phase high performance liquid chromatography, RP-HPLC, or sodium dodecyl sulfate-polyacrylamide gel electrophoresis, SDS-PAGE) or at least 95% pure, or at least 96% pure, or at least 97% pure, or at least 98% pure, or at least 99% or greater pure. Regardless of the exact numerical value of the purity of the targeting polypeptide of the ADC, the targeting polypeptide of the ADC is sufficiently pure for use as a pharmaceutical product or for further processing, such as conjugation with a water soluble polymer such as PEG.

Certain ADC molecules may be used as therapeutic agents in the absence of other active ingredients or proteins (other than excipients, carriers, and stabilizers, serum albumin and the like), or they may be complexed with another protein or a polymer.

Previously, it has been shown that non-natural amino acids can be site-specifically incorporated into proteins in vitro by the addition of chemically aminoacylated suppressor tRNAs to protein synthesis reactions programmed with a gene containing a desired amber nonsense mutation. Using these approaches, one can substitute a number of the common twenty amino acids with close structural homologues, e.g., fluorophenylalanine for phenylalanine, using strains auxotropic for a particular amino acid. See, e.g., Noren, C. J., Anthony-Cahill, Griffith, M. C., Schultz, P. G. A general method for site-specific incorporation of unnatural amino acids into proteins, Science, 244: 182-188 (1989); M. W. Nowak, et al., Science 268:439-42 (1995); Bain, J. D., Glabe, C. G., Dix, T. A., Chamberlin, A. R., Diala, E. S. Biosynthetic site-specific Incorporation of a non-natural amino acid into a polypeptide, J. Am Chem Soc, 111:8013-8014 (1989); N. Budisa et al., FASEB J. 13:41-51 (1999); Ellman, J. A., Mendel, D., Anthony-Cahill, S., Noren, C. J., Schultz, P. G. Biosynthetic method for introducing unnatural amino acids site-specifically into proteins, Methods in Enz., vol. 202, 301-336 (1992); and, Mendel, D., Cornish, V. W. & Schultz, P. G. Site-Directed Mutagenesis with an Expanded Genetic Code, Annu Rev Biophys. Biomol Struct. 24, 435-62 (1995).

For example, a suppressor tRNA was prepared that recognized the stop codon UAG and was chemically aminoacylated with a non-natural amino acid. Conventional site-directed mutagenesis was used to introduce the stop codon TAG, at the site of interest in the protein gene. See, e.g., Sayers, J. R., Schmidt, W. Eckstein, F. 5′-3′ Exonucleases in phosphorothioate-based oligonucleotide-directed mutagenesis, Nucleic Acids Res, 16(3):791-802 (1988). When the acylated suppressor tRNA and the mutant gene were combined in an in vitro transcription/translation system, the non-natural amino acid was incorporated in response to the UAG codon which gave a protein containing that amino acid at the specified position. Experiments using [³H]-Phe and experiments with α-hydroxy acids demonstrated that only the desired amino acid is incorporated at the position specified by the UAG codon and that this amino acid is not incorporated at any other site in the protein. See, e.g., Noren, et al, supra; Kobayashi et al., (2003) Nature Structural Biology 10(6):425-432; and, Ellman, J. A., Mendel, D., Schultz, P. G. Site-specific incorporation of novel backbone structures into proteins, Science, 255(5041):197-200 (1992).

A tRNA may be aminoacylated with a desired amino acid by any method or technique, including but not limited to, chemical or enzymatic aminoacylation.

Aminoacylation may be accomplished by aminoacyl tRNA synthetases or by other enzymatic molecules, including but not limited to, ribozymes. The term “ribozyme” is interchangeable with “catalytic RNA.” Cech and coworkers (Cech, 1987, Science, 236:1532-1539; McCorkle et al., 1987, Concepts Biochem. 64:221-226) demonstrated the presence of naturally occurring RNAs that can act as catalysts (ribozymes). However, although these natural RNA catalysts have only been shown to act on ribonucleic acid substrates for cleavage and splicing, the recent development of artificial evolution of ribozymes has expanded the repertoire of catalysis to various chemical reactions. Studies have identified RNA molecules that can catalyze aminoacyl-RNA bonds on their own (2′)3′-termini (Illangakekare et al., 1995 Science 267:643-647), and an RNA molecule which can transfer an amino acid from one RNA molecule to another (Lohse et al., 1996, Nature 381:442-444).

U.S. Patent Application Publication 2003/0228593, which is incorporated by reference herein, describes methods to construct ribozymes and their use in aminoacylation of tRNAs with naturally encoded and non-naturally encoded amino acids. Substrate-immobilized forms of enzymatic molecules that can aminoacylate tRNAs, including but not limited to, ribozymes, may enable efficient affinity purification of the aminoacylated products. Examples of suitable substrates include agarose, sepharose, and magnetic beads. The production and use of a substrate-immobilized form of ribozyme for aminoacylation is described in Chemistry and Biology 2003, 10:1077-1084 and U.S. Patent Application Publication 2003/0228593, which are incorporated by reference herein.

Chemical aminoacylation methods include, but are not limited to, those introduced by Hecht and coworkers (Hecht, S. M. Acc. Chem. Res. 1992, 25, 545; Heckler, T. G.; Roesser, J. R.; Xu, C.; Chang, P.; Hecht, S. M. Biochemistry 1988, 27, 7254; Hecht, S. M.; Alford, B. L.; Kuroda, Y.; Kitano, S. J. Biol. Chem. 1978, 253, 4517) and by Schultz, Chamberlin, Dougherty and others (Cornish, V. W.; Mendel, D.; Schultz, P. G. Angew. Chem. Int. Ed. Engl. 1995, 34, 621; Robertson, S. A.; Ellman, J. A.; Schultz, P. G. J. Am. Chem. Soc. 1991, 113, 2722; Noren, C. J.; Anthony-Cahill, S. J.; Griffith, M. C.; Schultz, P. G. Science 1989, 244, 182; Bain, J. D.; Glabe, C. G.; Dix, T. A.; Chamberlin, A. R. J. Am. Chem. Soc. 1989, 111, 8013; Bain, J. D. et al. Nature 1992, 356, 537; Gallivan, J. P.; Lester, H. A.; Dougherty, D. A. Chem. Biol. 1997, 4, 740; Turcatti, et al. J. Biol. Chem. 1996, 271, 19991; Nowak, M. W. et al. Science, 1995, 268, 439; Saks, M. E. et al. J. Biol. Chem. 1996, 271, 23169; Hohsaka, T. et al. J. Am. Chem. Soc. 1999, 121, 34), which are incorporated by reference herein, to avoid the use of synthetases in aminoacylation. Such methods or other chemical aminoacylation methods may be used to aminoacylate tRNA molecules.

Methods for generating catalytic RNA may involve generating separate pools of randomized ribozyme sequences, performing directed evolution on the pools, screening the pools for desirable aminoacylation activity, and selecting sequences of those ribozymes exhibiting desired aminoacylation activity.

Reconstituted translation systems may also be used. Mixtures of purified translation factors have also been used successfully to translate mRNA into protein as well as combinations of lysates or lysates supplemented with purified translation factors such as initiation factor-1 (IF-1), IF-2, IF-3 (a or P), elongation factor T (EF-Tu), or termination factors. Cell-free systems may also be coupled transcription/translation systems wherein DNA is introduced to the system, transcribed into mRNA and the mRNA translated as described in Current Protocols in Molecular Biology (F. M. Ausubel et al. editors, Wiley Interscience, 1993), which is hereby specifically incorporated by reference. RNA transcribed in eukaryotic transcription system may be in the form of heteronuclear RNA (hnRNA) or 5′-end caps (7-methyl guanosine) and 3′-end poly A tailed mature mRNA, which can be an advantage in certain translation systems. For example, capped mRNAs are translated with high efficiency in the reticulocyte lysate system.

### Macromolecular Polymers Coupled to ADC Polypeptides

Various modifications to the non-natural amino acid polypeptides described herein can be effected using the compositions, methods, techniques and strategies described herein. These modifications include the incorporation of further functionality onto the non-natural amino acid component of the polypeptide, including but not limited to, a label; a dye; a polymer; a water-soluble polymer; a derivative of polyethylene glycol; a photocrosslinker; a radionuclide; a cytotoxic compound; a drug; an affinity label; a photoaffinity label; a reactive compound; a resin; a second protein or polypeptide or polypeptide analog; an antibody or antibody fragment; a metal chelator; a cofactor; a fatty acid; a carbohydrate; a polynucleotide; a DNA; a RNA; an antisense polynucleotide; a saccharide; a water-soluble dendrimer; a cyclodextrin; an inhibitory ribonucleic acid; a biomaterial; a nanoparticle; a spin label; a fluorophore, a metal-containing moiety; a radioactive moiety; a novel functional group; a group that covalently or noncovalently interacts with other molecules; a photocaged moiety; an actinic radiation excitable moiety; a photoisomerizable moiety; biotin; a derivative of biotin; a biotin analogue; a moiety incorporating a heavy atom; a chemically cleavable group; a photocleavable group; an elongated side chain; a carbon-linked sugar; a redox-active agent; an amino thioacid; a toxic moiety; an isotopically labeled moiety; a biophysical probe; a phosphorescent group; a chemiluminescent group; an electron dense group; a magnetic group; an intercalating group; a chromophore; an energy transfer agent; a biologically active agent; a detectable label; a small molecule; a quantum dot; a nanotransmitter; a radionucleotide; a radiotransmitter; a neutron-capture agent; or any combination of the above, or any other desirable compound or substance. As an illustrative, non-limiting example of the compositions, methods, techniques and strategies described herein, the following description will focus on adding macromolecular polymers to the non-natural amino acid polypeptide with the understanding that the compositions, methods, techniques and strategies described thereto are also applicable (with appropriate modifications, if necessary and for which one of skill in the art could make with the disclosures herein) to adding other functionalities, including but not limited to those listed above.

A wide variety of macromolecular polymers and other molecules can be linked to ADC polypeptides of the present disclosure to modulate biological properties of the ADC polypeptide, and/or provide new biological properties to the ADC molecule. These macromolecular polymers can be linked to the ADC polypeptide via a naturally encoded amino acid, via a non-naturally encoded amino acid, or any functional substituent of a natural or non-natural amino acid, or any substituent or functional group added to a natural or non-natural amino acid. The molecular weight of the polymer may be of a wide range, including but not limited to, between about 100 Da and about 100,000 Da or more. The molecular weight of the polymer may be between about 100 Da and about 100,000 Da, including but not limited to, 100,000 Da, 95,000 Da, 90,000 Da, 85,000 Da, 80,000 Da, 75,000 Da, 70,000 Da, 65,000 Da, 60,000 Da, 55,000 Da, 50,000 Da, 45,000 Da, 40,000 Da, 35,000 Da, 30,000 Da, 25,000 Da, 20,000 Da, 15,000 Da, 10,000 Da, 9,000 Da, 8,000 Da, 7,000 Da, 6,000 Da, 5,000 Da, 4,000 Da, 3,000 Da, 2,000 Da, 1,000 Da, 900 Da, 800 Da, 700 Da, 600 Da, 500 Da, 400 Da, 300 Da, 200 Da, and 100 Da. In some embodiments, the molecular weight of the polymer is between about 100 Da and about 50,000 Da. In some embodiments, the molecular weight of the polymer is between about 100 Da and about 40,000 Da. In some embodiments, the molecular weight of the polymer is between about 1,000 Da and about 40,000 Da. In some embodiments, the molecular weight of the polymer is between about 5,000 Da and about 40,000 Da. In some embodiments, the molecular weight of the polymer is between about 10,000 Da and about 40,000 Da.

The present disclosure provides substantially homogenous preparations of polymer:protein conjugates. “Substantially homogenous” as used herein means that polymer:protein conjugate molecules are observed to be greater than half of the total protein. The polymer:protein conjugate has biological activity and the present “substantially homogenous” PEGylated ADC polypeptide preparations provided herein are those which are homogenous enough to display the advantages of a homogenous preparation, e.g., ease in clinical application in predictability of lot to lot pharmacokinetics.

One may also choose to prepare a mixture of polymer:protein conjugate molecules, and the advantage provided herein is that one may select the proportion of mono-polymer:protein conjugate to include in the mixture. Thus, if desired, one may prepare a mixture of various proteins with various numbers of polymer moieties attached (i.e., di-, tri-, tetra-, etc.) and combine said conjugates with the mono-polymer:protein conjugate prepared using the methods of the present disclosure, and have a mixture with a predetermined proportion of mono-polymer:protein conjugates.

The polymer selected may be water soluble so that the protein to which it is attached does not precipitate in an aqueous environment, such as a physiological environment. The polymer may be branched or unbranched. For therapeutic use of the end-product preparation, the polymer will be pharmaceutically acceptable.

Examples of polymers include but are not limited to polyalkyl ethers and alkoxy-capped analogs thereof (e.g., polyoxyethylene glycol, polyoxyethylene/propylene glycol, and methoxy or ethoxy-capped analogs thereof, especially polyoxyethylene glycol, the latter is also known as polyethyleneglycol or PEG); polyvinylpyrrolidones; polyvinylalkyl ethers; polyoxazolines, polyalkyl oxazolines and polyhydroxyalkyl oxazolines; polyacrylamides, polyalkyl acrylamides, and polyhydroxyalkyl acrylamides (e.g., polyhydroxypropylmethacrylamide and derivatives thereof); polyhydroxyalkyl acrylates; polysialic acids and analogs thereof; hydrophilic peptide sequences; polysaccharides and their derivatives, including dextran and dextran derivatives, e.g., carboxymethyldextran, dextran sulfates, aminodextran; cellulose and its derivatives, e.g., carboxymethyl cellulose, hydroxyalkyl celluloses; chitin and its derivatives, e.g., chitosan, succinyl chitosan, carboxymethylchitin, carboxymethylchitosan; hyaluronic acid and its derivatives; starches; alginates; chondroitin sulfate; albumin; pullulan and carboxymethyl pullulan; polyaminoacids and derivatives thereof, e.g., polyglutamic acids, polylysines, polyaspartic acids, polyaspartamides; maleic anhydride copolymers such as: styrene maleic anhydride copolymer, divinylethyl ether maleic anhydride copolymer; polyvinyl alcohols; copolymers thereof; terpolymers thereof, mixtures thereof; and derivatives of the foregoing.

The proportion of polyethylene glycol molecules to protein molecules will vary, as will their concentrations in the reaction mixture. In general, the optimum ratio (in terms of efficiency of reaction in that there is minimal excess unreacted protein or polymer) may be determined by the molecular weight of the polyethylene glycol selected and on the number of available reactive groups available. As relates to molecular weight, typically the higher the molecular weight of the polymer, the fewer number of polymer molecules which may be attached to the protein. Similarly, branching of the polymer should be taken into account when optimizing these parameters. Generally, the higher the molecular weight (or the more branches) the higher the polymer:protein ratio.

As used herein, and when contemplating PEG:ADC polypeptide conjugates, the term “therapeutically effective amount” refers to an amount which gives the desired benefit to a patient. The amount will vary from one individual to another and will depend upon a number of factors, including the overall physical condition of the patient and the underlying cause of the condition to be treated. The amount of ADC polypeptide used for therapy gives an acceptable rate of change and maintains desired response at a beneficial level. A therapeutically effective amount of the present compositions may be readily ascertained by one of ordinary skill in the art using publicly available materials and procedures.

The water soluble polymer may be any structural form including but not limited to linear, forked or branched. Typically, the water soluble polymer is a poly(alkylene glycol), such as poly(ethylene glycol) (PEG), but other water soluble polymers can also be employed. By way of example, PEG is used to describe certain embodiments of this disclosure.

PEG is a well-known, water soluble polymer that is commercially available or can be prepared by ring-opening polymerization of ethylene glycol according to methods known to those of ordinary skill in the art (Sandler and Karo, Polymer Synthesis, Academic Press, New York, Vol. 3, pages 138-161). The term “PEG” is used broadly to encompass any polyethylene glycol molecule, without regard to size or to modification at an end of the PEG, and can be represented as linked to the ADC polypeptide by the formula:

XO—(CH₂CH₂O)ₙ—CH₂CH₂—Y

where n is 2 to 10,000 and X is H or a terminal modification, including but not limited to, a C1-4 alkyl, a protecting group, or a terminal functional group.

In some cases, a PEG used in the disclosure terminates on one end with hydroxy or methoxy, i.e., X is H or CH₃ (“methoxy PEG”). Alternatively, the PEG can terminate with a reactive group, thereby forming a bifunctional polymer. Typical reactive groups can include those reactive groups that are commonly used to react with the functional groups found in the 20 common amino acids (including but not limited to, maleimide groups, activated carbonates (including but not limited to, p-nitrophenyl ester), activated esters (including but not limited to, N-hydroxysuccinimide, p-nitrophenyl ester) and aldehydes) as well as functional groups that are inert to the 20 common amino acids but that react specifically with complementary functional groups present in non-naturally encoded amino acids (including but not limited to, azide groups, alkyne groups). It is noted that the other end of the PEG, which is shown in the above formula by Y, will attach either directly or indirectly to a ADC polypeptide via a naturally-occurring or non-naturally encoded amino acid. For instance, Y may be an amide, carbamate or urea linkage to an amine group (including but not limited to, the epsilon amine of lysine or the N-terminus) of the polypeptide. Alternatively, Y may be a maleimide linkage to a thiol group (including but not limited to, the thiol group of cysteine). Alternatively, Y may be a linkage to a residue not commonly accessible via the 20 common amino acids. For example, an azide group on the PEG can be reacted with an alkyne group on the ADC polypeptide to form a Huisgen [3+2] cycloaddition product. Alternatively, an alkyne group on the PEG can be reacted with an azide group present in a non-naturally encoded amino acid to form a similar product. In some embodiments, a strong nucleophile (including but not limited to, hydrazine, hydrazide, hydroxylamine, semicarbazide) can be reacted with an aldehyde or ketone group present in a non-naturally encoded amino acid to form a hydrazone, oxime or semicarbazone, as applicable, which in some cases can be further reduced by treatment with an appropriate reducing agent. Alternatively, the strong nucleophile can be incorporated into the ADC polypeptide via a non-naturally encoded amino acid and used to react preferentially with a ketone or aldehyde group present in the water soluble polymer.

Any molecular mass for a PEG can be used as practically desired, including but not limited to, from about 100 Daltons (Da) to 100,000 Da or more as desired (including but not limited to, sometimes 0.1-50 kDa or 10-40 kDa). The molecular weight of PEG may be of a wide range, including but not limited to, between about 100 Da and about 100,000 Da or more. PEG may be between about 100 Da and about 100,000 Da, including but not limited to, 100,000 Da, 95,000 Da, 90,000 Da, 85,000 Da, 80,000 Da, 75,000 Da, 70,000 Da, 65,000 Da, 60,000 Da, 55,000 Da, 50,000 Da, 45,000 Da, 40,000 Da, 35,000 Da, 30,000 Da, 25,000 Da, 20,000 Da, 15,000 Da, 10,000 Da, 9,000 Da, 8,000 Da, 7,000 Da, 6,000 Da, 5,000 Da, 4,000 Da, 3,000 Da, 2,000 Da, 1,000 Da, 900 Da, 800 Da, 700 Da, 600 Da, 500 Da, 400 Da, 300 Da, 200 Da, and 100 Da. In some embodiments, PEG is between about 100 Da and about 50,000 Da. In some embodiments, PEG is between about 100 Da and about 40,000 Da. In some embodiments, PEG is between about 1,000 Da and about 40,000 Da. In some embodiments, PEG is between about 5,000 Da and about 40,000 Da. In some embodiments, PEG is between about 10,000 Da and about 40,000 Da. Branched chain PEGs, including but not limited to, PEG molecules with each chain having a molecular weight ranging from 1-100 kDa (including but not limited to, 1-50 kDa or 5-20 kDa) can also be used. The molecular weight of each chain of the branched chain PEG may be, including but not limited to, between about 1,000 Da and about 100,000 Da or more. The molecular weight of each chain of the branched chain PEG may be between about 1,000 Da and about 100,000 Da, including but not limited to, 100,000 Da, 95,000 Da, 90,000 Da, 85,000 Da, 80,000 Da, 75,000 Da, 70,000 Da, 65,000 Da, 60,000 Da, 55,000 Da, 50,000 Da, 45,000 Da, 40,000 Da, 35,000 Da, 30,000 Da, 25,000 Da, 20,000 Da, 15,000 Da, 10,000 Da, 9,000 Da, 8,000 Da, 7,000 Da, 6,000 Da, 5,000 Da, 4,000 Da, 3,000 Da, 2,000 Da, and 1,000 Da. In some embodiments, the molecular weight of each chain of the branched chain PEG is between about 1,000 Da and about 50,000 Da. In some embodiments, the molecular weight of each chain of the branched chain PEG is between about 1,000 Da and about 40,000 Da. In some embodiments, the molecular weight of each chain of the branched chain PEG is between about 5,000 Da and about 40,000 Da. In some embodiments, the molecular weight of each chain of the branched chain PEG is between about 5,000 Da and about 20,000 Da. A wide range of PEG molecules are described in, including but not limited to, the Shearwater Polymers, Inc. catalog, Nektar Therapeutics catalog, incorporated herein by reference.

Generally, at least one terminus of the PEG molecule is available for reaction with the non-naturally-encoded amino acid. For example, PEG derivatives or cytotoxic tubulin inhibitor-linker derivatives bearing alkyne and azide moieties for reaction with amino acid side chains can be used to attach PEG to non-naturally encoded amino acids as described herein. If the non-naturally encoded amino acid comprises an azide, then the PEG will typically contain either an alkyne moiety to effect formation of the [3+2] cycloaddition product or an activated PEG species (i.e., ester, carbonate) containing a phosphine group to effect formation of the amide linkage. Alternatively, if the non-naturally encoded amino acid comprises an alkyne, then the PEG will typically contain an azide moiety to effect formation of the [3+2]Huisgen cycloaddition product. If the non-naturally encoded amino acid comprises a carbonyl group, the PEG will typically comprise a potent nucleophile (including but not limited to, a hydrazide, hydrazine, hydroxylamine, or semicarbazide functionality) in order to effect formation of corresponding hydrazone, oxime, and semicarbazone linkages, respectively. In other alternatives, a reverse of the orientation of the reactive groups described above can be used, i.e., an azide moiety in the non-naturally encoded amino acid can be reacted with a PEG derivative containing an alkyne.

In some embodiments, the ADC polypeptide with a PEG derivative contains a chemical functionality that is reactive with the chemical functionality present on the side chain of the non-naturally encoded amino acid.

The disclosure provides in some embodiments azide- and acetylene-containing polymer derivatives comprising a water soluble polymer backbone having an average molecular weight from about 800 Da to about 100,000 Da. The polymer backbone of the water-soluble polymer can be poly(ethylene glycol). However, it should be understood that a wide variety of water soluble polymers including but not limited to poly(ethylene)glycol and other related polymers, including poly(dextran) and poly(propylene glycol), are also suitable for use in the practice of this disclosure and that the use of the term PEG or poly(ethylene glycol) is intended to encompass and include all such molecules. The term PEG includes, but is not limited to, poly(ethylene glycol) in any of its forms, including bifunctional PEG, multiarmed PEG, derivatized PEG, forked PEG, branched PEG, pendent PEG (i.e. PEG or related polymers having one or more functional groups pendent to the polymer backbone), or PEG with degradable linkages therein.

PEG is typically clear, colorless, odorless, soluble in water, stable to heat, inert to many chemical agents, does not hydrolyze or deteriorate, and is generally non-toxic. Poly(ethylene glycol) is considered to be biocompatible, which is to say that PEG is capable of coexistence with living tissues or organisms without causing harm. More specifically, PEG is substantially non-immunogenic, which is to say that PEG does not tend to produce an immune response in the body. When attached to a molecule having some desirable function in the body, such as a biologically active agent, the PEG tends to mask the agent and can reduce or eliminate any immune response so that an organism can tolerate the presence of the agent. PEG conjugates tend not to produce a substantial immune response or cause clotting or other undesirable effects. PEG having the formula —CH₂CH₂O—(CH₂CH₂O)ₙ—CH₂CH₂—, where n is from about 3 to about 4000, typically from about 20 to about 2000, is suitable for use in the present disclosure. PEG having a molecular weight of from about 800 Da to about 100,000 Da are in some embodiments of the present disclosure particularly useful as the polymer backbone. The molecular weight of PEG may be of a wide range, including but not limited to, between about 100 Da and about 100,000 Da or more. The molecular weight of PEG may be between about 100 Da and about 100,000 Da, including but not limited to, 100,000 Da, 95,000 Da, 90,000 Da, 85,000 Da, 80,000 Da, 75,000 Da, 70,000 Da, 65,000 Da, 60,000 Da, 55,000 Da, 50,000 Da, 45,000 Da, 40,000 Da, 35,000 Da, 30,000 Da, 25,000 Da, 20,000 Da, 15,000 Da, 10,000 Da, 9,000 Da, 8,000 Da, 7,000 Da, 6,000 Da, 5,000 Da, 4,000 Da, 3,000 Da, 2,000 Da, 1,000 Da, 900 Da, 800 Da, 700 Da, 600 Da, 500 Da, 400 Da, 300 Da, 200 Da, and 100 Da. In some embodiments, the molecular weight of PEG is between about 100 Da and about 50,000 Da. In some embodiments, the molecular weight of PEG is between about 100 Da and about 40,000 Da. In some embodiments, the molecular weight of PEG is between about 1,000 Da and about 40,000 Da. In some embodiments, the molecular weight of PEG is between about 5,000 Da and about 40,000 Da. In some embodiments, the molecular weight of PEG is between about 10,000 Da and about 40,000 Da.

The polymer backbone can be linear or branched. Branched polymer backbones are generally known in the art. Typically, a branched polymer has a central branch core moiety and a plurality of linear polymer chains linked to the central branch core. PEG is commonly used in branched forms that can be prepared by addition of ethylene oxide to various polyols, such as glycerol, glycerol oligomers, pentaerythritol and sorbitol. The central branch moiety can also be derived from several amino acids, such as lysine. The branched poly(ethylene glycol) can be represented in general form as R(-PEG-OH)m in which R is derived from a core moiety, such as glycerol, glycerol oligomers, or pentaerythritol, and m represents the number of arms. Multi-armed PEG molecules, such as those described in U.S. Pat. Nos. 5,932,462; 5,643,575; 5,229,490; 4,289,872; U.S. Pat. Appl. 2003/0143596; WO 96/21469; and WO 93/21259, each of which is incorporated by reference herein in its entirety, can also be used as the polymer backbone.

Branched PEG can also be in the form of a forked PEG represented by PEG(-YCHZ₂)ₙ, where Y is a linking group and Z is an activated terminal group linked to CH by a chain of atoms of defined length. Yet another branched form, the pendant PEG, has reactive groups, such as carboxyl, along the PEG backbone rather than at the end of PEG chains.

In addition to these forms of PEG, the polymer can also be prepared with weak or degradable linkages in the backbone. For example, PEG can be prepared with ester linkages in the polymer backbone that are subject to hydrolysis. As shown below, this hydrolysis results in cleavage of the polymer into fragments of lower molecular weight:

-PEG-CO₂—PEG-+H₂O→PEG-CO₂H+HO-PEG-

It is understood by those of ordinary skill in the art that the term poly(ethylene glycol) or PEG represents or includes all the forms known in the art including but not limited to those disclosed herein.

Many other polymers are also suitable for use in the present disclosure. In some embodiments, polymer backbones that are water-soluble, with from 2 to about 300 termini, are particularly useful in the disclosure. Examples of suitable polymers include, but are not limited to, other poly(alkylene glycols), such as poly(propylene glycol) (“PPG”), copolymers thereof (including but not limited to copolymers of ethylene glycol and propylene glycol), terpolymers thereof, mixtures thereof, and the like. Although the molecular weight of each chain of the polymer backbone can vary, it is typically in the range of from about 800 Da to about 100,000 Da, often from about 6,000 Da to about 80,000 Da. The molecular weight of each chain of the polymer backbone may be between about 100 Da and about 100,000 Da, including but not limited to, 100,000 Da, 95,000 Da, 90,000 Da, 85,000 Da, 80,000 Da, 75,000 Da, 70,000 Da, 65,000 Da, 60,000 Da, 55,000 Da, 50,000 Da, 45,000 Da, 40,000 Da, 35,000 Da, 30,000 Da, 25,000 Da, 20,000 Da, 15,000 Da, 10,000 Da, 9,000 Da, 8,000 Da, 7,000 Da, 6,000 Da, 5,000 Da, 4,000 Da, 3,000 Da, 2,000 Da, 1,000 Da, 900 Da, 800 Da, 700 Da, 600 Da, 500 Da, 400 Da, 300 Da, 200 Da, and 100 Da. In some embodiments, the molecular weight of each chain of the polymer backbone is between about 100 Da and about 50,000 Da. In some embodiments, the molecular weight of each chain of the polymer backbone is between about 100 Da and about 40,000 Da. In some embodiments, the molecular weight of each chain of the polymer backbone is between about 1,000 Da and about 40,000 Da. In some embodiments, the molecular weight of each chain of the polymer backbone is between about 5,000 Da and about 40,000 Da. In some embodiments, the molecular weight of each chain of the polymer backbone is between about 10,000 Da and about 40,000 Da.

Those of ordinary skill in the art will recognize that the foregoing list for substantially water soluble backbones is by no means exhaustive and is merely illustrative, and that all polymeric materials having the qualities described above are contemplated as being suitable for use in the present disclosure.

In some embodiments of the present disclosure the polymer derivatives are “multi-functional”, meaning that the polymer backbone has at least two termini, and possibly as many as about 300 termini, functionalized or activated with a functional group. Multifunctional polymer derivatives include, but are not limited to, linear polymers having two termini, each terminus being bonded to a functional group which may be the same or different.

The term “protected” refers to the presence of a protecting group or moiety that prevents reaction of the chemically reactive functional group under certain reaction conditions. The protecting group will vary depending on the type of chemically reactive group being protected. For example, if the chemically reactive group is an amine or a hydrazide, the protecting group can be selected from the group of tert-butyloxycarbonyl (t-Boc) and 9-fluorenylmethoxycarbonyl (Fmoc). If the chemically reactive group is a thiol, the protecting group can be orthopyridyldisulfide. If the chemically reactive group is a carboxylic acid, such as butanoic or propionic acid, or a hydroxyl group, the protecting group can be benzyl or an alkyl group such as methyl, ethyl, or tert-butyl. Other protecting groups known in the art may also be used in the present disclosure.

Specific examples of terminal functional groups in the literature include, but are not limited to, N-succinimidyl carbonate (see e.g., U.S. Pat. Nos. 5,281,698, 5,468,478), amine (see, e.g., Buckmann et al. Makromol. Chem. 182:1379 (1981), Zalipsky et al. Eur. Polym. J. 19:1177 (1983)), hydrazide (See, e.g., Andresz et al. Makromol. Chem. 179:301 (1978)), succinimidyl propionate and succinimidyl butanoate (see, e.g., Olson et al. in Poly(ethylene glycol) Chemistry & Biological Applications, pp 170-181, Harris & Zalipsky Eds., ACS, Washington, D.C. , 1997; see also U.S. Pat. No. 5,672,662), succinimidyl succinate (See, e.g., Abuchowski et al. Cancer Biochem. Biophys. 7:175 (1984) and Joppich et al. Makromol. Chem. 180:1381 (1979), succinimidyl ester (see, e.g., U.S. Pat. No. 4,670,417), benzotriazole carbonate (see, e.g., U.S. Pat. No. 5,650,234), glycidyl ether (see, e.g., Pitha et al. Eur. J Biochem. 94:11 (1979), Elling et al., Biotech. Appl. Biochem. 13:354 (1991), oxycarbonylimidazole (see, e.g., Beauchamp, et al., Anal. Biochem. 131:25 (1983), Tondelli et al. J. Controlled Release 1:251 (1985)), p-nitrophenyl carbonate (see, e.g., Veronese, et al., Appl. Biochem. Biotech., 11: 141 (1985); and Sartore et al., Appl. Biochem. Biotech., 27:45 (1991)), aldehyde (see, e.g., Harris et al. J. Polym. Sci. Chem. Ed. 22:341 (1984), U.S. Pat. Nos. 5,824,784, 5,252,714), maleimide (see, e.g., Goodson et al. Biotechnology (NY) 8:343 (1990), Romani et al. in Chemistry of Peptides and Proteins 2:29 (1984)), and Kogan, Synthetic Comm. 22:2417 (1992)), orthopyridyl-disulfide (see, e.g., Woghiren, et al. Bioconj. Chem. 4:314(1993)), acrylol (see, e.g., Sawhney et al., Macromolecules, 26:581 (1993)), vinylsulfone (see, e.g., U.S. Pat. No. 5,900,461). All of the above references and patents are incorporated herein by reference.

PEGylation (i.e., addition of any water soluble polymer) of ADC polypeptides containing a non-naturally encoded amino acid, such as p-azido-L-phenylalanine, is carried out by any convenient method. For example, ADC polypeptide is PEGylated with an alkyne-terminated mPEG derivative. Briefly, an excess of solid mPEG(5000)-O—CH₂—C═CH is added, with stirring, to an aqueous solution of p-azido-L-Phe-containing ADC polypeptide at room temperature. Typically, the aqueous solution is buffered with a buffer having a pKa near the pH at which the reaction is to be carried out (generally about pH 4-10). Examples of suitable buffers for PEGylation at pH 7.5, for instance, include, but are not limited to, HEPES, phosphate, borate, TRIS-HCl, EPPS, and TES. The pH is continuously monitored and adjusted if necessary. The reaction is typically allowed to continue for between about 1-48 hours.

The reaction products are subsequently subjected to hydrophobic interaction chromatography to separate the PEGylated ADC polypeptides from free mPEG(5000)-O—CH₂— C═CH and any high-molecular weight complexes of the pegylated ADC polypeptide which may form when unblocked PEG is activated at both ends of the molecule, thereby crosslinking ADC polypeptide molecules. The conditions during hydrophobic interaction chromatography are such that free mPEG(5000)-O—CH₂—C═CH flows through the column, while any crosslinked PEGylated ADC polypeptide complexes elute after the desired forms, which contain one ADC polypeptide molecule conjugated to one or more PEG groups. Suitable conditions vary depending on the relative sizes of the cross-linked complexes versus the desired conjugates and are readily determined by those of ordinary skill in the art. The eluent containing the desired conjugates is concentrated by ultrafiltration and desalted by diafiltration.

Substantially purified PEG-ADC can be produced using the elution methods outlined above where the PEG-ADC produced has a purity level of at least about 30%, at least about 35%, at least about 40%, at least about 45%, at least about 50%, at least about 55%, at least about 60%, at least about 65%, at least about 70%, specifically, a purity level of at least about 75%, 80%, 85%, and more specifically, a purity level of at least about 90%, a purity level of at least about 95%, a purity level of at least about 99% or greater as determined by appropriate methods such as SDS/PAGE analysis, RP-HPLC, SEC, and capillary electrophoresis. If necessary, the PEGylated ADC polypeptide obtained from the hydrophobic chromatography can be purified further by one or more procedures known to those of ordinary skill in the art including, but are not limited to, affinity chromatography; anion- or cation-exchange chromatography (using, including but not limited to, DEAE SEPHAROSE); chromatography on silica; reverse phase HPLC; gel filtration (using, including but not limited to, SEPHADEX G-75); hydrophobic interaction chromatography; size-exclusion chromatography, metal-chelate chromatography; ultrafiltration/diafiltration; ethanol precipitation; ammonium sulfate precipitation; chromatofocusing; displacement chromatography; electrophoretic procedures (including but not limited to preparative isoelectric focusing), differential solubility (including but not limited to ammonium sulfate precipitation), or extraction. Apparent molecular weight may be estimated by GPC by comparison to globular protein standards (Preneta, AZ in PROTEIN PURIFICATION METHODS, A PRACTICAL APPROACH (Harris & Angal, Eds.) IRL Press 1989, 293-306). The purity of the ADC-PEG conjugate can be assessed by proteolytic degradation (including but not limited to, trypsin cleavage) followed by mass spectrometry analysis. Pepinsky RB., et al., J. Pharmcol. & Exp. Ther. 297(3):1059-66 (2001).

A water soluble polymer linked to an amino acid of a targeting polypeptide of the ADC polypeptide of the disclosure can be further derivatized or substituted without limitation.

### Azide-Containing PEG Derivatives or Cytotoxic Tubulin Inhibitor-Linker Derivatives

In another embodiment of the disclosure, a targeting polypeptide of the ADC is modified with a PEG derivative that contains an alkyne moiety that will react with an azide moiety present on the side chain of the non-naturally encoded amino acid.

In some embodiments, the alkyne-terminal PEG derivative will have the following structure:

RO—(CH₂CH₂O)ₙ—O—(CH₂)ₘ—C═CH

where R is a simple alkyl (methyl, ethyl, propyl, etc.), m is 2-10 and n is 100-1,000 (i.e., average molecular weight is between 5-40 kDa).

In another embodiment of the disclosure, a targeting polypeptide of the ADC comprising an alkyne-containing non-naturally encoded amino acid is modified with a PEG derivative that contains a terminal azide or terminal alkyne moiety that is linked to the PEG backbone by means of an amide linkage.

In some embodiments, the alkyne-terminal PEG derivative will have the following structure:

RO—(CH₂CH₂O)ₙ—O—(CH₂)ₘ—NH—C(O)—(CH₂)ₚ—C≡CH

where R is a simple alkyl (methyl, ethyl, propyl, etc.), m is 2-10, p is 2-10 and n is 100-1,000.

In another embodiment of the disclosure, a targeting polypeptide of the ADC comprising an azide-containing amino acid is modified with a branched PEG derivative that contains a terminal alkyne moiety, with each chain of the branched PEG having a molecular weight ranging from 10-40 kDa and may be from 5-20 kDa. For instance, in some embodiments, the alkyne-terminal PEG derivative will have the following structure:

[RO—(CH₂CH₂O)ₙ—O—(CH₂)₂—NH—C(O)]₂CH(CH₂)ₘ—X—(CH₂)ₚ—C≡CH

where R is a simple alkyl (methyl, ethyl, propyl, etc.), m is 2-10, p is 2-10, and n is 100-1,000, and X is optionally an O, N, S or carbonyl group (C═O), or not present.

### Phosphine-Containing PEG Derivatives or Cytotoxic Tubulin Inhibitor-Linker Derivatives

In another embodiment of the disclosure, a targeting polypeptide of the ADC is modified with a PEG derivative that contains an activated functional group (including but not limited to, ester, carbonate) further comprising an aryl phosphine group that will react with an azide moiety present on the side chain of the non-naturally encoded amino acid. In general, the PEG derivatives or cytotoxic tubulin inhibitor-linker derivatives will have an average molecular weight ranging from 1-100 kDa and, in some embodiments, from 10-40 kDa.

In some embodiments, the PEG derivative will have the structure:

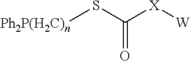

wherein n is 1-10; X can be O, N, S or not present, Ph is phenyl, and W is a water soluble polymer.

In some embodiments, the PEG derivative will have the structure:

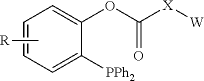

wherein X can be O, N, S or not present, Ph is phenyl, W is a water soluble polymer and R can be H, alkyl, aryl, substituted alkyl and substituted aryl groups. Exemplary R groups include but are not limited to —CH₂, —C(CH₃)₃, —OR′, —NR′R″, —SR′, -halogen, —C(O)R′, —CONR′R″, —S(O)₂R′, —S(O)₂NR′R″, —CN and —NO₂. R′, R″, R′″ and R″″ each independently refer to hydrogen, substituted or unsubstituted heteroalkyl, substituted or unsubstituted aryl, including but not limited to, aryl substituted with 1-3 halogens, substituted or unsubstituted alkyl, alkoxy or thioalkoxy groups, or arylalkyl groups. When a compound of the disclosure includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (including but not limited to, —CF₃ and —CH₂CF₃) and acyl (including but not limited to, —C(O)CH₃, —C(O)CF₃, —C(O)CH₂OCH₃, and the like).

### Other PEG Derivatives or Cytotoxic Tubulin Inhibitor-Linker Derivatives and General Conjugation Techniques

Other exemplary PEG molecules that may be linked to ADC polypeptides, as well as PEGylation methods include, but are not limited to, those described in, e.g., U.S. Patent Publication No. 2004/0001838; 2002/0052009; 2003/0162949; 2004/0013637; 2003/0228274; 2003/0220447; 2003/0158333; 2003/0143596; 2003/0114647; 2003/0105275; 2003/0105224; 2003/0023023; 2002/0156047; 2002/0099133; 2002/0086939; 2002/0082345; 2002/0072573; 2002/0052430; 2002/0040076; 2002/0037949; 2002/0002250; 2001/0056171; 2001/0044526; 2001/0021763; U.S. Pat. Nos. 6,646,110; 5,824,778; 5,476,653; 5,219,564; 5,629,384; 5,736,625; 4,902,502; 5,281,698; 5,122,614; 5,473,034; 5,516,673; 5,382,657; 6,552,167; 6,610,281; 6,515,100; 6,461,603; 6,436,386; 6,214,966; 5,990,237; 5,900,461; 5,739,208; 5,672,662; 5,446,090; 5,808,096; 5,612,460; 5,324,844; 5,252,714; 6,420,339; 6,201,072; 6,451,346; 6,306,821; 5,559,213; 5,747,646; 5,834,594; 5,849,860; 5,980,948; 6,004,573; 6,129,912; WO 97/32607, EP 229,108, EP 402,378, WO 92/16555, WO 94/04193, WO 94/14758, WO 94/17039, WO 94/18247, WO 94/28024, WO 95/00162, WO 95/11924, WO95/13090, WO 95/33490, WO 96/00080, WO 97/18832, WO 98/41562, WO 98/48837, WO 99/32134, WO 99/32139, WO 99/32140, WO 96/40791, WO 98/32466, WO 95/06058, EP 439 508, WO 97/03106, WO 96/21469, WO 95/13312, EP 921 131, WO 98/05363, EP 809 996, WO 96/41813, WO 96/07670, EP 605 963, EP 510 356, EP 400 472, EP 183 503 and EP 154 316, which are incorporated by reference herein. Any of the PEG molecules described herein may be used in any form, including but not limited to, single chain, branched chain, multiarm chain, single functional, bi-functional, multi-functional, or any combination thereof.

Additional polymer and PEG derivatives or cytotoxic tubulin inhibitor-linker derivatives including but not limited to, hydroxylamine (aminooxy) PEG derivatives or cytotoxic tubulin inhibitor-linker derivatives, are described in the following patent applications which are all incorporated by reference in their entirety herein: U.S. Patent Publication No. 2006/0194256, U.S. Patent Publication No. 2006/0217532, U.S. Patent Publication No. 2006/0217289, U.S. Provisional Patent No. 60/755,338; U.S. Provisional Patent No. 60/755,711; U.S. Provisional Patent No. 60/755,018; International Patent Application No. PCT/US06/49397; WO 2006/069246; U.S. Provisional Patent No. 60/743,041; U.S. Provisional Patent No. 60/743,040; International Patent Application No. PCT/US06/47822; U.S. Provisional Patent No. 60/882,819; U.S. Provisional Patent No. 60/882,500; and U.S. Provisional Patent No. 60/870,594.

### Glycosylation of ADC Polypeptides

Glycosylation can dramatically affect the physical properties (e.g., solubility) of polypeptides such as ADC polypeptides and can also be important in protein stability, secretion, and subcellular localization. Glycosylated polypeptides can also exhibit enhanced stability or can improve one or more pharmacokinetic properties, such as half-life. In addition, solubility improvements can, for example, enable the generation of formulations more suitable for pharmaceutical administration than formulations comprising the non-glycosylated polypeptide.

The disclosure includes ADC polypeptides incorporating one or more non-naturally encoded amino acids bearing saccharide residues. The saccharide residues may be either natural (including but not limited to, N-acetylglucosamine) or non-natural (including but not limited to, 3-fluorogalactose). The saccharides may be linked to the non-naturally encoded amino acids either by an N- or O-linked glycosidic linkage (including but not limited to, N-acetylgalactose-L-serine) or a non-natural linkage (including but not limited to, an oxime or the corresponding C- or S-linked glycoside).

The saccharide (including but not limited to, glycosyl) moieties can be added to ADC polypeptides either in vivo or in vitro. In some embodiments of the disclosure, a targeting polypeptide of the ADC comprising a carbonyl-containing non-naturally encoded amino acid is modified with a saccharide derivatized with an aminooxy group to generate the corresponding glycosylated polypeptide linked via an oxime linkage. Once attached to the non-naturally encoded amino acid, the saccharide may be further elaborated by treatment with glycosyltransferases and other enzymes to generate an oligosaccharide bound to the ADC polypeptide. See, e.g., H. Liu, et al. J. Am. Chem. Soc. 125: 1702-1703 (2003).

In some embodiments of the disclosure, a ADC polypeptide comprising a carbonyl-containing non-naturally encoded amino acid is modified directly with a glycan with defined structure prepared as an aminooxy derivative. One of ordinary skill in the art will recognize that other functionalities, including azide, alkyne, hydrazide, hydrazine, and semicarbazide, can be used to link the saccharide to the non-naturally encoded amino acid.

In some embodiments of the disclosure, a targeting polypeptide of the ADC comprising an azide or alkynyl-containing non-naturally encoded amino acid can then be modified by, including but not limited to, a Huisgen [3+2] cycloaddition reaction with, including but not limited to, alkynyl or azide derivatives, respectively. This method allows for proteins to be modified with extremely high selectivity.

### HER2 Target

HER2 polypeptide activity can be determined using standard or known in vitro or in vivo assays. ADC may be analyzed for biological activity by suitable methods known in the art. Such assays include, but are not limited to, activation of responsive genes, receptor binding assays, anti-viral activity assays, cytopathic effect inhibition assays, anti-proliferative assays, immunomodulatory assays and assays that monitor the induction of MHC molecules.

HER2 polypeptides may be analyzed for their ability to activate sensitive signal transduction pathways. One example is the interferon-stimulated response element (ISRE) assay. Cells which constitutively express the HER2 receptor are transiently transfected with an ISRE-luciferase vector (pISRE-luc, Clontech). After transfection, the cells are treated with a targeting polypeptide of the ADC. A number of protein concentrations, for example from 0.0001-10 ng/mL, are tested to generate a dose-response curve. If the ADC polypeptide binds and activates the HER2 receptor, the resulting signal transduction cascade induces luciferase expression. Luminescence can be measured in a number of ways, for example by using a TopCount™ or Fusion™ microplate reader and Steady-Glo® Luciferase Assay System (Promega).

HER2 polypeptides may be analyzed for their ability to bind to the HER2 receptor. For a non-PEGylated or PEGylated ADC polypeptide comprising a non-natural amino acid, the affinity of HER2 for its receptor can be measured by using a BIAcore™ biosensor (Pharmacia). Suitable binding assays include, but are not limited to, BIAcore assays (Pearce et al., Biochemistry 38:81-89 (1999)) and AlphaScreen™ assays (PerkinElmer).

Regardless of which methods are used to create the targeting polypeptides, the targeting polypeptides are subject to assays for biological activity. In general, the test for biological activity should provide analysis for the desired result, such as increase or decrease in biological activity (as compared to modified ADC), different biological activity (as compared to modified ADC), receptor or binding partner affinity analysis, conformational or structural changes of the ADC itself or its receptor (as compared to the modified ADC), or serum half-life analysis.

### Measurement of Potency, Functional In Vivo Half-Life, and Pharmacokinetic Parameters

An important aspect of the disclosure is the prolonged biological half-life that is obtained by construction of the ADC with or without conjugation of the polypeptide to a water soluble polymer moiety. The rate of post administration decrease of ADC serum concentrations may make it important to evaluate biological responses to treatment with conjugated and non-conjugated ADC polypeptide and variants thereof. The conjugated and non-conjugated ADC polypeptide and variants thereof of the present disclosure may have prolonged serum half-lives also after administration via, e.g. subcutaneous or i.v. administration, making it possible to measure by, e.g. ELISA method or by a primary screening assay. ELISA or RIA kits from commercial sources may be used such as Invitrogen (Carlsbad, CA). Measurement of in vivo biological half-life is carried out as described herein.

The potency and functional in vivo half-life of a targeting polypeptide of the ADC comprising a non-naturally encoded amino acid can be determined according to protocols known to those of ordinary skill in the art.

Pharmacokinetic parameters for a targeting polypeptide of the ADC comprising a non-naturally encoded amino acid can be evaluated in normal Sprague-Dawley male rats (N═5 animals per treatment group). Animals receive either a single dose of 25 ug/rat iv or 50 ug/rat sc, and approximately 5-7 blood samples are taken according to a pre-defined time course, generally covering about 6 hours for a targeting polypeptide of the ADC comprising a non-naturally encoded amino acid not conjugated to a water soluble polymer and about 4 days for a targeting polypeptide comprising a non-naturally encoded amino acid and conjugated to a water soluble polymer. Pharmacokinetic data for a targeting polypeptide without a non-naturally encoded amino acid can be compared directly to the data obtained for targeting polypeptides comprising a non-naturally encoded amino acid.

### Administration and Pharmaceutical Compositions

The polypeptides or proteins of the disclosure (including but not limited to, synthetases, proteins comprising one or more non-natural amino acid, etc.) are optionally employed for therapeutic uses, including but not limited to, in combination with a suitable pharmaceutical carrier. Such compositions, for example, comprise a therapeutically effective amount of the compound, and a pharmaceutically acceptable carrier or excipient. Such a carrier or excipient includes, but is not limited to, saline, buffered saline, dextrose, water, glycerol, ethanol, and/or combinations thereof. The formulation is made to suit the mode of administration. In general, methods of administering proteins are known to those of ordinary skill in the art and can be applied to administration of the polypeptides of the disclosure. Compositions may be in a water-soluble form, such as being present as pharmaceutically acceptable salts, which is meant to include both acid and base addition salts.

Therapeutic compositions comprising one or more polypeptide of the disclosure are optionally tested in one or more appropriate in vitro and/or in vivo animal models of disease, to confirm efficacy, tissue metabolism, and to estimate dosages, according to methods known to those of ordinary skill in the art. In particular, dosages can be initially determined by activity, stability or other suitable measures of unnatural herein to natural amino acid homologues (including but not limited to, comparison of a targeting polypeptide of the ADC modified to include one or more non-natural amino acids to a natural amino acid of the ADC's targeting polypeptide and comparison of a targeting polypeptide of the ADC modified to include one or more non-natural amino acids to a currently available ADC treatment), i.e., in a relevant assay.

Administration is by any of the routes normally used for introducing a molecule into ultimate contact with blood or tissue cells. The non-natural amino acid polypeptides of the disclosure are administered in any suitable manner, optionally with one or more pharmaceutically acceptable carriers. Suitable methods of administering such polypeptides in the context of the present disclosure to a patient are available, and, although more than one route can be used to administer a particular composition, a particular route can often provide a more immediate and more effective action or reaction than another route.

Pharmaceutically acceptable carriers are determined in part by the particular composition being administered, as well as by the particular method used to administer the composition. Accordingly, there is a wide variety of suitable formulations of pharmaceutical compositions of the present disclosure.

ADCs of the disclosure may be administered by any conventional route suitable for proteins or peptides, including, but not limited to parenterally, e.g. injections including, but not limited to, subcutaneously or intravenously or any other form of injections or infusions. ADCs and compositions thereof can be administered by a number of routes including, but not limited to oral, intravenous, intraperitoneal, intramuscular, transdermal, subcutaneous, topical, sublingual, or rectal means. Compositions comprising non-natural amino acid polypeptides, modified or unmodified, can also be administered via liposomes. Such administration routes and appropriate formulations are generally known to those of skill in the art. The ADC may be used alone or in combination with other suitable components such as a pharmaceutical carrier. The ADC may be used in combination with other agents or therapeutics.

The ADC comprising a non-natural amino acid, alone or in combination with other suitable components, can also be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations of ADC can be presented in unit-dose or multi-dose sealed containers, such as ampules and vials.

Parenteral administration and intravenous administration are preferred methods of administration. In particular, the routes of administration already in use for natural amino acid homologue therapeutics (including but not limited to, those typically used for EPO, GH, G-CSF, GM-CSF, IFNs e.g., interleukins, antibodies, FGFs, and/or any other pharmaceutically delivered protein), along with formulations in current use, provide preferred routes of administration and formulation for the polypeptides of the disclosure.

The dose administered to a patient, in the context of the present disclosure, is sufficient to have a beneficial therapeutic response in the patient over time, or other appropriate activity, depending on the application. The dose is determined by the efficacy of the particular vector, or formulation, and the activity, stability or serum half-life of the non-natural amino acid polypeptide employed and the condition of the patient, as well as the body weight or surface area of the patient to be treated. The size of the dose is also determined by the existence, nature, and extent of any adverse side-effects that accompany the administration of a particular vector, formulation, or the like in a particular patient.

In determining the effective amount of the vector or formulation to be administered in the treatment or prophylaxis of disease (including but not limited to, neutropenia, aplastic anemia, cyclic neutropenia, idiopathic neutropenia, Chdiak-Higashi syndrome, systemic lupus erythematosus (SLE), leukemia, myelodysplastic syndrome and myelofibrosis, or the like), the physician evaluates circulating plasma levels, formulation toxicities, and disease progression.

The dose administered, for example, to a 70 kilogram patient, is typically in the range equivalent to dosages of currently-used therapeutic proteins, adjusted for the altered activity or serum half-life of the relevant composition. The vectors or pharmaceutical formulations of this disclosure can supplement treatment conditions by any known conventional therapy, including antibody administration, vaccine administration, administration of cytotoxic agents, natural amino acid polypeptides, nucleic acids, nucleotide analogues, biologic response modifiers, and the like.

For administration, formulations of the present disclosure are administered at a rate determined by the LD-50 or ED-50 of the relevant formulation, and/or observation of any side-effects of the non-natural amino acid polypeptides at various concentrations, including but not limited to, as applied to the mass and overall health of the patient. Administration can be accomplished via single or divided doses.

If a patient undergoing infusion of a formulation develops fevers, chills, or muscle aches, he/she receives the appropriate dose of aspirin, ibuprofen, acetaminophen or other pain/fever controlling drug. Patients who experience reactions to the infusion such as fever, muscle aches, and chills are premedicated 30 minutes prior to the future infusions with either aspirin, acetaminophen, or, including but not limited to, diphenhydramine. Meperidine is used for more severe chills and muscle aches that do not quickly respond to antipyretics and antihistamines. Cell infusion is slowed or discontinued depending upon the severity of the reaction.

Human forms of a targeting polypeptide of the ADCs of the disclosure can be administered directly to a mammalian subject. Administration is by any of the routes normally used for introducing an ADC or polypeptide to a subject. The ADC compositions according to embodiments of the present disclosure include those suitable for oral, rectal, topical, inhalation (including but not limited to, via an aerosol), buccal (including but not limited to, sub-lingual), vaginal, parenteral (including but not limited to, subcutaneous, intramuscular, intradermal, intraarticular, intrapleural, intraperitoneal, intracerebral, intraarterial, or intravenous), topical (i.e., both skin and mucosal surfaces, including airway surfaces), pulmonary, intraocular, intranasal, and transdermal administration, although the most suitable route in any given case may depend on the nature and severity of the condition being treated. Administration can be either local or systemic. The formulations of compounds can be presented in unit-dose or multi-dose sealed containers, such as ampoules and vials. ADC of the disclosure can be prepared in a mixture in a unit dosage injectable form (including but not limited to, solution, suspension, or emulsion) with a pharmaceutically acceptable carrier. ADC of the disclosure can also be administered by continuous infusion (using, including but not limited to, minipumps such as osmotic pumps), single bolus or slow-release depot formulations.

Formulations suitable for administration include aqueous and non-aqueous solutions, isotonic sterile solutions, which can contain antioxidants, buffers, bacteriostats, and solutes that render the formulation isotonic, and aqueous and non-aqueous sterile suspensions that can include suspending agents, solubilizers, thickening agents, stabilizers, and preservatives. Solutions and suspensions can be prepared from sterile powders, granules, and tablets of the kind previously described.

Freeze-drying is a commonly employed technique for presenting proteins which serves to remove water from the protein preparation of interest. Freeze-drying, or lyophilization, is a process by which the material to be dried is first frozen and then the ice or frozen solvent is removed by sublimation in a vacuum environment. An excipient may be included in pre-lyophilized formulations to enhance stability during the freeze-drying process and/or to improve stability of the lyophilized product upon storage. Pikal, M. Biopharm. 3(9) 26-30 (1990) and Arakawa et al. Pharm. Res. 8(3):285-291 (1991).

The spray drying of pharmaceuticals is also known to those of ordinary skill in the art. For example, see Broadhead, J. et al., “The Spray Drying of Pharmaceuticals,” in Drug Dev. Ind. Pharm, 18 (11 & 12), 1169-1206 (1992). In addition to small molecule pharmaceuticals, a variety of biological materials have been sprayed dry and these include: enzymes, sera, plasma, micro-organisms and yeasts. Spray drying is a useful technique because it can convert a liquid pharmaceutical preparation into a fine, dustless or agglomerated powder in a one-step process. The basic technique comprises the following four steps: a) atomization of the feed solution into a spray; b) spray-air contact; c) drying of the spray; and d) separation of the dried product from the drying air. U.S. Pat. Nos. 6,235,710 and 6,001,800, which are incorporated by reference herein, describe the preparation of recombinant erythropoietin by spray drying.

The pharmaceutical compositions and formulations of the disclosure may comprise a pharmaceutically acceptable carrier, excipient, or stabilizer. Pharmaceutically acceptable carriers are determined in part by the particular composition being administered, as well as by the particular method used to administer the composition. Accordingly, there is a wide variety of suitable formulations of pharmaceutical compositions (including optional pharmaceutically acceptable carriers, excipients, or stabilizers) of the present disclosure (see, e.g., Remington's Pharmaceutical Sciences, 17^{th} ed. 1985)).

Suitable carriers include but are not limited to, buffers containing succinate, phosphate, borate, HEPES, citrate, histidine, imidazole, acetate, bicarbonate, and other organic acids; antioxidants including but not limited to, ascorbic acid; low molecular weight polypeptides including but not limited to those less than about 10 residues; proteins, including but not limited to, serum albumin, gelatin, or immunoglobulins; hydrophilic polymers including but not limited to, polyvinylpyrrolidone; amino acids including but not limited to, glycine, glutamine, asparagine, arginine, histidine or histidine derivatives, methionine, glutamate, or lysine; monosaccharides, disaccharides, and other carbohydrates, including but not limited to, trehalose, sucrose, glucose, mannose, or dextrins; chelating agents including but not limited to, EDTA and edentate disodium; divalent metal ions including but not limited to, zinc, cobalt, or copper; sugar alcohols including but not limited to, mannitol or sorbitol; salt-forming counter ions including but not limited to, sodium and sodium chloride; fillers such as microcrystalline cellulose, lactose, corn and other starches; binding agents; sweeteners and other flavoring agents; coloring agents; and/or nonionic surfactants including but not limited to Tween™ (including but not limited to, Tween 80 (polysorbate 80) and Tween 20 (polysorbate 20), Pluronics™ and other pluronic acids, including but not limited to, pluronic acid F68 (poloxamer 188), or PEG. Suitable surfactants include for example but are not limited to polyethers based upon poly(ethylene oxide)-poly(propylene oxide)-poly(ethylene oxide), i.e., (PEO-PPO-PEO), or poly(propylene oxide)-poly(ethylene oxide)-poly(propylene oxide), i.e., (PPO-PEO-PPO), or a combination thereof. PEO-PPO-PEO and PPO-PEO-PPO are commercially available under the trade names Pluronics™, R-Pluronics™, Tetronics™ and R-Tetronics™ (BASF Wyandotte Corp., Wyandotte, Mich.) and are further described in U.S. Pat. No. 4,820,352 incorporated herein in its entirety by reference. Other ethylene/polypropylene block polymers may be suitable surfactants. A surfactant or a combination of surfactants may be used to stabilize PEGylated ADC against one or more stresses including but not limited to stress that results from agitation. Some of the above may be referred to as “bulking agents.” Some may also be referred to as “tonicity modifiers.” Antimicrobial preservatives may also be applied for product stability and antimicrobial effectiveness; suitable preservatives include but are not limited to, benzyl alcohol, benzalkonium chloride, metacresol, methyl/propyl parabene, cresol, and phenol, or a combination thereof. U.S. Pat. No. 7,144,574, which is incorporated by reference herein, describe additional materials that may be suitable in pharmaceutical compositions and formulations of the disclosure and other delivery preparations.

ADCs of the disclosure, including those linked to water soluble polymers such as PEG can also be administered by or as part of sustained-release systems. Sustained-release compositions include, including but not limited to, semi-permeable polymer matrices in the form of shaped articles, including but not limited to, films, or microcapsules. Sustained-release matrices include from biocompatible materials such as poly(2-hydroxyethyl methacrylate) (Langer et al., J Biomed. Mater. Res., 15: 267-277 (1981); Langer, Chem. Tech., 12: 98-105 (1982), ethylene vinyl acetate (Langer et al., supra) or poly-D-(−)-3-hydroxybutyric acid (EP 133,988), polylactides (polylactic acid) (U.S. Pat. No. 3,773,919; EP 58,481), polyglycolide (polymer of glycolic acid), polylactide co-glycolide (copolymers of lactic acid and glycolic acid) polyanhydrides, copolymers of L-glutamic acid and gamma-ethyl-L-glutamate (Sidman et al., Biopolymers, 22, 547-556 (1983), poly(ortho)esters, polypeptides, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. Sustained-release compositions also include a liposomally entrapped compound. Liposomes containing the compound are prepared by methods known per se: DE 3,218,121; Eppstein et al., Proc. Natl. Acad Sci. U.S.A., 82: 3688-3692 (1985); Hwang et al., Proc. Natl. Acad Sci. U.S.A., 77: 4030-4034 (1980); EP 52,322; EP 36,676; U.S. Pat. No. 4,619,794; EP 143,949; U.S. Pat. No. 5,021,234; Japanese Pat. Appln. 83-118008; U.S. Pat. Nos. 4,485,045 and 4,544,545; and EP 102,324. All references and patents cited are incorporated by reference herein.

Liposomally entrapped ADC can be prepared by methods described in, e.g., DE 3,218,121; Eppstein et al., Proc. Natl. Acad Sci. U.S.A., 82: 3688-3692 (1985); Hwang et al., Proc. Natl. Acad Sci. U.S.A., 77: 4030-4034 (1980); EP 52,322; EP 36,676; U.S. Pat. No. 4,619,794; EP 143,949; U.S. Pat. No. 5,021,234; Japanese Pat. Appln. 83-118008; U.S. Pat. Nos. 4,485,045 and 4,544,545; and EP 102,324. Composition and size of liposomes are well known or able to be readily determined empirically by one of ordinary skill in the art. Some examples of liposomes as described in, e.g., Park J W, et al., Proc. Natl. Acad Sci. USA 92:1327-1331 (1995); Lasic D and Papahadjopoulos D (eds): Medical Applications of Liposomes (1998); Drummond D C, et al., Liposomal drug delivery systems for cancer therapy, in Teicher B (ed): Cancer Drug Discovery and Development (2002); Park J W, et al., Clin. Cancer Res. 8:1172-1181 (2002); Nielsen U B, et al., Biochim. Biophys. Acta 1591(1-3):109-118 (2002); Mamot C, et al., Cancer Res. 63: 3154-3161 (2003). All references and patents cited are incorporated by reference herein.

### Therapeutic Uses of ADC of the Disclosure

The ADC of the disclosure are useful for treating a wide range of disorders. The disclosure also includes a method of treating a mammal that is at risk for, is having, and/or has had a cancer responsive to HER2 overexpression, amplification, mutation and/or targeted therapies. Administration of ADCs may result in a short term effect, i.e., an immediate beneficial effect on several clinical parameters observed and this may 12 or 24 hours from administration, and, on the other hand, may also result in a long term effect, a beneficial slowing of progression of tumor growth, reduction in tumor size, and/or increased circulating CD8+ T cell levels and the ADC of the present disclosure may be administered by any means known to those skilled in the art, and may beneficially be administered via infusion, e.g. by arterial, intraperitoneal or intravenous injection and/or infusion in a dosage which is sufficient to obtain the desired pharmacological effect.

The dose administered to a patient in the context of the present disclosure can be sufficient to cause a beneficial response in the subject over time. The ADC dosage may range from 10-200 mg, or 40-80 mg ADC per kg body weight per treatment. For example, the dosage of ADC which is administered may be about 20-100 mg ADC per kg body weight given as a bolus injection and/or as an infusion for a clinically necessary period of time, e.g., for a period ranging from a few minutes to several hours, e.g., up to 24 hours. If necessary, the ADC administration may be repeated one or several times. In some embodiments the ADC is administered at a dose of 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0 mg/kg or greater. In some embodiments the ADC is administered at a dose of 0.33 mg/kg, 0.66 mg/kg, 0.88 mg/kg, 1.1 mg/kg, 1.2 mg/ml, 1.3 mg/kg, 1.4 mg/kg, 1.5 mg/kg, 1.6 mg/kg, 1.7 mg/kg, 1.8 mg/kg 1.9 mg/kg, 2.0 mg/kg or greater. In some embodiments the ADC is administered at a dose of 0.33 mg/kg, 0.66 mg/kg, 0.88 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg or greater Q2W. In some embodiments the ADC is administered at a dose of 0.33 mg/kg, 0.66 mg/kg, 0.88 mg/kg, 1.1 mg/kg, 1.2 mg/ml, 1.3 mg/kg, 1.4 mg/kg, 1.5 mg/kg, 1.6 mg/kg, 1.7 mg/kg, 1.8 mg/kg, 1.9 mg/kg, 2.0 mg/kg or greater Q3W. In some embodiments the ADC is administered at a dose of 0.33 mg/kg, 0.66 mg/kg, 0.88 mg/kg, 1.1 mg/kg, 1.2 mg/ml, 1.3 mg/kg, 1.4 mg/kg, 1.5 mg/kg, 1.6 mg/kg, 1.7 mg/kg, 1.8 mg/kg, 1.9 mg/kg, 2 mg/kg or greater Q4W. In some embodiments the ADC is administered at a dose of 0.33 mg/kg, 0.66 mg/kg, 0.88 mg/kg, 1.1 mg/kg, 1.2 mg/ml, 1.3 mg/kg, 1.4 mg/kg, 1.5 mg/kg, 1.6 mg/kg, 1.7 mg/kg, 1.8 mg/kg, 1.9 mg/kg, 2 mg/kg or greater Q6W.

In other embodiments, the dose is between 0.05 and 2 mg/kg or greater or any value in between. In other embodiments, the dose is 0.05 mg/kg or greater. In other embodiments, the dose is 0.1 mg/kg or greater. In other embodiments, the dose is 0.2 mg/kg or greater. In other embodiments, the dose is 0.33 mg/kg or greater. In other embodiments, the dose is 0.4 mg/kg or greater. In other embodiments, the dose is 0.5 mg/kg or greater. In other embodiments, the dose is 0.6 mg/kg or greater. In other embodiments, the dose is 0.66 mg/kg or greater. In other embodiments, the dose is 0.7 mg/kg or greater. In other embodiments, the dose is 0.8 mg/kg or greater. In other embodiments, the dose is 0.88 mg/kg or greater. In other embodiments, the dose is 0.9 mg/kg or greater. In other embodiments, the dose is 1.1 mg/kg or greater. In other embodiments, the dose is 1.2 mg/kg or greater. In other embodiments, the dose is 1.3 mg/kg or greater. In other embodiments, the dose is 1.4 mg/kg or greater. In other embodiments, the dose is 1.5 mg/kg or greater. In other embodiments, the dose is 1.6 mg/kg or greater. In other embodiments, the dose is 1.7 mg/kg or greater. In other embodiments, the dose is 1.8 mg/kg or greater. In other embodiments, the dose is 1.9 mg/kg or greater. In other embodiments, the dose is 2 0.0 mg/kg or greater. In some embodiments the ADC is administered at hourly, daily, weekly, monthly or yearly dosage. In some embodiments, the doses of ADC to a subject can be chosen in accordance with different dose parameters. Based on a response in a subject at the initial doses applied, higher or lower doses may be employed to the extent that patient tolerance permits.

In one embodiment, the ADC is administered to a subject at an initial dose on day 1 followed by a second dose. In some embodiments the second dose is administered once weekly, once every 2 weeks, once every 3 weeks, once every 4 weeks, once every 5 weeks, once every 6 weeks or greater. In one embodiment, the ADC is administered to a subject at an initial dose on day 1 of first cycle of 2-week followed by a second cycle of 2-week. In some embodiments the second cycle of 2-week comprises the same or different dose of first cycle of 2-week. In one embodiment, the ADC is administered to a subject at an initial dose on day 1 of first cycle of 3-week followed by a second cycle of 3-week. In some embodiments the second cycle of 3-week comprises the same or different dose of first cycle of 3-week. In one embodiment, the ADC is administered to a subject at a dose on day 1 of first cycle of 4-week followed by a second cycle of 4-week. In one embodiment, the ADC is administered to a subject at a dose on day 1 of first cycle of 6-week followed by a second cycle of 6-week. In some embodiments the second cycle of 6-week comprises the same or different dose of first cycle of 6-week. In one embodiment, the ADC is administered to a subject at a dose on day 1 of first cycle of 8-week or greater followed by a second cycle of 8-week or greater. In some embodiments the second cycle of 8-week or greater comprises the same or different dose of first cycle of 8-week or greater. In another embodiment, the ADC is administered to a subject as a single dose or at an initial dose of about 2.0 mg/kg followed by a second dose of 0.33 mg/kg, 0.66 mg/kg, 0.88 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, or greater once weekly, once every 2 weeks, once every 3 weeks, once every 4 weeks, once every 5 weeks, once every 6 weeks or greater. In another embodiment, the ADC is administered to a subject as a single dose or at an initial dose of about 1.9 mg/kg followed by a second dose of 0.33 mg/kg, 0.66 mg/kg, 0.88 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, or greater once weekly, once every 2 weeks, once every 3 weeks, once every 4 weeks, once every 5 weeks, once every 6 weeks or greater. In another embodiment, the ADC is administered to a subject as a single dose or at an initial dose of about 1.8 mg/kg followed by a second dose of 0.33 mg/kg, 0.66 mg/kg, 0.88 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, or greater once weekly, once every 2 weeks, once every 3 weeks, once every 4 weeks, once every 5 weeks, once every 6 weeks or greater. In another embodiment, the ADC is administered to a subject as a single dose or at an initial dose of about 1.7 mg/kg followed by a second dose of 0.33 mg/kg, 0.66 mg/kg, 0.88 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, or greater once weekly, once every 2 weeks, once every 3 weeks, once every 4 weeks, once every 5 weeks, once every 6 weeks or greater. In another embodiment, the ADC is administered to a subject as a single dose or at an initial dose of about 1.6 mg/kg followed by a second dose of 0.33 mg/kg, 0.66 mg/kg, 0.88 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, or greater once weekly, once every 2 weeks, once every 3 weeks, once every 4 weeks, once every 5 weeks, once every 6 weeks or greater. In another embodiment, the ADC is administered to a subject as a single dose or at an initial dose of about 1.5 mg/kg followed by a second dose of 0.33 mg/kg, 0.66 mg/kg, 0.88 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, or greater once weekly, once every 2 weeks, once every 3 weeks, once every 4 weeks, once every 5 weeks, once every 6 weeks or greater. In another embodiment, the ADC is administered to a subject as a single dose or at an initial dose of about 1.4 mg/kg followed by a second dose of 0.33 mg/kg, 0.66 mg/kg, 0.88 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, or greater once weekly, once every 2 weeks, once every 3 weeks, once every 4 weeks, once every 5 weeks, once every 6 weeks or greater. In another embodiment, the ADC is administered to a subject as a single dose or at an initial dose of about 1.3 mg/kg followed by a second dose of 0.33 mg/kg, 0.66 mg/kg, 0.88 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, or greater once weekly, once every 2 weeks, once every 3 weeks, once every 4 weeks, once every 5 weeks, once every 6 weeks or greater. In another embodiment, the ADC is administered to a subject as a single dose or at an initial dose of about 1.2 mg/kg followed by a second dose of 0.33 mg/kg, 0.66 mg/kg, 0.88 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, or greater once weekly, once every 2 weeks, once every 3 weeks, once every 4 weeks, once every 5 weeks, once every 6 weeks or greater. In another embodiment, the ADC is administered to a subject as a single dose or at an initial dose of about 1.1 mg/kg followed by a second dose of 0.33 mg/kg, 0.66 mg/kg, 0.88 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, or greater once weekly, once every 2 weeks, once every 3 weeks, once every 4 weeks, once every 5 weeks, once every 6 weeks or greater. In another embodiment, the ADC is administered to a subject as a single dose or at an initial dose of about 1.0 mg/kg followed by a second dose of 0.33 mg/kg, 0.66 mg/kg, 0.88 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, or greater once weekly, once every 2 weeks, once every 3 weeks, once every 4 weeks, once every 5 weeks, once every 6 weeks or greater. In another embodiment, the ADC is administered to a subject as a single dose or at an initial dose of about 0.88 mg/kg followed by a second dose of 0.33 mg/kg, 0.66 mg/kg, 0.88 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, or greater once weekly, once every 2 weeks, once every 3 weeks, once every 4 weeks, once every 5 weeks, once every 6 weeks or greater. In another embodiment, the ADC is administered to a subject as a single dose or at an initial dose of about 0.66 mg/kg followed by a second dose of 0.33 mg/kg, 0.66 mg/kg, 0.88 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, or greater once weekly, once every 2 weeks, once every 3 weeks, once every 4 weeks, once every 5 weeks, once every 6 weeks or greater. In another embodiment, the ADC is administered to a subject as a single dose or at an initial dose of about 0.33 mg/kg followed by a second dose of 0.33 mg/kg, 0.66 mg/kg, 0.88 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, or greater once weekly, once every 2 weeks, once every 3 weeks, once every 4 weeks, once every 5 weeks, once every 6 weeks or greater.

In another embodiment, the ADC is administered to a subject at a dose of about 1.8 mg/kg of the first cycle of 4-week, wherein the administering further comprises a dose of about 1.7 mg/kg on day 1 of the second cycle of 4-week. In another embodiment, the ADC is administered to a subject at a dose of about 1.8 mg/kg of the first cycle of 4-week, wherein the administering further comprises a dose of about 1.6 mg/kg on day 1 of the second cycle of 4-week. In another embodiment, the ADC is administered to a subject at a dose of about 1.8 mg/kg of the first cycle of 4-week, wherein the administering further comprises a dose of about 1.5 mg/kg on day 1 of the second cycle of 4-week. In another embodiment, the ADC is administered to a subject at a dose of about 1.8 mg/kg of the first cycle of 4-week, wherein the administering further comprises a dose of about 1.4 mg/kg on day 1 of the second cycle of 4-week. In another embodiment, the ADC is administered to a subject at a dose of about 1.8 mg/kg of the first cycle of 4-week, wherein the administering further comprises a dose of about 1.3 mg/kg on day 1 of the second cycle of 4-week. In another embodiment, the ADC is administered to a subject at a dose of about 1.8 mg/kg of the first cycle of 4-week, wherein the administering further comprises a dose of about 1.2 mg/kg on day 1 of the second cycle of 4-week. In another embodiment, the ADC is administered to a subject at a dose of about 1.8 mg/kg of the first cycle of 4-week, wherein the administering further comprises a dose of about 1.1 mg/kg on day 1 of the second cycle of 4-week. In another embodiment, the ADC is administered to a subject at a dose of about 1.8 mg/kg of the first cycle of 4-week, wherein the administering further comprises a dose of about 0.88 mg/kg on day 1 of the second cycle of 4-week. In another embodiment, the ADC is administered to a subject at a dose of about 1.8 mg/kg of the first cycle of 4-week, wherein the administering further comprises a dose of about 0.66 mg/kg on day 1 of the second cycle of 4-week. In another embodiment, the ADC is administered to a subject at a dose of about 1.8 mg/kg of the first cycle of 4-week, wherein the administering further comprises a dose of about 0.33 mg/kg on day 1 of the second cycle of 4-week. In another embodiment, the ADC is administered to a subject at a dose of about 1.7 mg/kg of the first cycle of 4-week, wherein the administering further comprises a dose of about 1.6 mg/kg on day 1 of the second cycle of 4-week. In another embodiment, the ADC is administered to a subject at a dose of about 1.7 mg/kg of the first cycle of 4-week, wherein the administering further comprises a dose of about 1.5 mg/kg on day 1 of the second cycle of 4-week. In another embodiment, the ADC is administered to a subject at a dose of about 1.7 mg/kg of the first cycle of 4-week, wherein the administering further comprises a dose of about 1.4 mg/kg on day 1 of the second cycle of 4-week. In another embodiment, the ADC is administered to a subject at a dose of about 1.7 mg/kg of the first cycle of 4-week, wherein the administering further comprises a dose of about 1.3 mg/kg on day 1 of the second cycle of 4-week. In another embodiment, the ADC is administered to a subject at a dose of about 1.7 mg/kg of the first cycle of 4-week, wherein the administering further comprises a dose of about 1.2 mg/kg on day 1 of the second cycle of 4-week. In another embodiment, the ADC is administered to a subject at a dose of about 1.7 mg/kg of the first cycle of 4-week, wherein the administering further comprises a dose of about 1.1 mg/kg on day 1 of the second cycle of 4-week. In another embodiment, the ADC is administered to a subject at a dose of about 1.7 mg/kg of the first cycle of 4-week, wherein the administering further comprises a dose of about 0.88 mg/kg on day 1 of the second cycle of 4-week. In another embodiment, the ADC is administered to a subject at a dose of about 1.7 mg/kg of the first cycle of 4-week, wherein the administering further comprises a dose of about 0.66 mg/kg on day 1 of the second cycle of 4-week. In another embodiment, the ADC is administered to a subject at a dose of about 1.7 mg/kg of the first cycle of 4-week, wherein the administering further comprises a dose of about 0.33 mg/kg on day 1 of the second cycle of 4-week. In another embodiment, the ADC is administered to a subject at an initial dose of about 1.6 mg/kg followed by a second dose of 0.33 mg/kg, 0.66 mg/kg, 0.88 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, or greater once weekly, once every 2 weeks, once every 3 weeks, once every 4 weeks, once every 5 weeks, once every 6 weeks or greater. In another embodiment, the ADC is administered to a subject 1.6 mg/kg of the first cycle of 4-week, wherein the administering further comprises about 1.5 mg/kg on day 1 of the second cycle of 4-week. In another embodiment, the ADC is administered to a subject 1.6 mg/kg of the first cycle of 4-week, wherein the administering further comprises about 1.4 mg/kg on day 1 of the second cycle of 4-week. In another embodiment, the ADC is administered to a subject 1.6 mg/kg of the first cycle of 4-week, wherein the administering further comprises about 1.3 mg/kg on day 1 of the second cycle of 4-week. In another embodiment, the ADC is administered to a subject 1.6 mg/kg of the first cycle of 4-week, wherein the administering further comprises about 1.2 mg/kg on day 1 of the second cycle of 4-week. In another embodiment, the ADC was administered to a subject 1.6 mg/kg of the first cycle of 4-week, wherein the administering further comprises about 1.1 mg/kg on day 1 of the second cycle of 4-week. In another embodiment, the ADC was administered to a subject 1.6 mg/kg of the first cycle of 4-week, wherein the administering further comprises about 0.88 mg/kg on day 1 of the second cycle of 4-week. In another embodiment, the ADC was administered to a subject 1.6 mg/kg of the first cycle of 4-week, wherein the administering further comprises about 0.66 mg/kg on day 1 of the second cycle of 4-week. In another embodiment, the ADC was administered to a subject 1.6 mg/kg of the first cycle of 4-week, wherein the administering further comprises about 0.33 mg/kg on day 1 of the second cycle of 4-week. In another embodiment, the ADC is administered to a subject at an initial dose of about 1.5 mg/kg followed by a second dose of 0.33 mg/kg, 0.66 mg/kg, 0.88 mg/kg, 1.10 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, or greater once weekly, once every 2 weeks, once every 3 weeks, once every 4 weeks, once every 5 weeks, once every 6 weeks or greater. In another embodiment, the ADC is administered to a subject 1.5 mg/kg of the first cycle of 4-week, wherein the administering further comprises about 1.4 mg/kg on day 1 of the second cycle of 4-week. In another embodiment, the ADC is administered to a subject 1.5 mg/kg of the first cycle of 4-week, wherein the administering further comprises about 1.3 mg/kg on day 1 of the second cycle of 4-week. In another embodiment, the ADC is administered to a subject 1.5 mg/kg of the first cycle of 4-week, wherein the administering further comprises about 1.2 mg/kg on day 1 of the second cycle of 4-week. In another embodiment, the ADC was administered to a subject 1.5 mg/kg of the first cycle of 4-week, wherein the administering further comprises about 1.1 mg/kg on day 1 of the second cycle of 4-week. In another embodiment, the ADC was administered to a subject 1.5 mg/kg of the first cycle of 4-week, wherein the administering further comprises about 0.88 mg/kg on day 1 of the second cycle of 4-week. In another embodiment, the ADC was administered to a subject 1.5 mg/kg of the first cycle of 4-week, wherein the administering further comprises about 0.66 mg/kg on day 1 of the second cycle of 4-week. In another embodiment, the ADC was administered to a subject 1.5 mg/kg of the first cycle of 4-week, wherein the administering further comprises about 0.33 mg/kg on day 1 of the second cycle of 4-week.

The administration of an ADC of the disclosure may be combined with the administration of other pharmaceutical agents such as therapeutic agents including chemotherapeutic agents, immunotherapeutic agents, hormonal agents, antitumor agents, immunostimulatory agents, immunomodulators, or combination thereof. The administration of an ADC of the disclosure may be combined with the administration of a checkpoint inhibitor, a HER2 kinase inhibitor, a cyclin-dependent kinase inhibitor, a tyrosine kinase inhibitor, a small-molecule kinase inhibitor, or a platinum-based therapeutic. The administration of an ADC of the disclosure may be combined with the administration of a HER2 targeted therapeutic including, but not limited to, lapatinib (Tykerb®), pertuzumab (Perjeta®), and ado-trastuzumab emtansine (Kadcyla®), also known as T-DM1. Additional HER2 targeted therapeutic may include pyrotinib, a small irreversible inhibitor of HER1, HER2, and HER4 that has demonstrated good preclinical activity in blocking HER2-mediated downstream signaling and tumor growth in breast cancer cell lines and xenograft mode. Furthermore, the present disclosure relates to a method for prophylaxis and/or treatment of cancer comprising administering a subject in need thereof an effective amount of ADC.

Average quantities of the ADC may vary and in particular can be based upon the recommendations and prescription of a qualified physician. The exact amount of ADC is a matter of preference subject to such factors as the exact type of condition being treated, the condition of the patient being treated, as well as the other ingredients in the composition. The disclosure also provides for administration of a therapeutically effective amount of another active agent. The amount to be given may be readily determined by one of ordinary skill in the art based upon therapy with ADCs.

### EXAMPLES

The following examples are offered to illustrate, but not to limit the claimed disclosure.

### Example 1: ADC Drug Product

An ADC of the disclosure, (for example ARX788), is a novel antibody drug conjugate (ADC) that consists of a human epidermal growth factor receptor 2 (HER2) directed monoclonal antibody (mAb) linked to the cytotoxic payload AS269, a highly potent tubulin inhibitor that inhibits cell growth. Through our proprietary technology, a non-natural amino acid, para-acetylphenylalanine (pAF), is precisely incorporated into the pre-determined site on the heavy chain of the anti-HER2 mAb, and AS269 is specifically conjugated to the non-natural amino acid pAF on the mAb via a highly stable oxime bond or double bond to produce the ADC (one payload per heavy chain). The resulting ADC has a homogeneous drug to antibody ratio (DAR) of 1.9 to 2.0 (theoretical maximal DAR of 2.0). The mechanism of action of the ADC is achieved through multiple sequential steps. The ADC specifically binds to HER2 on the surface of cancer cells, is rapidly internalized, transports to the lysosome, and is metabolized inside the lysosome to release pAF-AS269, which binds to microtubules and induces cell cycle arrest and cell death. Given the highly stable linkage between the HER2 antibody and the payload AS269 in the ADC, no free AS269 was detected after the ADC was incubated in plasma samples. Consequently, release of free AS269 is not expected in the circulation after its IV administration. The ADC specifically binds to HER2 on the surface of cancer cells, followed by rapid internalization into the cancer cells, then transports to lysosomes and is metabolized by target cells to release a potent tubulin inhibitor, pAF-AS269 that can inhibit proliferation of cancer cells.

The ADC is designed to kill HER2 overexpressing tumors through multiple sequential steps, including binding to HER2 on the surface of cancer cells, rapidly internalizing, trafficking to the lysosome, and metabolizing inside the lysosome to release para-acetylphenylalanine (pAF)-AS269, which binds to microtubules and induces cancer cell cycle arrest and cell death. Several other HER2-targeting therapeutic agents, antibodies, small molecules, and ADCs including Kadcyla® (Trastuzumab emtansine, T-DM1) and Enhertu® (Trastuzumab deruxtecan, T-DXd) have demonstrated clinical activity in cancers with HER2 over-expression.

### Example 2: Drug Used in the Study Shown in FIG. 1

The active pharmaceutical ingredient (API) is the ADC, ARX788. The ADC consists of a humanized anti-HER2 monoclonal antibody (IgG1κ) covalently conjugated to two microtubule-disrupting payloads AS269 (small molecule cytotoxic drug), with the coupling ratio fixed at 1:2. The 2 components of the ADC are: 1) the anti-HER2 mAb with specificity for human HER2, contains non-natural amino acid pAF at amino acid position 121 in the heavy chains of the anti-HER2 mAb; and 2) the cytotoxic payload AS269, a tubulin inhibitor that is highly potent for inhibiting cell growth and can be directly conjugated to the mAb. The amino-oxy group of AS269 is specifically conjugated to pAF through a stable oxime bond (one payload per heavy chain). There are two formulations of the study drug, injection liquid and freeze-dried lyophilized powder. The main drug used in the clinical trials is the lyophilized ADC.

pAF containing anti-HER2 antibody was concentrated to 10-20 mg/mL in conjugation buffer (30 mmol/L sodium acetate, pH 4.0). Acetic hydrazide and 10 molar equivalents AS269 (comprising a of hydroxyl-amine functionality for the conjugation) were added to the antibody. The conjugation reaction was continued for 18-20 hours at 30° C. followed by purification over a Capto SP Impres Column (GE Healthcare) to remove excess reagents. The purified ADC was buffer exchanged into formulation buffer and stored at <65° C. until further use. AS269 can be synthesized according to the experimental procedures disclosed in U.S. Pat. No. 8,476,411, the content of which is entirely incorporated herein by reference.

The ADC drug product manufacturing process is straightforward, well known in the art, and involves the steps that are commonly used in an aseptic fill-finish and lyophilization process. The ADC drug product manufacturing process includes thawing ADC bulk drug substance, pooling, sterile filtration, aseptic filling into vials, partial stoppering, freezing, primary and secondary drying, full stoppering and capping, followed by external vial washing and visual inspection, package labelling and storage.

Optimization of lyophilized formulation included screening ADC protein and polysorbate 80 concentration, buffer concentration and ADC protein and trehalose concentration. Optimization of the lyophilization cycle was focused on pre-freezing parameters, primary drying temperature, vacuum pressure and the time of secondary drying.

The final formulation selected for the ADC drug product is a lyophilized powder containing 20 mg/mL ADC in 5 mM L-histidine pH 6.0, with 6% trehalose, and 0.02% polysorbate 80 after reconstitution with sterile water for injection. The lyophilized drug product is a white or off-white cake or powder with no meltback or collapse. The reconstituted drug product solution is a clear to slightly opalescent, colorless to light yellow liquid, essentially free of visible particles, containing 20 mg/mL ADC in a sterile solution of 5 mM L-histidine with 6% trehalose and 0.02% polysorbate 80, pH 6.0. The lyophilized ADC is reconstituted by adding WFI (water for injection), and the calculated amount of ADC is transferred to a 250 mL infusion bag of 0.9% saline to prepare the infusion bag.

The ADC is also formulated in a liquid containing 54 mg/5.4 ml ARX788 in a 10 mL glass vial. The formulation after reconstitution is 5 mM histidine, 6% trehalose, 0.02% polysorbate 80, pH 6.0 with a nominal ADC concentration of 20 mg/mL.

### Example 3

This Example discloses various methodologies and techniques used in the present disclosure.

### Molecular Cloning—

CHO cell codon-optimized antibody heavy chain and light chain cDNA sequences were obtained from commercial DNA synthesis service (IDT, San Diego, CA). The synthesized DNA fragments were digested with Hind III and EcoR I (both from New England Biolabs (NEB), Ipswich, MA) and purified by PCR purification kit (Qiagen, Valencia, CA). The digested antibody gene fragments were ligated into the expression vector via quick ligation kit (NEB) to yield the constructs for expression of wild type antibody heavy chain and light chain. The resulting plasmids were propagated in E. coli and verified by DNA sequencing service (Eton).

### Generation of Amber Codon-Containing Mutants—

Based on the crystal structure of anti-HER2 Fab, 10 different surface-accessible sites located at light chain constant region were chosen to genetically incorporate non-natural amino acid (for example, para-acetyl-phenylalanine (pAF), or para-azido-phenylalanine). Those sites are not critical for antigen-antibody binding. Each genetic codon of the chosen site was then mutated to amber codon (TAG) via site-directed mutagenesis to generate expression plasmid for that antibody mutant. Primers were purchased from IDT. All site directed mutagenesis experiments were carried out using Q5 site-directed mutagenesis kit following instruction manuals (NEB). The expression plasmids for the mutants were propagated in E. coli and verified by DNA sequencing service (Eton). Table 3 provides a list of amber mutations sites in the heavy chain and/or light chain constant region of anti-HER2 Fab with their Kabat numbering and the corresponding amino acid sequences, SEQ ID NOs: 2, and 4 to 11. SEQ ID NOs: 1 and 3 shows the wild type heavy and light chains of anti-HER2 Fab, respectively. Anti-HER2 Fabs include the heavy chain and light chain sequences of: SEQ ID NO: 2 and SEQ ID NO: 3; SEQ ID NO: 2 and SEQ ID NO: 4; SEQ ID NO: 2 and SEQ ID NO: 5; SEQ ID NO: 2 and SEQ ID NO: 6; SEQ ID NO: 2 and SEQ ID NO: 7; SEQ ID NO: 2 and SEQ ID NO: 8; SEQ ID NO: 2 and SEQ ID NO: 9; SEQ ID NO: 2 and SEQ ID NO: 10; SEQ ID NO: 2 and SEQ ID NO: 11; SEQ ID NO: 2 and SEQ ID NO: 12; SEQ ID NO: 2 and SEQ ID NO: 13.

Table 3. Anti-HER2 Fab heavy chain (HC) and light chain (LC) amino acid sequences with Amber sites for non-natural amino acid incorporation. Also disclosed are: all of the sequences in Table 3, wherein X is replaced by any non-natural amino acid; all of the sequences in Table 3, wherein any amino acid is replaced by any non-natural amino acid; and all of the sequences in Table 3, wherein X is pAF.

| SEQ ID | | |
|---|---|---|
| NO | Description | Sequence |
| 1 | Heavy chain | EVQLVESGGGLVQPGGSLRLSCAASGFNIK |
| | wild type | DTYIHWVRQAPGKGLEWVARIYPTNGYTR |
| | | YADSVKGRFTISADTSKNTAYLQMNSLRAE |
| | | DTAVYYCSRWGGDGFYAMDYWGQGTLVT |
| | | VSSASTKGPSVFPLAPSSKSTSGGTAALGCL |
| | | VKDYFPEPVTVSWNSGALTSGVHTFPAVLQ |
| | | SSGLYSLSSVVTVPSSSLGTQTYICNVNHKP |
| | | SNTKVDKKVEPKSCDKTHTCPPCPAPELLG |
| | | GPSVFLFPPKPKDTLMISRTPEVTCVVVDV |
| | | SHEDPEVKFNWYVDGVEVHNAKTKPREE |
| | | QYNSTYRVVSVLTVLHQDWLNGKEYKCK |
| | | VSNKALPAPIEKTISKAKGQPREPQVYTLPP |
| | | SRDELTKNQVSLTCLVKGFYPSDIAVEWES |
| | | NGQPENNYKTTPPVLDSDGSFFLYSKLTVD |
| | | KSRWQQGNVFSCSVMHEALHNHYTQKSL |
| | | SLSPG |
| 2 | Heavy Chain | EVQLVESGGGLVQPGGSLRLSCAASGFNIK |
| | A114 mutation | DTYIHWVRQAPGKGLEWVARIYPTNGYTR |
| | | YADSVKGRFTISADTSKNTAYLQMNSLRAE |
| | | DTAVYYCSRWGGDGFYAMDYWGQGTLVT |
| | | VSSXSTKGPSVFPLAPSSKSTSGGTAALGCL |
| | | VKDYFPEPVTVSWNSGALTSGVHTFPAVLQ |
| | | SSGLYSLSSVVTVPSSSLGTQTYICNVNHKP |
| | | SNTKVDKKVEPKSCDKTHTCPPCPAPELLG |
| | | GPSVFLFPPKPKDTLMISRTPEVTCVVVDV |
| | | SHEDPEVKFNWYVDGVEVHNAKTKPREE |
| | | QYNSTYRVVSVLTVLHQDWLNGKEYKCK |
| | | VSNKALPAPIEKTISKAKGQPREPQVYTLPP |
| | | SRDELTKNQVSLTCLVKGFYPSDIAVEWES |
| | | NGQPENNYKTTPPVLDSDGSFFLYSKLTVD |
| | | KSRWQQGNVFSCSVMHEALHNHYTQKSL |
| | | SLSPG |
| 3 | Light Chain | DIQMTQSPSSLSASVGDRVTITCRASQDVN |
| | wild type | TAVAWYQQKPGKAPKLLIYSASFLYSGVPS |
| | | RFSGSRSGTDFTLTISSLQPEDFATYYCQQH |
| | | YTTPPTFGQGTKVEIKRTVAAPSVFIFPPSD |
| | | EQLKSGTASVVCLLNNFYPREAKVQWKVD |
| | | NALQSGNSQESVTEQDSKDSTYSLSSTLTL |
| | | SKADYEKHKVYACEVTHQGLSSPVTKSFN |
| | | RGEC |
| 4 | Light Chain | DIQMTQSPSSLSASVGDRVTITCRASQDVN |
| | V110 mutation | TAVAWYQQKPGKAPKLLIYSASFLYSGVPS |
| | | RFSGSRSGTDFTLTISSLQPEDFATYYCQQH |
| | | YTTPPTFGQGTKVEIKRTXAAPSVFIFPPSD |
| | | EQLKSGTASVVCLLNNFYPREAKVQWKVD |
| | | NALQSGNSQESVTEQDSKDSTYSLSSTLTL |
| | | SKADYEKHKVYACEVTHQGLSSPVTKSFN |
| | | RGEC |
| 5 | Light Chain | DIQMTQSPSSLSASVGDRVTITCRASQDVN |
| | A112 mutation | TAVAWYQQKPGKAPKLLIYSASFLYSGVPS |
| | | RFSGSRSGTDFTLTISSLQPEDFATYYCQQH |
| | | YTTPPTFGQGTKVEIKRTVAXPSVFIFPPSD |
| | | EQLKSGTASVVCLLNNFYPREAKVQWKVD |
| | | NALQSGNSQESVTEQDSKDSTYSLSSTLTL |
| | | SKADYEKHKVYACEVTHQGLSSPVTKSFN |
| | | RGEC |
| 6 | Light Chain | DIQMTQSPSSLSASVGDRVTITCRASQDVN |
| | S114 mutation | TAVAWYQQKPGKAPKLLIYSASFLYSGVPS |
| | | RFSGSRSGTDFTLTISSLQPEDFATYYCQQH |
| | | YTTPPTFGQGTKVEIKRTVAAPXVFIFPPSD |
| | | EQLKSGTASVVCLLNNFYPREAKVQWKVD |
| | | NALQSGNSQESVTEQDSKDSTYSLSSTLTL |
| | | SKADYEKHKVYACEVTHQGLSSPVTKSFN |
| | | RGEC |
| 7 | Light Chain | DIQMTQSPSSLSASVGDRVTITCRASQDVN |
| | S121 mutation | TAVAWYQQKPGKAPKLLIYSASFLYSGVPS |
| | | RFSGSRSGTDFTLTISSLQPEDFATYYCQQH |
| | | YTTPPTFGQGTKVEIKRTVAAPSVFIFPPXD |
| | | EQLKSGTASVVCLLNNFYPREAKVQWKVD |
| | | NALQSGNSQESVTEQDSKDSTYSLSSTLTL |
| | | SKADYEKHKVYACEVTHQGLSSPVTKSFN |
| | | RGEC |
| 8 | Light Chain | DIQMTQSPSSLSASVGDRVTITCRASQDVN |
| | S127 mutation | TAVAWYQQKPGKAPKLLIYSASFLYSGVPS |
| | | RFSGSRSGTDFTLTISSLQPEDFATYYCQQH |
| | | YTTPPTFGQGTKVEIKRTVAAPSVFIFPPSD |
| | | EQLKXGTASVVCLLNNFYPREAKVQWKV |
| | | DNALQSGNSQESVTEQDSKDSTYSLSSTLT |
| | | LSKADYEKHKVYACEVTHQGLSSPVTKSF |
| | | NRGEC |
| 9 | Light Chain | DIQMTQSPSSLSASVGDRVTITCRASQDVN |
| | K149 mutation | TAVAWYQQKPGKAPKLLIYSASFLYSGVPS |
| | | RFSGSRSGTDFTLTISSLQPEDFATYYCQQH |
| | | YTTPPTFGQGTKVEIKRTVAAPSVFIFPPSD |
| | | EQLKSGTASVVCLLNNFYPREAKVQWXVD |
| | | NALQSGNSQESVTEQDSKDSTYSLSSTLTL |
| | | SKADYEKHKVYACEVTHQGLSSPVTKSFN |
| | | RGEC |
| 10 | Light Chain | DIQMTQSPSSLSASVGDRVTITCRASQDVN |
| | S156 mutation | TAVAWYQQKPGKAPKLLIYSASFLYSGVPS |
| | | RFSGSRSGTDFTLTISSLQPEDFATYYCQQH |
| | | YTTPPTFGQGTKVEIKRTVAAPSVFIFPPSD |
| | | EQLKSGTASVVCLLNNFYPREAKVQWKVD |
| | | NALQXGNSQESVTEQDSKDSTYSLSSTLTL |
| | | SKADYEKHKVYACEVTHQGLSSPVTKSFN |
| | | RGEC |
| 11 | Light Chain | DIQMTQSPSSLSASVGDRVTITCRASQDVN |
| | S168 mutation | TAVAWYQQKPGKAPKLLIYSASFLYSGVPS |
| | | RFSGSRSGTDFTLTISSLQPEDFATYYCQQH |
| | | YTTPPTFGQGTKVEIKRTVAAPSVFIFPPSD |
| | | EQLKSGTASVVCLLNNFYPREAKVQWKVD |
| | | NALQSGNSQESVTEQDXKDSTYSLSSTLTL |
| | | SKADYEKHKVYACEVTHQGLSSPVTKSFN |
| | | RGEC |
| 12 | Light Chain | DIQMTQSPSSLSASVGDRVTITCRASQDVN |
| | S202 mutation | TAVAWYQQKPGKAPKLLIYSASFLYSGVPS |
| | | RFSGSRSGTDFTLTISSLQPEDFATYYCQQH |
| | | YTTPPTFGQGTKVEIKRTVAAPSVFIFPPSD |
| | | EQLKSGTASVVCLLNNFYPREAKVQWKVD |
| | | NALQSGNSQESVTEQDSKDSTYSLSSTLTL |
| | | SKADYEKHKVYACEVTHQGLXSPVTKSFN |
| | | RGEC |
| 13 | Light Chain | DIQMTQSPSSLSASVGDRVTITCRASQDVN |
| | V205 mutation | TAVAWYQQKPGKAPKLLIYSASFLYSGVPS |
| | | RFSGSRSGTDFTLTISSLQPEDFATYYCQQH |
| | | YTTPPTFGQGTKVEIKRTVAAPSVFIFPPSD |
| | | EQLKSGTASVVCLLNNFYPREAKVQWKVD |
| | | NALQSGNSQESVTEQDSKDSTYSLSSTLTL |
| | | SKADYEKHKVYACEVTHQGLSSPXTKSFN |
| | | RGEC |

X Denotes non-natural amino acid (nnAA); underlined denotes Fc mutation in Table 3; mutations positions are described according to Kabat numbering, as well known to the skilled artisan.

In addition to an amber mutation in the heavy chain at position 114 (Kabat numbering), Fc mutations may also be engineered at various positions of the anti-HER2 antibody or antibody fragment to improve the pharmacokinetics and/or enhance antibody dependent cellular phagocytosis (ADCP) and/or antibody dependent cellular cytotoxicity ADCC activity, (Table 4).

TABLE 4
anti-HER2 Fc mutations

| | Targeting Fc | |
|---|---|---|
| Fc Mutations | Receptor | Purpose |
| WT | N/A | Control |
| E233P/L234V/L235A | FcγRIII | ADCC Null |
| N434A | FcRn | Improved PK |
| M252Y/S254T/T256E | FcRn | Improved PK |
| M252Y/S254T/T256E | FcRn/FcγRII/FcγRIII | PK and ADCP enhance |
| G236A/S239D/I332E | | |
| G236A/S239D/I332E | FcγRII/RIII | ADCP/ADCC enhance |
| G236A | FcγRII | ADCP enhance |
| D270E | FcγRII | ADCP enhance |
| Y300L | FcγRII | ADCP enhance |

### Example 4: Treatment for Breast Cancer

Phase 2 Study of ARX788 in HER2-positive Metastatic Breast Cancer Patients Whose Disease is Resistant or Refractory to T-DM1, and/or T-DXd, and/or Tucatinib-containing Regimens.

Primary Objective: The confirmed objective response rate (ORR) of ARX788 by blinded independent central review (BICR) based on RECIST v1.1 in subjects with HER2-positive breast cancer whose disease is resistant or refractory to T-DM1, and/or T-DXd, and/or tucatinib-containing regimens.

Secondary Objectives: (1)To evaluate duration of response (DOR), best percent change in the sum of the longest diameters of measurable tumors, best overall response (BOR), disease control rate (DCR), progression-free survival (PFS), and overall survival (OS); (2) To evaluate the safety, tolerability, and immunogenicity profile of the ADC (ARX788); (3) determine the pharmacokinetics (PK) of ARX788.

Exploratory Objectives: evaluate the time to response (TTR) and to identify potential predictive and/or prognostic biomarkers

Study Design: A Phase 2 single arm study assessed the anticancer activity, in addition to the tolerability, safety, and PK of the ADC in subjects with HER2-positive (IHC 3+ or IHC2+ and ISH+) advanced breast cancer post T-DM1, and/or T-DXd, and/or tucatinib-based therapies, or other available and accessible HER2-directed therapies or investigational therapies. The target population is presented in Table 5.

TABLE 5
Target Population

| Study | Primary | Cancer | Subject | HER2 |
|---|---|---|---|---|
| Phase | Objective | Type | Number | Status |
| Phase | Assess | Advanced breast | Approximately | HER2-positive |
| 2 | anticancer | cancer which | 200 subjects | based |
| | activity | is resistant/ | | on the 2018 |
| | | refractory to T- | | ASCO/CAP |
| | | DM1, and/ | | guideline |
| | | or T-DXd, | | by central |
| | | and/or tucatinib- | | |
| | | containing | | |
| | | regimens. | | |

The subjects receive ADC at 1.5 mg/kg as the initial dose on Day 1 of the first 4-week cycle and followed by 1.3 mg/kg every subsequent 4-week cycle until intolerable toxicity, disease progression, discontinuation due to Investigator's clinical judgment, discontinuation due to subject's choice, or sponsor's decision to stop the study.

### Dosage and Administration

One treatment cycle is 21 or 28 days. Dose levels can include dosages of 0.33 mg/kg, 0.66 mg/kg, 0.88 mg/kg, 1.1 mg/kg, 1.2 mg/kg, 1.3 mg/kg, 1.5 mg/kg, 1.7 mg/kg. 1.8 mg/kg, 1.9 mg/kg or greater.

ARX788 can be administered as an IV infusion Q3W/Q4W. The initial infusion time is 90±10 minutes, and subsequent infusions can be reduced to 60±10 minutes at the discretion of the investigator if well tolerated and there is no history of infusion reactions. Refer to the concomitant medications section of the protocol for details on premedication.

In some embodiments subjects received ARX788 at 1.7 mg/kg as the initial dose on Day 1 of the first 4-week cycle followed by 1.5 mg/kg every subsequent 4-week cycle in accordance with the schedule of administration. In other embodiments, subjects receive ARX788 at 1.5 mg/kg as the initial dose on Day 1 of the first 4-week cycle followed by 1.3 mg/kg every subsequent 4-week cycle in accordance with the schedule of administration. Infusion bags are prepared by adding the appropriate amount of ARX788 to a 250 mL bag of 0.9% saline for injection. When tolerated, the entire contents of the bag are administered (with an in-line 0.22 μm filter) to the subject to deliver each dose.

Infusion time for the initial dose of ARX788 is 90 minutes and may be reduced to 60 minutes in subsequent infusions, if well tolerated, with no history of infusion reactions and based upon the Investigator's judgment. Date and time of each dose administration including start and stop time of the infusion and any infusion interruptions are recorded in the electronic case report forms (eCRFs). Cycle durations are 28 days (Q4W) and cycles continue as long as the subject is eligible to continue treatment.

### Method of Assigning Subjects to Treatment Groups

Approximately 200 subjects are enrolled to assess anticancer activity and safety of ARX788.

### Study Schedules

### Treatment Cycles

The treatment cycles are shown below:

| Q4W Infusion Schedule | | | | | | | | | | | | | | | |
|---|---|---|---|---|---|---|---|---|---|---|---|---|---|---|---|
| | | | | | | | | | | | | | | Cycle 5 | |
| Screening | | Cycle 1 | | | | | Cycle 2 | Cycle 3 | | | | | Cycle 4 | Onward | EOT ^{z} |
| Cycle Day | | | | | | | | | | | | | | | |
| | | | | | | | | | | | | | | | 30 days |
| Within | Within | | | | | | | | | | | | | | after |
| 28 days | 7 days | 1 | 2 | 4 | 8 | 15 | 1 | 1 | 2 | 4 | 8 | 15 | 1 | 1 | last dose |

Permitted visit window (days)

| | | | | | | | | | |
|---|---|---|---|---|---|---|---|---|---|
| 2 | ±2 | ±2 | 2 | 2 | ±2 | ±2 | 2 | 2 | ±7 |

| | | | | | |
|---|---|---|---|---|---|
| Administration of | X | X | X | X | X |
| ARX788 infusion ^{A} | | | | | |

On Day 1 of each treatment cycle, subjects are administered an infusion of ARX788. Assessments and examinations are completed at each visit within treatment cycles and at the EOT Visit.

On Day 1 of each treatment cycle, subjects are administered an infusion of ARX788. Assessments and examinations are completed at each visit within treatment cycles and at the EOT Visit. Assessments and examinations are per study protocol/guidelines and include tumor biopsies for the HER2 status evaluation and biomarker analysis; ECOG performance status before ARX788 administration of each cycle beginning from cycle 2 and at EOT; urinalysis; ophthalmologic exam; tumor imaging—including Contrast enhanced CT of the chest, CT/MRI of the abdomen, pelvis, and all other known and/or suspected sites of disease via CT/MRI at Screening, then Q8W (±7 days) independent of treatment cycle, and at EOT Visit.; Brain imaging -MRI, (or CT if MRI is contraindicated), of the brain without and with contrast is required for ALL participants during screening to rule out brain metastases; Chest X-Ray (CXR); Pulmonary Function Test (PFT); Echocardiograms; Echocardiograms; vital signs including hematology/blood work; renal function analysis, immunogenicity testing; observation for AEs/SAE, Survival follow-up.

### Endpoints

Primary Endpoints: The confirmed objective response rate (ORR) of ARX788 by blinded independent central review (BICR) based on RECIST v 1.1 in HER2-positive metastatic breast cancer subjects whose disease is resistant/refractory to T-DM1, and/or T-DXd, and/or tucatinib-containing regimens.

### Secondary Endpoints:

### Efficacy:

Anticancer activity of ARX788 is assessed as a secondary endpoint. Anticancer activity parameters that are assessed by BICR and/or Investigators include: (1) Duration of response (DOR); (2) Best percent change in the sum of the longest diameters of measurable tumors; (3) Best overall response (BOR) (complete response (CR), partial response (PR), stable disease (SD), or progressive disease); (4) Disease control rate/clinical benefit rate (DCR/CBR=CR+PR+SD); (5) Progression-free survival (PFS); (6) Overall survival (OS).

### Safety:

The safety and tolerability, and immunogenicity profile in participating subjects are assessed by AE, vital signs, ECG (RR, PR, QT intervals and QRS duration), LVEF, clinical laboratory tests, ADA assays, physical examination, medical history and prior and concomitant medication, and where applicable, changes from baseline are analyzed.

### Pharmacokinetics:

Serum concentrations of ARX788 (intact ADC), total antibody, and metabolite pAF-AS269 are evaluated over the administered dose level of ARX788. The PK and PDx characteristics of ARX788 and its major metabolites are further assessed. The PK parameters are assessed.

### Immunogenicity:

Presence of ADA and their potential impact on the PK, efficacy, and safety of ARX788 are assessed.

Exploratory Endpoints: Time to response (TTR) and Specific biomarker-related PDx effects of ARX788 are assessed based on a sample of tumor tissue and on blood sampling.

### Example 5: Efficacy of ARX788 in ARX788-101 Study

ARX788-101, a Phase 1, multicenter, open-label, multiple dose escalation study of ARX788, intravenously administered as a single agent in subjects with advanced cancers with HER2 expression, was the first-in-human study evaluating ARX788. Tumor responses (PRs and stable disease) were observed in 7 of 7 breast cancer patients with evaluable tumors, for a DCR of 100%. The best overall tumor responses were PRs that were observed in 3 of the 7 subjects, while stable disease was reported for 4 subjects. No subjects had progression of disease. For the 6 subjects whose baseline and post-baseline tumor size data were available, a decrease in tumor size of varying magnitude was observed.

### Example 6: Efficacy of ARX788 in ACE-Breast-01 Study (ARX788-111) and ACE-Pan Tumor-01 Study (ARX788-1711)

In ACE-Breast-01 Study (ARX788-111), a Phase 1, multicenter, open-label, multiple dose-escalation study of ARX788, intravenously administered as a single agent in subjects with advanced breast cancers with HER2 expression showed preliminary efficacy data from the 59 (of 60) evaluable subjects including a total of 28 PRs (and 27 patients with stable disease), yielding a dose-escalation study-wide ORR of 47.4% and a DCR of 93.2% thus far. For subjects dosed at 1.5 mg/kg Q3W, there were 19 PRs (18 confirmed) and 5 cases of stable disease reported out of 29 evaluable patients, for an ORR of 66% (confirmed ORR 62%) and a DCR of 100%. These patients were heavily pretreated, with a median of 5 prior lines of therapy (range: 2-11). The study is continued and a summary of efficacy data from all studies (including those described above) is presented in Table 6.

Table 6. Summary of ARX788 Clinical Efficacy Data in Ongoing Breast Cancer and Pan Tumor Studies

| (China Breast Cancer, ACE-Breast-01) | | | | (US/AUS Phase 1, ACE-Pan tumor-01) | | | |
|---|---|---|---|---|---|---|---|
| Dose Level | ORR^{a} | ORR | DCR | Dose Level | ORR^{a} | ORR | DCR |
| 0.33 & 0.66 mg/kg | 0% | 0% | 83.3% | 0.66 mg/kg | 20% | 20% | 60% |
| Q3W | | | | Q3W & Q4W | | | |
| (N = 6) | | | | (N = 5) | | | |
| 0.88 mg/kg | 14% | 14% | 86% | 0.88 mg/kg | 11% | 22% | 78% |
| Q3W & Q4W | | | | Q3W & Q4W | | | |
| (N = 7) | | | | (N = 9) | | | |
| 1.1 mg/kg Q3W & | 27% | 36% | 100% | 1.1 mg/kg Q4W | 33% | 33% | 67% |
| Q4W (N = 11) | | | | (N = 3) | | | |
| 1.3 mg/kg Q3W & | 50% | 56% | 88% | 1.3 mg/kg | 10% | 25% | 100% |
| Q4W (N = 16) | | | | Q3W & Q4W | | | |
| | | | | (N = 4) | | | |
| 1.5 mg/kg Q3W | 62% | 66%* | 100% | 1.5 mg/kg Q4W | 67% | 67% | 100% |
| (N = 29) | | | | (N = 3)** | | | |

Note:
ORR^{a} is confirmed ORR.
*Among 19 PR, 18 are confirmed, one patient's PR is still pending confirmation with subsequent scan. The confirmed ORR is 62%, the unconfirmed ORR is 66%
**Phase 1b dose expansion cohorts enrolled additional 5 patients, whose data are not mature yet (1SD, 1 subject with only one tumor scan which is SD; 3 subjects with no tumor scan yet)

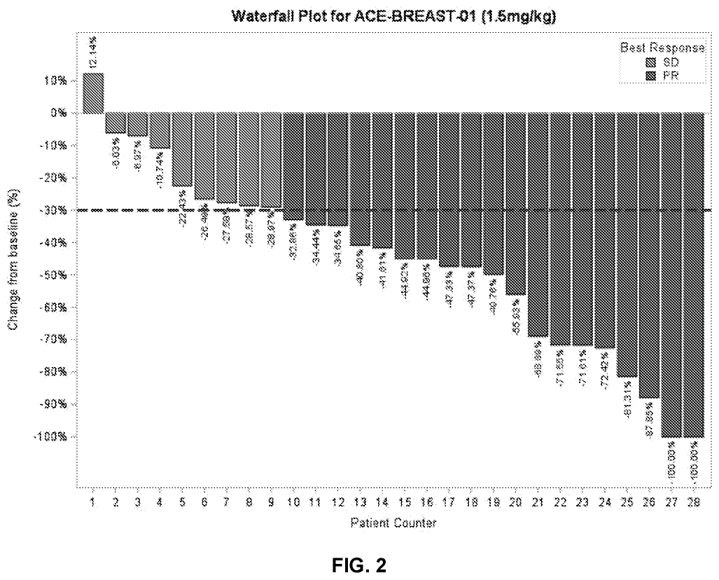

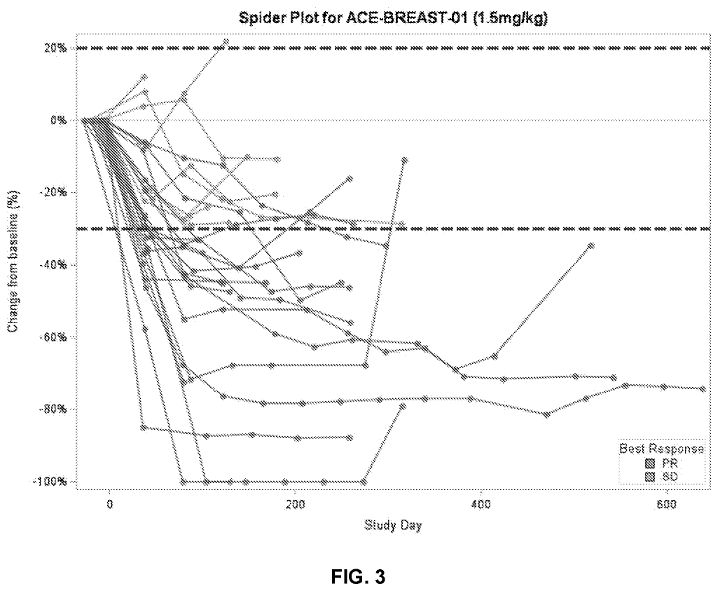

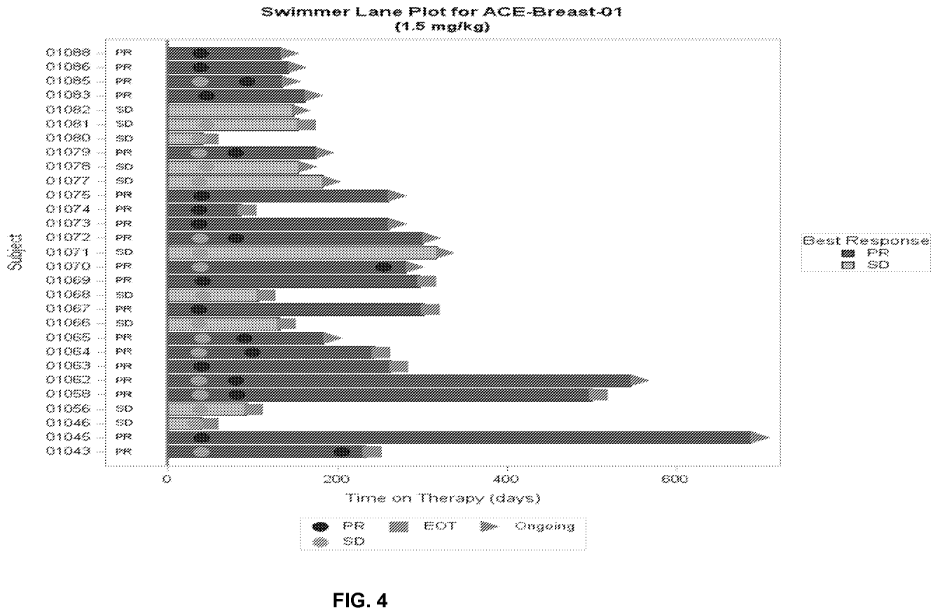

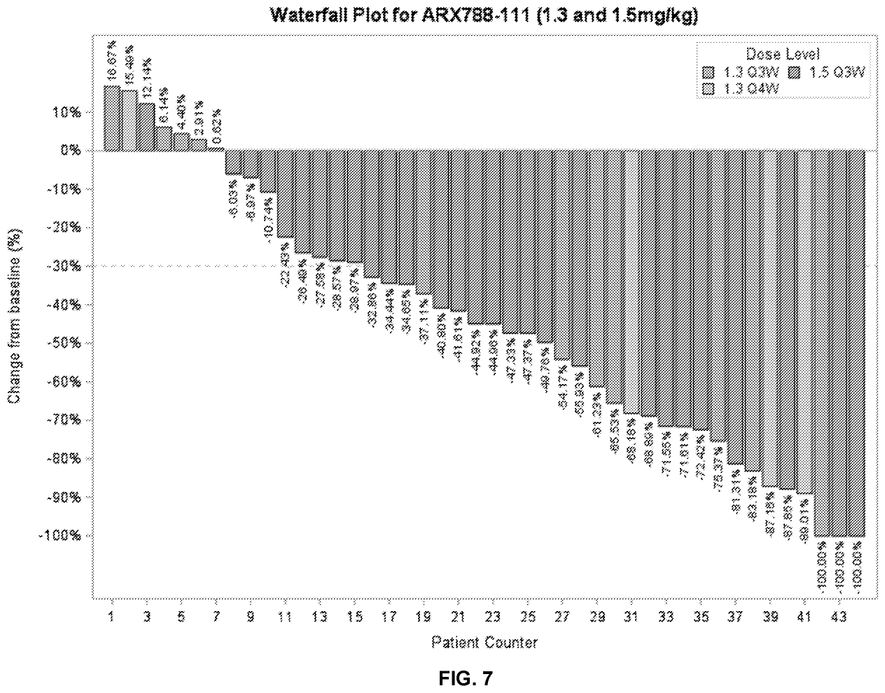

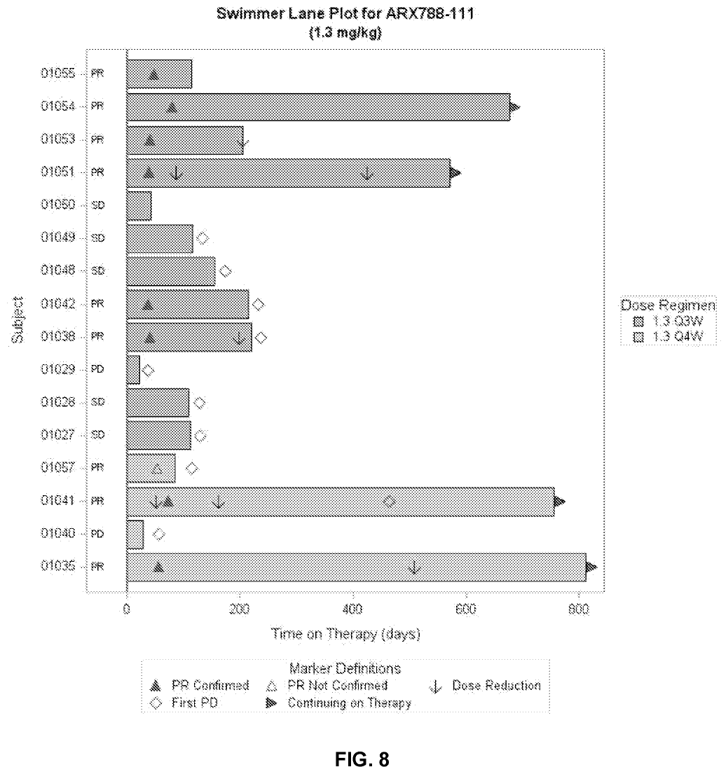

For The ACE-Breast-01 Study: A waterfall plot of the overall reduction in tumor volume at these doses in Study (ACE-Breast-01) is shown in FIG. 2 (ARX788: 62% Confirmed ORR and 100% DCR in ACE-Breast Cancer-01 (1.5 mg/kg cohort)). The spider plot in FIG. 3 (ARX788 Showed Rapid, Deep and Durable Response in ACE-Breast-01 (1.5 mg/kg cohort)) shows the response of each subject at each timepoint. The swimmer plot shown in FIG. 4 (ARX788 Showed Durable Response in ACE-Breast-01 (1.5 mg/kg cohort)) illustrates the duration of the response for each subject. Although the median DOR and median PFS had not been reached at the 1.5 mg/kg Q3W dose in the study, the data suggest that the efficacy observed in these heavily pre-treated patients remains durable. FIG. 7 shows another A waterfall plot of the overall reduction in tumor volume at these doses in Study (ACE-Breast-01). The swimmer plot shown in FIG. 8 (ARX788 Showed Durable Response in ACE-Breast-01 (1.3 mg/kg cohort)) illustrates the duration of the response for each subject

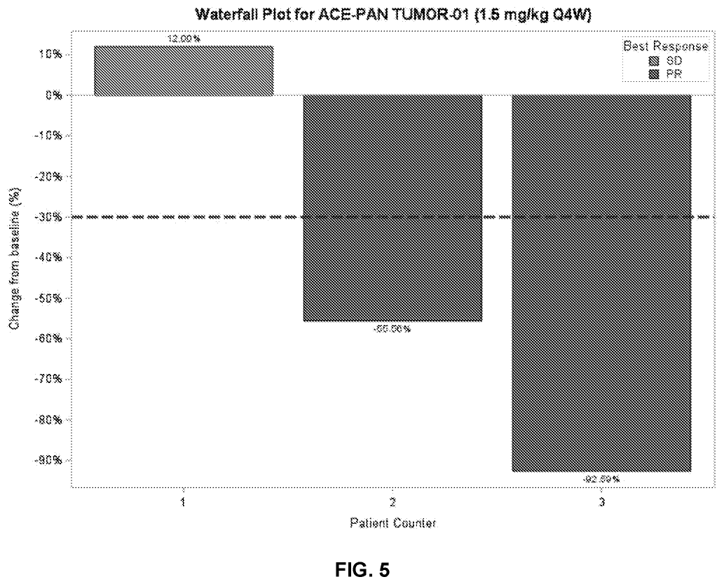

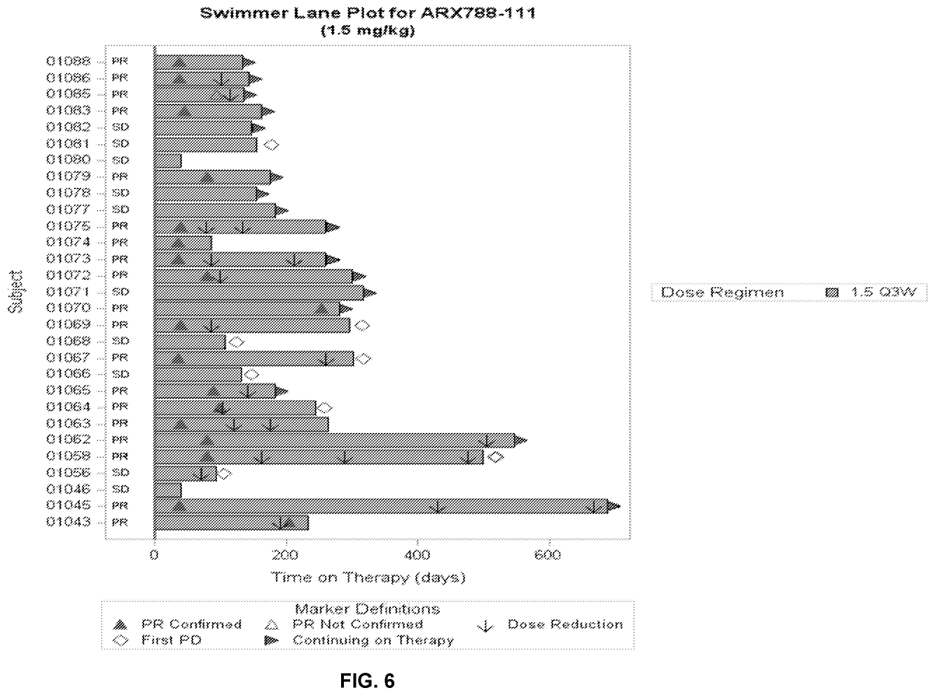

For The ACE-Pan tumor-01 Study: A waterfall plot of the overall reduction in tumor volume at these doses in Study (ACE-Pan tumor-01) is shown in FIG. 5. The spider plot in FIG. 6 shows the response of each subject at each timepoint.

### Safety of ARX788

In terms of safety, based on cumulative data from the dosed populations) in clinical studies ((ARX788-1711 (ACE-Pan-tumor-01) and ARX788-111 (ACE-Breast-01), ARX788 is generally well tolerated with most adverse events being mild (Grade 1) or moderate (Grade 2) and manageable.

The adverse events (AEs) that may be possibly related to ARX788 treatment included pneumonitis, ocular events, and alopecia. Pneumonitis may be the most significant, delayed SAE occurring at doses of 1.3 mg/kg or higher after 4-5 cycles of administration.

The vast majority of the AEs in Studies ARX788-1711 (ACE-Pan-tumor-01, U.S. and Australia), and ARX788-111 (ACE-Breast-01, China) are Grade 1 (mild) or Grade 2 (moderate) in severity and are manageable. See Table 7.

TABLE 7
Safety data of ARX788

| | ARX788 (1.5 mg/kg) | | |
|---|---|---|---|
| | Number of patients (%) | | |
| | ACE- | ACE-Pan | ACE- |
| | Breast-01 | tumor-01 | Gastric-01 |
| | (N = 29) | (N = 8) | (N = 14) |
| All AEs (regardless of causality) | 29 (100%) | 8 (100%) | 14 (100%) |
| Drug-related AEs (any grade) | 29 (100%) | 7 (88%) | 13 (92.9%) |
| All AEs ≥Grade 3 | 6 (20.7%) | 1 (13%) | 3 (21.4%) |
| Drug-related ≥Grade 3 AEs | 5 (17.2%) | 1 (13%) | 0 |
| All SAEs | 2 (6.9%) | 0 | 2 (15.4%) |
| Drug-related SAEs | 1 (3.4%) | 0 | 0 |
| AEs leading to discontinuation | 1 (3.4%) | 0 | 0 |
| Drug-related Deaths | 0 | 0 | 0 |

Adverse event of special interest (AESI) is shown in Tables 8 and 9.

TABLE 8
AESI

| | ARX788 (1.5 mg/kg) | | | | | | | | |
|---|---|---|---|---|---|---|---|---|---|
| | Number of patients (%) | | | | | | | | |
| | ACE-Breast-01 | | | | ACE-Pan tumor-01 | | ACE-Gastric-01 | | |
| | (N = 29) | | | | (N = 5) | | (N = 14) | | |
| | | | Grade 3 | | | Grade 3 | | | Grade 3 |
| | All Grades | | or 4 | | All Grades | or 4 | All Grades | | or 4 |
| Pneumonitis/ILD | 11 | (37.9%) | 1 | (3.4) | 0 | 0 | 0 1 | | 0 |
| AST increase* | 22 | (75.9%) | 0 | | 1 (20%) | 0 | 9 | (63.3%) | 0 |
| ALT increase* | 16 | (55.2%) | 0 | | 1 (20%) | 0 | 8 | (57.1%) | 0 |
| Dry eye | 5 | (17.2%) | 0 | | 1 (20%) | 0 | 10 | (71.4%) | 0 |
| Blurred vision | 7 | (24.1%) | 2 | (6.9%) | 1 (20%) | 0 | 2 | (14.3%) | 0 |
| Keratopathy | 25 | (86.2%) | 3 | (10.3%) | 0 | 0 | 3 | (21.4%) | 0 |

| | | | | | | 4 | |
|---|---|---|---|---|---|---|---|
| Eye discomfort | 1 | (3.4%) | 0 | 1 (20%) | 0 | 0 | 0 |

*Short-period of ALT or AST increases of mild to moderate intensity

TABLE 9
AESI

| | ARX788 (1.5 mg/kg) | | | | | | | | |
|---|---|---|---|---|---|---|---|---|---|
| | Number of patients (%) | | | | | | | | |
| | ACE-Breast-01 | | | ACE-Pan tumor-01 | | | ACE-Gastric-01 | | |
| | (N = 29) | | | (N = 8) | | | (N = 14) | | |
| | | Grade 3 | | | | Grade 3 | | | Grade 3 |
| | All Grades | or 4 | | All Grades | | or 4 | All Grades | | or 4 |
| Pneumonitis/ | 14 (48.3%) | 1 | (3.4%) | 0 | | 0 | 1 | (7.1%) | 0 |
| ILD | | | | | | | | | |
| AST | 22 (75.9%) | 0 | | 2 | (25%) | 0 | 9 | (63.3%) | 0 |
| increased* | | | | | | | | | |
| ALT | 17 (55.2%) | 0 | | 2 | (25%) | 0 | 8 | (57.1%) | 0 |
| increased* | | | | | | | | | |
| Ocular | 28 (96.6%) | 4 | (13.8%) | 3 | (37.5%) | 0 | 12 | (85.7%) | 0 |
| AEs** | | | | | | | | | |

*Short-period of ALT or AST increases of mild to moderate intensity
**Ocular adverse events as a composite term included dry eyes, keratopathy, blurred vision, corneal epithelial injury, eye discharge, xerophthalmia, watery eyes, lacrimation increased, ocular discomfort, eye pruritus, eyelids pruritus, eye pain, conjunctivitis, conjunctival hyperemia, photophobia, diplopia and vison impaired.

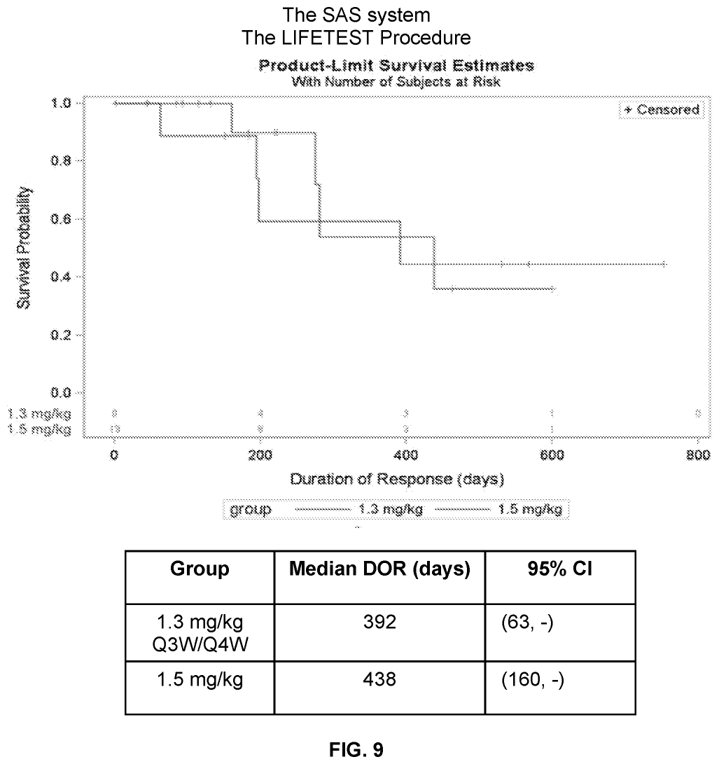

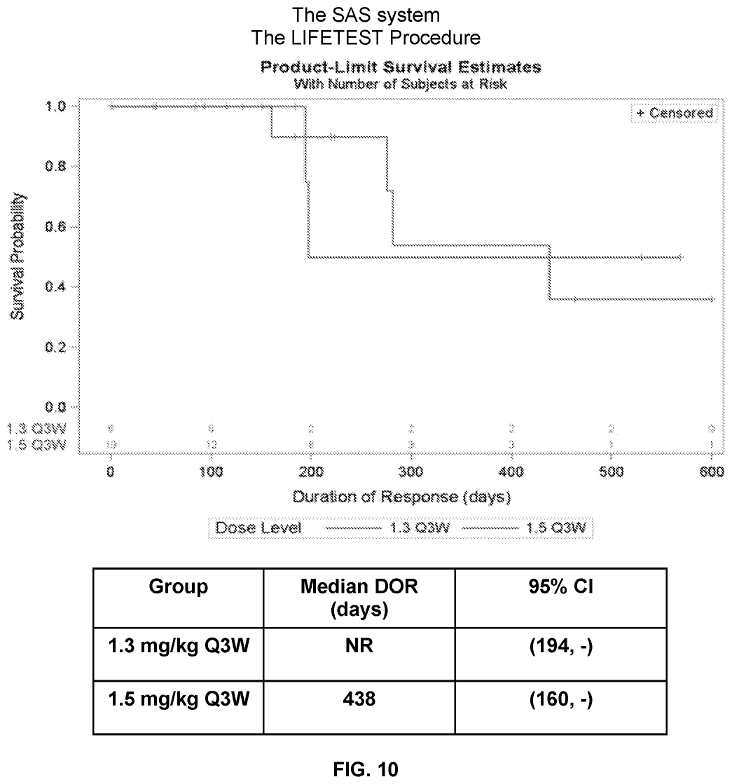

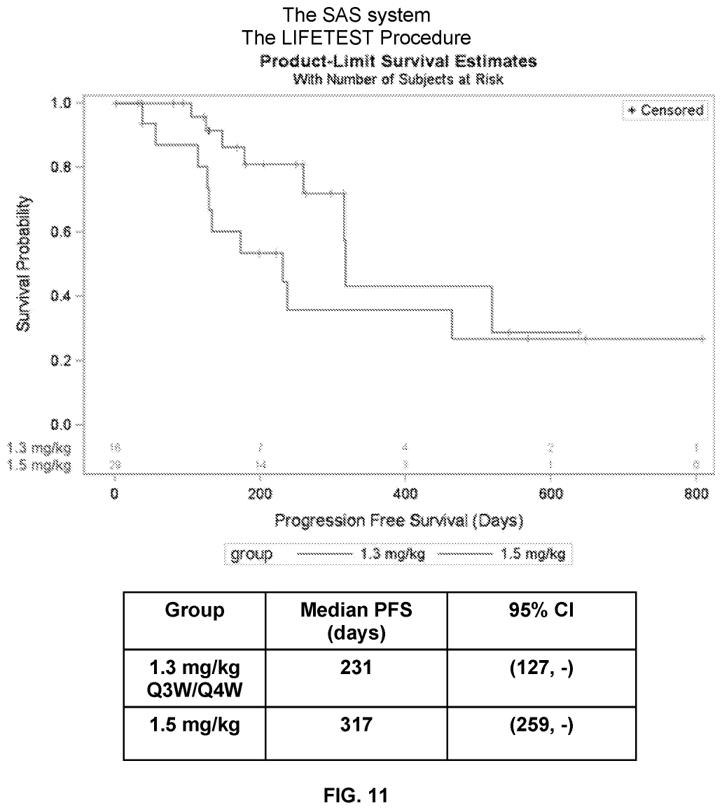

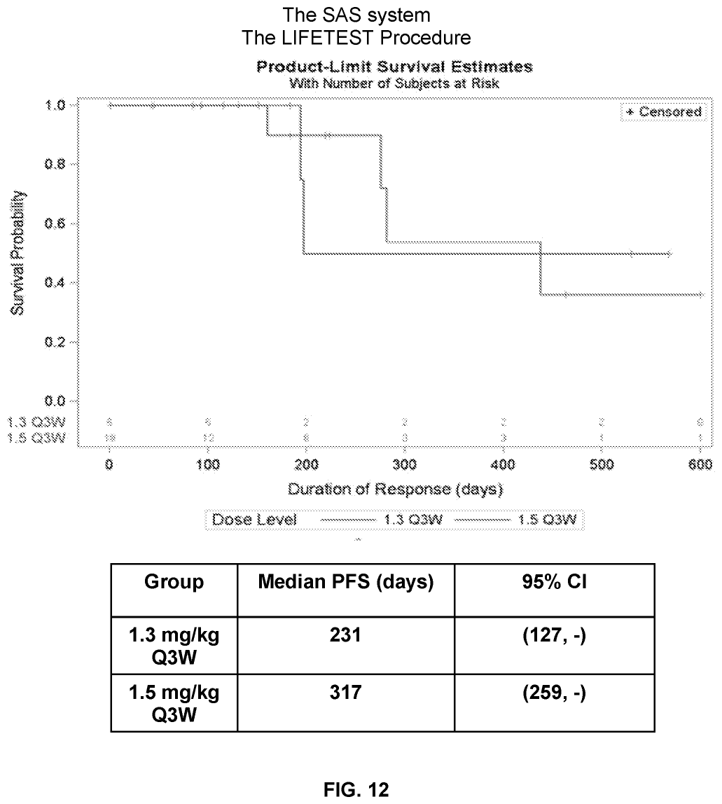

FIGS. 9-13 show different product-limit survival estimate with number of subjects at risk shown according to the LIFETEST procedure using the SAS system.

### Example 7: Another Clinical Study

Based on clinical data generated to date, a global trial for HER2-positive metastatic breast cancer, ACE-Breast-03, has been initiated, illustrated below. Trial for ARX788 in HER2-positive metastatic breast cancer patients whose diseases have failed T-DM1, and/or T-DXd, and/or tucatinib-containing regimens is described below.

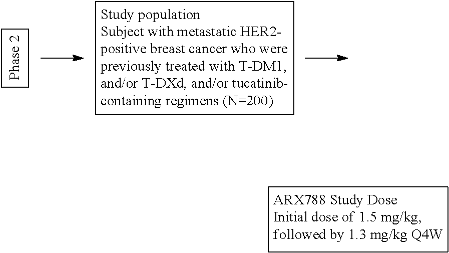

| Dose Reduction | Dose |
|---|---|
| Starting regimen | An initial dose of 1.5 mg/kg |
| | at Cycle 1 Day 1 in a 4- |
| | week cycle followed by |
| | subsquent doses of 1.3 |
| | mg/kg Q4W |

After receiving 1.3 mg/kg dose:

| | |
|---|---|
| One (1) dose reduction (Dose Level—1) | 1.1 mg/kg Q4W |
| Two (2) dose reduction (Dose Level—2) | 0.88 mg/kg Q4W |
| Three (3) dose reduction (Dose Level—3) | 0.66 mg/kg Q4W |
| No additional dose reduction after | Discontinue treatment |
| third reduction | |

### Example 8: Another Clinical Study

Based on response in a subject at the initial doses applied subsequent dosing can be modulated for administration to achieve the desired outcome. In some instance, lower or higher effective doses may be employed to the extent that patient tolerance permits. For example, based on subject response, an initial dose of 1.7 mg/kg on day 1 of a 4-week cycle can be administered followed by subsequent doses of 1.5 mg/kg or 1.3 mg or 0.88 mg/kg or 0.66 mg/kg as illustrated below.

### Gastric Cancer Clinical Trial of ARX788

A HER2-positive metastatic gastric/GEJ cancer trial, ACE-Gastric-01 Phase 1 clinical trial of ARX788, where patients have failed other available therapies, including trastuzumab, is ongoing. The safety and efficacy of ARX788 in patients with HER2-positive advanced gastric cancer or gastroesophageal junction adenocarcinoma (GC/GEJ) was evaluated. In this trial, promising anti-tumor activity in the cohorts of patients receiving 1.5 mg/kg of ARX788 Q3W, with a 46% (6 of 13 patients) confirmed ORR, and a 43% (3 of 7 patients) confirmed ORR in the cohort of patients receiving 1.3 mg/kg of ARX788 at Q3W has been observed, Table 10.

TABLE 10
Clinical Efficacy-ACE-Gastric-01

| Dose Level | | Confirmed | |
|---|---|---|---|
| (N: response evaluable) | OOR | ORR | DCR |
| 1.3 mg/kg (N = 7) | 43% | 43% | 57% |
| 1.5 mg/kg (N = 13) | 46% | 46% | 46% |

Additional clinical trials of ARX788, ACE-Gastric-02 global Phase 2/3, for HER2-positive advanced gastric cancer, including at the GEJ are ongoing. Subjects will be randomized in a 2:1 ratio and will receive ARX788 1.7 mg/kg at Q3W. In additional clinical trials for HER2-positive advanced gastric cancer, including at the GEJ, subjects will receive ARX788 1.8 mg/kg at Q3W.

ARX788 may be used to provide benefit to a broader spectrum of patients, including but not limited to, HER2-low breast cancer patients and those with gastric/GEJ, non-small cell lung cancer (NSCLC), urothelial, colon, ovarian, biliary tract or pancreatic cancers and solid tumors.

### Example 9

Phase 1 Study of ARX788 as Monotherapy in Advanced Solid Tumors with HER2-expression or mutation

A global, dose-finding, ACE-Pan tumor-01 phase 1 study to assess efficacy and safety of ARX788 in patients with metastatic HER2+breast cancer (BC), HER2-low breast cancer, gastric cancer, other solid tumors, or HER2 activating mutations is ongoing. ARX788 is administered as an IV in Q3W cycle at 1.6 kg/mg or 1.7 kg/mg. Efficacy is assessed every Q6W using RECIST 1.1 criteria and endpoints include ORR, DOR, TTR, BOR, DCR, PFS, and OS. The safety is assessed by PE, AEs, VS, ECG, clinical laboratory tests, and immunogenicity. PK and biomarkers are also evaluated.

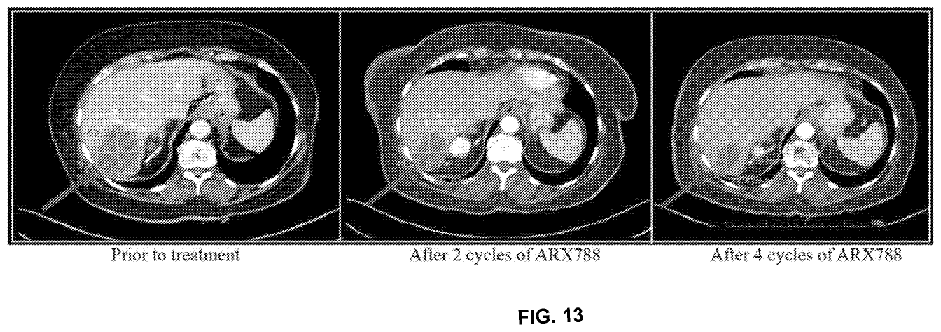

FIG. 13 depicts an 85-year-old HR+/HER2-low metastatic breast cancer patient (HER2 IHC1+) with partial response in the first tumor assessment scan. Liver metastasis was shown to decrease 30% in size after 2 cycles of ARX788 and subsequently decreased another 59% (PR) after 4 cycles of ARX788. Observed brain metastases were found to be completed resolved after 8cycles at 0.66 mg/kg.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1.-61. (canceled)

62. A formulation comprising: (i) about 20 mg/mL antibody-drug conjugate (ADC) comprising a cytotoxic tubulin analog having the structure: 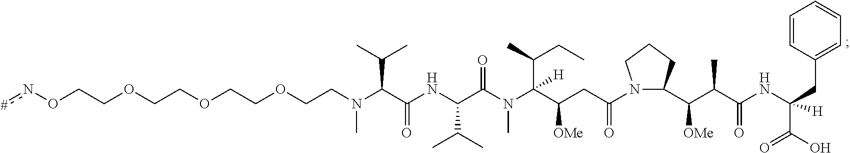
and
an anti-HER2 antibody or antibody fragment comprising SEQ ID NO: 2 and SEQ ID NO: 3,
wherein [symbol] represents a single bond or a double bond, and wherein # represents a connection to the anti-HER2 antibody or antibody fragment.
(ii) about 5 mM histidine buffer at about pH 6.0,
(iii) about 6% w/v trehalose, and
(iv) about 0.02% w/v polysorbate 80.

63. The formulation of claim 62, wherein the histidine is L-histidine.

64. The formulation of claim 62, wherein the anti-HER2 antibody or antibody fragment comprises one or more non-naturally encoded amino acids incorporated in a heavy chain, a light chain, or both the heavy chain and the light chain of the ADC.

65. The formulation of claim 64, wherein the one or more non-naturally encoded amino acids are para-acetyl phenylalanine.

66. The formulation of claim 64, wherein a light chain of the anti-HER2 antibody or antibody fragment is any one of SEQ ID NOs: 3, 4, 5, 6, 7, 8, 9, 10, 11, 12 and 13.

67. A composition comprising an antibody-drug conjugate of claim 62.

68. The composition of claim 67, further comprising an additional therapeutic agent.

69. The composition of claim 68, wherein the additional therapeutic agent is an immunotherapeutic agent, chemotherapeutic agent, hormonal agent, antitumor agent, immunostimulatory agent or immunomodulator, or a combination thereof.

70. The composition of claim 68, wherein the additional therapeutic agent is a HER2 targeted therapeutic.

71. The composition of claim 68, wherein the additional therapeutic agent is a checkpoint inhibitor, a HER2 kinase inhibitor, cyclin-dependent kinase inhibitor, tyrosine kinase inhibitor, small-molecule kinase inhibitor or a platinum-based therapeutic, or a combination thereof.

72. A method of treating a tumor or cancer in a human subject in need thereof, wherein the method comprises administering to the human subject an effective amount of a formulation according to claim 62.

73. The method of claim 72, wherein the effective amount is about 0.22, 0.33, 0.44, 0.55, 0.66, 0.77, 0.88, 0.99, 1.0, 1.1, 1.2, 1.3, 1.4, 1.5, 1.6, 1.7, 1.8, 1.9 or 2.0 mg per kilogram body weight (mg/kg) of the human subject.

74. The method of claim 72, wherein the administering is once or more than once within a 1, 2, 3, 4, 5, 6, 7 or 8-week cycle.

75. The method of claim 72, wherein the administering is once every two weeks for more than 8 weeks or lasts two or more 3-week cycles.

76. The method of claim 72, wherein the tumor or cancer is a HER-2 expressing cancer.

77. The method of claim 76, wherein the cancer is a HER-2 low expressing cancer, HER2 moderate expressing cancer, or HER2 high expressing cancer.

78. The method of claim 72, wherein the subject to be treated has a HER-2 expressing cancer and/or cancer metastases from a same or different cancer.

79. The method of claim 72, wherein the tumor or cancer is breast cancer, ovarian cancer, gastric cancer, gastro-esophageal junction adenocarcinoma, cervical cancer, uterine cancer, endometrial cancer, testicular cancer, prostate cancer, colorectal cancer, esophageal cancer, bladder cancer, non-small cell lung cancer (NSCLC), urothelial carcinoma, cholangiocarcinoma, colon biliary tract cancer, pancreatic cancer, or solid tumor.

80. The method of claim 79, wherein the tumor or cancer is breast cancer, ovarian cancer or gastric cancer.

81. A pharmaceutical composition or salt of an ADC according to claim 62.


SEQUENCE LISTING (13 sequences)

SEQ ID NO: 1 - protein, 449 aa, Artificial Sequence
  Heavy Chain Wild Type
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 449 aa, Artificial Sequence
  Heavy Chain A114 Mutation
  misc_feature (121)..(121): Xaa = non-natural amino acid
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSXSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 214 aa, Artificial Sequence
  Light Chain Wild Type
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 214 aa, Artificial Sequence
  Light Chain V110 Mutation
  misc_feature (110)..(110): Xaa = non-natural amino acid
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTXAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 214 aa, Artificial Sequence
  Light Chain A112 Mutation
  misc_feature (112)..(112): Xaa = non-natural amino acid
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAXPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 214 aa, Artificial Sequence
  Light Chain S114 Mutation
  misc_feature (114)..(114): Xaa = non-natural amino acid
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPXVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 214 aa, Artificial Sequence
  Light Chain S121 Mutation
  misc_feature (121)..(121): Xaa = non-natural amino acid
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPXDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 214 aa, Artificial Sequence
  Light Chain S127 Mutation
  misc_feature (127)..(127): Xaa = non-natural amino acid
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKXGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 214 aa, Artificial Sequence
  Light Chain K149 Mutation
  misc_feature (149)..(149): Xaa = non-natural amino acid
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWXVDNALQSGNSQESVTEQDSKDSTYSLSSTLTLSKADYEKHKVYACEVTHQGLSSPVTKSFNRGEC

SEQ ID NO: 10 - protein, 214 aa, Artificial Sequence
  Light Chain S156 Mutation
  misc_feature (156)..(156): Xaa = non-natural amino acid
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQXGNSQESVTEQDSKDSTYSLSSTLTLSKADYEKHKVYACEVTHQGLSSPVTKSFNRGEC

SEQ ID NO: 11 - protein, 214 aa, Artificial Sequence
  Light Chain S168 Mutation
  misc_feature (168)..(168): Xaa = non-natural amino acid
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDXKDSTYSLSSTLTLSKADYEKHKVYACEVTHQGLSSPVTKSFNRGEC

SEQ ID NO: 12 - protein, 214 aa, Artificial Sequence
  Light Chain S202 Mutation
  misc_feature (202)..(202): Xaa = non-natural amino acid
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLXSPVTKSFNRGEC

SEQ ID NO: 13 - protein, 214 aa, Artificial Sequence
  Light Chain V205 Mutation
  misc_feature (205)..(205): Xaa = non-natural amino acid
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPXTKSFNRGEC